(19) United States
(12) Patent Application Publication
(10) Pub. No.: US 2022/0056438 A1
(43) Pub. Date: Feb. 24, 2022

(54) GENE-EDITING COMPOSITIONS AND METHODS TO MODULATE FAAH FOR TREATMENT OF NEUROLOGICAL DISORDERS

(71) Applicant: CRISPR THERAPEUTICS AG, Zug (CH)

(72) Inventors: Seshidhar Reddy Police, Cambridge, MA (US); Tony Ho, Cambridge, MA (US); Yanfei Yang, Cambridge, MA (US); Hemangi Chaudhari, Cambridge, MA (US); Anandan Paldurai, Cambridge, MA (US)

(21) Appl. No.: 17/407,690

(22) Filed: Aug. 20, 2021

Related U.S. Application Data
(63) Continuation of application No. 17/380,173, filed on Jul. 20, 2021, now abandoned.
(60) Provisional application No. 63/054,580, filed on Jul. 21, 2020.

(51) Int. Cl. C12N 15/11; C12N 15/86; A61K 38/46; A61K 31/7105; C12N 9/22

(52) U.S. Cl. CPC C12N 15/11; C12N 15/86; A61K 38/465; A61K 31/7105; C12N 2320/31; C12N 2310/20; C12N 2750/14143; C12N 2800/80; C12N 9/22

(57) ABSTRACT

The disclosure provides systems (e.g., CRISPR/Cas systems) for introducing an edit in a genomic DNA molecule comprising the fatty acid amide hydrolase gene (FAAH) and/or the FAAH pseudogene (FAAH-OUT). Also provided are methods for use of the systems, nucleic acids, delivery systems, and/or compositions described for genome editing to modulate the expression and/or activity of FAAH, for example, in a method of treating chronic pain.

The present application is a continuation of U.S. application Ser. No. 17/380,173, filed Jul. 20, 2021, and which claims the benefit of priority to U.S. Provisional Application No. 63/054,580 filed Jul. 21, 2020, the disclosures of which are incorporated herein by reference in their entireties.

### INCORPORATION OF MATERIAL SUBMITTED ELECTRONICALLY INCORPORATION BY REFERENCE OF INFORMATION SUBMITTED ELECTRONICALLY

This application contains, as a separate part of the disclosure, a Sequence Listing in computer readable form (Filename: CT138A_Seqlisting.txt; Size: 737,441 bytes; Created: Aug. 20, 2021), which is incorporated by reference in its entirety.

### BACKGROUND

Pain is a normal protective and adaptive reaction to an injury or illness and functions as a signal for damaged tissues that triggers repair processes. Pain may be caused by tissue inflammation (nociceptive) or dysfunctional nerves (neuropathic pain). Normally, pain is alleviated when the injury or illness heals or subsides. However, pain can remain sustained for long periods, even after the damaged tissues have healed. Chronic pain refers to pain that is sustained for three months or longer following the tissue injury and is a common and disabling condition. Treatment options for chronic pain, including opioids, electrical stimulation, surgery, acupuncture, and cognitive behavioral therapy, are often inadequate for effective pain management. Additionally, use of opioids to treat chronic pain is associated with serious addiction and drug-abuse liabilities. Thus, there remains an urgent need for safe and effective methods for pain treatment.

Use of cannabinoids for treatment of chronic pain are well-established. The primary bioactive constituent of cannabis is delta9-tetrahydro-cannbinol (THC). The discovery of THC led to the identification of two endogenous cannabinoid G-protein coupled receptors (GPCRs) responsible for its pharmacological actions, namely CB1 and CB2 (Goya et al (2000) EXP OPIN THER PATENTS 10:1529). These discoveries further led to identification of endogenous agonists of these receptors, or “endocannabinoids”. The first endocannabinoid identified is arachidonoylethanolamine (anandamide; AEA) (Devane, et al (1992) SCIENCE 258:1946). AEA elicits many of the pharmacological effects of exogenous cannabinoids (Piornelli et al (2003) NAT REV NEUROSCI 4:873). For example, elevated AEA levels have known effects on nociception, fear-extinction memory, anxiety, and depression (Woodhams, et al (2015) HANDB EXP PHARMACOL 227:119; Mechoulam, et al (2013) ANNU REV PSYCHOL 64:21). However, external administration of endocannabinoids has limited efficacy as they are rapidly degraded in vivo.

The major catabolic enzyme of AEA is fatty acid amide hydrolase (FAAH) (Dinh, et al (2002) PNAS 99:10819). FAAH is also the major catabolic enzyme for other bioactive fatty acid amides (FAAs), such as N-palmitoylethanolamine (PEA) (Lo Verme, et al (2005) Mol Pharmacol 67:15), oleamide (Cravatt, (1995) SCIENCE 268:1506), and N-oleoylethanolamine (OEA) (Rodrigues de Fonesca (2001) NATURE 414:209. PEA for example, is an agonist of the PPARalpha receptor and has demonstrated biological effects in animal models of inflammation (Holt et al (2005) BR J PHARMACOL 146:467).

Genetic or pharmacological inactivation of FAAH has been demonstrated to prolong and enhance the beneficial effects of AEA. For example, FAAH knockout mice have significantly elevated levels of AEA throughout the nervous system and display an analgesic phenotype (see, e.g., Huggins, et al (2012) PAIN 153:1837; Kerbrat, et al (2016) N Engl J Med 375:1717). Additionally, homozygous carriers of a hypomorphic single nucleotide polymorphism (SNP; C385A) allele in humans showed significantly lower pain sensitivity and less need for postoperative analgesia (Cajanus, et al (2016) PAIN 157:361). Knock-in mice carrying the SNP also display decreased anxiety-linked behaviors (Dincheva, et al (2015) NAT COMMUN 6:6395). Given the potential therapeutic benefits of diminishing FAAH enzymatic activity, small molecule inhibitors of FAAH have been developed. However clinical evaluation of these inhibitors for treatment of chronic pain failed due to lack of efficacy at tolerated dose levels (Huggins, et al 2012 PAIN 153:1837).

Accordingly, there remains a need for improved methods to modulate FAAH activity in vivo, thereby providing strategies to better manage pain and other neurological disorders.

### SUMMARY OF THE DISCLOSURE

In some aspects, the disclosure provides a system for introducing a deletion in a genomic DNA molecule comprising a fatty-acid amide hydrolase gene (FAAH) upstream a FAAH pseudogene (FAAH-OUT) in a cell, the system comprising: (i) a site-directed endonuclease in the form of protein, an mRNA encoding the site-directed endonuclease, or a recombinant expression vector comprising a nucleotide sequence encoding the site-directed endonuclease; (ii) a first gRNA molecule comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with a site-directed endonuclease that recognizes the PAM, the first gRNA combines with the site-directed endonuclease to induce cleavage proximal the first target sequence with a cleavage efficiency of at least 15%, 20%, 25%, or 30%; and (iii) a second gRNA molecule comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 15%, 20%, 25%, or 30%, wherein when the system is introduced to the cell with the site-directed endonuclease, the first gRNA and second gRNA combine with the site-directed endonuclease to induce cleavage proximal the first and second target sequences, to introduce an approximately 2-10 kb deletion in the genomic DNA molecule resulting in a full or a partial removal of a FAAH-OUT promoter (FOP) and a FAAH-OUT conserved (FOC) element, thereby resulting in a reduction or elimination of FAAH mRNA expression in the cell. In some aspects, the first PAM and the second PAM are both NNGG, NGG, or NNGRRT. In some aspects, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof. In some aspects, the site-directed endonuclease is a SpCas9 polypeptide or functional derivative thereof, an mRNA encoding the SpCas9 endonuclease or functional derivative thereof, or a recombinant expression vector comprising a nucleotide sequence encoding the SpCas9 endonuclease or functional derivative thereof. In some aspects, the site-directed endonuclease is a SaCas9 polypeptide or functional derivative thereof, an mRNA encoding the SaCas9 endonuclease or functional derivative thereof, or a recombinant expression vector comprising a nucleotide sequence encoding the SaCas9 endonuclease or functional derivative thereof.

In some aspects, the disclosure provides a system for introducing a deletion in a genomic DNA molecule comprising a fatty-acid amide hydrolase gene (FAAH) upstream a FAAH pseudogene (FAAH-OUT) in a cell, the system comprising: (i) a site-directed endonuclease wherein the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof; (ii) a first gRNA molecule comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with a site-directed endonuclease that recognizes the PAM, the first gRNA combines with the site-directed endonuclease to induce cleavage proximal the first target sequence with a cleavage efficiency of at least 15%, 20%, 25%, or 30%; and (iii) a second gRNA molecule comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 15%, 20%, 25%, or 30%, wherein when the system is introduced to the cell with the site-directed endonuclease, the first gRNA and second gRNA combine with the site-directed endonuclease to induce cleavage proximal the first and second target sequences, to introduce an approximately 2-10 kb deletion in the genomic DNA molecule resulting in a full or a partial removal of a FAAH-OUT promoter (FOP) and a FAAH-OUT conserved (FOC) element, thereby resulting in a reduction or elimination of FAAH mRNA expression in the cell. In some aspects, the first PAM and the second PAM are both NNGG.

In any of the foregoing or related aspects, the deletion in the genomic DNA molecule is approximately 2-7.5 kb, approximately 2-7 kb, approximately 2-6 kb, approximately 2-5 kb, approximately 2-4 kb, approximately 3-8 kb, approximately 3-7 kb, approximately 3-6 kb, approximately 3-5 kb, approximately 4-8 kb, approximately 4-7 kb, approximately 4-6 kb, approximately 5-8 kb, or approximately 5-7 kb. In some aspects, the first target sequence is (i) within a region of the genomic DNA molecule that is at least about 5 kb, about 5.5 kb, about 6 kb, about 6.5 kb, about 7 kb, about 7.5 kb, about 8 kb, about 8.5 kb, about 9 kb, or about 9.5 kb downstream the 3′ terminus of FAAH; (ii) within a region of the genomic DNA molecule that is at least about 200 bp, about 300 bp, about 400 bp, about 500 bp, about 600 bp, about 700 bp, about 800 bp, about 900 bp, about 1 kb, about 2 kb, about 3 kb, or about 4 kb upstream the transcriptional start site of FAAH-OUT; (iii) within a region of the genomic DNA molecule between about 46,418,846 to about 46,422,883 of chromosome 1, according to human reference genome Hg38; or (iv) a combination of (i)-(iii). In some aspects, the second target sequence is (i) within a region of the genomic DNA molecule that is about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, or about 3.3 kb downstream the transcriptional start site of FAAH-OUT; (ii) within a region of the genomic DNA molecule that is about 5.8 kb, about 5.9 kb, about 6 kb, about 6.1 kb, about 6.2 kb, about 6.3 kb, about 6.4 kb, about 6.5 kb, about 6.6 kb, about 6.7 kb, about 6.8 kb, about 6.9 kb, about 7 kb, about 7.1 kb, about 7.2 kb, or about 7.3 kb upstream the 5′ end of exon 3 of FAAH-OUT; (iii) within a region of the genomic DNA molecule between about 46,424,697 to about 46,426,377 of chromosome 1, according to human reference genome Hg38; or (iv) a combination of (i)-(iii).

In any of the foregoing or related aspects, the deletion in the genomic DNA molecule is approximately 5 kb, approximately 5.5 kb, approximately 6 kb, approximately 6.5 kb, approximately 7 kb, approximately 7.5 kb, or approximately 8 kb. In some aspects, the deletion results in removal of FOP. In some aspects, the first spacer sequence comprises: a nucleotide sequence having up to 1, 2, or 3 nucleotide deletions or substitutions relative to SEQ ID NO: 750 or SEQ ID NO: 765. In some aspects, the deletion results in removal of FOC. In some aspects, the second spacer sequence comprises: a nucleotide sequence having up to 1, 2, or 3 nucleotide deletions or substitutions relative to any one of SEQ ID NOs: 878, 888, 891, 895, 898, and 909. In some aspects, the second spacer sequence comprises a nucleotide sequence set forth in SEQ ID NO: 878, 888, 891, 895, 898, or 909. In some aspects, the deletion results in a partial removal of FOC. In some aspects, the second spacer sequence comprises: a nucleotide sequence having up to 1, 2 or 3 nucleotide deletions or substitutions relative to any one of SEQ ID NOs: 815, 816, 830, and 862. In some aspects, the second spacer sequence comprises a nucleotide sequence set forth in SEQ ID NO: 815, 816, 830, or 862.

In any of the foregoing or related aspects, the deletion in the genomic DNA molecule is approximately 2 kb, approximately 2.5 kb, approximately 3 kb, approximately 3.5 kb, approximately 4 kb, approximately 4.5 kb, approximately 5 kb, or approximately 5.5 kb. In some aspects, the deletion results in a partial removal of FOP. In some aspects, the first spacer sequence comprises: a nucleotide sequence having up to 1, 2, or 3 nucleotide deletions or substitutions relative to SEQ ID NO: 801 or SEQ ID NO: 807. In some aspects, the first spacer sequence comprises a nucleotide sequence set forth in SEQ ID NO: 801 or 807. In some aspects, the deletion results in removal of FOC. In some aspects, the second spacer sequence comprises: a nucleotide sequence having up to 1, 2 or 3 nucleotide deletions or substitutions relative to any one of SEQ ID NOs: 878, 888, 891, 895, 898, and 909. In some aspects, the second spacer sequence comprises a nucleotide sequence set forth in SEQ ID NO: 878, 888, 891, 895, 898, and 909. In some aspects, the deletion results in a partial removal of FOC. In some aspects, the second spacer sequence comprises: a nucleotide sequence having up to 1, 2 or 3 nucleotide deletions or substitutions relative to any one of SEQ ID NOs: 815, 816, 830, and 862. In some aspects, the second spacer comprises a nucleotide sequence comprises SEQ ID NO: 815, 816, 830, or 862.

In some aspects, the disclosure provides a system for introducing a deletion in a genomic DNA molecule comprising a fatty-acid amide hydrolase gene (FAAH) upstream a FAAH pseudogene (FAAH-OUT) in a cell, the system comprising: (i) a site-directed endonuclease wherein the site-directed endonuclease is a SpCas9 polypeptide or functional derivative thereof, an mRNA encoding the SpCas9 endonuclease or functional derivative thereof, or a recombinant expression vector comprising a nucleotide sequence encoding the SpCas9 endonuclease or functional derivative thereof; (ii) a first gRNA molecule comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with a site-directed endonuclease that recognizes the PAM, the first gRNA combines with the site-directed endonuclease to induce cleavage proximal the first target sequence with a cleavage efficiency of at least 15%, 20%, 25%, or 30%; and (iii) a second gRNA molecule comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 15%, 20%, 25%, or 30%, wherein when the system is introduced to the cell with the site-directed endonuclease, the first gRNA and second gRNA combine with the site-directed endonuclease to induce cleavage proximal the first and second target sequences, to introduce an approximately 2-10 kb deletion in the genomic DNA molecule resulting in a full or a partial removal of a FAAH-OUT promoter (FOP) and a FAAH-OUT conserved (FOC) element, thereby resulting in a reduction or elimination of FAAH mRNA expression in the cell. In some aspects, the first PAM and the second PAM are both NGG. In some aspects, the deletion results in full removal of FOP.

In any of the foregoing or related aspects, the deletion in the genomic DNA molecule is approximately 3-9 kb, approximately 3-8 kb, approximately 3-7 kb, approximately 3-6 kb, approximately 3-5 kb, approximately 4-10 kb, approximately 4-9 kb, approximately 4-8 kb, approximately 4-7 kb, approximately 4-6 kb, approximately 5-10 kb, approximately 5-9 kb, approximately 5-8 kb, approximately 5-7 kb, approximately 6-10 kb, approximately 6-9 kb, approximately 6-8 kb, or approximately 8-10 kb. In some aspects, the first target sequence is (i) within a region of the genomic DNA molecule that is at least about 4.5 kb, about 5 kb, about 5.5 kb, about 6 kb, about 6.5 kb, about 7 kb, about 7.5 kb, about 7.5 kb, or about 8 kb downstream the 3′ terminus of FAAH; (ii) within a region of the genomic DNA molecule that is at least about 1.5 kb, about 2 kb, about 2.5 kb, about 3 kb, about 3.5, about 4 kb, about 4.5 kb, or about 5 kb upstream the transcriptional start site of FAAH-OUT; (iii) within a region of the genomic DNA molecule between about 46,418,391 to about 46,421,122 of chromosome 1, according to human reference genome Hg38; or (iv) a combination of (i)-(iii). In some aspects, the second target sequence is (i) within a region of the genomic DNA molecule that is at least about 1.8 kb, about 1.9 kb, about 2 kb, about 2.5 kb, about 3 kb, about 3.5 kb, about 4 kb, about 4.5 kb, about 5 kb, or about 5.5 kb downstream the transcriptional start site of FAAH-OUT; (ii) within a region of the genomic DNA molecule that is at least about 3.5 k, about 4 kb, about 4.5 kb, about 5 kb, about 5.5 kb, about 6 kb, about 6.5 kb, about 7 kb, or about 7.5 kb upstream the 5′ end of exon 3 of FAAH-OUT; (iii) within a region of the genomic DNA molecule between about 46,424,651 to about 46,428,274 of chromosome 1, according to human reference genome Hg38; or (iv) a combination of (i)-(iii).

In any of the foregoing or related aspects, the deletion in the genomic DNA molecule is approximately 8 kb, approximately 8.5 kb, approximately 9 kb, approximately 9.5 kb, or approximately 10 kb. In some aspects, the deletion results in full removal of FOC. In some aspects, the first spacer sequence comprises: a nucleotide sequence having 1, 2, or 3 nucleotide deletions or substitutions relative to any one of SEQ ID NOs: 374, 378, and 406; and wherein the second spacer sequence comprises: a nucleotide sequence having 1, 2, or 3 nucleotide deletions or substitutions relative to SEQ ID NO: 550.

In any of the foregoing or related aspects, the deletion in the genomic DNA molecule is approximately 5 kb, approximately 5.5 kb, approximately 6 kb, approximately 6.5 kb, approximately 7 kb, approximately 7.5 kb, or approximately 8 kb. In some aspects, the deletion results in full removal of FOC. In some aspects, the first spacer sequence comprises: a nucleotide sequence having 1, 2, or 3 nucleotide deletions or substitutions relative to any one of SEQ ID NOs: 374, 378, and 406; and wherein the second spacer sequence comprises: a nucleotide sequence having 1, 2, or 3 nucleotide deletions or substitutions relative to any one of SEQ ID NOs: 533, 534, 538, and 540. In some aspects, the second spacer sequence comprises a nucleotide sequence set forth in SEQ ID NO: 533, 534, 538, and 540. In some aspects, the first spacer sequence comprises: a nucleotide sequence having 1, 2, or 3 nucleotide deletions or substitutions relative to SEQ ID NO: 421; and wherein the second spacer sequence comprises: a nucleotide sequence having 1, 2, or 3 nucleotide deletions or substitutions relative to SEQ ID NO: 550. In some aspects, the first spacer sequence comprises: a nucleotide sequence set forth in SEQ ID NO: 421; and wherein the second spacer sequence comprises: a nucleotide sequence set forth in SEQ ID NO: 550. In some aspects, the deletion results in partial removal of FOC. In some aspects, the first spacer sequence comprises: a nucleotide sequence having 1, 2, or 3 nucleotide deletions or substitutions relative to any one of SEQ ID NOs: 374, 378, and 406; and wherein the second spacer sequence comprises: a nucleotide sequence having 1, 2 or 3 nucleotide deletions or substitutions relative to any one of SEQ ID NOs: 475, 487, 491, and 502. In some aspects, the first spacer sequence comprises: a nucleotide sequence set forth in SEQ ID NOs: 374, 378, or 406; and wherein the second spacer sequence comprises: a nucleotide sequence set forth in SEQ ID NOs: 475, 487, 491, and 502.

In any of the foregoing or related aspects, the deletion in the genomic DNA molecule is approximately 3 kb, approximately 3.5 kb, approximately 4 kb, approximately 4.5 kb, approximately 5 kb, or approximately 5.5 kb. In some aspects, the first spacer sequence comprises: a nucleotide sequence having 1, 2, or 3 nucleotide deletions or substitutions relative to SEQ ID NO: 421. In some aspects, the deletion results in full removal of FOC. In some aspects, the second spacer sequence comprises: a nucleotide sequence having 1, 2 or 3 nucleotide deletions or substitutions relative to any one of SEQ ID NOs: 533, 534, 538, and 540. In some aspects, the second spacer comprises a nucleotide sequence set forth in SEQ ID NO: 533, 534, 538, and 540. In some aspects, the deletion results in partial removal of FOC. In some aspects, the second spacer sequence comprises: a nucleotide sequence having 1, 2 or 3 nucleotide deletions or substitutions relative to any one of SEQ ID NOs: 475, 487, 491, and 502. In some aspects, the second spacer sequence comprises a nucleotide sequence set forth in SEQ ID NO: 475, 487, 491, and 502.

In some aspects, the disclosure provides a system for introducing a deletion in a genomic DNA molecule comprising a fatty-acid amide hydrolase gene (FAAH) upstream a FAAH pseudogene (FAAH-OUT) in a cell, the system comprising: (i) a site-directed endonuclease wherein the site-directed endonuclease is a SaCas9 polypeptide or functional derivative thereof, an mRNA encoding the SaCas9 endonuclease or functional derivative thereof, or a recombinant expression vector comprising a nucleotide sequence encoding the SaCas9 endonuclease or functional derivative thereof; (ii) a first gRNA molecule comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with a site-directed endonuclease that recognizes the PAM, the first gRNA combines with the site-directed endonuclease to induce cleavage proximal the first target sequence with a cleavage efficiency of at least 15%, 20%, 25%, or 30%; and (iii) a second gRNA molecule comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 15%, 20%, 25%, or 30%, wherein when the system is introduced to the cell with the site-directed endonuclease, the first gRNA and second gRNA combine with the site-directed endonuclease to induce cleavage proximal the first and second target sequences, to introduce an approximately 2-10 kb deletion in the genomic DNA molecule resulting in a full or a partial removal of a FAAH-OUT promoter (FOP) and a FAAH-OUT conserved (FOC) element, thereby resulting in a reduction or elimination of FAAH mRNA expression in the cell. In some aspects, the first PAM and the second PAM are both NNGRRT. In some aspects, the deletion results in full removal of FOP.

In any of the foregoing or related aspects, the deletion in the genomic DNA molecule is at least about approximately 3-9 kb, approximately 3-8 kb, approximately 3-7 kb, approximately 3-6 kb, approximately 3-5 kb, approximately 4-10 kb, approximately 4-9 kb, approximately 4-8 kb, approximately 4-7 kb, approximately 4-6 kb, approximately 5-10 kb, approximately 5-9 kb, approximately 5-8 kb, approximately 5-7 kb, approximately 6-10 kb, approximately 6-9 kb, approximately 6-8 kb, or approximately 8-10 kb. In some aspects, the first target sequence is (i) within a region of the genomic DNA molecule that is at least about 4.5 kb, about 5 kb, about 5.5 kb, about 6 kb, about 6.5 kb, about 7 kb, about 7.5 kb, about 8 kb, about 8.5 kb, or about 9 kb downstream the 3′ terminus of FAAH; (ii) within a region of the genomic DNA molecule that is at least about 0.8 kb, about 0.9 kb, about 1 kb, about 1.5 kb, about 2 kb, about 2.5 kb, about 3 kb, about 3.5, about 4 kb, about 4.5 kb, or about 5 kb upstream the transcriptional start site of FAAH-OUT; (iii) within a region of the genomic DNA molecule between about 46,418,168 to about 46,422,208 of chromosome 1, according to human reference genome Hg38; or (iv) a combination of (i)-(iii). In some aspects, the second target sequence is (i) within a region of the genomic DNA molecule that is at least about 1.5 kb, about 2 kb, about 2.5 kb, about 3 kb, about 3.5 kb, about 4 kb, about 4.5 kb, about 5 kb, or about 5.5 kb downstream the transcriptional start site of FAAH-OUT; (ii) within a region of the genomic DNA molecule that is at least about 3.5 kb, about 4 kb, about 4.5 kb, about 5 kb, about 5.5 kb, about 6 kb, about 6.5 kb, about 7 kb, or about 7.5 kb upstream the 5′ end of exon 3 of FAAH-OUT; (iii) within a region of the genomic DNA molecule between about 46,424,887 to about 46,428,508 of chromosome 1, according to human reference genome Hg38; or (iv) a combination of (i)-(iii).

In any of the foregoing or related aspects, the deletion in the genomic DNA molecule is at least about approximately 8 kb, approximately 8.5 kb, approximately 9 kb, approximately 9.5 kb, or approximately 10 kb. In some aspects, the deletion results in removal of FOC. In some aspects, the first spacer sequence comprises: a nucleotide sequence having 1, 2, or 3 nucleotide deletions or substitutions relative to any one of SEQ ID NOs: 1102, 1104, 1111, and 1114; and wherein the second spacer sequence comprises: a nucleotide sequence having 1, 2, or 3 nucleotide deletions or substitutions relative to SEQ ID NO: 1259 or SEQ ID NO: 1264. In some aspects, the first spacer sequence comprises: a nucleotide sequence set forth in SEQ ID NOs: 1102, 1104, 1111, or 1114; and wherein the second spacer sequence comprises: a nucleotide sequence set forth in SEQ ID NO: 1259 or SEQ ID NO: 1264.

In any of the foregoing or related aspects, the deletion in the genomic DNA molecule is approximately 5 kb, approximately 5.5 kb, approximately 6 kb, approximately 6.5 kb, approximately 7 kb, approximately 7.5 kb, or approximately 8 kb. In some aspects, the deletion results in full removal of FOC. In some aspects, the first spacer sequence comprises: a nucleotide sequence having 1, 2, or 3 nucleotide deletions or substitutions relative to any one of SEQ ID NOs: 1102, 1104, 1111, 1114, 1119, 1121, and 1128; and wherein the second spacer sequence comprises: a nucleotide sequence having 1, 2, or 3 nucleotide deletions or substitutions relative to any one of SEQ ID NO: 1245. In some aspects, the first spacer sequence comprises: a nucleotide sequence set forth in SEQ ID NO: 1102, 1104, 1111, 1114, 1119, 1121, or 1128; and wherein the second spacer sequence comprises: a nucleotide sequence set forth in SEQ ID NO: 1245. In some aspects, the first spacer sequence comprises: a nucleotide sequence having 1, 2, or 3 nucleotide deletions or substitutions relative to any one of SEQ ID NOs: 1119, 1121, 1128, 1132, 1139, 1140, 1148, and 1152; and wherein the second spacer sequence comprises: a nucleotide sequence having 1, 2, or 3 nucleotide deletions or substitutions relative to any one of SEQ ID NO: 1259 or SEQ ID NO: 1264. In some aspects, the first spacer sequence comprises: a nucleotide sequence set forth in SEQ ID NOs: 1119, 1121, 1128, 1132, 1139, 1140, 1148, or 152; and wherein the second spacer sequence comprises: a nucleotide sequence set forth in SEQ ID NO: 1259 or SEQ ID NO: 1264. In some aspects, the deletion results in partial removal of FOC. In some aspects, the first spacer sequence comprises: a nucleotide sequence having 1, 2, or 3 nucleotide deletions or substitutions relative to any one of SEQ ID NOs: 1102, 1104, and 1111; and wherein the second spacer sequence comprises: a nucleotide sequence having 1, 2 or 3 nucleotide deletions or substitutions relative to any one of SEQ ID NO: 1218. In some aspects, the first spacer sequence comprises: a nucleotide sequence set forth in SEQ ID NOs: 1102, 1104, or 1111; and wherein the second spacer sequence comprises: a nucleotide sequence set forth in SEQ ID NO: 1218.

In any of the foregoing or related aspects, the deletion in the genomic DNA molecule is approximately 3 kb, approximately 3.5 kb, approximately 4 kb, approximately 4.5 kb, approximately 5 kb, or approximately 5.5 kb. In some aspects, the deletion results in full removal of FOC. In some aspects, the first spacer sequence comprises: a nucleotide sequence having 1, 2, or 3 nucleotide deletions or substitutions relative to any one of SEQ ID NOs: 1132, 1139, 1140, 1148, and 1152; and wherein the second spacer sequence comprises: a nucleotide sequence having 1, 2, or 3 nucleotide deletions or substitutions relative to SEQ ID NO: 1245. In some aspects, the first spacer sequence comprises: a nucleotide sequence set forth in SEQ ID NOs: 1132, 1139, 1140, 1148, or 1152; and wherein the second spacer sequence comprises: a nucleotide sequence set forth in SEQ ID NO: 1245. In some aspects, the deletion results in partial removal of FOC. In some aspects, the first spacer sequence comprises: a nucleotide sequence having 1, 2, or 3 nucleotide deletions or substitutions relative to any one of SEQ ID NOs: 1114, 1119, 1121, 1128, 1132, 1139, 1140, 1148, and 1152; and wherein the second spacer sequence comprises: a nucleotide sequence having 1, 2 or 3 nucleotide deletions or substitutions relative to SEQ ID NO: 1218. In some aspects, the first spacer sequence comprises: a nucleotide sequence set forth in SEQ ID NOs: 1114, 1119, 1121, 1128, 1132, 1139, 1140, 1148, or 1152; and wherein the second spacer sequence comprises: a nucleotide sequence set forth in SEQ ID NO: 1218.

In some aspects, the disclosure provides a system for use with a site-directed endonuclease to introduce a deletion in a genomic DNA molecule comprising a fatty-acid amide hydrolase gene (FAAH) upstream a FAAH pseudogene (FAAH-OUT) in a cell, the system comprising: (i) a first gRNA molecule targeting a target site in the genomic DNA molecule, the first gRNA comprising a spacer sequence corresponding to a first target sequence consisting of a nucleotide sequence selected from any one of SEQ ID NOs: 564, 579, 615, and 621; and (ii) a second gRNA molecule targeting a target site in the genomic DNA molecule, the second gRNA comprising a spacer sequence corresponding to a second target sequence consisting of a nucleotide sequence selected from any one of SEQ ID NOs: 629, 630, 644, 676, 692, 702, 705, 709, 712, and 723. In some aspects, the system comprises a site directed endonuclease which recognizes a PAM NNGG. In some aspects, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof. In some aspects, the first target sequence and second target sequences are selected from: (i) the nucleotide sequence of SEQ ID NO: 564 and the nucleotide sequence of SEQ ID NO: 629, 630, 644, 676, 692, 702, 705, 709, 712, or 723; (ii) the nucleotide sequence of SEQ ID NO: 579 and the nucleotide sequence of SEQ ID NO: 629, 630, 644, 676, 692, 702, 705, 709, 712, or 723; (ii) the nucleotide sequence of SEQ ID NO: 615 and the nucleotide sequence of SEQ ID NO: 629, 630, 644, 676, 692, 702, 705, 709, 712, 723; and (iv) the nucleotide sequence of SEQ ID NO: 621 and the nucleotide sequence of SEQ ID NO: 629, 630, 644, 676, 692, 702, 705, 709, 712, 723.

In some aspects, the disclosure provides a system for use with a site-directed endonuclease to introduce a deletion in a genomic DNA molecule comprising a fatty-acid amide hydrolase gene (FAAH) upstream a FAAH pseudogene (FAAH-OUT) in a cell, the system comprising: (i) a first gRNA molecule targeting a target site in the genomic DNA molecule, the first gRNA comprising a first spacer sequence comprising a nucleotide sequence selected from any one of SEQ ID NOs: 750, 765, 801, and 807; and (ii) a second gRNA molecule targeting a target site in the genomic DNA molecule, the second gRNA comprising a second spacer sequence comprising a nucleotide sequence selected from any one of SEQ ID NOs: 815, 816, 830, 862, 878, 888, 891, 895, 898, and 909. In some aspects, the system comprises a site directed endonuclease which recognizes a PAM NNGG. In some aspects, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof. In some aspects, the first and second spacer sequences are selected from: (i) the nucleotide sequence of SEQ ID NO: 750 and the nucleotide sequence of SEQ ID NO: 815, 816, 830, 862, 888, 891, 895, 898, or 909; (ii) the nucleotide sequence of SEQ ID NO: 765 and the nucleotide sequence of SEQ ID NO: 815, 816, 830, 862, 888, 891, 895, 898, or 909; (iii) the nucleotide sequence of SEQ ID NO: 801 and the nucleotide sequence of SEQ ID NO: 815, 816, 830, 862, 888, 891, 895, 898, or 909; and (iv) the nucleotide sequence of SEQ ID NO: 807 and the nucleotide sequence of SEQ ID NO: 815, 816, 830, 862, 888, 891, 895, 898, or 909.

In some aspects, the disclosure provides a system for use with a site-directed endonuclease to introduce a deletion in a genomic DNA molecule comprising a fatty-acid amide hydrolase gene (FAAH) upstream a FAAH pseudogene (FAAH-OUT) in a cell, the system comprising: (i) a first gRNA molecule targeting a target site in the genomic DNA molecule, the first gRNA comprising a spacer sequence corresponding to a first target sequence consisting of a nucleotide sequence selected from any one of SEQ ID NOs: 189, 193, 221, and 236; and (ii) a second gRNA molecule targeting a target site in the genomic DNA molecule, the second gRNA comprising a spacer sequence corresponding to a second target sequence consisting of a nucleotide sequence selected from SEQ ID NO: 290, 302, 306, 317, 348, 349, 353, 355, and 365. In some aspects, the system comprises a site directed endonuclease which recognizes a PAM NGG. In some aspects, the site-directed endonuclease is a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof. In some aspects, the first and second target sequences are selected from (i) the nucleotide sequence of SEQ ID NO: 189 and the nucleotide sequence of SEQ ID NO: 290, 302, 306, 317, 348, 349, 353, 355, or 365; (ii) the nucleotide sequence of SEQ ID NO: 193 and the nucleotide sequence of SEQ ID NO: 290, 302, 306, 317, 348, 349, 353, or 355; (iii) the nucleotide sequence of SEQ ID NO: 221 and the nucleotide sequence of SEQ ID NO: 290, 302, 306, 317, 348, 349, 353, or 355; and (iv) the nucleotide sequence of SEQ ID NO: 236 and the nucleotide sequence of SEQ ID NO: 290, 302, 306, 348, 349, or 355.

In some aspects, the disclosure provides a system for use with a site-directed endonuclease to introduce a deletion in a genomic DNA molecule comprising a fatty-acid amide hydrolase gene (FAAH) upstream a FAAH pseudogene (FAAH-OUT) in a cell, the system comprising: (i) a first gRNA molecule targeting a target site in the genomic DNA molecule, the first gRNA comprising a first spacer sequence selected from any one of SEQ ID NOs: 374, 378, 406, and 421; and (ii) a second gRNA molecule targeting a target site in the genomic DNA molecule, the second gRNA comprising a second spacer sequence selected from any one of SEQ ID NOs: 475, 487, 491, 502, 533, 534, 538, 540 and 550. In some aspects, the system comprises a site directed endonuclease which recognizes a PAM NGG. In some aspects, the site-directed endonuclease is a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof. In some aspects, the first and second spacer sequences are selected from: (i) the nucleotide sequence of SEQ ID NO: 374 and the nucleotide sequence of SEQ ID NO: 475, 487, 491, 502, 533, 534, 538, 540, or 550; (ii) the nucleotide sequence of SEQ ID NO: 378 and the nucleotide sequence of SEQ ID NO: 475, 487, 491, 502, 533, 534, 538, or 540; (iii) the nucleotide sequence of SEQ ID NO: 406 and the nucleotide sequence of SEQ ID NO: 475, 487, 491, 502, 533, 534, 538, or 540; and (iv) the nucleotide sequence of SEQ ID NO: 421 and the nucleotide sequence of SEQ ID NO: 475, 491, 533, 534, or 540.

In some aspects, the disclosure provides a system for use with a site-directed endonuclease to introduce a deletion in a genomic DNA molecule comprising a fatty-acid amide hydrolase gene (FAAH) upstream a FAAH pseudogene (FAAH-OUT) in a cell, the system comprising: (i) a first gRNA molecule targeting a target site in the genomic DNA molecule, the first gRNA comprising a spacer sequence corresponding to a first target sequence consisting of a nucleotide sequence selected from any one of SEQ ID NOs: 930, 932, 939, 942, 947, 949, 956, 960, 967, 968, 976, and 980; and (ii) a second gRNA molecule targeting a target site in the genomic DNA molecule, the second gRNA comprising a spacer sequence corresponding to a second target sequence consisting of a nucleotide sequence selected from any one of SEQ ID NOs: 1046, 1073, 1087, and 1092. In some aspects, the system comprises a site directed endonuclease which recognizes a PAM NNGRRT. In some aspects, the site-directed endonuclease is a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof. In some aspects, the first and second target sequences are selected from (i) the nucleotide sequence of SEQ ID NO: 930, 932, 939, 942, 947, 949, 956, 960, 967, 968, 976, or 980 and the nucleotide sequence of SEQ ID NO: 1046; (ii) the nucleotide sequence of SEQ ID NO: 930, 932, 939, 942, 947, 949, 956, 960, 967, 968, 976, or 980 and the nucleotide sequence of SEQ ID NO: 1073; (iii) the nucleotide sequence of SEQ ID NO: 930, 932, 939, 942, 947, 949, 956, 960, 967, 968, 976, or 980 and the nucleotide sequence of SEQ ID NO: 1087; and (iv) the nucleotide sequence of SEQ ID NO: 930, 956, 960, 967, 968, 976, or 980 and the nucleotide sequence of SEQ ID NO: 1092.

In some aspects, the disclosure provides a system for use with a site-directed endonuclease to introduce a deletion in a genomic DNA molecule comprising a fatty-acid amide hydrolase gene (FAAH) upstream a FAAH pseudogene (FAAH-OUT) in a cell, the system comprising: (i) a first gRNA molecule targeting a target site in the genomic DNA molecule, the first gRNA comprising a first spacer sequence comprising a nucleotide sequence selected from any one of SEQ ID NOs: 1102, 1104, 1111, 1114, 1119, 1121, 1128, 1132, 1139, 1140, 1148, and 1152; and (ii) a second gRNA molecule targeting a target site in the genomic DNA molecule, the second gRNA comprising a second spacer sequence comprising a nucleotide sequence selected from any one of SEQ ID NOs: 1218, 1245, 1259, and 1264. In some aspects, the system comprises a site directed endonuclease which recognizes a PAM NNGRRT. In some aspects, the site-directed endonuclease is a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof. In some aspects, the first and second spacer sequences are selected from (i) the nucleotide sequence of SEQ ID NO: 1102, 1104, 1111, 1119, 1121, 1128, 1132, 1139, 1140, 1148, or 1152 and the nucleotide sequence of SEQ ID NO: 1218; (ii) the nucleotide sequence of SEQ ID NO: 1102, 1104, 1111, 1119, 1121, 1128, 1132, 1139, 1140, 1148, or 1152 and the nucleotide sequence of SEQ ID NO: 1245; (iii) the nucleotide sequence of SEQ ID NO: 1102, 1104, 1111, 1119, 1121, 1128, 1132, 1139, 1140, 1148, or 1152 and the nucleotide sequence of SEQ ID NO: 1259; and (iv) the nucleotide sequence of SEQ ID NO: 1102, 1128, 1132, 1139, 1140, 1148, or 1152 and the nucleotide sequence of SEQ ID NO: 1264.

In any of the foregoing or related aspects, the deletion results in: (i) a genomic DNA molecule deficient in a transcriptional regulatory element that enables or promotes FAAH-OUT expression; (ii) a genomic DNA molecule with reduced rate of transcription of FAAH mRNA; (iii) a reduced amount of FAAH mRNA transcript; (iv) an increased rate of degradation of FAAH mRNA transcript; (v) a reduced amount of FAAH polypeptide product; or (vi) any combination of (i)-(v).

In any of the foregoing or related aspects, wherein the system is introduced to a population of cells comprising the genomic DNA molecule, the system results in a proportion of edited cells comprising the deletion that is at least about 20%, about 25%, about 30%, about 35%, about 40%, about 45%, or about 50% of the total population of cells. In some aspects, the system results in (i) a reduction of FAAH-OUT mRNA expression by at least about 15%, about 20%, about 25%, about 30%, about 35%, about 40%, about 45%, or about 50% relative to a population of unmodified cells; (ii) a reduction of FAAH mRNA expression by at least about 15%, about 20%, about 25%, about 30%, about 35%, about 40%, about 45%, or about 50% relative to a population of unmodified cells; (iii) a reduction of FAAH polypeptide by at least about 15%, about 20%, about 25%, about 30%, about 35%, about 40%, about 45%, or about 50% relative to a population of unmodified cells; or (iv) a combination of (i)-(iii).

In any of the foregoing or related aspects, the system comprises a recombinant expression vector comprising a nucleotide sequence encoding the site directed endonuclease. In some aspects, the system comprises a recombinant expression vector comprising (i) a nucleotide sequence encoding the site directed endonuclease, and (ii) a nucleotide sequence encoding the first gRNA, a nucleotide sequence encoding the second gRNA, or both. In some aspects, the system comprises a first recombinant expression vector comprising a nucleotide sequence encoding the site-directed endonuclease, and a second recombinant expression vector comprising a nucleotide sequence encoding the first gRNA, a nucleotide sequence encoding the second gRNA, or both. In some aspects, the vector is a viral vector. In some aspects, the vector is an AAV vector. In some aspects, the first gRNA, the second gRNA, and the site-directed endonuclease are individually formulated or co-formulated in a lipid nanoparticle. In some aspects, the system comprises the mRNA encoding the site-directed endonuclease. In some aspects, the system comprises the site-directed endonuclease. In some aspects, the system comprises: (i) a ribonucleoprotein complex of the first gRNA and the site-directed endonuclease; (ii) a ribonucleoprotein complex of the second gRNA and the site-directed endonuclease; or (iii) a ribonucleoprotein complex of the first gRNA, the second gRNA, and the site-directed endonuclease. In some aspects, the first gRNA, the second gRNA, and the site-directed nuclease are individually formulated or co-formulated in a lipid nanoparticle.

In some aspects, the disclosure provides a nucleic acid molecule comprising a nucleotide sequence encoding one or more gRNA molecules targeting a target site in a genomic DNA molecule comprising a fatty-acid amide hydrolase gene (FAAH) upstream a FAAH pseudogene (FAAH-OUT) in a cell, the gRNA(s) selected from: (i) a gRNA comprising a spacer sequence corresponding to a target sequence consisting of a nucleotide sequence selected from any one of SEQ ID NOs: 564, 579, 615, and 621; (ii) a gRNA comprising a spacer sequence corresponding to a target sequence consisting of a nucleotide sequence selected from any one of SEQ ID NOs: 629, 630, 644, 676, 692, 702, 705, 709, 712, and 723; (iii) a gRNA molecule comprising a spacer sequence comprising a nucleotide sequence selected from any one of SEQ ID NOs: 750, 765, 801, and 807; (iv) a gRNA molecule comprising a spacer sequence comprising a nucleotide sequence selected from any one of SEQ ID NOs: 815, 816, 830, 862, 878, 888, 891, 895, 898, and 909; (v) a combination of a gRNA of (i) and a gRNA of (ii); and (vi) a combination of a gRNA of (iii) and a gRNA of (iv).

In some aspects, the disclosure provides a nucleic acid molecule comprising a nucleotide sequence encoding a first gRNA and a nucleotide sequence encoding a second gRNA, each independently targeting a target site in a genomic DNA molecule comprising a fatty-acid amide hydrolase gene (FAAH) upstream a FAAH pseudogene (FAAH-OUT) in a cell, wherein the first and second target sequences are selected from: (i) the nucleotide sequence of SEQ ID NO: 564 and the nucleotide sequence of SEQ ID NO: 629, 630, 644, 676, 692, 702, 705, 709, 712, or 723; (ii) the nucleotide sequence of SEQ ID NO: 579 and the nucleotide sequence of SEQ ID NO: 629, 630, 644, 676, 692, 702, 705, 709, 712, or 723; (ii) the nucleotide sequence of SEQ ID NO: 615 and the nucleotide sequence of SEQ ID NO: 629, 630, 644, 676, 692, 702, 705, 709, 712, 723; and (iv) the nucleotide sequence of SEQ ID NO: 621 and the nucleotide sequence of SEQ ID NO: 629, 630, 644, 676, 692, 702, 705, 709, 712, 723.

In some aspects, the disclosure provides a nucleic acid molecule comprising a nucleotide sequence encoding a first gRNA and a nucleotide sequence encoding a second gRNA, each independently targeting a target site in a genomic DNA molecule comprising a fatty-acid amide hydrolase gene (FAAH) upstream a FAAH pseudogene (FAAH-OUT) in a cell, wherein the first gRNA comprises a first spacer sequence and the second gRNA comprises a second spacer sequence, wherein the first and second spacer sequences are selected from: (i) the nucleotide sequence of SEQ ID NO: 750 and the nucleotide sequence of SEQ ID NO: 815, 816, 830, 862, 888, 891, 895, 898, or 909; (ii) the nucleotide sequence of SEQ ID NO: 765 and the nucleotide sequence of SEQ ID NO: 815, 816, 830, 862, 888, 891, 895, 898, or 909; (iii) the nucleotide sequence of SEQ ID NO: 801 and the nucleotide sequence of SEQ ID NO: 815, 816, 830, 862, 888, 891, 895, 898, or 909; and (iv) the nucleotide sequence of SEQ ID NO: 807 and the nucleotide sequence of SEQ ID NO: 815, 816, 830, 862, 888, 891, 895, 898, or 909.

In some aspects, the disclosure provides a nucleic acid molecule comprising: (i) a nucleotide sequence encoding a first gRNA comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with the site-directed endonuclease, the first gRNA combines with the site-directed endonuclease to induce cleavage proximal the first target sequence with a cleavage efficiency of at least 30%; and (ii) a and a nucleotide sequence encoding a second gRNA comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 30%, wherein when the first and second gRNAs are introduced into a cell with a SluCas9 endonuclease or functional variant thereof, result in an approximate 2-8 kb deletion in a in a genomic DNA molecule comprising FAAH upstream FAAH-OUT, wherein the deletion results in full or partial removal of a FAAH-OUT promoter (FOP) and a FAAH-OUT conserved (FOC) element in the genomic DNA molecule.

In any of the foregoing or related aspects, the disclosure provides a recombinant expression vector comprising a nucleic acid molecule of the disclosure. In some aspects, the recombinant expression vector comprises a nucleotide sequence encoding a SluCas9 endonuclease or a functional variant thereof. In some aspects, the vector is a viral vector. In some aspects, the vector is an AAV vector. In some aspects, the recombinant expression vector is formulated in a lipid nanoparticle.

In some aspects, the disclosure provides a nucleic acid molecule comprising a nucleotide sequence encoding one or more gRNA molecules targeting a target site in a genomic DNA molecule comprising a fatty-acid amide hydrolase gene (FAAH) upstream a FAAH pseudogene (FAAH-OUT) in a cell, the gRNA(s) selected from: (i) a gRNA comprising a spacer sequence corresponding to a target sequence consisting of a nucleotide sequence selected from any one of SEQ ID NOs: 189, 193, 221, and 236; (ii) a gRNA comprising a spacer sequence corresponding to a target sequence consisting of a nucleotide sequence selected from any one of SEQ ID NOs: 290, 302, 306, 317, 348, 349, 353, 355, and 365; (iii) a gRNA molecule comprising a spacer sequence comprising a nucleotide sequence selected from any one of SEQ ID NOs: 374, 378, 406, and 421; (iv) a gRNA molecule comprising a spacer sequence comprising a nucleotide sequence selected from any one of SEQ ID NOs: 475, 487, 491, 502, 533, 534, 538, 540 and 550; (v) a combination of a gRNA of (i) and a gRNA of (ii); and (vi) a combination of a gRNA of (iii) and a gRNA of (iv).

In some aspects, the disclosure provides A nucleic acid molecule comprising a nucleotide sequence encoding a first gRNA and a nucleotide sequence encoding a second gRNA, each independently targeting a target site in a genomic DNA molecule comprising a fatty-acid amide hydrolase gene (FAAH) upstream a FAAH pseudogene (FAAH-OUT) in a cell, wherein the first and second target sequences are selected from: (i) the nucleotide sequence of SEQ ID NO: 189 and the nucleotide sequence of SEQ ID NO: 290, 302, 306, 317, 348, 349, 353, 355, or 365; (ii) the nucleotide sequence of SEQ ID NO: 193 and the nucleotide sequence of SEQ ID NO: 290, 302, 306, 317, 348, 349, 353, or 355; (iii) the nucleotide sequence of SEQ ID NO: 221 and the nucleotide.

In some aspects, the disclosure provides a nucleic acid molecule comprising a nucleotide sequence encoding a first gRNA and a nucleotide sequence encoding a second gRNA, each independently targeting a target site in a genomic DNA molecule comprising a fatty-acid amide hydrolase gene (FAAH) upstream a FAAH pseudogene (FAAH-OUT) in a cell, wherein the first gRNA comprises a first spacer sequence and the second gRNA comprises a second spacer sequence, wherein the first and second spacer sequences are selected from: (i) the nucleotide sequence of SEQ ID NO: 374 and the nucleotide sequence of SEQ ID NO: 475, 487, 491, 502, 533, 534, 538, 540, or 550; (ii) the nucleotide sequence of SEQ ID NO: 378 and the nucleotide sequence of SEQ ID NO: 475, 487, 491, 502, 533, 534, 538, or 540; (iii) the nucleotide sequence of SEQ ID NO: 406 and the nucleotide sequence of SEQ ID NO: 475, 487, 491, 502, 533, 534, 538, or 540; and (iv) the nucleotide sequence of SEQ ID NO: 421 and the nucleotide sequence of SEQ ID NO: 475, 491, 533, 534, or 540.

In some aspects, the disclosure provides a nucleic acid molecule comprising: (i) a nucleotide sequence encoding a first gRNA comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with the site-directed endonuclease, the first gRNA combines with the site-directed endonuclease to induce cleavage proximal the first target sequence with a cleavage efficiency of at least 30%; and (ii) a and a nucleotide sequence encoding a second gRNA comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 30%, wherein when the first and second gRNAs are introduced into a cell with a SpCas9 endonuclease or functional variant thereof, result in an approximate 3-10 kb deletion in a in a genomic DNA molecule comprising FAAH upstream FAAH-OUT, wherein the deletion results in removal of a FAAH-OUT promoter (FOP) and a full or partial removal of a FAAH-OUT conserved (FOC) element in the genomic DNA molecule.

In some aspects, the disclosure provides a recombinant expression vector comprising a nucleic acid molecule of the disclosure. In some aspects, the recombinant expression vector comprises a nucleotide sequence encoding a SpCas9 endonuclease or a functional variant thereof. In some aspects, the vector is a viral vector. In some aspects, the vector is an AAV vector. In some aspects, the recombinant expression vector is formulated in a lipid nanoparticle.

In some aspects, the disclosure provides a nucleic acid molecule comprising a nucleotide sequence encoding one or more gRNA molecules targeting a target site in a genomic DNA molecule comprising a fatty-acid amide hydrolase gene (FAAH) upstream a FAAH pseudogene (FAAH-OUT) in a cell, the gRNA(s) selected from: (i) a gRNA comprising a spacer sequence corresponding to a target sequence consisting of a nucleotide sequence selected from any one of SEQ ID NOs: 930, 932, 939, 942, 947, 949, 956, 960, 967, 968, 976, and 980; (ii) a gRNA comprising a spacer sequence corresponding to a target sequence consisting of a nucleotide sequence selected from any one of SEQ ID NOs: 1046, 1073, 1087, and 1092; (iii) a gRNA molecule comprising a spacer sequence comprising a nucleotide sequence selected from any one of SEQ ID NOs: 1102, 1104, 1111, 1114, 1119, 1121, 1128, 1132, 1139, 1140, 1148, and 1152; (iv) a gRNA molecule comprising a spacer sequence comprising a nucleotide sequence selected from any one of SEQ ID NOs: 1218, 1245, 1259, and 1264; (v) a combination of a gRNA of (i) and a gRNA of (ii); and (vi) a combination of a gRNA of (iii) and a gRNA of (iv).

In some aspects, the disclosure provides a nucleic acid molecule comprising a nucleotide sequence encoding a first gRNA and a nucleotide sequence encoding a second gRNA, each independently targeting a target site in a genomic DNA molecule comprising a fatty-acid amide hydrolase gene (FAAH) upstream a FAAH pseudogene (FAAH-OUT) in a cell, wherein the first and second target sequences are selected from: (i) the nucleotide sequence of SEQ ID NO: 930, 932, 939, 942, 947, 949, 956, 960, 967, 968, 976, or 980 and the nucleotide sequence of SEQ ID NO: 1046; (ii) the nucleotide sequence of SEQ ID NO: 930, 932, 939, 942, 947, 949, 956, 960, 967, 968, 976, or 980 and the nucleotide sequence of SEQ ID NO: 1073; (iii) the nucleotide sequence of SEQ ID NO: 930, 932, 939, 942, 947, 949, 956, 960, 967, 968, 976, or 980 and the nucleotide sequence of SEQ ID NO: 1087; and (iv) the nucleotide sequence of SEQ ID NO: 930, 956, 960, 967, 968, 976, or 980 and the nucleotide sequence of SEQ ID NO: 1092.

In some aspects, the disclosure provides a nucleic acid molecule comprising a nucleotide sequence encoding a first gRNA and a nucleotide sequence encoding a second gRNA, each independently targeting a target site in a genomic DNA molecule comprising a fatty-acid amide hydrolase gene (FAAH) upstream a FAAH pseudogene (FAAH-OUT) in a cell, wherein the first gRNA comprises a first spacer sequence and the second gRNA comprises a second spacer sequence, wherein the first and second spacer sequences are selected from: (i) the nucleotide sequence of SEQ ID NO: 1102, 1104, 1111, 1119, 1121, 1128, 1132, 1139, 1140, 1148, or 1152 and the nucleotide sequence of SEQ ID NO: 1218; (ii) the nucleotide sequence of SEQ ID NO: 1102, 1104, 1111, 1119, 1121, 1128, 1132, 1139, 1140, 1148, or 1152 and the nucleotide sequence of SEQ ID NO: 1245; (iii) the nucleotide sequence of SEQ ID NO: 1102, 1104, 1111, 1119, 1121, 1128, 1132, 1139, 1140, 1148, or 1152 and the nucleotide sequence of SEQ ID NO: 1259; and (iv) the nucleotide sequence of SEQ ID NO: 1102, 1128, 1132, 1139, 1140, 1148, or 1152 and the nucleotide sequence of SEQ ID NO: 1264.

In some aspects, the disclosure provides a nucleic acid molecule comprising: (i) a nucleotide sequence encoding a first gRNA comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with a site-directed endonuclease, the first gRNA combines with the site-directed endonuclease to induce cleavage proximal the first target sequence with a cleavage efficiency of at least 15%; and (ii) a and a nucleotide sequence encoding a second gRNA comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with a site-directed endonuclease to induce cleavage proximal the second target sequence with a cleavage efficiency of at least 20%, wherein when the first and second gRNAs are introduced into a cell with a SaCas9 endonuclease or functional variant thereof, result in an approximate 3-10 kb deletion in a in a genomic DNA molecule comprising FAAH upstream FAAH-OUT, wherein the deletion results in removal of a FAAH-OUT promoter (FOP) and a full or partial removal of a FAAH-OUT conserved (FOC) element in the genomic DNA molecule.

In some aspects, the disclosure provides a recombinant expression vector comprising a nucleic acid molecule of the disclosure. In some aspects, the recombinant expression vector comprises a nucleotide sequence encoding a SaCas9 endonuclease or a functional variant thereof. In some aspects, the vector is a viral vector. In some aspects, the vector is an AAV vector. In some aspects, the recombinant expression vector is formulated in a lipid nanoparticle.

In any of the foregoing or related aspects, the disclosure provides a pharmaceutical composition comprising the system, the nucleic acid, or the recombinant expression vector of the disclosure, and a pharmaceutically acceptable carrier.

In any of the foregoing or related aspects, the disclosure provides a kit comprising a container comprising the system, the nucleic acid molecule, the recombinant expression vector, or the pharmaceutical composition of the disclosure for introducing a deletion in a genomic DNA molecule comprising FAAH upstream FAAH-OUT in a cell, and a package insert comprising instructions for use. In some aspects, the disclosure provides a kit comprising a container comprising the system, the nucleic acid molecule, the recombinant expression vector, or the pharmaceutical composition of the disclosure for reducing FAAH expression in a cell, and a package insert comprising instructions for use. In some aspects, the disclosure provides a kit comprising a container comprising the system, the nucleic acid molecule, the recombinant expression vector, or the pharmaceutical composition of the disclosure for use in treating chronic pain in a subject in need thereof, and a package insert comprising instructions for use.

In any of the foregoing or related aspects, the disclosure provides the system, the nucleic acid molecule, the recombinant expression vector, or the pharmaceutical composition of the disclosure, for use in treating a patient with chronic pain by reducing FAAH expression in a cell, the treatment comprising: administering to the patient an effective amount of the system, the nucleic acid molecule, the recombinant expression vector, or the pharmaceutical composition, wherein when the system, the nucleic acid molecule, the recombinant expression vector, or the pharmaceutical composition is administered, the first gRNA and second gRNA combine with the site-directed endonuclease to induce a deletion in the genomic DNA molecule comprising FAAH upstream FAAH-OUT in the cell, thereby reducing FAAH expression in the target cell.

In any of the foregoing or related aspects, the disclosure provides a method for reducing FAAH expression in a cell, the method comprising: contacting the cell with the system, the nucleic acid molecule, the recombinant expression vector, or the pharmaceutical composition of the disclosure, wherein when the system, the nucleic acid molecule, the recombinant expression vector, or the pharmaceutical composition contacts the cell, the first gRNA and second gRNA combine with the site-directed endonuclease to induce a deletion in the genomic DNA molecule comprising FAAH upstream FAAH-OUT in the cell, thereby resulting in reduced FAAH expression in the cell. In some aspects, wherein when the system, the nucleic acid molecule, the recombinant expression vector, or the pharmaceutical composition is contacted with a population of cells, the method results in: (i) a reduction of FAAH mRNA expression by at least about 15%, about 20%, about 25%, about 30%, about 35%, about 40%, about 45%, or about 50% relative to a population of unmodified cells; (ii) a reduction of FAAH polypeptide by at least about 15%, about 20%, about 25%, about 30%, about 35%, about 40%, about 45%, or about 50% relative to a population of unmodified cells; or (iv) a combination of (i)-(ii).

In any of the foregoing or related aspects, the disclosure provides a method of treating a patient with chronic pain by reducing FAAH expression in a target cell, the method comprising: administering to the patient an effective amount of the system, nucleic acid molecule, the recombinant expression vector, or the pharmaceutical composition of the disclosure, wherein when the system, the nucleic acid molecule, the recombinant expression vector, or the pharmaceutical composition is administered, the first gRNA and second gRNA combine with the site-directed endonuclease to induce a deletion in the genomic DNA molecule comprising FAAH upstream FAAH-OUT in the cell, thereby reducing FAAH expression in the target cell. In some aspects, the target cell resides in the brain. In some aspects, the target cell resides in the dorsal root ganglion (DRG). In some aspects, the target cell is a sensory neuron. In some aspects, the route of administration is intra-DRG, intraneural, intrathecal, intra-cisternamagna, and intravenous. In some aspects, the method results in reduced FAAH expression results in increased levels of one or more N-acyl ethanolamines one or more N-acyl taurines, and/or oleamide. In some aspects, the one or more N-acyl ethanolamine are selected from: N-arachidonoyl ethanolamine (AEA), palmitoylethanolamide (PEA), oleoylethanolamine (OEA), or combination thereof.

In some aspects, the disclosure provides a system for introducing a mutation in a genomic DNA molecule comprising FAAH in a cell, the system comprising: (i) a site-directed endonuclease in the form of protein, an mRNA encoding the site-directed endonuclease, or a recombinant expression vector comprising a nucleotide sequence encoding the site-directed endonuclease; and (ii) a gRNA molecule comprising a spacer sequence corresponding to a target sequence within or proximal exon 1, exon 2, exon 3, or exon 4 of the FAAH coding sequence, wherein when the gRNA is introduced into a cell with the site-directed endonuclease, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, 75%, 80%, 85%, or 90% in the cell. In some aspects, the PAM is NNGG, NGG, or NNGRRT. In some aspects, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof. In some aspects, the site-directed endonuclease is a SpCas9 polypeptide or functional derivative thereof, an mRNA encoding the SpCas9 endonuclease or functional derivative thereof, or a recombinant expression vector comprising a nucleotide sequence encoding the SpCas9 endonuclease or functional derivative thereof. In some aspects, the site-directed endonuclease is a SaCas9 polypeptide or functional derivative thereof, an mRNA encoding the SaCas9 endonuclease or functional derivative thereof, or a recombinant expression vector comprising a nucleotide sequence encoding the SaCas9 endonuclease or functional derivative thereof.

In some aspects, the disclosure provides system for introducing a mutation in a genomic DNA molecule comprising FAAH in a cell, the system comprising: (i) a site-directed endonuclease that is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof; and (ii) molecule comprising a spacer sequence corresponding to a target sequence within or proximal exon 1, exon 2, exon 3, or exon 4 of the FAAH coding sequence, wherein when the gRNA is introduced into a cell with the site-directed endonuclease, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, 75%, 80%, 85%, or 90% in the cell. In some aspects, the PAM is NNGG.

In any of the foregoing or related aspects, the target sequence is within exon 1 or exon 2 of FAAH. In some aspects, the mutation is an insertion or deletion (INDEL), optionally wherein the mutation is a frameshift mutation, introduction of a stop codon, or a point mutation. In some aspects, the spacer sequence comprises: a nucleotide sequence having up to 1, 2, or 3 nucleotide deletions or substitutions relative to any one of SEQ ID NOs: 116, 117, 119, 128, 135, 136, 140, and 147. In some aspects, the spacer sequence comprises: a nucleotide sequence set forth in any one of SEQ ID NOs: 116, 117, 119, 128, 135, 136, 140, and 147. In some aspects, target sequence is proximal exon 1 or exon 2 of FAAH. In some aspects, the mutation is an insertion or deletion (INDEL), optionally wherein the mutation is in a splicing element selected from: a 5′ splice site, a 3′ splice site, a branch point sequence, and a pyrimidine tract. In some aspects, the spacer sequence comprises: a nucleotide sequence having up to 1, 2, or 3 nucleotide deletions or substitutions relative to SEQ ID NO: 112 or SEQ ID NO: 133. In some aspects, the spacer sequence comprises: a nucleotide sequence set forth in SEQ ID NO: 112 or SEQ ID NO: 133.

In some aspects, the disclosure provides a system for introducing a mutation in a genomic DNA molecule comprising FAAH in a cell, the system comprising: (i) a site-directed endonuclease that is a SpCas9 polypeptide or functional derivative thereof, an mRNA encoding the SpCas9 endonuclease or functional derivative thereof, or a recombinant expression vector comprising a nucleotide sequence encoding the SpCas9 endonuclease or functional derivative thereof; and (ii) molecule comprising a spacer sequence corresponding to a target sequence within or proximal exon 1, exon 2, exon 3, or exon 4 of the FAAH coding sequence, wherein when the gRNA is introduced into a cell with the site-directed endonuclease, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, 75%, 80%, 85%, or 90% in the cell. In some aspects, the PAM is NGG.

In any of the foregoing or related aspects, the target sequence is within exon 1 or exon 2 of FAAH. In some aspects, the mutation is an insertion or deletion (INDEL), optionally wherein the mutation is a frameshift mutation, introduction of a stop codon, or a point mutation. In some aspects, the spacer sequence comprises: a nucleotide sequence having up to 1, 2, or 3 nucleotide deletions or substitutions relative to any one of SEQ ID NOs: 42, 43, 60, 63, 64, 65, 66, and 68. In some aspects, the spacer sequence comprises: a nucleotide sequence set forth in any one of SEQ ID NOs: 42, 43, 60, 63, 64, 65, 66, and 68. In some aspects, the target sequence is proximal exon 1 or exon 2 of FAAH. In some aspects, the mutation is an insertion or deletion (INDEL), optionally wherein the mutation is in a splicing element selected from: a 5′ splice site, a 3′ splice site, a branch point sequence, and a pyrimidine tract. In some aspects, the spacer sequence comprises: a nucleotide sequence having up to 1, 2, or 3 nucleotide deletions or substitutions relative to SEQ ID NO: 56 or SEQ ID NO: 57. In some aspects, the spacer sequence comprises: a nucleotide sequence set forth in SEQ ID NO: 56 or SEQ ID NO: 57.

In some aspects, the disclosure provides system for introducing a mutation in a genomic DNA molecule comprising FAAH in a cell, the system comprising: (i) a site-directed endonuclease that is a SaCas9 polypeptide or functional derivative thereof, an mRNA encoding the SaCas9 endonuclease or functional derivative thereof, or a recombinant expression vector comprising a nucleotide sequence encoding the SaCas9 endonuclease or functional derivative thereof; and (ii) molecule comprising a spacer sequence corresponding to a target sequence within or proximal exon 1, exon 2, exon 3, or exon 4 of the FAAH coding sequence, wherein when the gRNA is introduced into a cell with the site-directed endonuclease, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, 75%, 80%, 85%, or 90% in the cell. In some aspects, the PAM is NNGRRT.

In any of the foregoing or related aspects, the target sequence is within exon 1, exon 2, exon 3, or exon 4 of FAAH. In some aspects, the mutation is an insertion or deletion (INDEL), optionally wherein the mutation is a frameshift mutation, introduction of a stop codon, or a point mutation. In some aspects, the spacer sequence comprises: a nucleotide sequence having up to 1, 2, or 3 nucleotide deletions or substitutions relative to any one of SEQ ID NOs: 171, 172, 174, 175, 176, 177, 178, and 179. In some aspects, the spacer sequence comprises: a nucleotide sequence set forth in any one of SEQ ID NOs: 171, 172, 174, 175, 176, 177, 178, and 179. In some aspects, the target sequence is proximal exon 1, exon 2, exon 3, or exon 4 of FAAH. In some aspects, the mutation is an insertion or deletion (INDEL), optionally wherein the mutation is in a splicing element selected from: a 5′ splice site, a 3′ splice site, a branch point sequence, and a pyrimidine tract. In some aspects, the spacer sequence comprises: a nucleotide sequence having up to 1, 2, or 3 nucleotide deletions or substitutions relative to any one of SEQ ID NOs: 165, 166, 167, 169, and 180. In some aspects, the spacer sequence comprises: a nucleotide sequence set forth in any one of SEQ ID NOs: 165, 166, 167, 169, and 180. In some aspects, the spacer sequence comprises: a nucleotide sequence having up to 1, 2, or 3 nucleotide deletions or substitutions relative to any one of SEQ ID NOs: 165, 171, 175, 176, and 177. In some aspects, the spacer sequence comprises: a nucleotide sequence set forth in any one of SEQ ID NOs: 165, 171, 175, 176, and 177.

In some aspects, the disclosure provides a system for use with a site-directed endonuclease to introduce a mutation in a genomic DNA molecule comprising FAAH in a cell, the system comprising a gRNA molecule targeting a target site in the genomic DNA molecule, wherein the gRNA comprises: (i) a spacer sequence corresponding to a target sequence consisting of a nucleotide sequence selected from any one of SEQ ID NOs: 69, 70, 78, 89, 90, 92, and 102; (ii) a spacer sequence corresponding to a target sequence consisting of a nucleotide sequence selected from any one of SEQ ID NOs: 72, 76, 77, 79, 88, 93, 95, 96, 100, 103, 104, and 107; (iii) a spacer sequence comprising a nucleotide sequence selected from any one of SEQ ID NOs: 109, 110, 118, 129, 130, 132, and 142; or (iv) a spacer sequence comprising a nucleotide sequence selected from any one of SEQ ID NOs: 112, 116, 117, 119, 128, 133, 135, 136, 140, 143, 144, and 147. In some aspects, the system comprises a site directed endonuclease which recognizes a PAM NNGG. In some aspects, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof.

In some aspects, the disclosure provides a system for use with a site-directed endonuclease to introduce a mutation in a genomic DNA molecule comprising FAAH in a cell, the system comprising a gRNA molecule targeting a target site in the genomic DNA molecule, wherein the gRNA comprises: (i) a spacer sequence corresponding to a target sequence consisting of a nucleotide sequence selected from any one of SEQ ID NOs: 4, 5, 7, 14, and 20; (ii) a spacer sequence corresponding to a target sequence consisting of a nucleotide sequence selected from any one of SEQ ID NOs: 3, 6, 8-13, 16-19, 21-34; (iii) a spacer sequence comprising a nucleotide sequence selected from any one of SEQ ID NOs: 38, 39, 41, 48, and 54; and (iv) a spacer sequence comprising a nucleotide sequence selected from any one of SEQ ID NOs: 37, 40, 42-47, 50-53, 55-68. In some aspects, the system comprises a site directed endonuclease which recognizes a PAM NGG. In some aspects, the site-directed endonuclease is a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof.

In some aspects, the disclosure provides a system for use with a site-directed endonuclease to introduce a mutation in a genomic DNA molecule comprising FAAH in a cell, the system comprising a gRNA molecule targeting a target site in the genomic DNA molecule, wherein the gRNA comprises: (i) a spacer sequence corresponding to a target sequence consisting of a nucleotide sequence selected from any one of SEQ ID NOs: 149, 150, 151, 152, 153, 155, 156, 158, 159, 160, 161, 162, 163 and 164; or (ii) a spacer sequence comprising a nucleotide sequence selected from any one of SEQ ID NOs: 165, 166, 167, 168, 169, 171, 172, 174, 175, 176, 177, 178, 179, and 180. In some aspects, the system comprises a site directed endonuclease which recognizes a PAM NNGRRT. In some aspects, the site-directed endonuclease is a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof.

In any of the foregoing or related aspects, the mutation provides a FAAH allele resulting in: (i) a truncated FAAH protein or an altered open reading frame (ORF) relative to wild-type FAAH; (ii) a decreased rate of transcription relative to wild-type FAAH; (iii) a pre-mRNA transcript with improper splicing relative to a pre-mRNA transcribed from wild-type FAAH; (iv) a reduced amount of mRNA transcript relative to wild-type FAAH; (v) an mRNA transcript with increased rate of degradation and/or decreased half-life compared to wild-type FAAH mRNA; (vi) an mRNA transcript with a decreased rate of translation relative to wild-type FAAH mRNA; (vii) a reduced amount of polypeptide product compared to wild-type FAAH; (viii) a polypeptide product with one or more mutations relative to a wild-type FAAH polypeptide; (ix) a polypeptide with reduced enzymatic activity relative to wild-type FAAH polypeptide; or (x) any combination of (i)-(ix).

In any of the foregoing or related aspects, wherein when the system is introduced to a population of cells comprising the genomic DNA molecule, the system results in (i) a reduction of FAAH mRNA expression by at least about 15%, about 20%, about 25%, about 30%, about 35%, about 40%, about 45%, or about 50% relative to a population of unmodified cells; (ii) a reduction of FAAH polypeptide by at least about 15%, about 20%, about 25%, about 30%, about 35%, about 40%, about 45%, or about 50% relative to a population of unmodified cells; or (iii) a combination of (i)-(ii).

In any of the foregoing or related aspects, the system comprises a recombinant expression vector comprising a nucleotide sequence encoding the site directed endonuclease. In some aspects, the system comprises a recombinant expression vector comprising (i) a nucleotide sequence encoding the site directed endonuclease, and (ii) a nucleotide sequence encoding the gRNA. In some aspects, the system comprises a first recombinant expression vector comprising a nucleotide sequence encoding the site-directed endonuclease, and a second recombinant expression vector comprising a nucleotide sequence encoding the gRNA.

In some aspects, the system comprises a recombinant expression vector comprising (i) a nucleotide sequence encoding the site directed endonuclease, and (ii) a nucleotide sequence encoding the gRNA, wherein the gRNA comprises: (i) a gRNA molecule comprising a spacer sequence comprising a nucleotide sequence set forth in SEQ ID NO: 165, 171, 175, 176 or 177; or; (ii) a gRNA comprising a spacer sequence corresponding to a target sequence consisting of a nucleotide sequence set forth in SEQ ID NO: 149, 155, 159, 160 or 161.

In some aspects, the system comprises a recombinant expression vector comprising (i) a nucleotide sequence encoding the site directed endonuclease, and (ii) a nucleotide sequence encoding the gRNA, wherein the gRNA comprises: (i) a gRNA comprising a spacer sequence corresponding to a target sequence consisting of a nucleotide sequence set forth in SEQ ID NO: 29, 30, 31, 32 or 34; or (ii) a gRNA molecule comprising a spacer sequence comprising a nucleotide sequence set forth in SEQ ID NO: 63, 64, 65, 66 or 68.

In some aspects, the vector is a viral vector. In some aspects, the vector is an AAV vector. In some aspects, the gRNA and the site-directed endonuclease are individually formulated or co-formulated in a lipid nanoparticle. In some aspects, the system comprises an mRNA encoding the site-directed endonuclease. In some aspects, the system comprises the site-directed endonuclease. In some aspects, the system comprises ribonucleoprotein complex of the gRNA and the site-directed endonuclease. In some aspects, the gRNA and the site-directed nuclease are individually formulated or co-formulated in a lipid nanoparticle.

In some aspects, the disclosure provides a nucleic acid molecule comprising a nucleotide sequence encoding one or more gRNA molecules targeting a target site in a genomic DNA molecule comprising a fatty-acid amide hydrolase gene (FAAH) in a cell, the gRNA(s) selected from: (i) a gRNA comprising a spacer sequence corresponding to a target sequence consisting of a nucleotide sequence selected any one of SEQ ID NOs: 69, 70, 78, 89, 90, 92, and 102; (ii) a gRNA comprising a spacer sequence corresponding to a target sequence consisting of a nucleotide sequence selected from any one of SEQ ID NOs: 72, 76, 77, 79, 88, 93, 95, 96, 100, 103, 104, and 107; (iii) a gRNA molecule comprising a spacer sequence comprising a nucleotide sequence selected from any one of SEQ ID NOs: 109, 110, 118, 129, 130, 132, and 142; or (iv) a gRNA molecule comprising a spacer sequence comprising a nucleotide sequence selected from any one of SEQ ID NOs: 112, 116, 117, 119, 128, 133, 135, 136, 140, 143, 144, and 147.

In some aspects, the disclosure provides a nucleotide sequence encoding a gRNA comprising a spacer sequence corresponding to a target sequence within or proximal exon 1 or exon 2 of the FAAH coding sequence, wherein when the gRNA is introduced into a cell with a SluCas9 endonuclease or functional derivative thereof, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, or 75% in the cell.

In any of the foregoing or related aspects, a recombinant expression vector comprising a nucleic acid molecule of the disclosure. In some aspects, the recombinant expression vector comprises a nucleotide sequence encoding a SluCas9 endonuclease or a functional variant thereof. In some aspects, the vector is a viral vector. In some aspects, the vector is an AAV vector. In some aspects, the vector is formulated in a lipid nanoparticle.

In some aspects, the disclosure provides a nucleic acid molecule comprising a nucleotide sequence encoding one or more gRNA molecules targeting a target site in a genomic DNA molecule comprising a fatty-acid amide hydrolase gene (FAAH) in a cell, the gRNA(s) selected from: (i) a gRNA comprising a spacer sequence corresponding to a target sequence consisting of a nucleotide sequence selected from any one of SEQ ID NOs: 4, 5, 7, 14, and 20; (ii) a gRNA comprising a spacer sequence corresponding to a target sequence consisting of a nucleotide sequence selected from any one of SEQ ID NOs: 3, 6, 8-13, 16-19, 21-34; (iii) a gRNA molecule comprising a spacer sequence comprising a nucleotide sequence selected from any one of SEQ ID NOs: 38, 39, 41, 48, and 54; (iv) a gRNA molecule comprising a spacer sequence comprising a nucleotide sequence selected from any one of SEQ ID NOs: 37, 40, 42-47, 50-53, 55-68; (v) a gRNA molecule comprising a spacer sequence comprising a nucleotide sequence selected from any one of 42, 43, 60, 63, 64, 65, 66, and 68; or (vi) a gRNA molecule comprising a spacer sequence comprising a nucleotide sequence selected from any one of 63, 64, 65, 66 or 68.

In some aspects, the disclosure provides a nucleic acid molecule comprising: a nucleotide sequence encoding a gRNA comprising a spacer sequence corresponding to a target sequence within or proximal exon 1 or exon 2 of the FAAH coding sequence, wherein when the gRNA is introduced into a cell with a SpCas9 endonuclease or functional derivative thereof, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, or 75% in the cell.

In any of the foregoing or related aspects, a recombinant expression vector comprising a nucleic acid molecule of the disclosure. In some aspects, the recombinant expression vector comprises a nucleotide sequence encoding a SpCas9 endonuclease or a functional variant thereof. In some aspects, the vector is a viral vector. In some aspects, the vector is an AAV vector. In some aspects, the vector is formulated in a lipid nanoparticle.

In some aspects, the disclosure provides a nucleic acid molecule comprising a nucleotide sequence encoding one or more gRNA molecules targeting a target site in a genomic DNA molecule comprising a fatty-acid amide hydrolase gene (FAAH) in a cell, the gRNA(s) selected from: (i) a gRNA comprising a spacer sequence corresponding to a target sequence consisting of a nucleotide sequence selected from any one of SEQ ID NOs: 149, 150, 151, 152, 153, 155, 156, 158, 159, 160, 161, 162, 163 and 164; (ii) a gRNA molecule comprising a spacer sequence comprising a nucleotide sequence selected from any one of SEQ ID NOs: 165, 166, 167, 168, 169, 171, 172, 174, 175, 176, 177, 178, 179, and 180; or (iii) a g RNA molecule comprising a spacer sequence comprising a nucleotide sequence selected from any one of 165, 171, 175, 176 and 177.

In some aspects, the disclosure provides a nucleic acid molecule comprising: a nucleotide sequence encoding a gRNA comprising a spacer sequence corresponding to a target sequence within or proximal exon 1, exon 2, exon 3, or exon 4 of the FAAH coding sequence, wherein when the gRNA is introduced into a cell with a SaCas9 endonuclease or functional derivative thereof, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, 75%, 80%, 85%, or 90% in the cell.

In any of the foregoing or related aspects, a recombinant expression vector comprising a nucleic acid molecule of the disclosure. In some aspects, the recombinant expression vector comprises a nucleotide sequence encoding a SaCas9 endonuclease or a functional variant thereof. In some aspects, the vector is a viral vector. In some aspects, the vector is an AAV vector. In some aspects, the vector is formulated in a lipid nanoparticle.

In any of the foregoing or related aspects, the disclosure provides a pharmaceutical composition comprising the system, the nucleic acid, or the recombinant expression vector of the disclosure, and a pharmaceutically acceptable carrier.

In any of the foregoing or related aspects, the disclosure provides a kit comprising a container comprising the system, the nucleic acid molecule, the recombinant expression vector, or the pharmaceutical composition of the disclosure for introducing a mutation in a genomic DNA molecule comprising FAAH in a cell, and a package insert comprising instructions for use. In some aspects, the disclosure provides a kit comprising a container comprising the system, the nucleic acid molecule, the recombinant expression vector, or the pharmaceutical composition of the disclosure for reducing FAAH expression in a cell, and a package insert comprising instructions for use. In some aspects, the disclosure provides a kit comprising a container comprising the system, the nucleic acid molecule, the recombinant expression vector, or the pharmaceutical composition of the disclosure for use in treating chronic pain in a subject in need thereof, and a package insert comprising instructions for use.

In any of the foregoing or related aspects, the disclosure provides the system, the nucleic acid molecule, the recombinant expression vector, or the pharmaceutical composition of the disclosure for the manufacture of a medicament for use in treating a patient having chronic pain by introducing a genomic edit in a genomic molecule comprising FAAH upstream FAAH-OUT in a cell.

In any of the foregoing or related aspects, the disclosure provides the system, the nucleic acid molecule, the recombinant expression vector, or the pharmaceutical composition, for use in treating a patient with chronic pain by reducing FAAH expression in a cell, the treatment comprising: administering to the patient an effective amount of the system, the nucleic acid molecule, the recombinant expression vector, or the pharmaceutical composition, wherein when the system, the nucleic acid molecule, the recombinant expression vector, or the pharmaceutical composition is administered, the gRNA combines with the site-directed endonuclease to induce a mutation within or proximal one or more exons of the FAAH coding sequence selected from exon 1, exon 2, exon3, and exon 4, thereby reducing FAAH expression in the target cell.

In any of the foregoing or related aspects, the disclosure provides a method for reducing FAAH expression in a cell, the method comprising: contacting the cell with the system, the nucleic acid molecule, the recombinant expression vector, or the pharmaceutical composition, wherein when the system, the nucleic acid molecule, the recombinant expression vector, or the pharmaceutical composition contacts the cell, the gRNA combines with the site-directed endonuclease to induce a mutation within or proximal one or more exons of the FAAH coding sequence selected from exon 1, exon 2, exon 3, and exon 4, thereby resulting in reduced FAAH expression in the cell. In some aspects, wherein when the system, the nucleic acid molecule, the recombinant expression vector, or the pharmaceutical composition is contacted with a population of cells, the method results in: (i) a reduction of FAAH mRNA expression by at least about 15%, about 20%, about 25%, about 30%, about 35%, about 40%, about 45%, or about 50% relative to a population of unmodified cells; (ii) a reduction of FAAH polypeptide by at least about 15%, about 20%, about 25%, about 30%, about 35%, about 40%, about 45%, or about 50% relative to a population of unmodified cells; or (iii) a combination of (i)-(ii).

In any of the foregoing or related aspects, the disclosure provides a method of treating a patient with chronic pain by reducing FAAH expression in a target cell, the method comprising: administering to the patient an effective amount of the system, the nucleic acid molecule, the recombinant expression vector. or the pharmaceutical composition, wherein when the system, the nucleic acid molecule, the recombinant expression vector, or the pharmaceutical composition is administered, the gRNA combines with the site-directed endonuclease to induce a mutation within or proximal one or more exons of the FAAH coding sequence selected from exon 1, exon 2, exon 3, and exon 4, thereby reducing FAAH expression in the target cell. In some aspects, the target cell resides in the brain. In some aspects, the target cell resides in the dorsal root ganglion (DRG). In some aspects, the target cell is a sensory neuron. In some aspects, the route of administration is intra-DRG, intraneural, intrathecal, intra-cisternamagna, and intravenous. In some aspects, reduced FAAH expression results in increased levels of one or more N-acyl ethanolamines one or more N-acyl taurines, and/or oleamide. In some aspects, the one or more N-acyl ethanolamine are selected from: N-arachidonoyl ethanolamine (AEA), palmitoylethanolamide (PEA), oleoylethanolamine (OEA), or combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C provide bar graphs quantifying editing efficiency (FIG. 1A), FAAH mRNA levels (FIG. 1B), and FAAH protein levels (FIG. 1C) in cells electroporated with SpCas9 and indicated sgRNAs targeting within or proximal the human FAAH coding sequence (CDS). As shown in FIG. 1A, editing efficiency is measured by TIDE analysis, with guides ranked based on frequency of insertions or deletions (INDELs) that are expected to result in a frameshift mutation (“Frameshift INDELs”). Guides with cut locations located in intronic regions of FAAH are annotated by asterisk (*) and frameshift INDELs represents the total frequency of INDELs minus the frequency of INDELs that are a multiple of 3. As shown in FIG. 1B, FAAH mRNA levels are measured by quantitative PCR (qPCR) and represented as fold change for cells electroporated with SpCas9/sgRNA relative to control cells electroporated in PBS only. As shown in FIG. 1C, FAAH protein levels as measured by Simple Wes were normalized by internal control protein (GAPDH) levels and represented as fold change for cells electroporated with SpCas9/sgRNA relative to untreated control cells.

FIGS. 2A-2C provide bar graphs quantifying editing efficiency (FIG. 2A), FAAH mRNA levels (FIG. 2B), and FAAH protein levels (FIG. 2C) in cells electroporated with SluCas9 and indicated sgRNAs targeting within or proximal the human FAAH CDS. As shown in FIG. 2A, editing efficiency is measured by TIDE analysis, with guides ranked as described in FIG. 1A. As shown in FIG. 2B, FAAH mRNA levels are measured by qPCR and represented as fold change for cells electroporated with SluCas9/sgRNA relative to control cells electroporated in PBS only. As shown in FIG. 2C, FAAH protein levels as measured by Simple Wes were normalized by internal control protein (GAPDH) levels and represented as fold change for cells electroporated with SluCas9/sgRNA relative to untreated control cells.

FIGS. 3A-3B provides a bar graph quantifying editing efficiency (FIG. 3A) and FAAH mRNA levels (FIG. 3B) in cells electroporated with SaCas9 and indicated sgRNAs that target the human FAAH CDS. As shown in FIG. 3A, editing efficiency measured by TIDE analysis is shown as frequency of INDELs introducing a frameshift mutation. Guides with cut locations located in intronic regions of FAAH are annotated by asterisk (*) and frameshift INDELs represents the total frequency of INDELs minus the frequency of INDELs that are a multiple of 3. As shown in FIG. 3B, FAAH mRNA levels are measured by quantitative PCR (qPCR) and represented as fold change for cells electroporated with SaCas9/sgRNA relative to control cells electroporated with SaCas9 only.

FIG. 4 provides a schematic depicting FAAH and FAAH-OUT genomic DNA and location of gRNA target sequences (red) for creating a microdeletion in FAAH-OUT, which are shown relative to both the first exon (Ex1) and second exon (Ex2) of FAAH-OUT, as well as a FAAH-OUT promoter (FOP) and FAAH-OUT conserved (FOC) region.

FIGS. 5A-5C provide bar graphs quantifying percent genomic DNA with deletion in FAAH-OUT as measured by droplet digital PCR (ddPCR) (FIG. 5A), FAAH mRNA levels (FIG. 5B), and FAAH protein levels (FIG. 5C) in cells electroporated with SpCas9 and indicated dual sgRNAs targeting human FAAH-OUT. As shown in FIG. 5B, FAAH mRNA levels are measured by qPCR and represented as fold change for cells electroporated with SpCas9/sgRNAs relative to control cells electroporated with SpCas9 only. As shown in FIG. 5C, FAAH protein levels as measured by Simple Wes were normalized by internal control protein (GAPDH) levels and represented as fold change for cells electroporated with SpCas9/sgRNAs relative to untreated control cells.

FIG. 6 provides a bar graph quantifying frequency of INDELs measured by TIDE analysis in cells electroporated with SluCas9 and indicated sgRNAs that target human FAAH-OUT. sgRNAs with target sequences upstream or within FOP are shown in red and sgRNAs with target sequences within or downstream FOC are shown in blue.

FIGS. 7A-7C provide bar graphs quantifying percent genomic DNA with deletion in FAAH-OUT as measured by ddPCR (FIG. 7A), FAAH mRNA levels (FIG. 7B), and FAAH protein levels (FIG. 7C) in cells electroporated with SluCas9 and indicated dual sgRNAs targeting human FAAH-OUT. As shown in FIG. 7B, FAAH mRNA levels are measured by qPCR and represented as fold change for cells electroporated with SluCas9/sgRNAs relative to control cells electroporated with SluCas9 only. As shown in FIG. 7C, FAAH protein levels as measured by Simple Wes were normalized by internal control protein (GAPDH) levels and represented as fold change for cells electroporated with SluCas9/sgRNAs relative to untreated control cells.

FIGS. 8A-8B provide bar graphs quantifying percent genomic DNA with deletion in FAAH-OUT as measured by ddPCR (FIG. 8A) and FAAH mRNA levels (FIG. 8B) in cells electroporated with SaCas9 and indicated dual sgRNAs targeting human FAAH-OUT. As shown in FIG. 8B, FAAH mRNA levels are measured by qPCR and represented as fold change for cells electroporated with SaCas9/sgRNAs relative to control cells electroporated with SaCas9 only.

FIGS. 9A-9C provide bar graphs quantifying editing efficiency (FIG. 9A), FAAH mRNA levels (FIG. 9B), and FAAH protein levels (FIG. 9C) in cells electroporated with a subset of SpCas9 SaCas9 sgRNAs targeting within or proximal the human FAAH coding sequence (CDS). As shown in FIG. 9A, editing efficiency is measured by TIDE analysis, with guides ranked based on frequency of insertions or deletions (INDELs) that are expected to result in a frameshift mutation (“Frameshift INDELs”). Guides with cut locations located in intronic regions of FAAH are annotated by asterisk (*) and frameshift INDELs represents the total frequency of INDELs minus the frequency of INDELs that are a multiple of 3. As shown in FIG. 9B, FAAH mRNA levels are measured by quantitative PCR (qPCR) and represented as fold change for cells electroporated with SpCas9/sgRNA relative to control cells electroporated in PBS only. As shown in FIG. 9C, FAAH protein levels as measured by Simple Wes were normalized by internal control protein (GAPDH) levels and represented as fold change for cells electroporated with SpCas9/sgRNA relative to untreated control cells.

### DETAILED DESCRIPTION

### Overview

The present disclosure is based, at least in part, on the identification of gene editing approaches to modulate FAAH, for example, to treat a subject having a disorder or condition associated with chronic pain. In some aspects, the disclosure provides methods and compositions of gene editing, for example, based on a CRISPR/Cas system described herein, for introducing a gene-edit that results in modulated (e.g., decreased) expression and/or enzymatic activity of FAAH. In some embodiments, the disclosure provides nucleic acid molecules encoding components of a CRISPR/Cas system (e.g., gRNAs, a nucleic acid encoding a Cas nuclease, recombinant expression vector(s) encoding one or more gRNAs, a site-directed endonuclease, or both), for use in introducing a gene edit in a subject that results in modulated (e.g., decreased) expression and/or enzymatic activity of FAAH.

In some aspects, the disclosure provides methods and compositions of gene editing for introducing a deletion in a genomic region downstream the FAAH gene, wherein the genomic region comprises the FAAH pseudogene FAAH-OUT. In some embodiments, the disclosure provides a CRISPR/Cas system comprising dual guide RNAs directed to separate target sequences downstream FAAH, wherein combination of a Cas nuclease (e.g., Cas9 nuclease) with a first and a second gRNA mediates an upstream and downstream double-stranded break (DSB) in the genomic DNA molecule, thereby resulting in a deletion of a genomic region comprising a segment of FAAH-OUT. In some embodiments, the deletion results in removal of one or more genetic elements that regulate expression of FAAH and/or FAAH-OUT. For example, in some embodiments, the deletion results in a full or partial removal of a FAAH-OUT transcriptional regulatory element, such as a FAAH-OUT promoter (FOP), wherein the removal results in decreased expression of FAAH-OUT transcript. In some embodiments, the deletion results in a full or partial removal of a FAAH-OUT conserved (FOC) region that is 800 bp or approximately 800 bp in length. As described herein, the FOC region has significant sequence homology (e.g., approximately 70% sequence homology) to a region of the FAAH gene. Moreover, and without being bound by theory, the FOC region comprises one or more microRNA seed sites that are shared with the FAAH gene transcript, such that, for example, the FAAH-OUT gene transcript functions as a decoy mRNA to prevent degradation of the FAAH gene transcript by a microRNA-mediated degradation pathway. Thus, in some embodiments, and without being bound by theory, the FAAH-OUT transcript comprising a FOC region functions to extend the longevity and/or translation efficiency of the FAAH transcript, and removal of the FOC region from the FAAH-OUT transcript results in a more rapid degradation of the FAAH transcript.

Accordingly, the disclosure provides systems of gene editing (e.g., a CRISPR/Cas system) engineered to introduce a deletion resulting in at least a partial removal of FAAH-OUT, wherein the deletion results in reduced FAAH expression and/or activity. In some embodiments, the disclosure provides dual gRNAs for use with a CRISPR/Cas system, wherein when combined with a site-directed endonuclease (e.g., a Cas9 nuclease) in a cell or a population of cells, the dual gRNAs introduce a deletion of about 2 kb to about 10 kb resulting in at least a partial removal of FAAH-OUT. In some aspects the deletion is about 2 kb to about 5 kb, about 5 kb to about 8 kb, or about 8 kb to about 10 kb, resulting in at least a partial removal of FAAH-OUT. In some embodiments, the deletion results in a full or partial removal of FOP. In some embodiments, the deletion results in a full or partial removal of FOC. As described herein, a deletion of about 2 kb to about 10 kb (or about 2 kb to about 5 kb, or about 5 kb to about 8 kb, or about 8 kb to about 10 kb) comprising (i) full or partial removal of FOC, and/or (ii) a full or partial removal of FOP results in reduction of FAAH expression (e.g., reduced FAAH mRNA expression and/or FAAH polypeptide expression) by at least about 15% or more (e.g., about 15%, about 20%, about 25%, about 30%, about 35%, about 40%, about 45%, about 50%, or about 55%) compared to an unmodified population of cells. In some embodiments, the disclosure provides dual gRNAs for use with a site-directed endonuclease (e.g., a Cas9 nuclease), wherein dual gRNAs that introduce a deletion of about 2 kb to about 8 kb are more efficient than dual gRNAs that introduce a longer deletion of about 8 kb to about 10 kb. Without being bound by a theory, a combination of gRNAs of the disclosure that introduce a deletion of about 2 kb to about 8 kb when combined with a Cas nuclease described herein are particularly useful in some embodiments, as they introduce a deletion of sufficient length to remove FAAH-OUT regulatory elements (e.g., FOP and FOC) that contribute to FAAH expression, while resulting in an efficient deletion.

In some aspects, the disclosure provides methods and compositions of gene editing for introducing a mutation (e.g., an insertion or deletion) within or proximal the coding sequence of the FAAH gene, wherein the mutation results in decreased expression of FAAH transcript, decreased expression of FAAH polypeptide, and/or decreased enzymatic activity of FAAH polypeptide. In some embodiments, the disclosure provides gRNA molecules for use with a site-directed endonuclease (e.g., a Cas9 nuclease), wherein the gRNA comprises a spacer sequence corresponding to a target sequence within or proximal the coding sequence of FAAH (e.g., within or proximal exon 1, exon 2, exon 3, or exon 4 of FAAH). In some embodiments, the gRNAs combine with the Cas nuclease to introduce a DSB proximal the target sequence, wherein repair of the DSB introduces an INDEL that disrupts the FAAH ORF and/or removes a FAAH regulatory element (e.g., a splicing element). In some embodiments, the INDEL introduces a frameshift mutation that disrupts the FAAH ORF. In some embodiments, the INDEL introduces a premature stop codon. In some embodiments, the INDEL removes one or more splicing elements necessary for proper splicing of a precursor mRNA (pre-mRNA) transcribed from the FAAH ORF. As described herein, the disclosure provides CRISPR/Cas systems for introducing a mutation within or proximal the FAAH coding sequence in a population of cells, wherein the mutation results in expression of FAAH transcript and/or polypeptide that is decreased by at least about 15% or more (e.g., about 15%, about 20%, about 25%, about 30%, about 35%, about 40%, about 45%, about 50%, about 55%, about 60%, about 65%, about 70%, about 75%, about 80%, about 85%, or about 90%) compared to an unmodified population of cells.

In some aspects, the disclosure provides gene editing systems and compositions described herein (e.g., a CRISPR/Cas system) for use in gene editing to modulate (e.g., decrease) FAAH expression and/or activity for treatment of various disorders or conditions. In some embodiments, the gene editing systems described herein are used for analgesia (e.g., treatment of chronic pain), treatment of anxiety, and/or treatment of depression in a subject.

In some aspects, the disclosure provides compositions that are suitable for delivery of the system components for use in, for example, in vivo gene editing. In some embodiments, the disclosure provides nucleic acids encoding a site-directed endonuclease, one or more gRNAs, or both, or recombinant vectors comprising a nucleic acid encoding the site-directed endonuclease, a nucleic acid encoding the one or more gRNAs, or both that are suitable for use in, for example, in vivo editing of a genomic DNA molecule comprising FAAH and/or FAAH-OUT. In some embodiments, the disclosure further provides lipid compositions that are suitable for delivery of the system components for use in in vivo gene editing. In some embodiments, the delivery is suitable for administration (e.g., localized administration) of an in vivo gene editing system described herein to a target cell population and/or target tissue expressing FAAH. For example, in some embodiments, the target cell population are neurons (e.g., sensory neurons) and the target tissue is dorsal root ganglion (DRG) (e.g., lumbar DRG). In some embodiments, the disclosure provides methods for delivery of an in vivo gene editing system described herein to the DRG, wherein the gene-editing is localized to the DRG (e.g., lumbar DRG) and results in modulation of FAAH in the DRG. Without being bound by theory, modulation of FAAH in the DRG (e.g., lumbar DRG) reduces chronic pain, for example, by reducing pain stimuli perceived by sensory neurons located in the DRG.

### Systems for Gene Editing to Modulate FAAH

The disclosure provides methods and compositions for genome editing that modulate (e.g., decrease) FAAH expression and/or activity. As used herein, human “fatty acid amide hydrolase 1 (FAAH)” or “FAAH polypeptide” refers to a human enzyme that catalyzes hydrolysis of endogenous amidated lipids (e.g., OEA, AEA, PEA) to their corresponding fatty acids, thereby regulating the signaling functions of these molecules. The methods and compositions for genome editing describe herein comprise (i) introducing a deletion encompassing at least a portion of the FAAH-OUT gene, and (ii) introducing a loss of function mutation in the FAAH gene (e.g., within or proximal the FAAH coding sequence).

In some aspects, the disclosure provides methods and compositions of genome editing of e.g., FAAH and/or FAAH-OUT, using a site-directed endonuclease. Several site-directed endonucleases with capability to edit eukaryotic genomes are known in the art, for example, zinc finger nucleases, transcription activator-like effector nucleases (TALENs), MegaTal, and CRISPR-Cas systems. The CRISPR-Cas system has the advantage of enabling recognition of a genomic target sequence by formation of a ribonucleoprotein complex comprising a Cas nuclease and guide RNA (gRNA). Given gRNAs can be readily and inexpensively designed and evaluated for use with a given Cas nuclease, the CRISPR-Cas system enables a large number of genome targets to be rapidly screened to identify optimal target sites for introducing a desired gene edit (e.g., a mutation in the FAAH coding sequence, e.g., a deletion in FAAH-OUT). Additionally, the CRISPR-Cas system permits the Cas nuclease to combine with gRNAs of different specificity in the same cell, thus enabling the system to introduce multiple gene edits in a single genome.

The CRISPR-Cas system comprises one or more RNA molecules referred to as a guide RNAs (gRNAs) that direct a site-directed endonuclease that is a Cas nuclease (e.g., a Cas9 nuclease) to specific target sequences in a genomic DNA molecule. The targeting occurs by Watson-Crick base pairing between the gRNA molecule spacer sequence and a target sequence in the genomic DNA molecule. Once bound at a target site, the Cas nuclease cleaves both strands of the genomic DNA molecule, creating a DNA double-stranded break (DSB).

One requirement for designing a gRNA to a target sequence in the genomic DNA molecule is that the target sequence contain a protospacer adjacent motif (PAM) sequence. The PAM sequence is recognized by the Cas nuclease used in the CRISPR-Cas system. In some embodiments, a Cas nuclease for use in the present disclosure is a Cas9 nuclease from S. pyogenes (SpCas9), wherein the Cas9 nuclease recognizes the PAM sequence NGG (wherein N=A,C,G,T). In some embodiments, a Cas nuclease for use in the present disclosure is a Cas9 nuclease from S. lugdunensis (SluCas9), wherein the Cas9 nuclease recognizes the PAM sequence NNGG (wherein N=A,C,G,T). In some embodiments, a Cas nuclease for use in the present disclosure is a Cas9 nuclease from S. aureus Cas9 (SaCas9), wherein the Cas9 nuclease recognizes the PAM sequence NNGRRT (wherein N=A,C,G,T; and R=A,G).

### I. Gene Editing of FAAH Pseudogene (FAAH-OUT)

In some embodiments, the disclosure provides a CRISPR-Cas system comprising a site-directed endonuclease and dual gRNAs, wherein a first gRNA targets a first target sequence within the genomic region between the 3′end of FAAH and the FAAH-OUT transcriptional start site, wherein the second gRNA targets a second target sequence upstream exon 3 of FAAH-OUT, wherein the first gRNA and the second gRNA combine with the site-directed endonuclease (e.g., Cas9 nuclease) to introduce a pair of DSBs, i.e., the first DSB proximal the first target sequence and the second DSB proximal the second target sequence, thereby resulting in a deletion of at least a portion of FAAH-OUT in the genomic DNA molecule.

The human FAAH-OUT gene is located immediately downstream of FAAH on human chromosome 1. As used herein, the term “FAAH-OUT” or “FAAH pseudogene” encompasses the genomic region that includes FAAH-OUT regulatory promoters and enhancer sequences, the coding and noncoding intronic sequences (i.e., chr1:46,420,994-46,447,702 of human reference genome Hg38). The FAAH-OUT transcript is approximately 2,845 nt in length. In some embodiments, the FAAH-OUT transcript is a long non-coding RNA. The predicted translation product of FAAH-OUT is a protein of approximately 166 amino acid residues in length.

Certain therapeutic effects of a genomic deletion in FAAH-OUT are known in the art. For example, a microdeletion in FAAH-OUT was reported in a patient with clinical symptoms that included pain insensitivity, a non-anxious disposition, and fast wound healing, as described in WO2019158909 and Habib, et al (2019) BRITISH JOURNAL OF ANAESTHESIA 123:e249, each of which are incorporated herein by reference. The phenotype of the patient included diminished levels of FAAH protein and elevated levels of certain fatty acid amides degraded by FAAH, including AEA.

As used herein, the “PT microdeletion” refers to the reported ˜8 kb microdeletion. The 5′ end of the PT microdeletion is approximately 5.1 kb downstream the 3′ end of FAAH (3′ end of FAAH located at 46,413,575 of human chromosome 1, according to human reference genome Hg38). Moreover, the 5′ end of the PT microdeletion occurs upstream the FAAH-OUT transcriptional start site (TSS; 46,422,994 of human chromosome 1, according to human reference genome Hg38) and the 3′ end of the PT microdeletion is downstream the second exon of FAAH-OUT. Specifically, the 5′ end of the PT microdeletion is located at approximately 46,418,743 (e.g., ±50 bp, ±100 bp, ±200 bp, ±300 bp, ±400 bp, ±500 bp, ±600 bp) of human chromosome 1, according to human reference genome Hg38. The 3′end of the PT microdeletion is located at approximately 46,426,873 (e.g., ±50 bp, ±100 bp, ±200 bp, ±300 bp, ±400 bp, ±500 bp, ±600 bp) of human chromosome 1, according to human reference genome Hg38.

In some embodiments, the disclosure provides a genome editing system (e.g., a CRISPR-Cas system) for introducing a deletion comprising at least a portion of FAAH-OUT. In some embodiments, the genome editing system introduces a deletion in FAAH-OUT that is substantially equivalent in length and/or location relative to the PT microdeletion. For example, in some embodiments, the deletion has the same or similar length to the PT microdeletion (e.g., 8 kb±100 bp, ±200 bp, ±300 bp, ±400 bp, ±500 bp, ±600 bp). In some embodiments, the deletion is shorter than the PT microdeletion, e.g., about 1 kb, about 2 kb, about 3 kb, about 4 kb, about 5 kb, or about 6 kb shorter than the PT microdeletion. In some embodiments, the deletion is longer than the PT microdeletion, e.g., about 1 kb, about 2 kb, or about 3 kb longer than the PT microdeletion. In some embodiments, the deletion comprises a genomic region that is the same or similar to the PT microdeletion (e.g., a region encompassing approximately position 46,418,743 to approximately position 46,426,873 of chromosome 1, according to human reference genome hg38). In some embodiments, the 5′ terminus of the deletion is upstream or downstream (e.g., up to ±1 kb, ±2 kb, ±3 kb) the 5′ terminus of the PT microdeletion. In some embodiments, the 5′ terminus of the deletion is upstream the 5′ terminus of the PT microdeletion by approximately 1 kb, 900 bp, 800 bp, 700 bp, 600 bp, 500 bp, 400 bp, 300 bp, 200 bp, or 100 bp. In some embodiments, the 5′ terminus of the deletion is downstream the 5′ terminus of the PT microdeletion by approximately 100 bp, 200 bp, 300 bp, 400 bp, 500 bp, 600 bp, 700 bp, 800 bp, 900 bp, 1 kb, 1.5 kb, 2 kb, 2.5 kb, 3 kb, 3.5 kb, or 4 kb. In some embodiments, the 3′ terminus of the deletion is upstream or downstream (e.g., up to ±1 kb, ±2 kb, ±3 kb) the 3′ terminus of the PT microdeletion. In some embodiments, the 3′ terminus of the deletion is upstream the 3′ terminus of the PT microdeletion by approximately 2.5 kb, 2 kb, 1.5 kb, 1 kb, 900 bp, 800 bp, 700 bp, 600 bp, 500 bp, 400 bp, 300 bp, 200 bp, or 100 bp. In some embodiments, the 3′ terminus of the deletion is downstream the 3′ terminus of the PT microdeletion by approximately 100 bp, 200 bp, 300 bp, 400 bp, 500 bp, 600 bp, 700 bp, 800 bp, 900 bp, 1 kb, 1.5 kb, 2 kb, or 2.5 kb.

In some embodiments, disclosure provides a genome editing system (e.g., a CRISPR-Cas system) for introducing a deletion, wherein the deletion is at least about 2.0 kb, about 2.5 kb, about 3.0 kb, about 3.5 kb, about 4.0 kb, about 4.5 kb, about 5.0 kb, about 5.5 kb, about 6.0 kb, about 6.5 kb, about 7.0 kb, about 7.5 kb, about 8.0 kb, about 8.5 kb, or about 9.0 kb.

In some embodiments, the 5′ end of the deletion is between about 46,417,743 and about 46,419,743, according to human reference genome Hg38. In some embodiments, the 3′ end of the deletion is between about 46,425,873 and about 46,427,873, according to human reference genome Hg38.

In some embodiments, the deletion is of sufficient length to result in full or partial removal of one or more transcriptional regulatory elements of FAAH-OUT. In some embodiments, the transcriptional regulatory element that is removed by the deletion regulates expression of FAAH-OUT. In some embodiments, the transcriptional regulatory element is about 100 bp, about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 900 bp, about 950 bp, or about 1000 bp upstream the FAAH-OUT transcriptional start site. In some embodiments, the transcriptional regulatory element is about 100 bp, 200 bp, 300 bp, 400 bp, 500 bp, 600 bp, 700 bp, 800 bp, 900 bp, or 1000 bp in length. Methods of determining promoter regions that correspond to a target gene are known in the art, and include, for example, use of computational algorithms to predict promoter regions of a given target gene. Furthermore, methods to determine promoter activity are also known in the art, and include, for example, measuring expression of a reporter gene from the promoter of interest.

In some embodiments, the deletion results in partial removal of the transcriptional regulatory element. In some embodiments, the deletion results in full removal of the transcriptional regulatory element. In some embodiments, full or partial removal of the transcriptional regulatory element is sufficient to reduce FAAH-OUT expression, FAAH expression, or both.

In some embodiments, the transcriptional regulatory element is a FAAH-OUT promoter (FOP). As used herein, “FAAH-OUT promoter” or “FOP” refers to a genomic region that is located approximately 300 bp (e.g. ±50 bp, ±60 bp, ±70 bp, ±80 bp, ±90 bp, ±100 bp, ±150 bp) upstream the FAAH-OUT TSS. The 5′end of FOP is located at approximately 46,422,536 (e.g. ±50 bp, ±60 bp, ±70 bp, ±80 bp, ±90 bp, ±100 bp, ±150 bp) of human chromosome 1, according to human reference genome Hg38. The 3′end of FOP is located at approximately 46,422-695 (e.g. ±50 bp, ±60 bp, ±70 bp, ±80 bp, ±90 bp, ±100 bp, ±150 bp) of human chromosome 1, according to human reference genome Hg38. Without being bound by theory, FOP comprises a transcriptional regulatory element that promotes transcription of the FAAH-OUT coding sequence.

In some embodiments, the deletion introduced in FAAH-OUT according to the disclosure results in full removal of FOP. In some embodiments, the deletion results in partial removal of FOP. In some embodiments, full or partial removal of FOP results in decreased expression of FAAH-OUT transcript, FAAH transcript, or both. In some embodiments, full or partial removal of FOP results in decreased expression of FAAH polypeptide.

In some embodiments, the deletion is of sufficient length to result in full or partial removal of a FAAH-OUT conserved (FOC) region. As used herein, “FAAH-OUT′conserved region”, “FOC region”, or “FOC” each refer to a genomic region of approximately 800 bp (e.g., ±10 bp, ±20 bp, ±30 bp, ±40 bp, ±50 bp, ±60 bp, ±70 bp, ±80 bp, ±90 bp, ±100 bp) located within FAAH-OUT that shares approximately 70% (e.g., 63%, 64%, 65%, 66%, 67%, 68%, 69%, 70%, 71%, 72%, 73%, 74%, 75%, 76%, 77%) sequence identity with a genomic region located in FAAH. The 5′end of FOC is located at approximately 46,424,520 (e.g. ±50 bp, ±60 bp, ±70 bp, ±80 bp, ±90 bp, ±100 bp, ±150 bp) of human chromosome 1, according to human reference genome Hg38. The 3′end of FOC is located at approximately 46,425,325 (e.g. ±50 bp, ±60 bp, ±70 bp, ±80 bp, ±90 bp, ±100 bp, ±150 bp) of human chromosome 1, according to reference genome Hg38. Without being bound by theory, a transcript of the FOC region comprises one or more microRNA binding site that is shared with the FAAH transcript, wherein the FOC region of a FAAH-OUT transcript functions as a decoy for microRNAs target the FAAH transcript, thereby preventing and/or reduce microRNA-directed degradation of the FAAH transcript.

In some embodiments, the deletion introduced in FAAH-OUT according to the disclosure results in full removal of the FOC region. In some embodiments, the deletion results in partial removal of the FOC region. In some embodiments, full or partial removal of the FOC region results in decreased expression of FAAH-OUT transcript, FAAH transcript, or both. In some embodiments, full or partial removal of FOC results in decreased expression of FAAH polypeptide.

In some embodiments, the deletion comprising at least a portion of FAAH-OUT is sufficient to reduce expression of FAAH transcript and/or polypeptide by one or more mechanisms. In some embodiments, the deletion in FAAH-OUT results in (i) removal of genomic sequence comprising one or more transcriptional regulatory elements that contribute to transcription of FAAH (e.g., an enhancer sequence); (ii) reduced expression of a FAAH-OUT transcript that contributes to expression of FAAH polypeptide; (iii) prevents expression of a FAAH-OUT polypeptide that contributes to FAAH expression and/or enzymatic activity; (iv) results in mis-splicing of FAAH transcript, thereby producing a non-functional FAAH transcript; (v) or a combination of (i)-(iv).

In some embodiments, the deletion comprising a portion of FAAH-OUT results in (i) a genomic DNA molecule deficient in a transcriptional regulatory element that enables or promotes FAAH-OUT expression; (ii) a genomic DNA molecule with reduced rate of transcription of FAAH mRNA; (iii) a reduced amount of FAAH mRNA transcript; (iv) increased rate of degradation of FAAH mRNA transcript; (v) a reduced amount of FAAH polypeptide product; or (vi) any combination of (i)-(v).

II. Gene Editing of FAAH

In some aspects, the disclosure provides methods of gene editing to modulate (e.g., decrease) FAAH expression and/or activity by introducing a mutation within or proximal the FAAH coding sequence, wherein the mutation disrupts the FAAH ORF. As used herein, the term “FAAH gene” or “FAAH” encompasses the genomic region that includes FAAH regulatory promoters and enhancer sequences and the coding sequence (i.e., corresponding to approximately chr1:46,392,317-46,415,848 of human reference genome Hg38). The FAAH 5′UTR corresponds to chr1:46,394,317-46,394,348, the coding sequence corresponds to chr1: 46,394,349-46,413,575; and the 3′UTR corresponds to chr1:46,413,576-46,413,845, each according to human reference genome Hg38.

In some embodiments, the disclosure provides a CRISPR-Cas system comprising a site-directed endonuclease (e.g., Cas nuclease) and a gRNA, wherein the gRNA targets a target sequence within or proximal the coding sequence of FAAH, wherein the gRNA combines with the site-directed endonuclease to introduce a DSB proximal the target sequence, wherein repair of the DSB introduces mutation proximal the target sequence, thereby resulting in a mutation that disrupts the FAAH ORF, disrupts expression of FAAH transcript, disrupts expression of FAAH polypeptide, and/or disrupts enzymatic activity of FAAH polypeptide. In some embodiments, the mutation is a substitution, missense, nonsense, insertion, deletion, frameshift, or point mutation.

In some embodiments, the mutation provides a FAAH allele having: (i) a truncated or an altered open reading frame (ORF) relative to wild-type FAAH; (ii) a decreased rate of transcription relative to wild-type FAAH; (iii) a pre-mRNA transcript with improper splicing relative to a pre-mRNA transcribed from wild-type FAAH; (iv) a reduced amount of mRNA transcript relative to wild-type FAAH; (v) an mRNA transcript with increased rate of degradation and/or decreased half-life compared to wild-type FAAH mRNA; (vi) an mRNA transcript with a decreased rate of translation relative to wild-type FAAH mRNA; (vii) a reduced amount of polypeptide product compared to wild-type FAAH; (viii) a polypeptide product with one or more mutations relative to a wild-type FAAH polypeptide; (ix) a polypeptide with reduced enzymatic activity relative to wild-type FAAH polypeptide; or (x) any combination of (i)-(ix).

In some embodiments, the disclosure provides genome editing systems (e.g., CRISPR-Cas system) for introducing a mutation in FAAH for modulating FAAH expression and/or activity. In some embodiments, a CRISPR-Cas system is used to introduce a DSB in FAAH, wherein repair of the DSB by an endogenous DNA repair pathway introduces a mutation proximal the gRNA target sequence. In some embodiments, a non-homologous end joining (NHEJ) pathway repairs the DSB induced by the CRISPR-Cas system. NHEJ is an error-prone process in which a few base pairs are added or deleted at the site of the DSB, thereby creating changes to the original DNA sequence that are referred to as INDELs (insertions/deletions). In some embodiments, repair of the DSB introduces an INDEL proximal the target sequence. In some embodiments, the INDEL is at least ±1 nt (e.g., ±1 nt, ±2 nt, ±3 nt, ±4 nt, ±5 nt or more). In some embodiments, an INDELs is generated within the coding sequence of FAAH, or within a regulatory sequence of FAAH, wherein the INDEL results in a loss or change in expression of FAAH.

In some embodiments, the gRNA target sequence is within the coding sequence of FAAH, and INDELs introduced within the coding sequence of FAAH. In some embodiments, the target sequence is within exon 1, exon 2, exon 3, or exon 4 of FAAH, and INDELs is introduced within exon 1, exon 2, exon 3, or exon 4 of FAAH. In some embodiments, the target sequence is within exon 1 or exon 2 of FAAH, and an INDELs introduced within exon 1 or exon 2 of FAAH

In some embodiments, the INDELs introduces a mutation in the coding sequence of FAAH (e.g., within exon 1, exon 2, exon 3, or exon 4). In some embodiments, the t mutation results in (i) reduced transcription of FAAH, (ii) reduced or inhibited splicing of a FAAH pre-mRNA, (iii) reduced or inhibited translation of FAAH mRNA, (iv) reduced or inhibited enzymatic activity of FAAH polypeptide, or (v) a combination of (i)-(iv).

In some embodiments, the INDELs introduce a premature stop codon in the coding sequence of FAAH (e.g., within exon 1, exon 2, exon 3, or exon 4). In some embodiments, the premature stop codon results in a FAAH transcript encoding a FAAH polypeptide with reduced or inhibited enzymatic activity. In some embodiments, the premature stop codon results in a FAAH transcript that is unstable or has reduced half-life, for example, due to a mechanism of nonsense-mediated decay. In some embodiments, the premature stop codon results in reduced levels of FAAH transcript in the cell.

In some embodiments, the INDEL introduces a frameshift mutation in the coding sequence of FAAH (e.g., within exon 1, exon 2, exon 3, or exon 4). As used herein, a “frameshift mutation” refers to INDELs in the coding sequence of a gene that is not divisible by three, for example, and INDEL of ±1 nt, ±2 nt, ±4 nt, ±5 nt, ±7 nt, ±8 nt, etc, wherein the mutation results in a change in the reading frame of the gene. In some embodiments, the frameshift mutation results in (i) reduced stability of transcript FAAH transcript (e.g., due to a mechanism of nonsense mediated decay) (ii) reduced or inhibited splicing of a FAAH pre-mRNA, (iii) reduced or inhibited translation of FAAH mRNA, (iv) reduced or inhibited enzymatic activity of FAAH polypeptide, or (v) a combination of (i)-(iv).

In some embodiments, the target sequence is proximal the coding sequence of FAAH. In some embodiments, the target sequence is proximal exon 1, exon 2, exon 3, or exon 4 of FAAH. In some embodiments, the target sequence is proximal exon 1 or exon 2 of FAAH. In some embodiments, the target sequence is within a region upstream or downstream exon 1, exon 2, exon 3, or exon 4 of FAAH. In some embodiments, the target sequence is no more than 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, or 100 bp upstream or downstream exon 1, exon 2, exon 3, or exon 4 of FAAH. In some embodiments, the target sequence is no more than 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, or 100 bp upstream or downstream exon 1 or exon 2 of FAAH.

In some embodiments, repair of a DSB proximal the targets sequence results in INDELs proximal FAAH coding sequence. In some embodiments, the INDELs are within a regulatory sequence or transcriptional regulatory element of FAAH. In some embodiments, the INDELs are within a FAAH promoter or enhancer element. In some embodiments, the INDEL is within a splicing element of FAAH. In some embodiments, the splicing element is a 5′ splice site, a 3′ splice site, a polypyrimidine tract, a branch point, an exonic splicing enhancer, an intronic splicing enhancer (ISE), an exonic splicing silencer (ESS), or an intronic splicing silencer (ISS). In some embodiments, the INDEL proximal the FAAH coding sequence mutation results in (i) reduced transcription of FAAH, (ii) splicing of a FAAH pre-mRNA resulting in exon skipping, (iii) reduced or inhibited splicing of a FAAH pre-mRNA, (iv) reduced or inhibited translation of FAAH mRNA, (v) reduced or inhibited enzymatic activity of FAAH polypeptide, or (vi) a combination of (i)-(v).

### III. CRISPR/Cas Nuclease Systems

A. Guide RNA (gRNA)

Engineered CRISPR/Cas systems comprise at least two components: 1) a guide RNA (gRNA) molecule and 2) a Cas nuclease, which interact to form a gRNA/Cas nuclease complex. In an engineered CRISPR/Cas system, a gRNA/Cas nuclease complex is targeted to a specific target sequence of interest within a target nucleic acid (e.g. a genomic DNA molecule) by generating a gRNA comprising a spacer sequence that binds to the specific target sequence in a complementary fashion. Thus, the spacer provides the targeting function of the gRNA/Cas nuclease complex.

The spacer sequence is a sequence that defines the target sequence in a target nucleic acid (e.g., genomic DNA molecule comprising FAAH and/or FAAH-OUT). The target nucleic acid is a double-stranded molecule: one strand comprises the target sequence comprising a protospacer sequence adjacent to a PAM sequence and is referred to as the “PAM strand,” and the second strand is referred to as the “non-PAM strand” and is complementary to the PAM strand. Both the gRNA spacer sequence and the target sequence are complementary to the non-PAM strand of the target nucleic acid.

In some embodiments, the disclosure provides one or more gRNA molecules comprising a spacer sequence that corresponds to a target sequence in a genomic DNA molecule, wherein the genomic DNA molecule comprises FAAH and FAAH-OUT regions. As used herein, the term “corresponding to” a target sequence is used to reference any gRNA spacer sequence that hybridizes to the non-PAM strand of the given target sequence by Watson-Crick base-pairing, wherein the spacer sequence has sufficient complementary to the non-PAM strand of the target sequence, as to enable (i) targeting of a Cas nuclease to the target sequence in the genomic DNA molecule, and/or (ii) facilitate a DNA DSB proximal the target sequence, for example, with a cleavage efficiency that is at least 15%, at least 20%, at least 25%, at least 30%, at least 35%, at least 40%, at least 45%, at least 50%, or higher as measured by INDELs introduced proximal the target sequence. Methods of measuring INDEL formation proximal the target sequence are known in the art, and further described herein.

In some embodiments, a gRNA of the disclosure comprises a spacer sequence that is shorter than the target sequence in the target nucleic acid (e.g., genomic DNA molecule comprising FAAH and/or FAAH-OUT), for example, up to 1, 2, or 3 nucleotides shorter than the target sequence. In some embodiments, the target sequence is 18, 19, 20, 21, 22, or 23 nt in length, and the spacer sequence is shorter than the target sequence by up to 1, 2, or 3 nucleotides. In some embodiments, a gRNA of the disclosure comprises a spacer sequence that is longer than the target sequence in the target nucleic acid (e.g., genomic DNA molecule comprising FAAH and/or FAAH-OUT), for example, up to 1, 2, or 3 nucleotides longer than the target sequence. In some embodiments, the target sequence is 18, 19, 20, 21, 22, or 23 nt in length, and the spacer sequence is longer than the target sequence by up to 1, 2, or 3 nucleotides.

In some embodiments, a gRNA of the disclosure comprises a spacer sequence having up to 1, 2, or 3 mismatches relative to the target sequence in the target nucleic acid (e.g., genomic DNA molecule comprising FAAH and/or FAAH-OUT). In some embodiments, the spacer sequence has sufficient complementary to the non-PAM strand of the target sequence to enable targeting of a Cas nuclease to the target sequence in the target nucleic acid molecule and/or to facilitate a DNA DSB proximal the target sequence.

In some embodiments, the spacer sequence comprises a nucleotide sequence with up to 1, 2, or 3 nucleotides that are not complementary to the non-PAM strand of the target sequence, wherein the spacer sequence has sufficient complementary to the non-PAM strand of the target sequence to target a Cas nuclease to the target sequence in the target nucleic acid. In some embodiments, the spacer comprises 1 nucleotide that is not complementary with the non-PAM strand of the target sequence in the target nucleic acid. In some embodiments, the spacer sequence comprises 2 nucleotides that are not complementary with the non-PAM strand of the target sequence in the target nucleic acid. In some embodiments, the spacer sequence comprises 3 nucleotides that are not complementary with the non-PAM strand of the target sequence in the target nucleic acid.

In some embodiments, the spacer sequence comprises a nucleotide sequence having up to 1, 2, or 3 nucleotide deletions or substitutions relative to nucleotides located 5′ to 3′ at positions 1, 2, or 3 of the target sequence (e.g., positions 16, 17, 18, 19, 20, 21, 22, 23, 24 or 25 upstream the PAM).

(i) Dual gRNAs Targeting FAAH-OUT

In some embodiments, the disclosure provides dual gRNAs for use with a site-directed endonuclease (e.g., Cas nuclease) to introduce a deletion in a genomic DNA molecule comprising FAAH-OUT, wherein the deletion results in removal of a portion of FAAH-OUT. In some embodiments, the dual gRNAs comprise (i) a first gRNA molecule comprising a spacer sequence corresponding to a first target sequence which is downstream the 3′ terminus of FAAH and upstream the transcriptional start site of FAAH-OUT in the genomic DNA molecule; and (ii) a second gRNA molecule comprising a spacer sequence corresponding to a second target sequence which is downstream of the FAAH-OUT transcriptional start site in the genomic DNA molecule. In some embodiments, wherein when a system comprising the dual gRNAs is introduced to a cell with a site-directed endonuclease (e.g., Cas nuclease), the first gRNA combines with the site-directed endonuclease to induce cleavage proximal the first target sequence, the second gRNA combines with the site-directed endonuclease to induce cleavage proximal the second target sequence, wherein cleavage proximal the first target sequence and the second target sequence introduce a deletion comprising at least a portion of FAAH-OUT in the genomic DNA molecule.

In some embodiments, the first gRNA comprises a spacer sequence that corresponds to a first target sequence that is within a region of the genomic DNA molecule that is:

(i) about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 2 kb to about 11 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 3 kb to about 11 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 4 kb to about 11 kb, about 5 kb to about 6 kb, about 5 kb to about 7 kb, about 5 kb to about 8 kb, about 5 kb to about 9 kb, about 5 kb to about 10 kb, about 5 kb to about 11 kb, about 6 kb to about 7 kb, about 6 kb to about 8 kb, about 6 kb to about 9 kb, about 6 kb to about 10 kb, about 6 kb to about 11 kb, about 7 to about 8 kb, about 7 kb to about 9 kb, about 7 kb to about 10 kb, about 7 kb to about 11 kb, about 8 kb to about 9 kb, about 8 kb to about 10 kb, about 8 kb to about 11 kb, about 9 kb to about 10 kb, about 9 kb to about 11 kb, or about 10 kb to about 11 kb downstream the 3′ terminus of FAAH;

(ii) at least about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, about 3.3 kb, about 3.4 kb, about 3.5 kb, about 3.6 kb, 3.7 kb, about 3.8 kb, about 3.9 kb, about 4.0 kb, about 4.1 kb, about 4.2 kb, about 4.3, about 4.4 kb, about 4.5 kb, about 4.6 kb, about 4.7 kb, about 4.8 kb, about 4.9 kb, about 5.0 kb, about 5.1 kb, about 5.2 kb, about 5.3 kb, about 5.4 kb, about 5.5 kb, about 5.6 kb, about 5.7 kb, about 5.8 kb, about 5.9 kb, about 6.0 kb, about 6.1 kb, about 6.2 kb, about 6.3 kb, about 6.4 kb, about 6.5 kb, about 6.6 kb, about 6.7 kb, about 6.8 kb, about 7.1 kb, about 7.2 kb, about 7.3 kb, about 7.4 kb, about 7.5 kb, about 8 kb, about 8.5 kb, about 9 kb, about 9.5 kb, about 10 kb, about 10.5 kb, or about 11 kb downstream the 3′ terminus of FAAH;

(iii) no more than about 4 kb, about 4.5 kb, about 5 kb, about 5.5 kb, about 6 kb, about 6.5 kb, about 7 kb, about 7 kb, about 7.5 kb, about 8 kb, about 8.5 kb, about 9 kb, about 9.5 kb, about 10 kb, about 10.5 kb, about 11 kb, about 11.5 kb, or about 12 kb downstream the 3′ terminus of FAAH;

(iv) a combination of (i)-(iii).

In some embodiments, the first gRNA comprises a spacer sequence that corresponds to a first target sequence that is within a region of the genomic DNA molecule that is:

(i) at least about 100 bp, about 150 bp, about 200 bp, about 250 bp, about 300 bp, about 350 bp, about 400 bp, about 450 bp, or about 500 bp upstream the transcriptional start site of FAAH-OUT;

(ii) about 100 bp to about 200 bp, about 100 bp to about 300 bp, about 100 bp to about 400 bp, about 100 bp to about 500 bp, about 200 bp to about 300 bp, about 200 bp to about 400 bp, about 200 bp to about 600 bp, about 300 bp to about 400 bp, about 300 bp to about 500 bp, about 300 bp to about 600 bp, about 300 bp to about 700 bp, about 300 bp to about 800 bp, about 300 bp to about 900 bp, about 400 bp to about 500 bp, about 400 bp to about 600 bp, about 400 bp to about 700 bp, about 400 bp to about 800 bp, about 500 bp to about 900 bp, or about 400 bp to about 1000 bp, about 1.5 kb, about 2 kb, about 2.5 kb, about 3 kb, about 3.5 kb, about 4 kb, about 4.5, or about 5 kb upstream the transcriptional start site of FAAH-OUT;

(iii) no more than about 1 kb, about 1.5 kb, about 2 kb, about 2.5 kb, about 3 kb, about 3.5 kb, about 4 kb, about 4.5 kb, about 5 kb, or about 5.5. kb upstream the transcriptional start site of FAAH-OUT; or

(iv) a combination of (i)-(iii).

In some embodiments, the first gRNA comprises a spacer sequence that corresponds to a first target sequence that is within a region of the genomic DNA molecule that is:

(i) about 2 kb, about 2.5 kb, about 3 kb, about 3.5 kb, about 4 kb, about 4.5 kb, about 4.6 kb, about 5 kb, about 5.5 kb, about 6 kb, about 6.5 kb, about 7 kb, about 7.5 kb, or about 8 kb, downstream the 3′ terminus of FAAH; and

(ii) about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.5 kb, about 2 kb, about 2.5 kb, about 3 kb, about 3.5 kb, about 4 kb, about 4.5, or about 5 kb upstream the transcriptional start site of FAAH-OUT.

In some embodiments, the first gRNA comprises a spacer sequence that corresponds to a first target sequence that is

(i) within a region of the genomic DNA molecule between about 46,416,743 to about 46,420,743 of chromosome 1, according to human reference genome Hg38;

(ii) within a region of the genomic DNA molecule between about 46,417,743 to about 46,419,743 of chromosome 1, according to human reference genome Hg38;

(iii) within a region of the genomic DNA molecule between about 46,418,243 to about 46,419,243 of chromosome 1, according to human reference genome Hg38;

(iv) within a region of the genomic DNA molecule between about 46,418,846 to about 46,422,883 of chromosome 1, according to human reference genome Hg38;

(v) within a region of the genomic DNA molecule between about 46,418, 096 to about 46,422,633 of chromosome 1, according to human reference genome Hg38;

(vi) within a region of the genomic DNA molecule between about 46,419.046 to about 46,422,683 of chromosome 1, according to human reference genome Hg38;

(vii) within a region of the genomic DNA molecule between about 46,418,391 to about 46,421,122 of chromosome 1, according to human reference genome Hg38;

(viii) within a region of the genomic DNA molecule between about 46,418,141 to about 46,420,972 of chromosome 1, according to human reference genome Hg38;

(ix) within a region of the genomic DNA molecule between about 46,418,191 to about 46,420,922 of chromosome 1, according to human reference genome Hg38;

(x) within a region of the genomic DNA molecule between about 46,418,168 to about 46,422,208 of chromosome 1, according to human reference genome Hg38;

(xi) within a region of the genomic DNA molecule between about 46,418,318 to about 46,422,058 of chromosome 1, according to human reference genome Hg38; or

(xii) within a region of the genomic DNA molecule between about 46,418,368 to about 46,422,008 of chromosome 1, according to human reference genome Hg38.

In some embodiments, the first gRNA comprises a spacer sequence that corresponds to a first target sequence that is within a region of the genomic DNA molecule that is upstream or is within a transcriptional regulatory element of FAAH-OUT. In some embodiments, the first gRNA comprises a spacer sequence that corresponds to a first target sequence that is within a region of the genomic DNA molecule that is upstream or within FOP.

In some embodiments, the first gRNA molecule comprises a spacer sequence that corresponds to a target sequence comprising a NNGG PAM. In some embodiments, the target sequence consists of a nucleotide sequence as set forth in any one of SEQ ID NOs: 551-624. In some embodiments, the first gRNA comprises a spacer sequence comprising a nucleotide sequence as set forth in any one of SEQ ID NOs: 737-810, or a nucleotide sequence having up to 1, 2, or 3 nucleotide substitutions or deletions relative to a nucleotide sequence set forth in any one of SEQ ID NOs: 737-810.

In some embodiments, the first gRNA molecule comprises a spacer sequence that corresponds to a target sequence comprising an NGG PAM. In some embodiments, the target sequence consists of a nucleotide sequence as set forth in any one of SEQ ID NOs: 181-280. In some embodiments, the first gRNA comprises a spacer sequence comprising a nucleotide sequence as set forth in any one of SEQ ID NOs: 366-465, or a nucleotide sequence having up to 1, 2, or 3 nucleotide substitutions or deletions relative to a nucleotide sequence set forth in any one of SEQ ID NOs: 366-465.

In some embodiments, the first gRNA molecule comprises a spacer sequence that corresponds to a target sequence comprising a NNGRRT PAM. In some embodiments, the target sequence consists of a nucleotide sequence as set forth in any one of SEQ ID NOs: 923-1024. In some embodiments, the first gRNA comprises a spacer sequence comprising a nucleotide sequence as set forth in any one of SEQ ID NOs: 1095-1196, or a nucleotide sequence having up to 1, 2, or 3 nucleotide substitutions or deletions relative to a nucleotide sequence set forth in any one of SEQ ID NOs: 1095-1196.

In some embodiments, the second gRNA comprises a spacer sequence that corresponds to a second target sequence that is within a region of the genomic DNA molecule that is:

(i) at least about 1.5 kb, about 1.6 kb, about 1.7 kb, about 1.8 kb, about 1.9 kb, about 2.0 kb, about 2.1 kb, about 2.2 kb, about 2.3, about 2.4 kb, about 2.5 kb, about 2.6 kb, about 2.7 kb, about 2.8 kb, about 2.9 kb, about 3.0 kb, about 3.1 kb, about 3.2 kb, about 3.3, about 3.4 kb, about 3.5 kb, about 3.6 kb, at least about 3.7 kb, about 3.8 kb, about 3.9 kb, about 4.0 kb, about 4.1 kb, about 4.2 kb, about 4.3, about 4.4 kb, about 4.5 kb, about 4.6 kb, about 4.7 kb, about 4.8 kb, about 4.9 kb, about 5.0 kb, about 5.1 kb, about 5.2 kb, about 5.3 kb, about 5.4 kb, or about 5.5 kb downstream the transcriptional start site of FAAH-OUT;

(ii) about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 to about 8 kb downstream the transcriptional start site of FAAH-OUT;

(iii) no more than about 3 kb, about 3.5 kb, about 4 kb, about 4.5 kb, about 5 kb, about 5.5 kb, about 6 kb, about 6.5 kb, about 7 kb, about 7 kb, or about 7.5 kb downstream the transcriptional start site of FAAH-OUT;

(iv) a combination of (i)-(iii).

In some embodiments, the second gRNA comprises a spacer sequence that corresponds to a second target sequence that is within a region of the genomic DNA molecule that is:

(i) at least about 3 kb, about 3.5 kb, about 3.6 kb, about 3.7 kb, about 3.8 kb, about 3.9 kb, about 4.0 kb, about 4.1 kb, about 4.2 kb, about 4.3, about 4.4 kb, about 4.5 kb, about 4.6 kb, about 4.7 kb, about 4.8 kb, about 4.9 kb, about 5.0 kb, about 5.1 kb, about 5.2 kb, about 5.3 kb, about 5.4 kb, about 5.5 kb, about 5.6 kb, about 5.7 kb, about 5.8 kb, about 5.9 kb, about 6.0 kb, about 6.1 kb, about 6.2 kb, about 6.3 kb, about 6.4 kb, about 6.5 kb, about 6.6 kb, about 6.7 kb, about 6.8 kb, about 7.1 kb, about 7.2 kb, about 7.3 kb, about 7.4 kb, or about 7.5 kb upstream the 5′ terminus of exon 3 of FAAH-OUT;

(ii) about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 to about 8 kb upstream the 5′ terminus of exon 3 of FAAH-OUT;

(iii) no more than about 4 kb, about 4.5 kb, about 5 kb, about 5.5 kb, about 6 kb, about 6.5 kb, about 7 kb, about 7 kb, about 7.5 kb or about 8 kb upstream the 5′ terminus of exon 3 of FAAH-OUT;

(iv) a combination of (i)-(iii).

In some embodiments, the second gRNA comprises a spacer sequence that corresponds to a second target sequence that is

(i) within a region of the genomic DNA molecule between about 46,424,873 to about 46,428,873 of chromosome 1, according to human reference genome Hg38;

(ii) within a region of the genomic DNA molecule between about 46,425,873 to about 46,427,873 of chromosome 1, according to human reference genome Hg38;

(iii) within a region of the genomic DNA molecule between about 46,426,373 to about 46,427,373 of chromosome 1, according to human reference genome Hg38;

(iv) within a region of the genomic DNA molecule between about 46,424,697 to about 46,426,377 of chromosome 1, according to human reference genome Hg38;

(v) within a region of the genomic DNA molecule between about 46,424,847 to about 46,426,227 of chromosome 1, according to human reference genome Hg38;

(vi) within a region of the genomic DNA molecule between about 46,424,897 to about 46,426,177 of chromosome 1, according to human reference genome Hg38;

(vii) within a region of the genomic DNA molecule between about 46,424,651 to about 46,428,274 of chromosome 1, according to human reference genome Hg38;

(viii) within a region of the genomic DNA molecule between about 46,424,811 to about 46,428,124 of chromosome 1, according to human reference genome Hg38;

(ix) within a region of the genomic DNA molecule between about 46,424,851 to about 46,428,074 of chromosome 1, according to human reference genome Hg38;

(x) within a region of the genomic DNA molecule between about 46,424,887 to about 46,428,508 of chromosome 1, according to human reference genome Hg38;

(xi) within a region of the genomic DNA molecule between about 46,425,037 to about 46,428,268 of chromosome 1, according to human reference genome Hg38; or

(xii) within a region of the genomic DNA molecule between about 46,425,087 to about 46,428,308 of chromosome 1, according to human reference genome Hg38

In some embodiments, the second gRNA comprises a spacer sequence that corresponds to a second target sequence that is within a region of the genomic DNA molecule that is downstream or is within FOC.

In some embodiments, the second gRNA molecule comprises a spacer sequence that corresponds to a target sequence comprising a NNGG PAM. In some embodiments, the target sequence comprises a nucleotide sequence as set forth in SEQ ID NOs: 625-736. In some embodiments, the second gRNA comprises a spacer sequence comprising a nucleotide sequence as set forth in SEQ ID NOs: 811-922, or a nucleotide sequence having up to 1, 2, or 3 nucleotide substitutions or deletions relative to a nucleotide sequence set forth in any one of SEQ ID NOs: 811-922.

In some embodiments, the second gRNA molecule comprises a spacer sequence that corresponds to a target sequence comprising a NGG PAM. In some embodiments, the target sequence comprises a nucleotide sequence as set forth in SEQ ID NOs: 281-365. In some embodiments, the second gRNA comprises a spacer sequence comprising a nucleotide sequence as set forth in SEQ ID NOs: 466-550, or a nucleotide sequence having up to 1, 2, or 3 nucleotide substitutions or deletions relative to a nucleotide sequence set forth in any one of SEQ ID NOs: 466-550.

In some embodiments, the second gRNA molecule comprises a spacer sequence that corresponds to a target sequence comprising a NNGRRT PAM. In some embodiments, the target sequence comprises a nucleotide sequence as set forth in SEQ ID NOs: 1025-1094. In some embodiments, the second gRNA comprises a spacer sequence comprising a nucleotide sequence as set forth in SEQ ID NOs: 1197-1266, or a nucleotide sequence having up to 1, 2, or 3 nucleotide substitutions or deletions relative to a nucleotide sequence set forth in any one of SEQ ID NOs: 1197-1266.

In some embodiments, the disclosure provides a first gRNA and a second gRNA for use with a site-directed endonuclease (e.g., Cas nuclease) for introducing a deletion in a genomic DNA molecule comprising FAAH-OUT. In some embodiments, the first gRNA comprises a spacer sequence that corresponds to a first target sequence and the second gRNA comprises a spacer sequence that corresponds to a second target sequence.

In some embodiments, the disclosure provides a first gRNA and a second gRNA for use with a site-directed endonuclease (e.g., Cas nuclease) for introducing an approximately 2 kb to approximately 3 kb, approximately 2 kb to approximately 4 kb, approximately 2 kb to approximately 5 kb, approximately 2 kb to approximately 6 kb, approximately 2 kb to approximately 7 kb, approximately 2 kb to approximately 8 kb, approximately 2 kb to approximately 9 kb, or approximately 2 kb to approximately 10 kb deletion in a genomic DNA molecule comprising FAAH-OUT, wherein the deletion results in a partial removal of FOP and a partial removal of FOC.

In some embodiments, the disclosure provides a first gRNA and a second gRNA for use with a site-directed endonuclease (e.g., Cas nuclease) for introducing an approximately 2 kb to approximately 3 kb, approximately 2 kb to approximately 4 kb, approximately 2 kb to approximately 5 kb, approximately 2 kb to approximately 6 kb, approximately 2 kb to approximately 7 kb, approximately 2 kb to approximately 8 kb, approximately 2 kb to approximately 9 kb, or approximately 2 kb to approximately 10 kb in a genomic DNA molecule comprising FAAH-OUT, wherein the deletion results in a partial removal of FOP and a full removal of FOC.

In some embodiments, the disclosure provides a first gRNA and a second gRNA for use with a site-directed endonuclease (e.g., Cas nuclease) for introducing an approximately 2 kb to approximately 3 kb, approximately 2 kb to approximately 4 kb, approximately 2 kb to approximately 5 kb, approximately 2 kb to approximately 6 kb, approximately 2 kb to approximately 7 kb, approximately 2 kb to approximately 8 kb, approximately 2 kb to approximately 9 kb, or approximately 2 kb to approximately 10 kb deletion in a genomic DNA molecule comprising FAAH-OUT, wherein the deletion results in a full removal of FOP and a partial removal of FOC.

In some embodiments, the disclosure provides a first gRNA and a second gRNA for use with a site-directed endonuclease (e.g., Cas nuclease) for introducing an approximately 2 kb to approximately 3 kb, approximately 2 kb to approximately 4 kb, approximately 2 kb to approximately 5 kb, approximately 2 kb to approximately 6 kb, approximately 2 kb to approximately 7 kb, approximately 2 kb to approximately 8 kb, approximately 2 kb to approximately 9 kb, or approximately 2 kb to approximately 10 kb deletion in a genomic DNA molecule comprising FAAH-OUT, wherein the deletion results in a full removal of FOP and a full removal of FOC.

In some embodiments, the first gRNA molecule comprises a spacer sequence that corresponds to a first target sequence comprising a NNGG PAM and the second gRNA molecule comprises a spacer sequence that corresponds to a second target sequence comprising a NNGG PAM. In some embodiments, the first target sequence comprises a nucleotide sequence as set forth in any one of SEQ ID NOs: 551-624 and the second target sequence comprise a nucleotide sequence as set forth in any one of SEQ ID NOs: 625-736. In some embodiments, the first gRNA comprises a spacer sequence comprising a nucleotide sequence as set forth in any one of SEQ ID NOs: 737-810 and the second target sequence comprise a nucleotide sequence as set forth in any one of SEQ ID NOs: 811-922.

In some embodiments, the first gRNA molecule comprises a spacer sequence that corresponds to a first target sequence comprising a NGG PAM and the second gRNA molecule comprises a spacer sequence that corresponds to a second target sequence comprising a NGG PAM. In some embodiments, the first target sequence comprises a nucleotide sequence as set forth in any one of SEQ ID NOs: 181-280 and the second target sequence comprise a nucleotide sequence as set forth in any one of SEQ ID NOs: 281-365. In some embodiments, the first gRNA comprises a spacer sequence comprising a nucleotide sequence as set forth in any one of SEQ ID NOs: 366-465 and the second target sequence comprise a nucleotide sequence as set forth in any one of SEQ ID NOs: 466-550.

In some embodiments, the first gRNA molecule comprises a spacer sequence that corresponds to a first target sequence comprising a NNGRRT PAM and the second gRNA molecule comprises a spacer sequence that corresponds to a second target sequence comprising a NNGRRT PAM. In some embodiments, the first target sequence comprises a nucleotide sequence as set forth in any one of SEQ ID NOs: 923-1024 and the second target sequence comprise a nucleotide sequence as set forth in any one of SEQ ID NOs: 1025-1094. In some embodiments, the first gRNA comprises a spacer sequence comprising a nucleotide sequence as set forth in any one of SEQ ID NOs: 1095-1196 and the second target sequence comprise a nucleotide sequence as set forth in any one of SEQ ID NOs: 1197-1266.

(ii) gRNAs Targeting FAAH

In some embodiments, the disclosure provides gRNAs for use with a site-directed endonuclease to introduce a mutation in a genomic molecule comprising FAAH, wherein the mutation is introduced within or proximal the coding sequence of FAAH. In some embodiments, the gRNA molecule comprises a spacer sequence that corresponds to a target sequence that is within or proximal the FAAH coding sequence.

In some embodiments, the gRNA molecule comprises a spacer sequence that corresponds to a target sequence that is within the coding sequence of FAAH. In some embodiments, the target sequence is located within exon 1, exon 2, exon 3, or exon 4 of FAAH.

In some embodiments, the target sequence is located within exon 1 of FAAH, e.g., between about position 46,394,317 and about position 46,394,543 of chromosome 1, according to human reference genome Hg38. In some embodiments, the target sequence is located with exon 2 of FAAH, e.g., between about position 46,402,091 and about position 46,402,204 of chromosome 1, according to human reference genome Hg38. In some embodiments, the target sequence is located within exon 3 of FAAH, e.g., between about position 46,405,014 and about position 46,405,148 of human chromosome 1, according to human reference genome Hg38. In some embodiments, the target sequence is located within exon 4 of FAAH, e.g., between about position 46,405,372 and about position 46,405,505 of human chromosome 1, according to human reference genome Hg38.

In some embodiments, the gRNA molecule comprises a spacer sequence that corresponds to a target sequence that is proximal the coding sequence of FAAH. In some embodiments, the target sequence is proximal exon 1, exon 2, exon 3, or exon 4 of FAAH.

In some embodiments, the target sequence is located proximal to exon 1 of FAAH. In some embodiments, the 3′ terminus of the target sequence is located about 100 nt, about 90 nt, about 80 nt, about 70 nt, about 60 nt, about 50 nt, about 40 nt, about 30 nt, about 20 nt, or about 10 nt upstream position 46,394,317 of chromosome 1, according to human reference genome Hg38. In some embodiments, the 5′ terminus of the target sequence is located about 10 nt, about 20 nt, about 30 nt, about 40 nt, about 50 nt, about 60 nt, about 70 nt, about 80 nt, about 90 nt, or about 100 nt downstream position 46,394,543 of chromosome 1, according to human reference genome Hg38.

In some embodiments, the target sequence is located proximal to exon 2 of FAAH. In some embodiments, the 3′ terminus of the target sequence is located about 100 nt, about 90 nt, about 80 nt, about 70 nt, about 60 nt, about 50 nt, about 40 nt, about 30 nt, about 20 nt, or about 10 nt upstream position 46,402,091 of chromosome 1, according to human reference genome Hg38. In some embodiments, the 5′ terminus of the target sequence is located about 10 nt, about 20 nt, about 30 nt, about 40 nt, about 50 nt, about 60 nt, about 70 nt, about 80 nt, about 90 nt, or about 100 nt downstream position 46,402,204 of chromosome 1, according to human reference genome Hg38.

In some embodiments, the target sequence is located proximal to exon 3 of FAAH. In some embodiments, the 3′ terminus of the target sequence is located about 100 nt, about 90 nt, about 80 nt, about 70 nt, about 60 nt, about 50 nt, about 40 nt, about 30 nt, about 20 nt, or about 10 nt upstream position 46,405,014 of chromosome 1, according to human reference genome Hg38. In some embodiments, the 5′ terminus of the target sequence is located about 10 nt, about 20 nt, about 30 nt, about 40 nt, about 50 nt, about 60 nt, about 70 nt, about 80 nt, about 90 nt, or about 100 nt downstream position 46,405,148 of chromosome 1, according to human reference genome Hg38.

In some embodiments, the target sequence is located proximal to exon 4 of FAAH. In some embodiments, the 3′ terminus of the target sequence is located about 100 nt, about 90 nt, about 80 nt, about 70 nt, about 60 nt, about 50 nt, about 40 nt, about 30 nt, about 20 nt, or about 10 nt upstream position 46,405,372 of chromosome 1, according to human reference genome Hg38. In some embodiments, the 5′ terminus of the target sequence is located about 10 nt, about 20 nt, about 30 nt, about 40 nt, about 50 nt, about 60 nt, about 70 nt, about 80 nt, about 90 nt, or about 100 nt downstream position 46,405,505 of chromosome 1, according to human reference genome Hg38.

In some embodiments, the gRNA molecule comprises a spacer sequence that corresponds to a target sequence comprising a NNGG PAM. In some embodiments, the target sequence comprises a nucleotide sequence as set forth in any one of SEQ ID NOs: 69-108. In some embodiments, the gRNA comprises a spacer sequence comprising a nucleotide sequence as set forth in any one of SEQ ID NOs: 109-148, or a nucleotide sequence having up to 1, 2, or 3 nucleotide substitutions or deletions relative to a nucleotide sequence set forth in any one of SEQ ID NOs: 109-148.

In some embodiments, the gRNA molecule comprises a spacer sequence that corresponds to a target sequence comprising a NGG PAM. In some embodiments, the target sequence comprises a nucleotide sequence as set forth in any one of SEQ ID NOs: 1-34. In some embodiments, the gRNA comprises a spacer sequence comprising a nucleotide sequence as set forth in any one of SEQ ID NOs: 35-68, or a nucleotide sequence having up to 1, 2, or 3 nucleotide substitutions or deletions relative to a nucleotide sequence set forth in any one of SEQ ID NOs: 35-68.

In some embodiments, the gRNA molecule comprises a spacer sequence that corresponds to a target sequence comprising a NNGRRT PAM. In some embodiments, the target sequence comprises a nucleotide sequence as set forth in any one of SEQ ID NOs: 149-164. In some embodiments, the gRNA comprises a spacer sequence comprising a nucleotide sequence as set forth in any one of SEQ ID NOs: 165-180, or a nucleotide sequence having up to 1, 2, or 3 nucleotide substitutions or deletions relative to a nucleotide sequence set forth in any one of SEQ ID NOs: 165-180.

(iii) Methods of gRNA Selection

In some embodiments, the disclosure provides gRNA spacer sequences that target specific regions of the genome, e.g., a region within or proximal the FAAH coding sequence, e.g., a region within or proximal FAAH-OUT, that are designed in silico by locating targets sequences (e.g., a 19, 20, 21, 22 bp sequence) adjacent to a PAM sequence in the genomic region of interest.

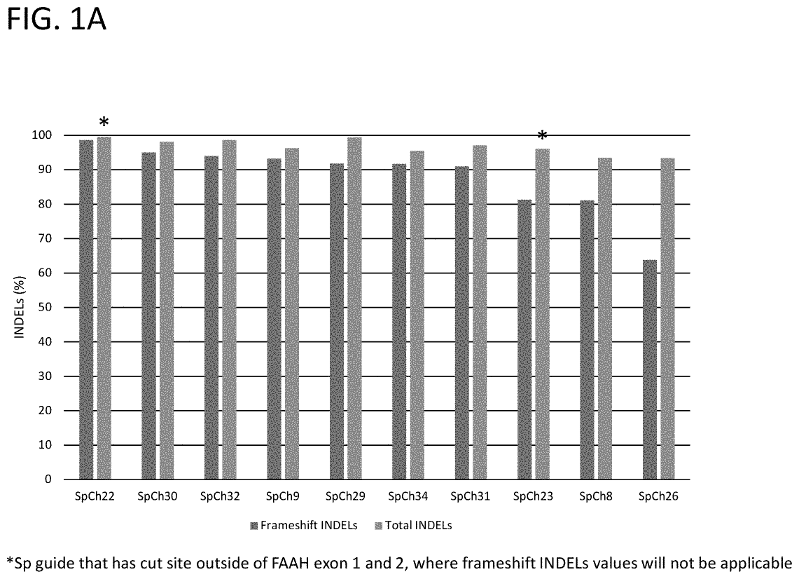

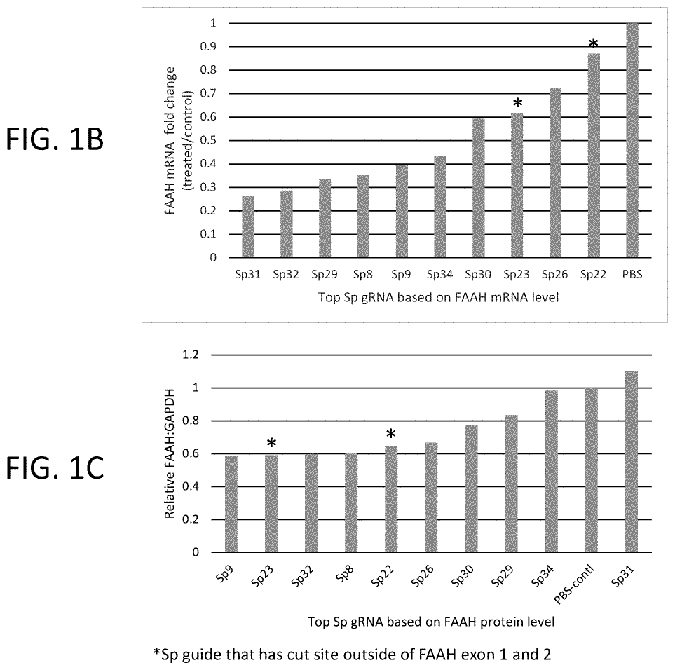

In some embodiments, the target sequence is adjacent to a PAM recognized by a Cas nuclease (e.g., Cas9 nuclease) described herein. In some embodiments, 3′ end of the target sequence is adjacent to or within 1, 2, or 3 nucleotide of the PAM. The length and the sequence of the PAM depends on the Cas9 nuclease used. For example, in some embodiments, the PAM is selected from a consensus PAM sequence or a particular PAM sequence recognized by a specific Cas9 nuclease, including those disclosed in FIG. 1 of Ran et al., (2015) Nature, 520:186-191 (2015), which is incorporated herein by reference.

In some embodiments, the PAM comprises 2, 3, 4, 5, 6, 7, 8, 9, or 10 nucleotides in length. Non-limiting exemplary PAM sequences include NGG (SpCas9 WT, SpCas9 nickase, dimeric dCas9-Fok1, SpCas9-HF1, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT or NNGRRN (SaCas9), NNNRRT (KKH SaCas9), NNNNRYAC (CjCas9), NNAGAAW (St1Cas9), NAAAAC (TdCas9), NGGNG (St3Cas9), NG (FnCas9), NAAAAN (TdCas9), NNAAAAW (StCas9), NNNNACA (CjCas9), GNNNCNNA (PmCas9), NNGG (SluCas9), and NNNNGATT (NmCas9) (see e.g., Cong et al., (2013) Science 339:819-823; Kleinstiver et al., (2015) Nat Biotechnol 33:1293-1298; Kleinstiver et al., (2015) Nature 523:481-485; Kleinstiver et al., (2016) Nature 529:490-495; Tsai et al., (2014) Nat Biotechnol 32:569-576; Slaymaker et al., (2016) Science 351:84-88; Anders et al., (2016) Mol Cell 61:895-902; Kim et al., (2017) Nat Comm 8:14500; Fonfara et al., (2013) Nucleic Acids Res 42:2577-2590; Garneau et al., (2010) Nature 468:67-71; Magadan et al., (2012) PLoS ONE 7:e40913; Esvelt et al., (2013) Nat Methods 10(11):1116-1121 (wherein N is defined as any nucleotide, W is defined as either A or T, R is defined as a purine (A) or (G), and Y is defined as a pyrimidine (C) or (T)).

In some embodiments, the PAM sequence is NGG. In some embodiments, the PAM sequence is NNGG. In some embodiments, the PAM is NNGRRT.

In some embodiments, the nucleotide sequence of the target sequence and the PAM comprises the formula 5′ N₁₉₋₂₁-N-G-G-3′ (SEQ ID NO: 1282), wherein N is any nucleotide, and wherein the three 3′ terminal nucleic acids, N-G-G represent the SpCas9 PAM. In some embodiments, the nucleotide sequence of the target sequence and the PAM comprises the formula 5′ N₁₉₋₂₂-N-N-G-G-3′ (SEQ ID NO: 1283), wherein N is any nucleotide, and wherein the four 3′ terminal nucleic acids, N-N-G-G represent the SluCas9 PAM. In some embodiments, the nucleotide sequence of the target sequence and the PAM comprises the formula 5′ N₁₉₋₂₂-N-N-G-R-R-T-3′ (SEQ ID NO: 1284), wherein N is any nucleotide, and wherein R is a nucleotide comprising the nucleobase adenine (A) or guanine (G), and wherein the six 3′ terminal nucleic acids, N-N-G-R-R-T represent the SaCas9 PAM.

In some embodiments, a target sequence that perfectly hybridizes with the gRNA spacer sequence occurs only once in a given eukaryotic genomes. In some embodiments, the genome comprises additional sequences that imperfectly hybridize with the gRNA spacer sequence, for example, sequences having one or more mismatches (e.g., 1, 2, 3, 4, or 5 mismatches) and/or bulges, relative to the gRNA spacer sequence. In some embodiments, the genome comprises sequences that hybridize the gRNA spacer sequence that are adjacent a PAM sequence having at least one mismatch relative to the canonical PAM sequence. Such genomic sequences (e.g., target sequences that imperfectly hybridize the gRNA spacer sequence or target sequences comprising a non-canonical PAM sequences) are referred to herein as off-target sites.

In some embodiments, the a method of in silico screening is used to predict cleavage efficiency of a gRNA spacer sequence at both on-target and off-target sites, thereby allowing selection of a gRNA with high cleavage efficiency at a target sequence in the genome comprising a target gene (e.g., sufficient to achieve a desired genomic edit of FAAH and/or FAAH-OUT), with low or minimal cutting efficiency at off-target sites in the genome (i.e., low or minimal frequency of DNA DSBs occurring at sites other than the selected target sequence).

As described herein, selection of gRNAs with a favorable off-target profile is critical for use in a therapeutic method of the disclosure, for example, to eliminate or reduce the risk of undesirable chromosomal rearrangements or off-target mutations. In some embodiments, a favorable off-target profile in one that minimizes or eliminates the number of off-target sites and/or the frequency of cutting at these sites. In some embodiments, a favorable off-target profile is one that minimizes or eliminates off-target sites in specific regions of the genome, for example within or proximal to an oncogene.

As is known in the art, the occurrence of off-target activity can be influenced by a number of factors including similarities and dissimilarities between the target site and various off-target sites, as well as the particular endonuclease used. For example, the ability of a given gRNA to promote cleavage at a target sequence in a genomic DNA molecule relates to, for example, the accessibility of the target sequence, which depends on one or more factors that include the chromatin structure of the genomic DNA molecule and/or proximity to transcription factor binding sites. For example, target sequences located within a region of the genomic DNA molecule having a high condensed chromatin structure are less accessible than target sequences located within a region of the genomic DNA molecule having an open chromatin structure. As a further example, target sequences proximal to a region of the genomic DNA molecule bound by a transcription factor or other regulatory protein may be less accessible than target sequences proximal a region of the genomic DNA molecule that is unbound by regulatory proteins. Moreover, the cell state and type of cell may influence the accessibility of target sequences, for example, by influencing the chromatin structure of genomic DNA.

In some embodiments, the nucleotide sequence of the spacer is designed or chosen using an algorithm or method known in the art. In some embodiments, the algorithm uses variables to screen for suitable gRNA spacer sequences and corresponding target sequences. Non-limiting examples of such variables include predicted melting temperature of the gRNA sequence, secondary structure formation of the gRNA sequence, predicted annealing temperature of the gRNA sequence, sequence identity, genomic context of the target sequence, chromatin accessibility of the target sequence, % GC, frequency of genomic occurrence of the target sequence (e.g., of sequences that are identical or are similar but vary in one or more spots as a result of mismatch, insertion or deletion), methylation status of the target sequence, and/or presence of SNPs within the target sequence.

In some embodiments, one or more bioinformatics tools known in the art are used to predict the off-target activity of a gRNA spacer sequence and/or identify the most likely sites of off-target activity. Non-limiting examples of bioinformatics tools for use in the present disclosure include CCTop, CRISPOR, and COSMID.

In some embodiments, identification of gRNA target sequences is best achieved through a combination of in silico selection and experimental evaluation. Experimental methods to evaluate, for example, gRNA on-target and off-target cleavage efficiency are known in the art and further described herein.

In some embodiments, cleavage efficiency is measured as frequency of INDELs proximal the target sequence targeted by the gRNA spacer sequence. Methods to measure frequency of INDELs at a particular target sequence in a genome are known in the art. An exemplary method to measure frequency of INDELs at a predicted cut site in a given target sequence comprises, (i) isolation of genomic DNA from the edited cell population and/or tissue, (ii) amplification of the DNA region comprising the target sequence (e.g., by PCR), (iii) sequencing of the amplified DNA region (e.g., by Sanger sequencing), and (iv) determining frequency of INDELs at the predicted cut site by Tracking of Indels decomposition (TIDE) assay, for example, as described by Brinkman, et al (2014) NUCLEIC ACIDS RESEARCH 42:e168. A further exemplary method comprises sequencing of the amplified DNA region by next-generation sequencing (NGS) and analysis of INDEL frequency at the predicted cut site in the target sequence, for example, as described by Bell et al (2014) BMC Genomics 15:1002.

In some embodiments, cleavage efficiency is measured as the frequency of total sequence reads having an INDEL of at least ±1 nt (e.g, ±1 nt, ±2 nt, ±3 nt, ±4 nt, ±5 nt, ±6 nt, ±7 nt, ±8 nt, or ±9 nt). In some embodiments, a gRNA is selected having cleavage efficiency within a desired target sequence (e.g., target sequence within or proximal the FAAH coding sequence; e.g., a target sequence within or proximal FAAH-OUT) of at least 15%, 20%, 25%, 30%, 35%, 40%, 45%, 50% or higher. In some embodiments, a gRNA is selected having cleavage efficiency of at least 15%. In some embodiments, a gRNA is selected having cleavage efficiency of at least 20%. In some embodiments, a gRNA is selected having cleavage efficiency of at least 25%. In some embodiments, a gRNA is selected having cleavage efficiency of at least 30%. In some embodiments, a gRNA is selected having cleavage efficiency of at least 35%. In some embodiments, a gRNA is selected having cleavage efficiency of at least 40%. In some embodiments, a gRNA is selected having cleavage efficiency of at least 45%. In some embodiments, a gRNA is selected having cleavage efficiency of at least 50%. In some embodiments, a gRNA is selected having cleavage efficiency of at least 55%. In some embodiments, a gRNA is selected having cleavage efficiency of at least 60%. In some embodiments, a gRNA is selected having cleavage efficiency of at least 65%. In some embodiments, a gRNA is selected having cleavage efficiency of at least 70%. In some embodiments, a gRNA is selected having cleavage efficiency of at least 75%. In some embodiments, a gRNA is selected having cleavage efficiency of at least 80%. In some embodiments, a gRNA is selected having cleavage efficiency of at least 85%. In some embodiments, a gRNA is selected having cleavage efficiency of at least 90% or higher. In some embodiments, cleavage efficiency is measured using TIDE analysis as described herein.

(iv) gRNA Components

A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g., a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA:tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA provided by the disclosure comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the tracrRNA may comprise all or a portion of a wild-type tracrRNA sequence from a naturally-occurring CRISPR/Cas system. In some embodiments, the tracrRNA may comprise a truncated or modified variant of the wild-type tracr RNA. The length of the tracr RNA may depend on the CRISPR/Cas system used. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA may comprise certain secondary structures, such as, e.g., one or more hairpins or stem-loop structures, or one or more bulge structures.

Single Guide RNA (sgRNA)

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

The sgRNA can comprise a 20 nucleotide spacer sequence at the 5′ end of the sgRNA sequence. The sgRNA can comprise a less than 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence as set forth by SEQ ID NOs: 1285, 1286, and 1287.

The sgRNA can comprise no uracil at the 3′ end of the sgRNA sequence. The sgRNA can comprise one or more uracil at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

In some embodiments, the sgRNA comprises unmodified or modified nucleotides. For example, in some embodiments, the sgRNA comprises one or more 2′-O-methyl phosphorothioate nucleotides.

Spacers

In some embodiments, the gRNAs provided by the disclosure comprise a spacer sequence. A spacer sequence is a sequence that defines the target site of a target nucleic acid (e.g.: DNA). The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. In some embodiments, a spacer sequence corresponding to a target sequence adjacent to a PAM sequence is complementary to the non-PAM strand of the target nucleic acid. Thus, in some embodiments, a spacer sequence which corresponds to a target sequence adjacent to a PAM sequence is identical to the PAM strand. The gRNA spacer sequence hybridizes to the complementary strand (e.g.: the non-PAM strand of the target nucleic acid/target site). In some embodiments, the spacer is sufficiently complementary to the complementary strand of the target sequence (e.g.: non-PAM strand), as to target a Cas nuclease to the target nucleic acid. In some embodiments, the spacer is at least 80%, 85%, 90% or 95% complementary to the non-PAM strand of the target nucleic acid. In some embodiments, the spacer is 100% complementary to the non-PAM strand of the target nucleic acid. In some embodiments, the spacer comprises 1, 2, 3, 4, 5, 6 or more nucleotides that are not complementary with the non-PAM strand of the target nucleic acid. In some embodiments, the spacer comprises 1 nucleotide that is not complementary with the non-PAM strand of the target nucleic acid. In some embodiments, the spacer comprises 2 nucleotides that are not complementary with the non-PAM strand of the target nucleic acid.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the spacer comprises at least one or more modified nucleotide(s) such as those described herein. In some embodiments, the disclosure provides gRNA molecules comprising a spacer which comprise the nucleobase uracil (U), while any DNA encoding a gRNA comprising a spacer comprising the nucleobase uracil (U) will comprise the nucleobase thymine (T) in the corresponding position(s).

### (v) Methods of Making Guide RNAs

The gRNAs of the present disclosure are produced by a suitable means available in the art, including but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some aspects, non-natural modified nucleobases are introduced into polynucleotides, e.g., gRNA, during synthesis or post-synthesis. In certain embodiments, modifications are on internucleoside linkages, purine or pyrimidine bases, or sugar. In particular embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some aspects, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

Certain embodiments of the invention also provide nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, the gRNAs provided by the disclosure are synthesized by enzymatic methods (e.g., in vitro transcription, IVT).

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### B. Cas Nuclease

In some embodiments, the disclosure provides compositions and systems (e.g., an engineered CRISPR/Cas system) comprising a site-directed nuclease, wherein the site-directed nuclease is a Cas nuclease. The Cas nuclease may comprise at least one domain that interacts with a guide RNA (gRNA). Additionally, the Cas nuclease are directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the CRISPR/Cas system comprise components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9, and contains a RuvC-like nuclease domain.

In some embodiments, the Cas nuclease are from a Type-II CRISPR/Cas system (e.g., a Cas9 protein from a CRISPR/Cas9 system). In some embodiments, the Cas nuclease are from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptoccoccus lugdunensis, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein is from S. lugdunensis (SluCas9). In some embodiments, the Cas9 protein are from Staphylococcus aureus (SaCas9). In some embodiments, a suitable Cas9 protein for use in the present disclosure is any disclosed in WO2019/183150 and WO2019/118935, each of which is incorporate herein by reference.

In some embodiments, a suitable Cas9 nuclease for use in the present disclosure is a wild-type SpCas9 nuclease. The terms “wild-type SpCas9 nuclease” and “wild-type SpCas9” refer to a polypeptide having the amino acid sequence of SEQ ID NO: 1268 that forms an active CRISPR/Cas endonuclease system when combined with a suitable gRNA molecule (e.g., a sgRNA molecule comprising the nucleotide sequence set forth by SEQ ID NO: 1267), wherein the system cleaves a genomic DNA molecule proximal a target sequence comprising a SpCas9 PAM sequence (e.g., NGG) that is targeted by the gRNA molecule. In some embodiments, a suitable Cas9 nuclease for use in the present disclosure is a functional derivative of SpCas9 nuclease. In some embodiments, a functional derivative of SpCas9 nuclease for use in the present disclosure is any variant of wild-type SpCas9 nuclease having equivalent or similar functional properties. For example, a functional derivative of SpCas9 is any variant of wild-type SpCas9 that combines with a suitable gRNA molecule (e.g., a sgRNA molecule comprising the nucleotide sequence set forth by SEQ ID NO: 1267) in a cell to cleave a genomic DNA molecule proximal a target sequence comprising a SpCas9 PAM sequence (e.g., NGG) that is targeted by the gRNA molecule. In some embodiments, the functional derivative of SpCas9 nuclease has substantial sequence homology with wild-type SpCas9 (e.g., at least about 80%, about 85%, about 90%, about 95%, about 96%, about 97%, about 98%, or about 99%). In some embodiments, the functional derivative of SpCas9 nuclease has substantially equivalent cleavage efficiency (e.g., as measured by frequency of INDELs at a target site directed by the gRNA) relative to wild-type SpCas9. In some embodiments, a functional derivative of SpCas9 nuclease comprises one or more mutations relative to wild-type SpCas9 that result in increased cleavage efficiency (e.g., as measured by frequency of INDELs at a target site directed by the gRNA) relative to wild-type SpCas9. In some embodiments, a functional derivative of SpCas9 nuclease comprises one or more mutations relative to wild-type SpCas9 that result in increased fidelity, as further described herein. In some embodiments, a functional derivative of SpCas9 nuclease comprises one or more mutations relative to wild-type SpCas9 that result in recognition of a PAM sequence other than the canonical SpCas9 PAM (i.e., NGG). In some embodiments, a functional derivative of SpCas9 nuclease has one or more nuclease domains replaced with a nuclease domain from another site-directed endonuclease (e.g., Cas9 nuclease) relative to wild-type SpCas9. In some embodiments, a functional derivative of SpCas9 is a modified nuclease (e.g., a modified nuclease comprising a nuclear localization domain) relative to wild-type SpCas9, as further described herein.

In some embodiments, a suitable Cas9 nuclease for use in the present disclosure is a wild-type SluCas9 nuclease. The terms “wild-type SluCas9 nuclease” and “wild-type SluCas9” refer to a polypeptide having the amino acid sequence of SEQ ID NO: 1270 that forms an active CRISPR/Cas endonuclease system when combined with a suitable gRNA molecule (e.g., a sgRNA molecule comprising the nucleotide sequence set forth by SEQ ID NO: 1269), wherein the system cleaves a genomic DNA molecule proximal a target sequence comprising a SluCas9 PAM sequence (e.g., NNGG) that is targeted by the gRNA molecule. In some embodiments, a suitable Cas9 nuclease for use in the present disclosure is a functional derivative of SluCas9 nuclease. In some embodiments, a functional derivative of SluCas9 nuclease for use in the present disclosure is any variant of wild-type SluCas9 nuclease having equivalent or similar functional properties. For example, a functional derivative of SluCas9 is any variant of wild-type SluCas9 that combines with a suitable gRNA molecule (e.g., a sgRNA molecule comprising the nucleotide sequence set forth by SEQ ID NO: 1269) in a cell to cleave a genomic DNA molecule proximal a target sequence comprising a SluCas9 PAM sequence (e.g., NNGG) that is targeted by the gRNA molecule. In some embodiments, the functional derivative of SluCas9 nuclease has substantial sequence homology with wild-type SluCas9 (e.g., at least about 80%, about 85%, about 90%, about 95%, about 96%, about 97%, about 98%, or about 99%). In some embodiments, the functional derivative of SluCas9 nuclease has substantially equivalent cleavage efficiency (e.g., as measured by frequency of INDELs at a target site directed by the gRNA) to wild-type SluCas9. In some embodiments, a functional derivative of SluCas9 nuclease comprises one or more mutations relative to wild-type SluCas9 that result in increased cleavage efficiency (e.g., as measured by frequency of INDELs at a target site directed by the gRNA) relative to wild-type SluCas9. In some embodiments, a functional derivative of SluCas9 nuclease comprises one or more mutations relative to wild-type SluCas9 that result in increased fidelity, as further described herein. In some embodiments, a functional derivative of SluCas9 nuclease comprises one or more mutations relative to wild-type SluCas9 that result in recognition of a PAM sequence other than the canonical SluCas9 PAM (i.e., NNGG). In some embodiments, a functional derivative of SluCas9 nuclease has one or more nuclease domains replaced with a nuclease domain from another site-directed endonuclease (e.g., Cas9 nuclease) relative to wild-type SluCas9. In some embodiments, a functional derivative of SluCas9 is a modified nuclease (e.g., a modified nuclease comprising a nuclear localization domain) relative to wild-type SluCas9, as further described herein.

In some embodiments, a suitable Cas9 nuclease for use in the present disclosure is a wild-type SaCas9 nuclease. The terms “wild-type SaCas9 nuclease” and “wild-type SaCas9” refer to a polypeptide having the amino acid sequence of SEQ ID NO: 1272 that forms an active CRISPR/Cas endonuclease system when combined with a suitable gRNA molecule (e.g., a sgRNA molecule comprising the nucleotide sequence set forth by SEQ ID NO: 1271), wherein the system cleaves a genomic DNA molecule proximal a target sequence comprising a SaCas9 PAM sequence (e.g., NNGRRT) that is targeted by the gRNA molecule. In some embodiments, a suitable Cas9 nuclease for use in the present disclosure is a functional derivative of SaCas9 nuclease. In some embodiments, a functional derivative of SaCas9 nuclease for use in the present disclosure is any variant of wild-type SaCas9 nuclease having equivalent or similar functional properties. For example, a functional derivative of SaCas9 is any variant of wild-type SaCas9 that combines with a suitable gRNA molecule (e.g., a sgRNA molecule comprising the nucleotide sequence set forth by SEQ ID NO: 1271) in a cell to cleave a genomic DNA molecule proximal a target sequence comprising a SaCas9 PAM sequence (e.g., NNGRRT) that is targeted by the gRNA molecule. In some embodiments, the functional derivative of SaCas9 nuclease has substantial sequence homology with wild-type SaCas9 (e.g., at least about 80%, about 85%, about 90%, about 95%, about 96%, about 97%, about 98%, or about 99%). In some embodiments, the functional derivative of SaCas9 nuclease has substantially equivalent cleavage efficiency (e.g., as measured by frequency of INDELs at a target site directed by the gRNA) to wild-type SaCas9. In some embodiments, a functional derivative of SaCas9 nuclease comprises one or more mutations relative to wild-type SaCas9 that result in increased cleavage efficiency (e.g., as measured by frequency of INDELs at a target site directed by the gRNA) relative to wild-type SaCas9. In some embodiments, a functional derivative of SaCas9 nuclease comprises one or more mutations relative to wild-type SaCas9 that result in increased fidelity, as further described herein. In some embodiments, a functional derivative of SaCas9 nuclease comprises one or more mutations relative to wild-type SaCas9 that result in recognition of a PAM sequence other than the canonical SaCas9 PAM (i.e., NNGRRT). In some embodiments, a functional derivative of SaCas9 nuclease has one or more nuclease domains replaced with a nuclease domain from another site-directed endonuclease (e.g., Cas9 nuclease) relative to wild-type SaCas9. In some embodiments, a functional derivative of SaCas9 is a modified nuclease (e.g., a modified nuclease comprising a nuclear localization domain) relative to wild-type SaCas9, as further described herein.

In some embodiments, a Cas nuclease comprises more than one nuclease domain. For example, in some embodiments, the Cas9 nuclease comprises at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain Exemplary amino acid substitutions in the RuvC-like nuclease domain include D10A (based on the S. pyogenes Cas9 nuclease). In some embodiments, the nickase comprises an amino acid substitution in the HNH-like nuclease domain Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fok1. A Cas9 nuclease is a modified nuclease.

In alternative embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

### (i) High Fidelity Variants of Cas Nucleases

In some embodiments, the disclosure provides a CRISPR/Cas system comprising a Cas nuclease engineered for increased fidelity. As used herein, the term “fidelity” when used in reference to a CRISPR/Cas system comprising a Cas nuclease and gRNA refers to the specificity of the system for a target site in a DNA molecule (e.g., genomic DNA molecule) that is homologous (e.g., perfect match) to the gRNA spacer sequence. In some embodiments, a CRISPR/Cas system with increased fidelity has reduced activity at off-target sites in the DNA molecule, i.e., sites that are an imperfect match to the gRNA spacer sequence.

In some embodiments, a CRISPR/Cas system of the disclosure comprises a Cas variant (e.g., a SpCas9 functional derivative, a SluCas9 functional derivative, a SaCas9 functional derivative) comprising one or more mutations for increased fidelity. In some embodiments, the one or more mutations result in reduced activity of the CRISPR/Cas system at off-target sites in the DNA molecule, for example, compared to a system comprising an unmodified version of the Cas nuclease (e.g., wild-type SpCas9 nuclease, wild-type SluCas9 nuclease, wild-type SaCas9 nuclease). In some embodiments, the CRISPR/Cas system has substantially equivalent activity for inducing cleavage at an on-target site in the DNA molecule, for example, as compared to the system comprising an unmodified version of the Cas nuclease.

Methods of making Cas variants with increased fidelity are known in the art. For example, in some embodiments, a method of structure-guided engineering is used to make a Cas variant with increased fidelity.

In some embodiments, a CRISPR/Cas system described herein comprises a Cas9 nuclease comprising one or more mutations for increased fidelity. In some embodiments, the Cas9 nuclease is derived from S. pyogenes, wherein the Cas nuclease comprises one or more mutations relative to wild-type SpCas9 for increased fidelity. In some embodiments, the Cas9 nuclease is derived from S. aureus, wherein the Cas nuclease comprises one or more mutations relative to wild-type SaCas9 for increased fidelity. In some embodiments, the Cas9 nuclease is derived from S. lugdunensis, wherein the Cas nuclease comprises one or more mutations relative to wild-type SluCas9 for increased fidelity.

A suitable Cas9 nuclease with increased fidelity for use in the present disclosure includes any one described US2019/0010471; US2018/0142222; U.S. Pat. No. 9,944,912; WO2020/057481; US2019/0177710; US2018/0100148; U.S. Pat. No. 10,526,591; and US20200149020; each of which is incorporated herein by reference in their entirety.

In some embodiments, a Cas nuclease engineered for increased fidelity reduces cleavage of one or more predicted off-target sites by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 115%, at least about 120%, at least about 125%, at least about 30%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%, relative to a Cas nuclease not engineered for increased fidelity (e.g. wild-type Cas nuclease). In some embodiments, a Cas nuclease engineered for increased fidelity reduces cleavage of one or more predicted off-target sites by about 10% to about 200%, about 20% to about 190%, about 30% to about 180%, about 40% to about 170%, about 50% to about 160%, about 60% to about 150%, about 70% to about 140%, about 80% to about 130%, about 90% to about 120%, about 100% to about 110%, relative to a Cas nuclease not engineered for increased fidelity (e.g. wild-type Cas nuclease).

In some embodiments, cleavage of an off-target or on-target site is determined based on the percentage of INDELs. In some embodiments, the percentage of INDELs generated at one or more off-target sites by a Cas nuclease engineered for increased fidelity is decreased relative to the percentage of INDELs generated by a Cas nuclease not engineered for increased fidelity (e.g., wild-type Cas nuclease).

In some embodiments, a Cas nuclease engineered for increased fidelity maintains the same level of cleavage at the on-target site, and reduces the cleavage of one or more predicted off-target sites compared to a Cas nuclease not engineered for increased fidelity (e.g., wild-type Cas nuclease).

### C. Exemplary CRISPR/Cas Systems for Gene Editing of FAAH-OUT

In some embodiments, the disclosure provides a system for use with a NNGG PAM for introducing a deletion in a genomic DNA molecule comprising at least a portion of FAAH-OUT, wherein the system comprises dual gRNAs and a site-directed endonuclease that recognizes an NNGG PAM. In some embodiments, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof. For example, in some embodiments, a functional derivative of SluCas9 endonuclease is one engineered for increased fidelity. In some embodiments, the deletion introduced is approximately 2-8 kb, approximately 2-7 kb, approximately 2-6 kb, approximately 2-5 kb, approximately 2-4 kb, approximately 3-8 kb, approximately 3-7 kb, approximately 3-6 kb, approximately 3-5 kb, approximately 4-8 kb, approximately 4-7 kb, approximately 4-6 kb, approximately 5-8 kb, or approximately 5-7 kb in length. In some embodiments, the deletion comprises a full or partial removal of FOP. In some embodiments, the deletion comprises a full or partial removal of FOC.

In some embodiments, the dual gRNAs of the system for use with a NNGG PAM comprise a first gRNA molecule. In some embodiments, the first gRNA molecule comprises a spacer sequence corresponding to a first target sequence, wherein the first target sequence is adjacent an NNGG PAM, and wherein the first target sequence is downstream the 3′ terminus of FAAH and upstream a transcriptional start site of FAAH-OUT. In some embodiments, the first target sequence is within a region of the genomic DNA molecule that is: (i) at least about 5.5 kb, about 6 kb, about 6.5 kb, about 7 kb, about 7.5 kb, about 8 kb, about 8.5 kb, about 9 kb, or about 9.5 kb downstream the 3′ terminus of FAAH; (ii) within a region of the genomic DNA molecule that is at least about 200 bp, about 300 bp, about 400 bp, about 500 bp, about 600 bp, about 700 bp, about 800 bp, about 900 bp, about 1 kb, about 2 kb, about 3 kb, or about 4 kb upstream the transcriptional start site of FAAH-OUT; (iii) within a region of the genomic DNA molecule between about 46,418,846 to about 46,422,883 of chromosome 1, according to human reference genome Hg38; or (iv) a combination of (i)-(iii).

In some embodiments, the dual gRNAs of the system for use with a NNGG PAM comprise a second gRNA molecule. In some embodiments, the second gRNA molecule comprises a spacer sequence corresponding to a second target sequence, wherein the second target sequence is adjacent an NNGG PAM, and wherein the second target sequence is downstream of the FAAH-OUT transcriptional start site and upstream an exon 3 of FAAH-OUT. In some embodiments, the second target sequence is (i) within a region of the genomic DNA molecule that is about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, or about 3.3 kb downstream the transcriptional start site of FAAH-OUT; (ii) within a region of the genomic DNA molecule that is about 5.8 kb, about 5.9 kb, about 6 kb, about 6.1 kb, about 6.2 kb, about 6.3 kb, about 6.4 kb, about 6.5 kb, about 6.6 kb, about 6.7 kb, about 6.8 kb, about 6.9 kb, about 7 kb, about 7.1 kb, about 7.2 kb, or about 7.3 kb upstream the 5′ end of exon 3 of FAAH-OUT; (iii) within a region of the genomic DNA molecule between about 46,424,697 to about 46,426,377 of chromosome 1, according to human reference genome Hg38; or (iv) a combination of (i)-(iii).

In some embodiments, the first gRNA of the system for use with a NNGG PAM, when introduced into a cell with the site-directed endonuclease that recognizes the NNGG PAM, combines with the site-directed endonuclease to induce cleavage proximal the first target sequence with a cleavage efficiency of at least 30%. In some embodiments, cleavage efficiency is measured as the frequency of INDELs induced proximal the target sequence (e.g., by TIDE analysis). In some embodiments, the cleavage efficiency is at least 30%, 35%, 40%, 45%, 50%, 55%, 60%, 65%, 70%, 75%, 80% or higher.

In some embodiments, the second gRNA of the system for use with a NNGG PAM, when introduced into a cell with the site-directed endonuclease that recognizes the NNGG PAM, combines with the site-directed endonuclease to induce cleavage proximal the first target sequence with a cleavage efficiency of at least 30%. In some embodiments, cleavage efficiency is measured as the frequency of INDELs induced proximal the target sequence (e.g., by TIDE analysis). In some embodiments, the cleavage efficiency is at least 30%, 35%, 40%, 45%, 50%, 55%, 60%, 65%, 70%, 75%, 80% or higher.

In some embodiments, the disclosure provides a system for use with a NNGG PAM comprising:

(i) a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 564 or SEQ ID NO: 579; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 692, 702, 705, 709, 712, and 723, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to SEQ ID NO: 750 or SEQ ID NO: 765. In some embodiments, the second spacer sequence comprises a nucleotide sequence having up to 1, 2, or 3 nucleotide deletions or substitutions relative to any one of SEQ ID NOs: 878, 888, 891, 895, 898, and 909.

In some embodiments, the disclosure provides a system for use with a NNGG PAM comprising:

(i) a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 564 or SEQ ID NO: 579; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 629, 630, 644, and 676, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a partial removal of the FOC region. In some embodiments, the first spacer sequence comprises a nucleotide sequence having up to 1, 2, or 3 nucleotide deletions or substitutions relative to SEQ ID NO: 750 or SEQ ID NO: 765. In some embodiments, the second spacer sequence comprises a nucleotide sequence having up to 1, 2, or 3 nucleotide deletions or substitutions relative to any one of SEQ ID NOs: 815, 816, 830, and 862.

In some embodiments, the disclosure provides a system for use with a NNGG PAM comprising:

(i) a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 615 or SEQ ID NO: 621; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 692, 702, 705, 709, 712, 723, wherein when the system is introduced to the cell, the first gRNA and second gRNA combine with the site-directed endonuclease to induce cleavage proximal the first and second target sequences, to introduce an approximately 2-5.5 kb deletion in the genomic DNA molecule resulting in a partial removal of FOP and a full removal of the FOC region. In some embodiments, the first spacer sequence comprises a nucleotide sequence having up to 1, 2, or 3 nucleotide deletions or substitutions relative to SEQ ID NO: 801 or SEQ ID NO: 807. In some embodiments, the second spacer sequence comprises a nucleotide sequence having up to 1, 2, or 3 nucleotide deletions or substitutions relative to any one of SEQ ID NOs: 878, 888, 891, 895, 898, and 909.

In some embodiments, the disclosure provides a system for use with a NNGG PAM comprising:

(i) a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 615 or SEQ ID NO: 621; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 629, 630, 644, and 676, wherein when the system is introduced to the cell, the first gRNA and second gRNA combine with the site-directed endonuclease to induce cleavage proximal the first and second target sequences, to introduce an approximately 2-5.5 kb deletion in the genomic DNA molecule resulting in a partial removal of FOP and a partial removal of the FOC region. In some embodiments, the first spacer sequence comprises a nucleotide sequence having up to 1, 2, or 3 nucleotide deletions or substitutions relative to SEQ ID NO: 801 or SEQ ID NO: 807. In some embodiments, the second spacer sequence comprises a nucleotide sequence having up to 1, 2, or 3 nucleotide deletions or substitutions relative to any one of SEQ ID NOs: 815, 816, 830, and 862.

In some embodiments, the disclosure provides a system for use with an NGG PAM for introducing a deletion in a genomic DNA molecule comprising at least a portion of FAAH-OUT, wherein the system comprises dual gRNAs and a site-directed endonuclease that recognizes an NGG PAM. In some embodiments, the site-directed endonuclease is a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof. For example, in some embodiments, a functional derivative of SpCas9 endonuclease is one engineered for increased fidelity. In some embodiments, the deletion introduced is approximately 3-10 kb, approximately 3-9 kb, approximately 3-8 kb, approximately 3-7 kb, approximately 3-6 kb, approximately 3-5 kb, approximately 4-10 kb, approximately 4-9 kb, approximately 4-8 kb, approximately 4-7 kb, approximately 4-6 kb, approximately 5-10 kb, approximately 5-9 kb, approximately 5-8 kb, approximately 5-7 kb, approximately 6-10 kb, approximately 6-9 kb, approximately 6-8 kb, or approximately 8-10 kb in length. In some embodiments, the deletion comprises a removal of FOP. In some embodiments, the deletion comprises a full or partial removal of FOC.

In some embodiments, the dual gRNAs of the system for use with an NGG PAM comprise a first gRNA molecule. In some embodiments, the first gRNA molecule comprises a spacer sequence corresponding to a first target sequence, wherein the first target sequence is adjacent an NGG PAM, and wherein the first target sequence is downstream the 3′ terminus of FAAH and upstream a transcriptional start site of FAAH-OUT. In some embodiments, the first target sequence is: (i) within a region of the genomic DNA molecule that is at least about 4.5 kb, about 5 kb, about 5.5 kb, about 6 kb, about 6.5 kb, about 7 kb, about 7.5 kb, about 7.5 kb, or about 8 kb downstream the 3′ terminus of FAAH; (ii) within a region of the genomic DNA molecule that is at least about 1.5 kb, about 2 kb, about 2.5 kb, about 3 kb, about 3.5, about 4 kb, about 4.5 kb, or about 5 kb upstream the transcriptional start site of FAAH-OUT; (iii) within a region of the genomic DNA molecule between about 46,418,391 to about 46,421,122 of chromosome 1, according to human reference genome Hg38; or (iv) a combination of (i)-(iii).

In some embodiments, the dual gRNAs of the system for use with an NGG PAM comprise a second gRNA molecule. In some embodiments, the second gRNA molecule comprises a spacer sequence corresponding to a second target sequence, wherein the second target sequence is adjacent an NGG PAM, and wherein the second target sequence is downstream of the FAAH-OUT transcriptional start site and upstream an exon 3 of FAAH-OUT. In some embodiments, the second target sequence is (i) within a region of the genomic DNA molecule that is at least about 1.8 kb, about 1.9 kb, about 2 kb, about 2.5 kb, about 3 kb, about 3.5 kb, about 4 kb, about 4.5 kb, about 5 kb, or about 5.5 kb downstream the transcriptional start site of FAAH-OUT; (ii) within a region of the genomic DNA molecule that is at least about 3.5 k, about 4 kb, about 4.5 kb, about 5 kb, about 5.5 kb, about 6 kb, about 6.5 kb, about 7 kb, or about 7.5 kb upstream the 5′ end of exon 3 of FAAH-OUT; (iii) within a region of the genomic DNA molecule between about 46,424,651 to about 46,428,274 of chromosome 1, according to human reference genome Hg38; or (iv) a combination of (i)-(iii).

In some embodiments, the first gRNA of the system for use with an NGG PAM, when introduced into a cell with the site-directed endonuclease that recognizes the NGG PAM, combines with the site-directed endonuclease to induce cleavage proximal the first target sequence with a cleavage efficiency of at least 30%. In some embodiments, cleavage efficiency is measured as the frequency of INDELs induced proximal the target sequence (e.g., by TIDE analysis). In some embodiments, the cleavage efficiency is at least 30%, 35%, 40%, 45%, 50%, 55%, 60%, 65%, 70%, 75%, 80%, 85% or higher.

In some embodiments, the second gRNA of the system for use with a NGG PAM, when introduced into a cell with the site-directed endonuclease that recognizes the NGG PAM, combines with the site-directed endonuclease to induce cleavage proximal the first target sequence with a cleavage efficiency of at least 30%. In some embodiments, cleavage efficiency is measured as the frequency of INDELs induced proximal the target sequence (e.g., by TIDE analysis). In some embodiments, the cleavage efficiency is at least 30%, 35%, 40%, 45%, 50%, 55%, 60%, 65%, 70%, 75%, 80%, 85% or higher.

In some embodiments, the disclosure provides a system for use with a NGG PAM comprising:

(i) a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 189, 193, and 221; and

(iii) a second gRNA molecule targeting a target site in the genomic DNA molecule, the second gRNA comprising a second spacer sequence corresponding to a second target sequence consisting of a nucleotide sequence selected from SEQ ID NO: 365, wherein when the system is introduced to the cell, the first gRNA and second gRNA combine with the site-directed endonuclease to induce cleavage proximal the first and second target sequences, to introduce an approximately 8-10 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to SEQ ID NOs: 374, 378, and 406. In some embodiments, the second spacer sequence comprises a nucleotide sequence having up to 1, 2, or 3 nucleotide deletions or substitutions relative to any one of SEQ ID NO: 550.

In some embodiments, the disclosure provides a system for use with a NGG PAM comprising:

(i) a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 189, 193, and 221; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 348, 349, 353, and 355, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to any one of SEQ ID NOs: 374, 378, and 406. In some embodiments, the second spacer sequence comprises a nucleotide sequence having up to 1, 2, or 3 nucleotide deletions or substitutions relative to any one of SEQ ID NO: 533, 534, 538, and 540.

In some embodiments, the disclosure provides a system for use with a NGG PAM comprising:

(i) a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 236; and

(iii) a second gRNA molecule targeting a target site in the genomic DNA molecule, the second gRNA comprising a second spacer sequence corresponding to a second target sequence consisting of a nucleotide sequence selected from SEQ ID NO: 365, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to SEQ ID NOs: 421. In some embodiments, the second spacer sequence comprises a nucleotide sequence having up to 1, 2, or 3 nucleotide deletions or substitutions relative to any one of SEQ ID NO: 550.

In some embodiments, the disclosure provides a system for use with a NGG PAM comprising:

(i) a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 189, 193, and 221; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 290, 302, 306, and 317, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a partial removal of the FOC region. In some embodiments, the first spacer sequence comprises a nucleotide sequence having up to 1, 2, or 3 nucleotide deletions or substitutions relative to any one of SEQ ID NOs: 374, 378, and 406. In some embodiments, the second spacer sequence comprises a nucleotide sequence having up to 1, 2, or 3 nucleotide deletions or substitutions relative to any one of SEQ ID NO: 475, 487, 491, and 502.

In some embodiments, the disclosure provides a system for use with a NGG PAM comprising:

(i) a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 236; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 348, 349, 353, and 355, wherein when the system is introduced to the cell, the first gRNA and second gRNA combine with the site-directed endonuclease to induce cleavage proximal the first and second target sequences, to introduce an approximately 3-5.5 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to SEQ ID NOs: 421. In some embodiments, the second spacer sequence comprises a nucleotide sequence having up to 1, 2, or 3 nucleotide deletions or substitutions relative to any one of SEQ ID NOs: 533, 534, 538, and 540.

In some embodiments, the disclosure provides a system for use with a NGG PAM comprising:

(i) a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from SEQ ID NO: 236; and

(iii) a second gRNA molecule targeting a target site in the genomic DNA molecule, the second gRNA comprising a second spacer sequence corresponding to a second target sequence consisting of a nucleotide sequence selected from any one of SEQ ID NOs: 290, 302, 306, and 317, wherein when the system is introduced to the cell, the first gRNA and second gRNA combine with the site-directed endonuclease to induce cleavage proximal the first and second target sequences, to introduce an approximately 3-5.5 kb deletion in the genomic DNA molecule resulting in a full removal of FOP and a partial removal of the FOC region. In some embodiments, the first spacer sequence comprises a nucleotide sequence having up to 1, 2, or 3 nucleotide deletions or substitutions relative to SEQ ID NO: 421. In some embodiments, the second spacer sequence comprises a nucleotide sequence having up to 1, 2, or 3 nucleotide deletions or substitutions relative to any one of SEQ ID NOs: 475, 487, 491, and 502.

In some embodiments, the disclosure provides a system for use with a NNGRRT PAM for introducing a deletion in a genomic DNA molecule comprising FAAH-OUT, wherein the system comprises dual gRNAs and a site-directed endonuclease that recognizes an NNGRRT PAM. In some embodiments, the site-directed endonuclease is a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof. For example, in some embodiments, a functional derivative of SaCas9 endonuclease is one engineered for increased fidelity. In some embodiments, the deletion introduced is approximately 3-10 kb, approximately 3-9 kb, approximately 3-8 kb, approximately 3-7 kb, approximately 3-6 kb, approximately 3-5 kb, approximately 4-10 kb, approximately 4-9 kb, approximately 4-8 kb, approximately 4-7 kb, approximately 4-6 kb, approximately 5-10 kb, approximately 5-9 kb, approximately 5-8 kb, approximately 5-7 kb, approximately 6-10 kb, approximately 6-9 kb, approximately 6-8 kb, or approximately 8-10 kb in length. In some embodiments, the deletion comprises a removal of FOP. In some embodiments, the deletion comprises a full or partial removal of FOC.

In some embodiments, the dual gRNAs of the system for use with a NNGRRT PAM comprise a first gRNA molecule. In some embodiments, the first gRNA molecule comprises a spacer sequence corresponding to a first target sequence, wherein the first target sequence is adjacent an NNGRRT PAM, and wherein the first target sequence is downstream the 3′ terminus of FAAH and upstream a transcriptional start site of FAAH-OUT. In some embodiments, the first target sequence is: (i) within a region of the genomic DNA molecule that is at least about 4.5 kb, about 5 kb, about 5.5 kb, about 6 kb, about 6.5 kb, about 7 kb, about 7.5 kb, about 8 kb, about 8.5 kb, or about 9 kb downstream the 3′ terminus of FAAH; (ii) within a region of the genomic DNA molecule that is at least about 0.8 kb, about 0.9 kb, about 1 kb, about 1.5 kb, about 2 kb, about 2.5 kb, about 3 kb, about 3.5, about 4 kb, about 4.5 kb, or about 5 kb upstream the transcriptional start site of FAAH-OUT; (iii) within a region of the genomic DNA molecule between about 46,418,168 to about 46,422,208 of chromosome 1, according to human reference genome Hg38; or (iv) a combination of (i)-(iii).

In some embodiments, the dual gRNAs of the system for use with a NNGRRT PAM comprise a second gRNA molecule. In some embodiments, the second gRNA molecule comprises a spacer sequence corresponding to a second target sequence, wherein the second target sequence is adjacent an NNGRRT PAM, and wherein the second target sequence is downstream of the FAAH-OUT transcriptional start site and upstream an exon 3 of FAAH-OUT. In some embodiments, the second target sequence is (i) within a region of the genomic DNA molecule that is at least about 1.5 kb, about 2 kb, about 2.5 kb, about 3 kb, about 3.5 kb, about 4 kb, about 4.5 kb, about 5 kb, or about 5.5 kb downstream the transcriptional start site of FAAH-OUT; (ii) within a region of the genomic DNA molecule that is at least about 3.5 kb, about 4 kb, about 4.5 kb, about 5 kb, about 5.5 kb, about 6 kb, about 6.5 kb, about 7 kb, or about 7.5 kb upstream the 5′ end of exon 3 of FAAH-OUT; (iii) within a region of the genomic DNA molecule between about 46,424,887 to about 46,428,508 of chromosome 1, according to human reference genome Hg38; or (iv) a combination of (i)-(iii).

In some embodiments, the first gRNA of the system for use with a NNGRRT PAM, when introduced into a cell with the site-directed endonuclease that recognizes the NNGRRT PAM, combines with the site-directed endonuclease to induce cleavage proximal the first target sequence with a cleavage efficiency of at least 15%. In some embodiments, cleavage efficiency is measured as the frequency of INDELs induced proximal the target sequence (e.g., by TIDE analysis). In some embodiments, the cleavage efficiency is at least 15%, 20%, 25%, 30%, 35%, 40%, 45%, 50%, 55%, 60%, 65%, 70%, 75%, 80%, 85% or higher.

In some embodiments, the second gRNA of the system for use with a NNGRRT PAM, when introduced into a cell with the site-directed endonuclease that recognizes the NNGRRT PAM, combines with the site-directed endonuclease to induce cleavage proximal the first target sequence with a cleavage efficiency of at least 15%. In some embodiments, cleavage efficiency is measured as the frequency of INDELs induced proximal the target sequence (e.g., by TIDE analysis). In some embodiments, the cleavage efficiency is at least 15%, 20%, 25%, 30%, 35%, 40%, 45%, 50%, 55%, 60%, 65%, 70%, 75%, 80%, 85% or higher.

In some embodiments, the disclosure provides a system for use with a NNGRRT PAM comprising:

(i) a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 930, 932, 939, and 942; and

(iii) a second gRNA molecule targeting a target site in the genomic DNA molecule, the second gRNA comprising a second spacer sequence corresponding to a second target sequence consisting of a nucleotide sequence selected from SEQ ID NO: 1087 or SEQ ID NO: 1092, wherein when the system is introduced to the cell, the first gRNA and second gRNA combine with the site-directed endonuclease to induce cleavage proximal the first and second target sequences, to introduce an approximately 8-10 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to any one of SEQ ID NOs: 1102, 1104, 1111, and 1114. In some embodiments, the second spacer sequence comprises a nucleotide sequence having up to 1, 2, or 3 nucleotide deletions or substitutions relative to SEQ ID NO: 1259 or SEQ ID NO:1264.

In some embodiments, the disclosure provides a system for use with a NNGRRT PAM comprising:

(i) a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 930, 932, 939, 942, 947, 949, and 956; and

(iii) a second gRNA molecule targeting a target site in the genomic DNA molecule, the second gRNA comprising a second spacer sequence corresponding to a second target sequence consisting of a nucleotide sequence of SEQ ID NO: 1073, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to any one of SEQ ID NOs: 1102, 1104, 1111, 1114, 1119, 1121, and 1128. In some embodiments, the second spacer sequence comprises a nucleotide sequence having up to 1, 2, or 3 nucleotide deletions or substitutions relative to SEQ ID NO: 1245.

In some embodiments, the disclosure provides a system for use with a NNGRRT PAM comprising:

(i) a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 947, 949, 956, 960, 967, 968, 976, and 980; and

(iii) a second gRNA molecule targeting a target site in the genomic DNA molecule, the second gRNA comprising a second spacer sequence corresponding to a second target sequence consisting of a nucleotide sequence selected from SEQ ID NO: 1087 or SEQ ID NO: 1092, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to any one of SEQ ID NOs: 1119, 1121, 1128, 1132, 1139, 1140, 1148, and 1152. In some embodiments, the second spacer sequence comprises a nucleotide sequence having up to 1, 2, or 3 nucleotide deletions or substitutions relative to SEQ ID NO: 1259 or SEQ ID NO: 1264.

In some embodiments, the disclosure provides a system for use with a NNGRRT PAM comprising:

(i) a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 930, 932, and 939; and

(iii) a second gRNA molecule targeting a target site in the genomic DNA molecule, the second gRNA comprising a second spacer sequence corresponding to a second target sequence consisting of a nucleotide sequence of SEQ ID NO: 1046, wherein when the system is introduced to the cell, the first gRNA and second gRNA combine with the site-directed endonuclease to induce cleavage proximal the first and second target sequences, to introduce an approximately 5-8 kb deletion in the genomic DNA molecule resulting in a full removal of FOP and a partial removal of the FOC region. In some embodiments, the first spacer sequence comprises a nucleotide sequence having up to 1, 2, or 3 nucleotide deletions or substitutions relative to any one of SEQ ID NOs: 1102, 1104, and 1111. In some embodiments, the second spacer sequence comprises a nucleotide sequence having up to 1, 2, or 3 nucleotide deletions or substitutions relative to SEQ ID NO: 1218.

In some embodiments, the disclosure provides a system for use with a NNGRRT PAM comprising:

(i) a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 960, 967, 968, 976, and 980; and

(iii) a second gRNA molecule targeting a target site in the genomic DNA molecule, the second gRNA comprising a second spacer sequence corresponding to a second target sequence consisting of a nucleotide sequence of SEQ ID NO: 1073, wherein when the system is introduced to the cell, the first gRNA and second gRNA combine with the site-directed endonuclease to induce cleavage proximal the first and second target sequences, to introduce an approximately 3-5.5 kb deletion in the genomic DNA molecule resulting in a full removal of FOP and a full removal of the FOC region. In some embodiments, the first spacer sequence comprises a nucleotide sequence having up to 1, 2, or 3 nucleotide deletions or substitutions relative to any one of SEQ ID NOs: 1132, 1139, 1140, 1148, and 1152. In some embodiments, the second spacer sequence comprises a nucleotide sequence having up to 1, 2, or 3 nucleotide deletions or substitutions relative to SEQ ID NO: 1245.

In some embodiments, the disclosure provides a system for use with a NNGRRT PAM comprising:

(i) a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof;

(ii) a first gRNA molecule targeting a target site in the genomic DNA molecule, the first gRNA comprising a first spacer sequence corresponding to a first target sequence consisting of a nucleotide sequence selected from any one of SEQ ID NOs: 942, 947, 949, 956, 960, 967, 968, 976, and 980; and

(iii) a second gRNA molecule targeting a target site in the genomic DNA molecule, the second gRNA comprising a second spacer sequence corresponding to a second target sequence consisting of a nucleotide sequence of SEQ ID NO: 1046,

wherein when the system is introduced to the cell, the first gRNA and second gRNA combine with the site-directed endonuclease to induce cleavage proximal the first and second target sequences, to introduce an approximately 3-5.5 kb deletion in the genomic DNA molecule resulting in a full removal of FOP and a partial removal of the FOC region. In some embodiments, the first spacer sequence comprises a nucleotide sequence having up to 1, 2, or 3 nucleotide deletions or substitutions relative to any one of SEQ ID NOs: 1114, 1119, 1121, 1128, 1132, 1139, 1140, 1148, and 1152. In some embodiments, the second spacer sequence comprises a nucleotide sequence having up to 1, 2, or 3 nucleotide deletions or substitutions relative to SEQ ID NO: 1218.

D. Exemplary CRISPR/Cas Systems for Gene Editing of FAAH In some embodiments, the disclosure provides a system for use with a NNGG PAM for introducing a mutation in a genomic DNA molecule comprising FAAH, wherein the system comprises one or more gRNAs and a site-directed endonuclease that recognizes an NNGG PAM. In some embodiments, the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof. For example, in some embodiments, a functional derivative of SluCas9 endonuclease is one engineered for increased fidelity.

In some embodiments, the disclosure provides a system for use with a NNGG PAM comprising a gRNA molecule, wherein the gRNA molecule comprises a spacer sequence corresponding to a target sequence, wherein the target sequence is within exon 1 or exon 2 of FAAH. In some embodiments, wherein the gRNA is introduced into a cell with a site-directed endonuclease that recognizes an NNGG PAM (e.g., SluCas9 or functional derivative thereof), the gRNA and the site-directed endonuclease combine to introduce a DNA DSB proximal the target sequence (e.g., within exon 1 or exon 2 of FAAH). In some embodiments, repair of the DNA DSB (e.g., by an NEHJ repair pathway) introduces a mutation proximal the target sequence. In some embodiments, the mutation is an INDEL of at least ±1 nt (e.g., ±1, ±2, ±3, ±4, ±5, etc). In some embodiments, the INDEL disrupts the FAAH ORF, for example, by introducing a frameshift mutation in the FAAH coding sequence (e.g., within exon 1 or exon 2 of FAAH), wherein the disruption results in a FAAH transcript having an altered reading frame and/or a FAAH transcript encoding a mutated FAAH polypeptide with reduced or eliminated enzymatic activity. In some embodiments, the INDEL is a point mutation. In some embodiments, the INDEL introduces a premature stop codon in the FAAH coding sequence.

In some embodiments, the gRNA for use with a site-directed endonuclease that recognizes a NNGG PAM comprises a spacer sequence corresponding to a target sequence consisting of a nucleotide sequence as set forth by any one of SEQ ID NOs: 76, 77, 78, 79, 88, 89, 90, 92, 95, 96, 100, 102, 103, 104, and 107. In some embodiments the gRNA comprises a spacer sequence comprising up to 1, 2, or 3 nucleotide deletions or substitutions relative to any one of SEQ ID NOs: 116, 117, 118, 119, 128, 129, 130, 132, 135, 136, 140, 142, 143, 144, and 147.

In some embodiments, the target sequence is proximal exon 1 or exon 2 of FAAH. In some embodiments, the 3′ terminus of the target sequence is about 100 nt, about 90 nt, about 80 nt, about 70 nt, about 60 nt, about 50 nt, about 40 nt, about 30 nt, about 20 nt, or about 10 nt upstream the 5′ terminus of exon 1 or exon 2 of FAAH. In some embodiments, the 5′ terminus of the target sequence is about 10 nt, about 20 nt, about 30 nt, about 40 nt, about 50 nt, about 60 nt, about 70 nt, about 80 nt, about 90 nt, or about 100 nt downstream the 3′ terminus of exon 1 or exon 2 of FAAH. In some embodiments, wherein the gRNA is introduced into a cell with a site-directed endonuclease that recognizes an NNGG PAM (e.g., SluCas9), the gRNA and the site-directed endonuclease combine to introduce a DNA DSB proximal the target sequence (e.g., upstream the 5′ terminus of exon 1 or exon 2 of FAAH, e.g., downstream the 3′ terminus of exon 1 or exon 2 of FAAH). In some embodiments, the mutation is an INDEL of at least ±1 nt (e.g., ±1, ±2, ±3, ±4, ±5, etc). In some embodiments, the INDEL disrupts a regulatory sequence of FAAH, wherein the disrupts results in decreased expression of FAAH (e.g., decreased transcription of FAAH, decreased or inhibited splicing of FAAH pre-mRNA, decreased translation of FAAH transcript). In some embodiments, the INDEL disrupts a splicing element of FAAH.

In some embodiments, the gRNA for use with a site-directed endonuclease that recognizes a NNGG PAM comprises a spacer sequence corresponding to a target sequence consisting of a nucleotide sequence as set forth by any one of SEQ ID NOs: 69, 70, 72, and 93. In some embodiments the gRNA comprises a spacer sequence comprising up to 1, 2, or 3 nucleotide deletions or substitutions relative to any one of SEQ ID NOs: 109, 110, 112, and 133.

In some embodiments, the gRNA for use with a site-directed endonuclease that recognizes a NNGG PAM (e.g., SluCas9 or functional derivative thereof), when introduced into a population of cells with the site-directed endonuclease, combines with the site-directed endonuclease to introduce a DNA DSB proximal the gRNA target sequence within or proximal the FAAH coding sequence (e.g., exon 1 or exon 2 of FAAH), wherein the cleavage efficiency (e.g., as measured by TIDE analysis) is at least about 15%, about 20%, about 25%, about 30%, about 35%, about 40%, about 45%, about 50%, about 55%, about 60%, about 65%, about 70%, about 75%, about 80%, about 85% or higher. In some embodiments, repair of the DNA DSB introduces a mutation (e.g., an INDEL) resulting in decreased expression of FAAH mRNA (e.g., as measured by qPCR or ddPCR) by at least 15%, at least 20%, at least 25%, at least 30%, at least 35%, at least 40%, at least 45%, at least 50%, at least 55%, at least 60%, at least 65%, at least 70%, or at least 75% or more compared to an unmodified population of cells. In some embodiments, repair of the DNA DSB introduces a mutation (e.g., an INDEL) resulting in decreased expression of FAAH polypeptide (e.g., as measured by western blot) by at least 15%, at least 20%, at least 25%, at least 30%, at least 35%, at least 40%, 45% or more compared to an unmodified population of cells.

In some embodiments, the disclosure provides a system for use with a NGG PAM for introducing a mutation in a genomic DNA molecule comprising FAAH, wherein the system comprises one or more gRNAs and a site-directed endonuclease that recognizes an NGG PAM. In some embodiments, the site-directed endonuclease is a SpCas9 endonuclease or a functional derivative thereof, an mRNA encoding the SpCas9 endonuclease or functional derivative thereof, or a recombinant expression vector comprising a nucleotide sequence encoding the SpCas9 endonuclease or functional derivative thereof. For example, in some embodiments, a functional derivative of SpCas9 endonuclease is one engineered for increased fidelity.

In some embodiments, the disclosure provides a system for use with a NGG PAM comprising a gRNA molecule, wherein the gRNA molecule comprises a spacer sequence corresponding to a target sequence, wherein the target sequence is within exon 1 or exon 2 of FAAH. In some embodiments, wherein the gRNA is introduced into a cell with a site-directed endonuclease that recognizes an NGG PAM (e.g., SpCas9 or functional derivative thereof), the gRNA and the site-directed endonuclease combine to introduce a DNA DSB proximal the target sequence (e.g., within exon 1 or exon 2 of FAAH). In some embodiments, repair of the DNA DSB (e.g., by an NEHJ repair pathway) introduces a mutation proximal the target sequence. In some embodiments, the mutation is an INDEL of at least ±1 nt (e.g., ±1, ±2, ±3, ±4, ±5, etc). In some embodiments, the INDEL disrupts the FAAH ORF, for example, by introducing a frameshift mutation in the FAAH coding sequence (e.g., within exon 1 or exon 2 of FAAH), wherein the disruption results in a FAAH transcript having an altered reading frame and/or a FAAH transcript encoding a mutated FAAH polypeptide with reduced or eliminated enzymatic activity. In some embodiments, the INDEL is a point mutation. In some embodiments, the INDEL introduces a premature stop codon in the FAAH coding sequence.

In some embodiments, the gRNA for use with a site-directed endonuclease that recognizes a NGG PAM comprises a spacer sequence corresponding to a target sequence consisting of a nucleotide sequence as set forth by any one of SEQ ID NOs: 7-14, 16-21, 24-34. In some embodiments the gRNA comprises a spacer sequence comprising up to 1, 2, or 3 nucleotide deletions or substitutions relative to any one of SEQ ID NOs: 41-48, 50-55, 58-68.

In some embodiments, the target sequence is proximal exon 1 or exon 2 of FAAH. In some embodiments, the 3′ terminus of the target sequence is about 100 nt, about 90 nt, about 80 nt, about 70 nt, about 60 nt, about 50 nt, about 40 nt, about 30 nt, about 20 nt, or about 10 nt upstream the 5′ terminus of exon 1 or exon 2 of FAAH. In some embodiments, the 5′ terminus of the target sequence is about 10 nt, about 20 nt, about 30 nt, about 40 nt, about 50 nt, about 60 nt, about 70 nt, about 80 nt, about 90 nt, or about 100 nt downstream the 3′ terminus of exon 1 or exon 2 of FAAH. In some embodiments, wherein the gRNA is introduced into a cell with a site-directed endonuclease that recognizes an NGG PAM (e.g., SpCas9), the gRNA and the site-directed endonuclease combine to introduce a DNA DSB proximal the target sequence (e.g., upstream the 5′ terminus of exon 1 or exon 2 of FAAH, e.g., downstream the 3′ terminus of exon 1 or exon 2 of FAAH). In some embodiments, the mutation is an INDEL of at least ±1 nt (e.g., ±1, ±2, ±3, ±4, ±5, etc). In some embodiments, the INDEL disrupts a regulatory sequence of FAAH, wherein the disrupts results in decreased expression of FAAH (e.g., decreased transcription of FAAH, decreased or inhibited splicing of FAAH pre-mRNA, decreased translation of FAAH transcript). In some embodiments, the INDEL disrupts a splicing element of FAAH.

In some embodiments, the gRNA for use with a site-directed endonuclease that recognizes a NGG PAM comprises a spacer sequence corresponding to a target sequence consisting of a nucleotide sequence as set forth by any one of SEQ ID NOs: 3-6, 22, and 23. In some embodiments the gRNA comprises a spacer sequence comprising up to 1, 2, or 3 nucleotide deletions or substitutions relative to any one of SEQ ID NOs: 37-40, 56, and 57.

In some embodiments, the gRNA for use with a site-directed endonuclease that recognizes a NGG PAM (e.g., SpCas9 or functional derivative thereof), when introduced into a population of cells with the site-directed endonuclease, combines with the site-directed endonuclease to introduce a DNA DSB proximal the gRNA target sequence within or proximal the FAAH coding sequence (e.g., exon 1 or exon 2 of FAAH), wherein the cleavage efficiency (e.g., as measured by TIDE analysis) is at least about 15%, about 20%, about 25%, about 30%, about 35%, about 40%, about 45%, about 50%, about 55%, about 60%, about 65%, about 70%, about 75%, about 80%, about 85%, about 90%, about 95%, or higher. In some embodiments, repair of the DNA DSB introduces a mutation (e.g., an INDEL) resulting in decreased expression of FAAH mRNA (e.g., as measured by qPCR or ddPCR) by at least 15%, at least 20%, at least 25%, at least 30%, at least 35%, at least 40%, at least 45%, at least 50%, at least 55%, at least 60%, at least 65%, at least 70%, at least 75% or more compared to an unmodified population of cells. In some embodiments, repair of the DNA DSB introduces a mutation (e.g., an INDEL) resulting in decreased expression of FAAH polypeptide (e.g., as measured by western blot) by at least 15%, at least 20%, at least 25%, at least 30%, at least 35%, at least 40%, or more compared to an unmodified population of cells.

In some embodiments, the disclosure provides a system for use with a NNGRRT PAM for introducing a mutation in a genomic DNA molecule comprising FAAH, wherein the system comprises one or more gRNAs and a site-directed endonuclease that recognizes an NNGRRT PAM. In some embodiments, the site-directed endonuclease is a SaCas9 endonuclease or a functional derivative thereof, an mRNA encoding the SaCas9 endonuclease or functional derivative thereof, or a recombinant expression vector comprising a nucleotide sequence encoding the SaCas9 endonuclease or functional derivative thereof. For example, in some embodiments, a functional derivative of SaCas9 endonuclease is one engineered for increased fidelity.

In some embodiments, the disclosure provides a system for use with a NNGRRT PAM comprising a gRNA molecule, wherein the gRNA molecule comprises a spacer sequence corresponding to a target sequence, wherein the target sequence is within exon 1, exon 2, or exon 4 of FAAH. In some embodiments, wherein the gRNA is introduced into a cell with a site-directed endonuclease that recognizes an NNGRRT PAM (e.g., SaCas9 or functional derivative thereof), the gRNA and the site-directed endonuclease combine to introduce a DNA DSB proximal the target sequence (e.g., within exon 1, exon 2, or exon 4 of FAAH). In some embodiments, repair of the DNA DSB (e.g., by an NEHJ repair pathway) introduces a mutation proximal the target sequence. In some embodiments, the mutation is an INDEL of at least ±1 nt (e.g., ±1, ±2, ±3, ±4, ±5, etc). In some embodiments, the INDEL disrupts the FAAH ORF, for example, by introducing a frameshift mutation in the FAAH coding sequence (e.g., within exon 1, exon 2, or exon 4 of FAAH), wherein the disruption results in a FAAH transcript having an altered reading frame and/or a FAAH transcript encoding a mutated FAAH polypeptide with reduced or eliminated enzymatic activity. In some embodiments, the INDEL is a point mutation. In some embodiments, the INDEL introduces a premature stop codon in the FAAH coding sequence.

In some embodiments, the gRNA for use with a site-directed endonuclease that recognizes a NNGRRT PAM comprises a spacer sequence corresponding to a target sequence consisting of a nucleotide sequence as set forth by SEQ ID NOs: 152, 155, 156, 158, 159, 160, 161, 162, and 163. In some embodiments the gRNA comprises a spacer sequence comprising up to 1, 2, or 3 nucleotide deletions or substitutions relative to any one of SEQ ID NOs: 168, 171, 172, 174, 175, 176, 177, 178, and 179.

In some embodiments, the target sequence is proximal exon 1, exon 2, or exon 4 of FAAH. In some embodiments, the 3′ terminus of the target sequence is about 100 nt, about 90 nt, about 80 nt, about 70 nt, about 60 nt, about 50 nt, about 40 nt, about 30 nt, about 20 nt, or about 10 nt upstream the 5′ terminus of exon 1, exon 2, or exon 4 of FAAH. In some embodiments, the 5′ terminus of the target sequence is about 10 nt, about 20 nt, about 30 nt, about 40 nt, about 50 nt, about 60 nt, about 70 nt, about 80 nt, about 90 nt, or about 100 nt downstream the 3′ terminus of exon 1, exon 2, or exon 4 of FAAH. In some embodiments, wherein the gRNA is introduced into a cell with a site-directed endonuclease that recognizes an NNGRRT PAM (e.g., SaCas9 or functional derivative thereof), the gRNA and the site-directed endonuclease combine to introduce a DNA DSB proximal the target sequence (e.g., upstream the 5′ terminus of exon 1, exon 2, or exon 4 of FAAH, e.g., downstream the 3′ terminus of exon 1, exon 2, or exon 4 of FAAH). In some embodiments, the mutation is an INDEL of at least ±1 nt (e.g., ±1, ±2, ±3, ±4, ±5, etc). In some embodiments, the INDEL disrupts a regulatory sequence of FAAH, wherein the disrupts results in decreased expression of FAAH (e.g., decreased transcription of FAAH, decreased or inhibited splicing of FAAH pre-mRNA, decreased translation of FAAH transcript). In some embodiments, the INDEL disrupts a splicing element of FAAH.

In some embodiments, the gRNA for use with a site-directed endonuclease that recognizes a NNGRRT PAM comprises a spacer sequence corresponding to a target sequence consisting of a nucleotide sequence as set forth by any one of SEQ ID NOs: 149, 150, 151, 153, 164. In some embodiments the gRNA comprises a spacer sequence comprising up to 1, 2, or 3 nucleotide deletions or substitutions relative to any one of SEQ ID NOs: 165, 166, 167, 169, 180.

In some embodiments, the gRNA for use with a site-directed endonuclease that recognizes a NNGRRT PAM (e.g., SaCas9 or functional derivative thereof), when introduced into a population of cells with the site-directed endonuclease, combines with the site-directed endonuclease to introduce a DNA DSB proximal the gRNA target sequence within or proximal the FAAH coding sequence (e.g., exon 1 or exon 2 of FAAH), wherein the cleavage efficiency (e.g., as measured by TIDE analysis) is at least about 20%, about 25%, about 30%, about 35%, about 40%, about 45%, about 50%, about 55%, about 60%, about 65%, about 70%, about 75%, about 80%, about 85%, or higher. In some embodiments, repair of the DNA DSB introduces a mutation (e.g., an INDEL) resulting in decreased expression of FAAH mRNA (e.g., as measured by qPCR or ddPCR) by at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more compared to an unmodified population of cells. In some embodiments, repair of the DNA DSB introduces a mutation (e.g., an INDEL) resulting in decreased expression of FAAH polypeptide (e.g., as measured by western blot) by at least 15%, at least 20%, at least 25%, at least 30%, at least 35%, at least 40%, or more compared to an unmodified population of cells.

### V. Modified Nucleases

In certain embodiments, the disclosure provides gene-editing systems comprising a site-directed endonuclease, wherein the nuclease is optionally modified from its wild-type counterpart. In some embodiments, the nuclease is fused with at least one heterologous protein domain. At least one protein domain is located at the N-terminus, the C-terminus, or in an internal location of the nuclease. In some embodiments, two or more heterologous protein domains are at one or more locations on the nuclease.

In some embodiments, the protein domain may facilitate transport of the nuclease into the nucleus of a cell. For example, the protein domain is a nuclear localization signal (NLS). In some embodiments, the nuclease is fused with 1-10 NLS(s). In some embodiments, the nuclease is fused with 1-5 NLS(s). In some embodiments, the nuclease is fused with one NLS. In other embodiments, the nuclease is fused with more than one NLS. In some embodiments, the nuclease is fused with 2, 3, 4, or 5 NLSs. In some embodiments, the nuclease is fused with 2 NLSs. In some embodiments, the nuclease is fused with 3 NLSs. In some embodiments, the nuclease is fused with no NLS. In some embodiments, the NLS may be a monopartite sequence, such as, e.g., the SV40 NLS, PKKKRKV (SEQ ID NO: 1288) or PKKKRRV (SEQ ID NO: 1289). In some embodiments, the NLS is a bipartite sequence, such as, e.g., the NLS of nucleoplasmin, KRPAATKKAGQAKKKK (SEQ ID NO: 1290). In some embodiments, the NLS is genetically modified from its wild-type counterpart.

In additional embodiments, the protein domain may target the nuclease to a specific organelle, cell type, tissue, or organ.

In further embodiments, the protein domain is an effector domain. When the nuclease is directed to its target nucleic acid, e.g., when a Cas9 protein is directed to a target nucleic acid by a guide RNA, the effector domain may modify or affect the target nucleic acid. In some embodiments, the effector domain is chosen from a nucleic acid binding domain, a nuclease domain, an epigenetic modification domain, a transcriptional activation domain, or a transcriptional repressor domain. In some embodiments, the effector domain can be a nucleobase deaminase domain.

### VI. Target Sites

In some embodiments, the site-directed nucleases described herein are directed to and cleave (e.g., introduce a DSB) a target nucleic acid molecule (e.g., a target site within or proximal the FAAH coding sequence; e.g., a target site within or proximal FAAH-OUT). In some embodiments, a Cas nuclease is directed by a guide RNA to a target site of a target nucleic acid molecule (e.g., genomic DNA molecule), where the guide RNA hybridizes with the complementary strand of the target sequence and the Cas nuclease cleaves the target nucleic acid at the target site. In some embodiments, the complementary strand of the target sequence is complementary to the targeting sequence (e.g.: spacer sequence) of the guide RNA. In some embodiments, the degree of complementarity between a targeting sequence of a guide RNA and its corresponding complementary strand of the target sequence is about 50%, 55%, 60%, 65%, 70%, 75%, 80%, 85%, 90%, 95%, 97%, 98%, 99%, or 100%. In some embodiments, the complementary strand of the target sequence and the targeting sequence of the guide RNA is 100% complementary. In other embodiments, the complementary strand of the target sequence and the targeting sequence of the guide RNA contains at least one mismatch. For example, the complementary strand of the target sequence and the targeting sequence of the guide RNA contain 1, 2, 3, 4, 5, 6, 7, 8, 9, or 10 mismatches. In some embodiments, the complementary strand of the target sequence and the targeting sequence of the guide RNA contains 1-6 mismatches. In some embodiments, the complementary strand of the target sequence and the targeting sequence of the guide RNA contain 1, 2, or 3 mismatches.

The length of the target sequence may depend on the nuclease system used. For example, the target sequence for a CRISPR/Cas system comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the target sequence comprises 18-24 nucleotides in length. In some embodiments, the target sequence comprises 19-22 nucleotides in length. In some embodiments, the target sequence comprises 20 nucleotides in length. In some embodiments, the target sequence comprises 21 nucleotides in length. In some embodiments, the target sequence comprises 22 nucleotides in length.

### Nucleic Acids Encoding System Components

The present disclosure provides a nucleic acid comprising a nucleotide sequence encoding a gRNA molecule of the disclosure, a site-directed endonuclease of the disclosure, and/or any nucleic acid or proteinaceous molecule necessary to carry out the aspects of the methods of the disclosure. In some embodiments, the nucleic acid comprises a vector (e.g., a recombinant expression vector).

### I. Vectors

In some embodiments, the site-directed nuclease (e.g., Cas nuclease) and the one or more gRNAs of the disclosure are provided by one or more vectors. As used herein, the term “vector” refers to a nucleic acid molecule capable of transporting another nucleic acid to which it has been linked. In some embodiments, the vector is a DNA vector. In some embodiments, the vector is circular. In some embodiments, the vector is linear. Non-limiting exemplary vectors include plasmids, phagemids, cosmids, artificial chromosomes, minichromosomes, transposons, viral vectors, and expression vectors.

In some embodiments, the vector is an expression vector, wherein the expression vector is capable of directing the expression of nucleic acids to which it is operably linked. As used herein, an “expression vector” or “recombinant expression vector” refers to a replicon, such as plasmid, phage, virus, or cosmid, to which another DNA segment, i.e. an “insert”, is attached so as to bring about the replication of the attached segment in a cell.

In some embodiments, the vector or expression vector is a plasmid. As used herein, a “plasmid” refers to a circular double-stranded DNA loop into which additional nucleic acid segments are ligated.

In some embodiments, the vector or expression vector is a viral vector, wherein additional nucleic acid segments are ligated into the viral genome. Non-limiting exemplary viral vectors include viral vectors based on vaccinia virus; poliovirus; adenovirus; adeno-associated virus; SV40; herpes simplex virus; human immunodeficiency virus; picornaviruses. Non-limiting exemplary viral vectors also include viral vectors based on a retrovirus such as a Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus. In some embodiments, the vectors is for use in eukaryotic target cells and includes, but is not limited to, pXT1, pSG5, pSVK3, pBPV, pMSG, and pSVLSV40 (Pharmacia).

In some embodiments, the vector comprises one or more transcription and/or translation control elements. In some embodiments, the more transcription and/or translation control elements used depends on the target cell population and the vector system. In some embodiments, any number of suitable transcription and translation control elements, including constitutive and inducible promoters, transcription enhancer elements, transcription terminators, etc. are used in the expression vector, such as those further described below.

In some embodiments, a vector comprising a nucleic acid encoding a gRNA molecule of the disclosure and/or a site-directed endonuclease of the disclosure is operably linked to a control element, e.g., a transcriptional control element, such as a promoter. In some embodiments, the transcriptional control element is functional in a eukaryotic cell, e.g., a mammalian cell, e.g., a human cell. In some embodiments, the nucleotide sequence encoding the gRNA molecule and/or the site-directed endonuclease is operably linked to one or more control elements that enable expression of the nucleotide sequence encoding the gRNA and/or a site-directed endonuclease in eukaryotic cells, e g, mammalian cells, e.g., human cells.

In some embodiments, the promoter is a constitutively active promoter (i.e., a promoter that is constitutively in an active/“ON” state). In some embodiments, the promoter is an inducible promoter (i.e., a promoter whose state, active/“ON” or inactive/“OFF”, is controlled by an external stimulus, e.g., the presence of a particular temperature, compound, or protein). In some embodiments, the promoter is a spatially restricted promoter (i.e., transcriptional control element, enhancer, etc.) (e.g., tissue specific promoter, cell type specific promoter, etc.). In some embodiments, the promoter is temporally restricted promoter (i.e., the promoter is in the “ON” state or “OFF” state during specific stages of embryonic development or during specific stages of a biological process).

Suitable promoters for use in the present disclosure include those derived from viruses and are referred to herein as viral promoters, or they include those derived from an organism, including prokaryotic or eukaryotic organisms. In some embodiments, a suitable promoter for use in the present disclosure include any promoter that drives expression by an RNA polymerase (e.g., pol I, pol II, pol III).

Exemplary promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Exemplary eukaryotic promoters (i.e., promoters functional in a eukaryotic cell) include, but are not limited to, those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

In some embodiments, a gRNA molecule of the disclosure is encoded by vector comprising a RNA polymerase III promoter (e.g., U6 and H1). Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

In some embodiments, the expression vector comprises a ribosome binding site for translation initiation and a transcription terminator. In some embodiments, the expression vector comprises appropriate sequences for amplifying expression. In some embodiments, the expression vector comprises nucleotide sequences encoding non-native tags (e.g., histidine tag, hemagglutinin tag, green fluorescent protein, etc.), for example, that are operably-linked to a site-directed endonuclease, thereby providing a fusion protein of the site-directed endonuclease.

In some embodiments, the expression vector comprises a promoter that is an inducible promoter (e.g., a heat shock promoter, tetracycline-regulated promoter, steroid-regulated promoter, metal-regulated promoter, estrogen receptor-regulated promoter, etc.). In some embodiments, the promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.).

Examples of inducible promoters include, but are not limited to, T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), steroid-regulated promoter, metal-regulated promoter, estrogen receptor-regulated promoter, etc. In some embodiments, an inducible promoters is regulated by molecules including, but not limited to, doxycycline; RNA polymerase, e.g., T7 RNA polymerase; an estrogen receptor; an estrogen receptor fusion; etc.

Spatially restricted promoters can also be referred to as enhancers, transcriptional control elements, control sequences, etc. Any convenient spatially restricted promoter is suitable for use in the present disclosure, and the choice of a suitable promoter (e.g., a liver-specific promoter, a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a site-directed endonuclease and/or one or more gRNA molecules in a wide variety of different tissues and cell types, depending on the organism. Some spatially restricted promoters are also temporally restricted such that the promoter is in the “ON” state or “OFF” state during specific stages of embryonic development or during specific stages of a biological process.

For illustration purposes, examples of spatially restricted promoters include, but are not limited to, liver-specific promoters, neuron-specific promoters, adipocyte-specific promoters, cardiomyocyte-specific promoters, smooth muscle-specific promoters, photoreceptor-specific promoters, etc.

Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca²⁺-calmodulin-dependent protein kinase II-alpha (CamKIIa) promoter (see, e.g., Mayford et al. (1996) Proc. Natl. Acad. Sci. USA 93:13250; and Casanova et al. (2001) Genesis 31:37); a CMV enhancer/platelet-derived growth factor-0 promoter (see, e.g., Liu et al. (2004) Gene Therapy 11:52-60); and the like.

Methods of introducing a nucleic acid to a host cell or a population of host cells are known in the art, and any known method can be used to introduce a nucleic acid (e.g., an expression construct) into a cell. In some embodiments, a nucleic acid molecule encoding a guide RNA (introduced either as DNA or RNA) and/or a site-directed endonuclease (introduced as DNA or RNA) are provided to a population of cells using well-developed transfection techniques; see, e.g. Angel and Yanik (2010) PLoS ONE 5(7): e 11756, and the commercially available TransMessenger® reagents from Qiagen, Stemfect™ RNA Transfection Kit from Stemgent, and TransIT®-mRNA Transfection Kit from Mims Bio LLC (See, also Beumer et al. (2008). PNAS 105(50):19821-19826). In some embodiments, the nucleic acids encoding a guide RNA and/or a site-directed endonuclease are provided as a DNA vectors, e.g. plasmids, cosmids, minicircles, phage, viruses, etc. In some embodiments, the vectors comprising the nucleic acid(s) are maintained episomally, e.g. as plasmids, minicircle DNAs, viruses such cytomegalovirus, adenovirus, etc. In some embodiments, the vectors integrated into the host cell genome, through homologous recombination or random integration, e.g. retrovirus-derived vectors such as MMLV, HIV-1, ALV, etc.

### II. Messenger RNA Encoding Cas Nuclease

In some aspects, the disclosure provides an mRNA encoding a site-directed endonuclease (e.g., SluCas9, SpCas9, SaCas9), for use in methods of genome editing using a CRISPR/Cas system. In some embodiments, the mRNA comprises a 5′ UTR, an open reading frame (ORF) comprising a nucleotide sequence encoding the site-directed endonuclease, and a 3′ UTR.

In some embodiments, the mRNA comprises one or more modification to improve mRNA stability, increase mRNA translation efficiency, and/or reduce mRNA immunogenicity. In some embodiments, the one or more modification is sequence optimization of the mRNA and/or chemical modification of at least one nucleotide of the mRNA.

In some embodiments, the mRNA comprises a sequence-optimized nucleotide sequence. In some embodiments, the mRNA comprises a nucleotide sequence that is sequence optimized for expression in a target cell. In some embodiments, the target cell is a mammalian cell. In some embodiments, the target cell is a human cell, a murine cell, or a non-human primate (NHP) cell. Methods of sequence optimization are known in the art, and include known sequence optimization tools, algorithms and services. Non-limiting examples include services from GeneArt (Life Technologies), DNA2.0 (Menlo Park Calif.), Geneious®, GeneGPS® (Atum, Newark, Calif.), and/or proprietary methods. In some embodiments, the nucleotide sequence is (i) sequence-optimized based on codon usage bias in a host cell (e.g., mammalian cell, e.g., human cell, murine cell, non-human primate cell) relative to a reference sequence, (ii) uridine-depleted relative to a reference sequence, or (iii) a combination of (i) and (ii), using a method of sequence optimization (e.g., GeneGPS®, e.g., Geneious®).

In some embodiments, the mRNA has chemistries suitable for delivery, tolerability, and stability within cells, e.g., following in vivo or in vitro administration. In some embodiments, the mRNA is modified, e.g., comprises a modified sugar moiety, a modified internucleoside linkage, a modified nucleoside, a modified nucleotide and/or combinations thereof. In some embodiments, the modified mRNA exhibits one or more of the following properties: is not immune stimulatory; is nuclease resistant; has improved cell uptake; has increased half-life; has increased translation efficiency; and/or is not toxic to cells or mammals, e.g., following contact with cells in vivo or ex vivo or in vitro.

### A. Messenger RNA Components

In some embodiments, the disclosure provides an mRNA comprising an open-reading frame (ORF), wherein the ORF comprises a nucleotide sequence that encodes a site-directed endonuclease, such as a Cas nuclease.

In some embodiments, an mRNA of the disclosure comprises a 5′ untranslated region (5′ UTR), a 3′ untranslated region (3′ UTR), and an ORF comprising a nucleotide sequence encoding a site-directed endonuclease (e.g., Cas nuclease). In some embodiments, the mRNA further comprises a 5′ cap structure, a Kozak or Kozak-like sequence (also known as a Kozak consensus sequence), a polyA sequence (also known as a polyadenylation signal), a nucleotide sequence encoding a nuclear localization signal (NLS), a nucleotide sequence encoding a linker peptide, a nucleotide sequence encoding a tag peptide, or any combination thereof. In some embodiments, the consensus Kozak consensus sequence facilitates the initial binding of mRNA to ribosomes, thereby enhances its translation into a polypeptide product.

In some embodiments, an mRNA of the disclosure comprises any suitable number of base pairs, e.g., thousands (e.g., 4000, 5000, 6000, 7000, 8000, 9000, or 10,000) of base pairs. In some embodiments, the mRNA is about 4.2 kb, about 4.3 kb, about 4.4 kb, about 4.5 kb, about 4.6 kb, about 4.7 kb, about 4.8 kb, about 4.9 kb, about 5.0 kb, about 5.1 kb, about 5.2 kb, about 5.3 kb, about 5.4 kb, about 5.5 kb, or more in length.

In some embodiments, the 5′ UTR or 3′ UTR is derived from a human gene sequence. Non-limiting exemplary 5′ UTR and 3′ UTR include those derived from genes encoding α- and β-globin, albumin, HSD17B4, and eukaryotic elongation factor 1a. In addition, viral-derived 5′ UTR and 3′ UTRs can also be used and include orthopoxvirus and cytomegalovirus UTR sequences.

In some embodiments, an mRNA of the disclosure comprises a 5′ cap structure. A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. This cap is a cap-0 where nucleotide N does not contain 2′OMe, or cap-1 where nucleotide N contains 2′OMe, or cap-2 where nucleotides N and N+1 contain 2′OMe. This cap may also be of the structure m2 7′3 “G(5′)N as incorporated by the anti-reverse-cap analog (ARCA), and may also include similar cap-0, cap-1, and cap-2, etc., structures.

In some embodiments, an mRNA of the disclosure further comprises a nucleotide sequence encoding a nuclear localization signal (NLS). In some embodiments, the nuclease is fused with more than one NLS. In some embodiments, one or more NLS is operably-linked to the N-terminus, C-terminus, or both, of the site-directed endonuclease, optionally via a peptide linker. In some embodiments, the NLS comprises a nucleoplasmin NLS and/or a SV40 NLS. some embodiments, the mRNA comprises a nucleotide sequence encoding a nucleoplasmin NLS and a nucleotide sequence encoding an SV40 NLS.

In some embodiments, an mRNA of the disclosure comprises a poly(A) tail (i.e., polyA sequence, i.e., polyadenylation signal). In some embodiments, the polyA sequence comprises entirely or mostly of adenine nucleotides or analogs or derivatives thereof. In some embodiments, the polyA sequence is a tail located adjacent (e.g., towards the 3′ end) of a 3′ UTR of an mRNA. In some embodiments, the polyA sequence promotes or increases the nuclear export, translation, and/or stability of the mRNA.

In some embodiments, the poly(A) tail comprises a 3′ “cap” comprising modified or non-natural nucleobases or other synthetic moieties.

### III. Nucleic Acid Modifications

In some embodiments, a nucleic acid of the disclosure (e.g., gRNA and/or mRNA encoding a site-directed endonuclease) of the disclosure comprises one or more modified nucleobases, nucleosides, nucleotides or internucleoside linkages. In some embodiments, modified nucleic acids disclosure (e.g., gRNA and/or mRNA encoding a site-directed endonuclease) have useful properties, including enhanced stability, intracellular retention, enhanced translation, and/or the lack of a substantial induction of the innate immune response of a cell into which the nucleic acid is introduced, as compared to a reference unmodified nucleic acid. Therefore, use of modified nucleic acids may enhance the efficiency of protein production (e.g., expression of a site-directed endonuclease), intracellular retention of the nucleic acids, efficiency of a genome editing system comprising the nucleic acid, as well as possess reduced immunogenicity.

In some embodiments, a gRNA and/or mRNA of the disclosure comprises one or more (e.g., 1, 2, 3, 4, 5, 6, 7, 8, 9, 10, 20, 30, 40, 50, 60, 70, 80, 90, 100, or more) different modified nucleobases, nucleosides, nucleotides or internucleoside linkages. In some embodiments, the modified nucleic acid (e.g., gRNA, and/or mRNA) has reduced degradation in a cell into which the nucleic acid is introduced, relative to a corresponding unmodified nucleic acid.

In some embodiments, the modified nucleobase is a modified uracil, such as any modified uracil known in the art. In some embodiments, the modified nucleobase is a modified cytosine, such as any modified cytosine known in the art. In some embodiments, the modified nucleobase is modified adenine, such as any modified adenine known in the art. In some embodiments, the modified nucleobase is modified guanine, such as any modified guanine known in the art.

In some embodiments, a nucleic acid (e.g., mRNA and/or gRNA) of the disclosure includes a combination of one or more of the aforementioned modified nucleobases (e.g., a combination of 2, 3 or 4 of the aforementioned modified nucleobases).

In certain embodiments, a nucleic acid (e.g., mRNA and/or gRNA) of the disclosure is uniformly modified (i.e., fully modified, modified through-out the entire sequence) for a particular modification. For example, an mRNA can be uniformly modified with N1-methylpseudouridine (m¹ψ) or 5-methyl-cytidine (m⁵C), meaning that all uridines or all cytosine nucleosides in the mRNA sequence are replaced with N1-methylpseudouridine (m¹Ψ) or 5-methyl-cytidine (m⁵C). Similarly, a nucleic acid (e.g., mRNA and/or gRNA) of the disclosure can be uniformly modified for any type of nucleoside residue present in the sequence by replacement with a modified residue such as those set forth above.

### Delivery

In some embodiments, delivery of gene editing systems components described herein (e.g., gRNA and/or site-directed endonuclease) is performed by one or more methods described herein. In some embodiments, the system components, for example, one or more gRNA molecules and/or a site-directed endonuclease (e.g., Cas nuclease), are delivered by viral vectors, lipid nanoparticles (LNPs), synthetic polymers, or a combination thereof. In some embodiments, the methods of delivery described herein are suitable for administering a gene editing system of the disclosure to a target cell population or target tissue for the purpose of cellular, ex vivo, or in vivo gene editing.

In some embodiments, the delivery comprises administering the site-directed endonuclease as nucleic acid encoding the site-directed endonuclease (RNA or DNA). In some embodiments, the site-directed endonuclease is delivered as an mRNA or a recombinant expression vector comprising a nucleic acid encoding the site-directed endonuclease (e.g, plasmid, viral vector). In some embodiments, the delivery comprises administering the site-directed endonuclease as a polypeptide. In some embodiments, the delivery comprises administering one or more gRNAs or a nucleic acid encoding the one or more gRNAs. In some embodiments, the delivery comprises administering a recombinant expression vector comprising a nucleic acid encoding the one or more gRNAs (e.g., plasmid, viral vector).

In some embodiments, the delivery comprises administering the site-directed endonuclease as a mRNA. In some embodiments, the delivery comprises administering the mRNA, wherein the mRNA is formulated by LNP or another delivery vehicle, such as a polymeric nanoparticles. In some embodiments, the delivery comprises administering the mRNA separately formulated or co-formulated with one or more gRNAs. In some embodiments, the mRNA and the one or more gRNAs are separately formulated as an LNP or polymeric nanoparticle. In some embodiments, the mRNA and the one or more gRNAs are co-formulated as an LNP or polymeric nanoparticle.

In some embodiments, the delivery comprises administering a recombinant expression vector encoding the site-directed endonuclease. In some embodiments, the delivery comprises administering a recombinant expression vector encoding one or more gRNAs. In some embodiments, the delivery comprises administering a recombinant expression vector encoding the site-directed endonuclease and encoding one or more gRNAs, for example, on the same recombinant expression vector. In some embodiments, the delivery comprises administering the nucleic acid encoding the site-directed endonuclease and the nucleic acid encoding one or more gRNAs on different recombinant expression vectors, for example, up to 2, 3, or 4 recombinant expression vectors. In some embodiments, the recombinant expression vector is a non-viral vector (e.g., a plasmid). In some embodiments, the recombinant expression vector is a viral vector (e.g., an AAV). In some embodiments, the delivery comprises formulation of the one or more recombinant expression vectors using LNPs or polymeric nanoparticles.

In some embodiments, the delivery comprises administering the site-directed endonuclease as an mRNA, and administering the one or more gRNAs using a recombinant expression vector. In some embodiments, the delivery comprises administering the mRNA encoding the site-directed endonuclease formulated as an LNP or polymeric nanoparticle. In some embodiments, the delivery comprises administering the recombinant expression vector encoding the one or more gRNAs formulated as an LNP or polymeric nanoparticle. In some embodiments, the mRNA and the recombinant expression vector are separately formulated or co-formulated.

### I. Delivery of Complexes Comprising System Components

In some embodiments, the site-directed endonuclease is delivered as a polypeptide. In some embodiments, the site-directed endonuclease is delivered to a target cell population or target tissue ex vivo or in vivo as a polypeptide either alone or in combination with one or more gRNA molecules. In some embodiments, the site-directed endonuclease is delivered to target cell population or target tissue ex vivo or in vivo as a polypeptide that is pre-complexed with one or more guide RNAs. Such pre-complexed material is referred to herein as a “ribonucleoprotein particle” or “RNP”.

In some embodiments, the site-directed endonuclease is pre-complexed with one or more guide RNAs, or one or more sgRNAs. In some embodiments, the gene editing system comprises a ribonucleoprotein (RNP). In some embodiments, the gene editing system comprises a Cas9 RNP comprising a purified Cas9 protein (e.g., SpCas9, SluCas9, SaCas9) in complex with one or more gRNAs of the disclosure. The Cas9 protein can be expressed and purified by any means known in the art. In some embodiments, the ribonucleoprotein is assembled in vitro and delivered directly to cells using standard electroporation or transfection techniques known in the art. One benefit of the RNP is protection of the RNA from degradation.

In some embodiments, the site-directed endonuclease in the RNP is modified or unmodified. In some embodiments, the gRNA (e.g., crRNA, tracrRNA, or sgRNA) is modified or unmodified. Numerous modifications are known in the art and are suitable for use in the present disclosure.

In some embodiments, the site-directed endonuclease and the gRNA (e.g., sgRNA) are combined in an approximately 1:1 molar ratio. However, a wide range of molar ratios can be used to produce a RNP for use in the present disclosure.

In some embodiments, the RNP is delivered alone or using a delivery vehicle known in the art, for example, a lipid particle (e.g., LNP) or a synthetic nanoparticle (e.g., polymeric nanoparticle) or cell penetrating peptides (CPPs).

In some embodiments, ribonucleoprotein complexes comprising Cas9 protein (e.g., purified Cas9 protein) and one or more gRNA(s) are prepared for administration directly a target tissue. In some embodiments, RNP complexes comprising Cas9 protein (e.g., purified Cas9 protein), one or more gRNA(s), and one or more cell penetrating peptides are prepared for administration directly into a target tissue. Cell penetrating peptides for use in promoting RNP complex uptake by cells in a target tissue are known in the art. Non-limiting examples of CPPs for promoting cellular uptake of protein complexes include penetratin, R8, TAT, Transportan, Xentry, endo-porter, synthetic CPPs and cyclic derivatives thereof.

### II. Delivery of Nucleic Acids of the Disclosure

In some embodiments, the delivery comprises administering the site-directed endonuclease as a nucleic acid molecule (e.g., mRNA or recombinant expression vector). In some embodiments, delivery comprises administering one or more gRNAs or nucleic acid molecules encoding the one or more gRNAs (e.g., recombinant expression vector). In some embodiments, the nucleic acid molecules are delivered using a viral vector (e.g., AAV vector) or a non-viral delivery vehicle (e.g., LNP) known in the art. In some embodiments, a combination of a viral vector and a non-viral delivery vehicle are used.

In some embodiments, the nucleic acid molecules are delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Non-limiting exemplary non-viral delivery vehicles include those described in Peer and Lieberman, Gene Therapy, 18: 1127-1133 (2011) (which focuses on non-viral delivery vehicles for siRNA that are also useful for delivery of other polynucleotides).

In some embodiments, the nucleic acid molecules are delivered by viral delivery vehicles, such as AAV. In some embodiments, the cloning capacity of the viral vector requires more than one vector to deliver the components of a gene editing system as disclosed herein. For example, in some embodiments, one viral vector (e.g., AAV vector) comprises a nucleotide sequence encoding a site-directed endonuclease (e.g., Cas nuclease), while a second viral vector (e.g., AAV vector) comprises one or more nucleotide sequences encoding one or more gRNAs described herein. In some embodiments, the cloning capacity of the viral vector is sufficient to deliver all components of a gene editing system disclosed herein. For example, in some embodiments, one vector (e.g., AAV vector) comprises nucleotide sequence encoding a site-directed endonuclease (e.g., Cas nuclease) and one or more nucleotide sequences encoding one or more gRNAs described herein.

In some embodiments, a recombinant adeno-associated virus (rAAV) vector is used for delivery. Techniques to produce rAAV particles, in which an AAV genome to be packaged that includes the polynucleotide to be delivered (e.g., nucleic acid encoding one or more gRNAs and/or a site-directed endonuclease), rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 AAV rh.74 and tropism modified AAV vectors. Production of pseudotyped rAAV is disclosed in, for example, international patent application publication number WO 01/83692.

In some embodiments, a method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others (see Table 1).

TABLE 1

| Tissue/Cell Type | Serotype |
|---|---|
| Liver | AAV3, AAV5, AAV8, AAV9 |
| Skeletal muscle | AAV1, AAV7, AAV6, AAV8, AAV9 |
| Central nervous | AAV5, AAV1, AAV4, AAV8, AAV9 |
| system | |
| RPE | AAV5, AAV4, AAV2, AAV8, AAV9, AAVrh8R |
| Photoreceptor cells | AAV5, AAV8, AAV9, AAVrh8R |
| Lung | AAV9, AAV5 |
| Heart | AAV9 |
| Pancreas | AAV8 |
| Kidney | AAV2, AAV8 |

In some embodiments, the AAV vector serotype is matched to enable targeting of sensory neurons, for example, sensory neurons residing in the DRG (e.g., lumbar DRG). AAV serotypes are known for preferential tropism to different neuron sizes present in the DRG. For example, AAV-6 has been shown effective for transducing neurons with diameter less than approximately 300 μm²), AAV-5 has been shown effective for transducing neurons with diameter of approximately 300 to 700 μm², and AAV-8 has been shown effective for transducing neurons with diameter greater than approximately 700 μm² (see, e.g., Yu H, et al. (2013). PLoS One. 8(4):e61266; Jacques S J, et al (2012). Mol Cell Neurosci. 49(4):464-74; Xu Q, et al (2012) PLoS One 7(3):e32581). Accordingly, in some embodiments, an AAV serotype for use in the present disclosure is one having preferential tropism for neurons with diameter less than approximately 300 μm² (e.g., AAV-6), one having preferential tropism for neurons with diameter approximately 300 to 700 μm² (e.g., AAV-5), and/or one having preferential tropism for neurons with diameter greater than approximately 700 μm² (e.g., AAV-8).

In some embodiments, an AAV vector serotype for use in the present disclosure is one able to penetrate the blood brain barrier (BBB). As a non-limiting example, AAV9 has been shown to cross the BBB following in vivo administration, see, e.g., Bey, et al (2020) Mol Therapy: Methods & Clinical Development 17:771. In some embodiments, an AAV vector serotype for use in the present disclosure is AAV9.

In addition to adeno-associated viral vectors, other viral vectors can be used. Such viral vectors include, but are not limited to, adenovirus, lentivirus, alphavirus, enterovirus, pestivirus, baculovirus, herpesvirus, Epstein Barr virus, papovavirus, poxvirus, vaccinia virus, and herpes simplex virus.

### III. Nanoparticle Compositions

In some embodiments, the gene editing system components described herein, including polypeptides of the disclosure (e.g., site-directed endonuclease, Cas nuclease) and nucleic acids of the disclosure, e.g., gRNA(s), a recombinant expression vector encoding the gRNA(s) and/or a site-directed endonuclease, mRNA encoding a site-directed endonuclease, are delivered to a host cell or a patient by a lipid nanoparticle (LNP).

In some embodiments, the system components are formulated, individually or combined together, in nanoparticles or other delivery vehicles, (e.g., polymeric nanoparticles) to facilitate cellular uptake and/or to protect them from degradation when delivered to a subject.

In some embodiments, a nanoparticle composition comprises a lipid. Lipid nanoparticles include, but are not limited to, liposomes and micelles. Any number of lipids may be present, including cationic and/or ionizable lipids, anionic lipids, neutral lipids, amphipathic lipids, conjugated lipids (e.g., PEGylated lipids), and/or structural lipids. Such lipids can be used alone or in combination.

Nanoparticles are ultrafine particles typically ranging between 1 and 100 to 500 nanometers (nm) in size with a surrounding interfacial layer and often exhibiting a size-related or size-dependent property. Nanoparticle compositions are myriad and encompass lipid nanoparticles (LNPs), liposomes (e.g., lipid vesicles), and lipoplexes. For example, a nanoparticle composition can be a liposome having a lipid bilayer with a diameter of 500 nm or less. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, the nanoparticle composition comprises a site-directed endonuclease mRNA, gRNAs targeting one or more target sequences, recombinant expression vector(s) encoding the site-directed endonuclease and/or gRNA(s), or RNP comprising the site-directed endonuclease and gRNA(s). In some embodiments, the mRNA and gRNA(s) are each separately formulated for delivery, e.g., in lipid nanoparticles. In some embodiments, the mRNA and gRNA(s) are co-formulated for delivery, e.g., in a lipid nanoparticle. In some embodiments, the recombinant expression vector encoding a site-directed endonuclease and a recombinant expression vector encoding the gRNA(s) are separately formulated for delivery, e.g., in lipid nanoparticles. In some embodiments, the recombinant expression vector encoding a site-directed endonuclease and a recombinant expression vector encoding the gRNA(s) are co-formulated for delivery, e.g., in lipid nanoparticles. In some embodiments, the recombinant expression vector encoding a site-directed endonuclease and gRNA(s) is formulated for delivery, e.g, in a lipid nanoparticle.

In some embodiments, the disclosure provides LNP compositions comprising: (a) one or more nucleic acid molecules (e.g., mRNA, gRNA, recombinant expression vector) described herein or RNP described herein; and (b) one or more lipid moieties selected from the group consisting of amino lipids, helper lipids, structural lipids, phospholipids, ionizable lipids, PEG lipids, lipoid, and cholesterol or cholesterol derivatives. In some embodiments, the disclosure provides LNP compositions comprising: (a) one or more nucleic acid molecules (e.g., mRNA, gRNA, recombinant expression vector) described herein or RNP described herein; and (b) one or more lipid moieties selected from the group consisting of ionizable lipids, amino lipids, anionic lipids, neutral lipids, amphipathic lipids, helper lipids, structural lipids, PEG lipids, and lipoids, and optionally (c) targeting moieties.

In some embodiments, the LNP composition comprise one or more lipid moieties promote or enhances cellular uptake by the apolipoprotein E (apoE)-low density lipoprotein receptor (LDLR) pathway. For example, certain ionizable lipids are known in the art for increasing cellular uptake of LNPs by the apoE-LDLR pathway (see, e.g., Semple, et al (2010) NAT BIOTECH 28:172). In some embodiments, the LNP composition comprises one or more lipid moieties that promote or enhances cellular uptake by an apoE-LDLR independent pathway.

In some embodiments, the LNPs of the present disclosure are formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

### Pharmaceutical Compositions

In some embodiments, the disclosure provides pharmaceutical compositions comprising a gene editing system or system components described herein combined with an appropriate pharmaceutically acceptable carrier or diluent.

In some embodiments, the pharmaceutical composition comprises (1) one or more gRNAs described herein, and (2) a pharmaceutically acceptable carrier or diluent. In some embodiments, the pharmaceutical composition comprises (1) nucleic acid(s) encoding one or more gRNAs described herein, and (2) a pharmaceutically acceptable carrier or diluent. In some embodiments, the pharmaceutical composition comprises (1) recombinant expression vector(s) encoding one or more gRNAs described herein, and (2) a pharmaceutically acceptable carrier or diluent. In some embodiments, the pharmaceutical composition comprises one or more gRNAs, nucleic acid(s) encoding one or more gRNAs, or recombinant expression vector(s) (e.g., AAV) encoding one or more gRNAs formulated as a lipid composition (e.g., LNP), and (2) a pharmaceutically acceptable carrier or diluent. In some embodiments, the pharmaceutical composition comprises a therapeutically effective amount of the one or more gRNAs.

In some embodiments, the pharmaceutical composition comprises (1) a site-directed endonuclease (e.g., Cas nuclease) that is a polypeptide, and (2) a pharmaceutically acceptable carrier or diluent. In some embodiments, the pharmaceutical composition comprises (1) a nucleic acid molecule (e.g., mRNA) encoding a site-directed endonuclease (e.g., Cas nuclease), and (2) a pharmaceutically acceptable carrier or diluent. In some embodiments, the pharmaceutical composition comprises: (1) a recombinant expression vector (e.g., AAV) encoding a site-directed endonuclease (e.g., Cas nuclease), and (2) a pharmaceutically acceptable carrier or diluent. In some embodiments, the pharmaceutical composition comprises: (1) a site-directed endonuclease, a nucleic acid encoding a site-directed endonuclease, or a recombinant expression vector encoding the site-directed endonuclease formulated as a lipid composition (e.g., LNP), and (2) a pharmaceutically acceptable carrier or diluent. In some embodiments, the pharmaceutical composition comprises a therapeutically effective amount of the site-directed endonuclease.

In some embodiments, a pharmaceutical composition comprising the one or more gRNAs and the pharmaceutical composition comprising the site-directed endonuclease are the same pharmaceutical composition. In some embodiments, the pharmaceutical composition comprising the one or more gRNAs and the pharmaceutical composition comprising the site-directed endonuclease are different pharmaceutical compositions.

In some embodiments, the pharmaceutical composition comprises (1) (i) one or more gRNAs, (ii) a site-directed endonuclease (e.g., Cas nuclease) that is a polypeptide, and (2) a pharmaceutically acceptable carrier or diluent. In some embodiments, the pharmaceutical composition comprises (1), wherein (i) and (ii) are present as an RNP complex. In some embodiments, the RNP complex further comprises one or more cell penetrating peptides. In some embodiments, the pharmaceutical composition comprises (1), wherein (i) and/or (ii), or an RNP complex comprising (i) and (ii), are formulated as a lipid composition (e.g., LNP).

In some embodiments, the pharmaceutical composition comprises (1) (i) one or more gRNAs, (ii) a nucleic acid (e.g., mRNA) comprising a nucleotide sequence encoding a site-directed endonuclease (e.g., Cas nuclease), and (2) a pharmaceutically acceptable carrier or diluent. In some embodiments, the pharmaceutical composition comprises (1), wherein (i) and/or (ii) are formulated as a lipid composition (e.g., LNP).

In some embodiments, the pharmaceutical composition comprises (1) (i) one or more gRNAs, (ii) a recombinant expression vector (e.g., AAV) comprising a nucleotide sequence encoding a site-directed endonuclease (e.g., Cas nuclease), and (2) a pharmaceutically acceptable carrier or diluent. In some embodiments, the pharmaceutical composition comprises (1), wherein (i) and/or (ii) are formulated as a lipid composition (e.g., LNP).

In some embodiments, the pharmaceutical composition comprises (1) (i) a recombinant expression vector (e.g., AAV) comprising a nucleotide sequence encoding one or more gRNAs, (ii) a recombinant expression vector (e.g., AAV) comprising a nucleotide sequence encoding a site-directed endonuclease (e.g., Cas nuclease), and (2) a pharmaceutically acceptable carrier or diluent. In some embodiments, the recombinant expression vector of (i) and (ii) are the same recombinant expression vector. In some embodiments, the recombinant expression vector of (i) and (ii) are different recombinant expression vectors. In some embodiments, the recombinant expression vector(s) are formulated as a lipid composition (e.g., LNP).

Exemplary pharmaceutically acceptable excipients such as carriers, solvents, stabilizers, adjuvants, diluents, etc., depending upon the particular mode of administration and dosage form. Contemplated pharmaceutical compositions can be generally formulated to achieve a physiologically compatible pH, depending on the formulation and route of administration. In some embodiments, the compositions comprise a therapeutically effective amount of the one or more gRNAs, the site-directed endonuclease, the nucleic acid molecules, and/or the recombinant expression vectors, together with one or more pharmaceutically acceptable excipients.

Suitable excipients can include, for example, carrier molecules that include large, slowly metabolized macromolecules. Other exemplary excipients can include antioxidants, chelating agents, carbohydrates, stearic acid, liquids such as oils, water, saline, glycerol and ethanol, wetting or emulsifying agents, pH buffering substances, and the like.

Pharmaceutical compositions can be formulated into preparations in solutions, suppositories, injections. In some embodiments, the pharmaceutical composition is formulated to result in systemic administration of the one or more gRNAs, the site-directed endonuclease, the nucleic acid molecules, and/or the recombinant expression vectors, for example, following enteral or parenteral administration. In some embodiments, the pharmaceutical composition is formulated to result in localized administration of the one or more gRNAs, the site-directed endonuclease, the nucleic acid molecules, and/or the recombinant expression vectors, for example, following regional administration or implantation. In some embodiments, the pharmaceutical composition is formulated to result in localized administration to DRG (e.g., lumbar DRG) tissue following intra-DRG, intraneural, or intra-thecal administration or implantation. In some embodiments, the pharmaceutical composition is formulated for immediate activity or for sustained release of the one or more gRNAs, the site-directed endonuclease, the nucleic acid molecules, and/or the recombinant expression vectors.

In some embodiments, particularly wherein the pharmaceutical composition is formulated to target tissues of the central nervous system (CNS) following systemic administration, one more strategies are used to enable the components to cross the blood-brain barrier (BBB). For example, in some embodiments, the components (e.g., one or more gRNAs, site-directed endonuclease) are encoded by a delivery vehicle such as an AAV9 or derivatives thereof that result in passage through the BBB. One strategy for drug delivery through the BBB entails disruption of the BBB, either by osmotic means such as mannitol or leukotrienes, or biochemically using vasoactive substances such as bradykinin. In some embodiments, the BBB disrupting agent is co-administered with a pharmaceutical composition of the disclosure, e.g., by parenteral administration. Other strategies to go through the BBB entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. In some embodiments, active transport moieties are conjugated to the components (e.g., one or more gRNAs, site-directed endonuclease), or LNPs comprising the components, to facilitate transport across the endothelial wall of the blood vessel.

In some embodiments, a strategy for delivering the pharmaceutical composition behind the BBB comprises localized administration, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversibly affixed (see e.g. US Application Nos. 20080081064 and 20090196903, incorporated herein by reference).

Typically, an effective amount of a gene editing system comprising gRNA(s) and/or site-directed endonuclease described herein, or system components described herein, can be provided, for example, for use in a method of treating chronic pain. Methods of calculating the effective amount or effective dose are within the skill of one of ordinary skill in the art. The final amount to be administered is dependent upon the route of administration and upon the nature of the disorder that is to be treated. For example, in some embodiments, the final amount or dose of a gene editing system described herein is dependent upon the level of chronic pain experienced by the patient being treated. A competent clinician will be able to determine an effective amount of the gene editing system to administer to the patient to halt or reverse the progression of the disorder (e.g., to reduce or eliminate the level of chronic pain experienced by the patient).

In some embodiments, based on animal data (e.g., in animal models of acute inflammatory pain, post-surgical pain, osteoarthritic pain, neuropathic pain, and/or hypoalgesia), and other information available for the gene editing system, a clinician can determine the maximum safe dose for an individual, depending on the route of administration. For instance, an intravenously administered dose can be more than an intrathecally administered dose, given the greater body of fluid into which the therapeutic composition is being administered. Similarly, compositions which are rapidly cleared from the body can be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a gene editing system comprising gRNA(s) and/or site-directed endonuclease described herein, or system components described herein, can be obtained from a suitable commercial source. In some embodiments, therapies based on a gene editing system comprising gRNA(s) and/or site-directed endonuclease described herein, or system components described herein, i.e. preparations of gRNA(s) and/or site-directed endonuclease to be used for therapeutic administration, must be sterile. Therapeutic compositions can be generally placed into a container having a sterile access port, for example, an intravenous solution bag or vial having a stopper pierceable by a hypodermic injection needle. In some embodiments, the therapeutic components are stored in unit or multi-dose containers, for example, sealed ampules or vials, as an aqueous solution or as a lyophilized formulation for reconstitution.

### Methods of Use

In some embodiments, the disclosure provides cellular, ex vivo, and in vivo methods comprising use of the nucleic acid(s), system(s), expression vector(s), delivery system(s), or pharmaceutical composition(s) described herein to create a gene edit in one or more target genes (e.g., FAAH and FAAH-OUT) in the genome. In some embodiments, the methods comprise use of a site-directed endonuclease (e.g., Cas nuclease) and one or more gRNAs described herein, to introduce a mutation within or proximal the coding sequence of FAAH and/or introduce a deletion comprising a region of FAAH-OUT, wherein the mutation and/or deletion modulates (e.g., decreases) FAAH expression. In some embodiments, the disclosure provides methods of treating a patient with a disease or condition (e.g., chronic pain), wherein the method comprises administering nucleic acid(s), system(s), expression vector(s), delivery system(s), or pharmaceutical composition(s) described herein to introduce the desired gene edit in the genome of a target cell population and/or target tissue.

### I. Cellular Genome Editing

In some embodiments, the method comprises introducing a nucleic acid(s), system(s), expression vector(s), delivery system(s), or pharmaceutical composition(s) described herein to a cell or cell population. In some embodiments, the method comprises contacting the cell with a nucleic acid, system, expression vector, delivery system, or pharmaceutical composition described herein. In some embodiments, the method comprises generating a stable cell line comprising a genomic DNA molecule edited using a system of gene editing described herein. In some embodiments, the cell is a eukaryotic cell. In some embodiments, the eukaryotic cell is a mammalian cell. In some embodiments, the eukaryotic cell is a rodent cell. In some embodiments, the eukaryotic cell is a human cell. In some embodiments, the cell is a patient-derived cell.

The nucleic acid(s), system(s), expression vector(s), delivery system(s), or pharmaceutical composition(s) described herein may be introduced into the cell via any methods known in the art, such as, e.g., viral or bacteriophage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, shear-driven cell permeation, fusion to a cell-penetrating peptide followed by cell contact, microinjection, and nanoparticle-mediated delivery. In some embodiments, the vector system may be introduced into the cell via viral infection.

In some embodiments, the disclosure provides methods for inducing a double-stranded break (DSB) in a genomic DNA molecule, wherein the DSB is within or proximal one or more exons of the FAAH coding sequence in a cell, wherein repair of the DSB introduces a mutation in the FAAH coding sequence, and wherein the mutation disrupts FAAH expression in the cell. In some embodiments, the method comprises contacting the cell with one or more nucleic acid(s), system(s), expression vector(s), delivery system(s), or pharmaceutical composition(s) described herein comprising (i) a site-directed endonuclease and (ii) at least one gRNA directed to the FAAH gene; wherein when the system, the nucleic acid molecule, the expression vector, delivery system, or the pharmaceutical composition contacts the cell, the gRNA combines with the site-directed endonuclease to induce a mutation within or proximal one or more exons of the FAAH coding sequence, thereby resulting in reduced FAAH expression in the cell.

In some embodiments, the disclosure provides methods for inducing a deletion in a genomic DNA molecule comprising FAAH upstream FAAH-OUT, wherein the deletion disrupts FAAH-OUT and/or FAAH expression in the cell. In some embodiments, the method comprises contacting the cell with a one or more nucleic acid(s), system(s), expression vector(s), delivery system(s), or pharmaceutical composition(s) described herein comprising (i) a site-directed endonuclease; (ii) a first gRNA molecule comprising a spacer sequence corresponding to a first target sequence downstream the 3′ terminus of FAAH and upstream the transcriptional start site of FAAH-OUT in the genomic DNA molecule; and (iii) a second gRNA molecule comprising a spacer sequence corresponding to a second target sequence downstream the FAAH-OUT transcriptional start site and upstream exon 3 of FAAH-OUT in the genomic DNA molecule; wherein when the system, the nucleic acid molecule, the expression vector, the delivery system, or the pharmaceutical composition contacts the cell, the first and second gRNAs each independently combine with the site-directed endonuclease to induce a DSB proximal the first and second target sequences in the genomic DNA molecule, wherein the DSB proximal the first and second target sequences result in a deletion in the genomic DNA molecule, and wherein the deletion reduces results in reduced FAAH expression in the cell.

### II. In Vivo Genome Editing

Embodiments of the disclosure also encompass treating a patient with nucleic acid(s), system(s), expression vector(s), delivery system(s), or pharmaceutical composition(s) described herein. In some embodiments, the patient has chronic pain. Non-limiting examples of chronic pain include pain from conditions such as rheumatoid arthritis, peripheral neuropathy, idiopathic pain, or pain associated with cancer.

In some embodiments, the pain is nociceptive pain, neuropathic pain or inflammatory pain. In some embodiments, the nociceptive pain is due to a pathologically normal response to a noxious insult or injury of one or more tissues (e.g., skin tissue, muscle tissue, visceral organs, joints, tendons, bones). In some embodiments, the neuropathic pain is caused by damage or disease affecting the somatosensory nervous system. Non-limiting examples of such neuropathic pain include carpal tunnel syndrome, central pain syndrome, degenerative disc disease, diabetic neuropathy, phantom limb pain, shingles, pudendal neuralgia, sciatic, and trigeminal neuralgia. In some embodiments, neuropathic pain is associated with a disease or disorder, such as cancer, multiple sclerosis, kidney disease, infectious disease, spinal cord injury. In some embodiments, the neuropathic pain is post-surgical pain. In some embodiments, the pain is inflammatory pain caused by activation of nociceptive pathways as a result of tissue inflammation. Non-limiting examples of inflammatory pain include osteoarthritis, rheumatoid arthritis, Chron's disease, and fibromyalgia.

As used herein, “treating” a patient with chronic pain refers to a prevention of pain, a reduction or prevention of the development or progression of pain, and/or a reduction or elimination of existing pain. In some embodiments, a method of the disclosure is performed prior to or shortly after the onset of pain. In some embodiments, the method is performed following an extended duration of pain. In some embodiments, the method is performed in order to delay or prevent the onset of pain.

In some embodiments, the methods described herein are for use in treating a patient having a neurological disorder, such as anxiety, depression, or post traumatic stress disorders. In some embodiments, the methods described herein are for use in reducing or eliminating acute pain, for example, due to a wound or wound repair.

In some embodiments, the disclosure provides methods for treating a subject in need thereof (e.g., a subject with chronic pain) by reducing FAAH expression in a target tissue or cell population, the method comprising administering an effective amount of one or more nucleic acid(s), system(s), expression vector(s), delivery system(s), or pharmaceutical composition(s) described herein comprising (i) a site-directed endonuclease and (ii) at least one gRNA directed to the FAAH gene; wherein when the system, the nucleic acid molecule, the expression vector, the delivery system, or the pharmaceutical composition is administered, the gRNA combines with the site-directed endonuclease to induce a mutation within or proximal one or more exons of the FAAH coding sequence, thereby resulting in reduced FAAH expression in the target tissue or cell population.

In some embodiments, the disclosure provides methods for treating a subject in need thereof (e.g., a subject with chronic pain) by reducing FAAH expression in a target tissue or cell population, the method comprising administering an effective amount of one or more nucleic acid(s), system(s), expression vector(s), delivery system(s), or pharmaceutical composition(s) described herein comprising (i) a site-directed endonuclease; (ii) a first gRNA molecule comprising a spacer sequence corresponding to a first target sequence downstream the 3′ terminus of FAAH and upstream the transcriptional start site of FAAH-OUT in the genomic DNA molecule; and (iii) a second gRNA molecule comprising a spacer sequence corresponding to a second target sequence downstream the FAAH-OUT transcriptional start site and upstream exon 3 of FAAH-OUT in the genomic DNA molecule; wherein when the system, the nucleic acid molecule, the expression vector, the delivery system, or the pharmaceutical composition is administered, the first and second gRNAs each independently combine with the site-directed endonuclease to induce a DSB proximal the first and second target sequences in the genomic DNA molecule, wherein the DSB proximal the first and second target sequences result in a deletion in the genomic DNA molecule, and wherein the deletion reduces results in reduced FAAH expression in the target tissue or cell population.

### A. Administration

In some embodiments, the disclosure provides methods for modulating (e.g., decreasing) FAAH expression and/or activity in a subject in need thereof (e.g., a subject with chronic pain), the method comprising administering components of a gene editing system for editing FAAH and/or FAAH-OUT, or a pharmaceutical composition thereof, as described herein, wherein the components are administered together (e.g., sequentially or simultaneously).

In some embodiments, the target cell population or target tissue is any cell population or tissue known to express FAAH. For example, FAAH is highly expressed in multiple tissue types, including brain, small intestine, pancreas, skeletal muscle, and testis. Additionally, FAAH is further expressed in kidney, liver, lung, placenta, immune cells, and prostate tissue (see, e.g., Wei et al (2006) J BIOL CHEM 281:36569). FAAH is also expressed in adipose tissue, adrenal gland, bone marrow, fallopian, ovary, pituitary gland, rectum, stomach, thyroid, and tonsil tissues (see, eg., EMBL-EBI Expression Atlas Reference No. 30777892; Wang et al (2019) MOL SYSTEMS BIOL 15:e8503).

In some embodiments, the target tissue or cell population is found in the brain. In some embodiments, the target tissue or cell population is found in a dorsal root ganglion (DRG), for example, the lumbar DRG. In some embodiments, the target cell population are neurons. In some embodiments, the target cell population are sensory neurons, for example, sensory neurons of the DRG (e.g., lumbar DRG).

In some embodiments, the route of administration is any considered sufficient for delivery (e.g., localized delivery) of a gene-editing system described herein, or pharmaceutical composition thereof, to a desired target cell population (e.g., neurons) or target tissue (e.g., brain or DRG tissue) as ascertained by one of skill in the art. In some embodiments, the route of administration for delivery (e.g., localized delivery) of a gene-editing system described herein, or pharmaceutical composition thereof, to neurons of the DRG (e.g., lumbar DRG), is intra-DRG, intraneural, or intrathecal.

In some embodiments, the method comprises administering the system components by the same or different routes of administration. For example, in some embodiments, such as those for inducing a mutation within or proximal the FAAH coding sequence or for inducing a deletion comprising a region of FAAH-OUT, the gRNA(s) are administered by the same or different routes of administration as the site-directed endonuclease.

### B. Therapeutic Effects

In some embodiments, administration of the nucleic acid(s), system(s), expression vector(s), delivery system(s), or pharmaceutical composition(s) described herein results in a mutation comprising an insertion, deletion, or substitution of one or more nucleotides of a target gene (e.g., FAAH and/or FAAH-OUT) in a genomic DNA molecule in the patient, for example, in a target cell population and/or target tissue. In some embodiments, the mutation results in one or more amino acid changes in a protein expressed from the target gene, for example one or more amino acid changes in a FAAH-OUT and/or FAAH polypeptide expressed from the target gene. In some embodiments, the mutation results in one or more nucleotide changes in an RNA expressed from the target gene, such as an RNA expressed from the FAAH and/or FAAH-OUT target gene. In some embodiments, the mutation alters the expression level of the target gene, for example, altering or decreasing the expression level of FAAH and/or FAAH-OUT. In some embodiments, the mutation results in gene knockdown in the patient, for example, a gene knockdown of FAAH and/or FAAH-OUT. In some embodiments, the administration of the nucleic acid(s), system(s), expression vector(s), delivery system(s), or pharmaceutical composition(s) described herein results in a mutation (e.g., insertion, deletion) of an exon sequence, an intron sequence, a transcriptional control sequence, a translational control sequence, or a non-coding sequence of target gene (e.g. FAAH and/or FAAH-OUT).

In some embodiments, administration of the nucleic acid(s), system(s), expression vector(s), delivery system(s), or pharmaceutical composition(s) described herein results in deletion of a genomic DNA molecule comprising at least a portion of FAAH-OUT in a subject. Methods of measuring a deletion in a genome (e.g., an approximately 2-10 kb deletion comprising at least a portion of FAAH-OUT) are known in the art, and include, long-range PCR, digital droplet PCR (ddPCR), Anchor-Seq, and long-read sequencing.

In some embodiments, administration of the nucleic acid(s), system(s), expression vector(s), delivery system(s), or pharmaceutical composition(s) described herein results in decreased FAAH expression and/or activity in a subject. In some embodiments, a decrease in FAAH expression is measured as decreased expression of FAAH mRNA, FAAH polypeptide, or both. In some embodiments, a decrease in FAAH activity is measured as decreased catalytic hydrolysis of one or more FAAH substrates, e.g., AEA, OEA, or PEA.

In some embodiments, the level of FAAH expression (e.g., expression of FAAH mRNA and/or polypeptide) is decreased at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%, for example, relative to FAAH expression prior to the genome editing.

In some embodiments, FAAH expression is decreased in one or more tissues of a subject, including any tissue known to express FAAH. In some embodiments, FAAH expression is decreased in one or more regions of the brain (e.g., cerebral cortex, cerebellum, hippocampus). In some embodiments, FAAH expression is decreased in the thyroid gland, the adrenal gland, intestinal tissue, lung tissue, the esophagus, stomach tissue, a urinary tissue, a reproductive tissue, kidney tissue, liver tissue, or skin tissue.

Methods of measuring FAAH mRNA and/or polypeptide expression in a tissue are known in the art. A non-limiting exemplary method for measuring FAAH mRNA expression level in a tissue in a subject comprises obtaining a tissue sample from a subject (e.g., a biopsy tissue sample), isolating RNA from the tissue sample, and quantifying FAAH mRNA using quantitative PCR (qPCR) or digital droplet PCR, and in-situ hybridization. A non-limiting exemplary method for measuring FAAH polypeptide expression levels in a tissue in a subject comprises obtaining a tissue sample from a subject (e.g., a biopsy tissue sample), isolating protein from the tissue sample, and quantifying FAAH polypeptide using western blot, ELISA or LC-MS.

In some embodiments, decreased FAAH expression and/or activity results in increased levels of one or more FAAH substrates in the subject. In some embodiments, the level of the one or more FAAH substrates is increased relative to an untreated subject or to a subject prior to genomic editing. In some embodiments, the FAAH substrate is an N-acyl ethanolamine. In some embodiments, the FAAH substrate is an N-acyl taurine. In some embodiments, the FAAH substrate is oleamide. In some embodiments, the FAAH substrate that is an N-acyl ethanolamine is selected from AEA, PEA, and OEA.

In some embodiments, the one or more FAAH substrates is increased by about 30%, about 35%, about 40%, about 45%, about 50%, about 55%, about 60%, about 65%, about 70%, about 75%, about 80%, about 85%, about 90% or about 100%. In some embodiments, the one or more FAAH substrates is increased by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.5-fold, about 3-fold, about 3.5-fold, about 4-fold, about 4.5-fold, or about 5-fold. Methods of measuring the level of a FAAH substrate in a sample are known in the art. Non-limiting exemplary methods include obtaining a tissue sample (e.g., a blood sample) from a subject, and measuring level of a FAAH substrate (e.g., AEA, PEA, OEA) using LCMS.

In some embodiments, the disclosure provides methods of in vivo genomic editing for modulating (e.g., decreasing) FAAH expression and/or activity in a subject, wherein the method results in an analgesic effect (e.g., decreased pain). Methods of measuring reduction or elimination of pain in a subject are known in the art. Non-limiting examples of methods to measure pain include quantitative sensory testing (QTS), the McGill pain questionnaire, or the McGill pain index.

### C. Combination Therapy

In some embodiments, the method is used as a single therapy or in combination with other therapies available in the art.

In some embodiments, a gene editing system described herein is combined with one more inhibitors of FAAH and any pain medication known in the art and approved for human use.

Several classes of FAAH inhibitors are known (see, e.g., Deng, et al (2010) EXPERT OPIN DRUG DISC 5:961). These inhibitors include covalent irreversible inhibitors, covalent reversible inhibitors, and noncovalent reversible inhibitors.

Non-limiting examples of covalent reversible inhibitors include alpha-ketoheterocycles (see, e.g., Boger, et al (2000) PNAS 97:5044; Leung et al (2003) NAT BIOTECHNOL 21:687).

Non-limiting examples of covalent irreversible inhibitors include N-piperdine/N-piperazine carboxamides (see, e.g., Ahn, et al (2007) BIOCHEM 46:13019; Ahn et al (2009) CHEM BIOL 16:411; Johnson, et al (2009) BIOORG MED CHEM LETT 19:2865; Keith, et al (2008) BIOORG MED CHEM LETT 18:4838), carbamates (see, e.g., Timmons, et al (2008) BIOORG MED CHEM LETT 18:2109; Tarzia, et al (200) J MED CHEM 46:2352; Mor et al (2004) J MED CHEM 47:4998). Piperdine-based or piperazine-based urea derivatives that function as FAAH inhibitors are further disclosed by WO2009/127943 and WO2006/054652.

Non-limiting examples of noncovalent reversible inhibitors include ketobenzimidazoles (see, e.g., Min et al (2011) PNAS 108:7379).

### Kits

The present disclosure provides kits for carrying out the methods described herein. In some embodiments, the kit includes one or more gRNAs, nucleic acid(s) encoding the one or more gRNAs, a site-directed polypeptide, a nucleic acid encoding a site-directed polypeptide, recombinant expression vector(s) comprising the nucleic acids, delivery systems and/or any nucleic acid or proteinaceous molecule necessary to carry out the aspects of the methods described herein, or any combination thereof.

In some embodiments, a kit for use in the present disclosure comprises: (1) one or more gRNAs, and (2) reagents for reconstitution and/or dilution of (1). In some embodiments, a kit for use in the present disclosure comprises: (1) nucleic acid (s) encoding one or more gRNAs, and (2) reagents for reconstitution and/or dilution of (1). In some embodiments, a kit for use in the present disclosure comprises: (1) recombinant expression vector(s) encoding one or more gRNAs, and (2) reagents for reconstitution and/or dilution of (1). In some embodiments, a kit for use in the present disclosure comprises: (1) one or more gRNAs, nucleic acid(s) encoding one or more gRNAs, or recombinant expression vector(s) encoding one or more gRNAs formulated as an LNP, and (2) reagents for reconstitution and/or dilution of (1).

In some embodiments, a kit for use in the present disclosure comprises: (1) a site-directed endonuclease that is a polypeptide, and (2) reagents for reconstitution and/or dilution of (1). In some embodiments, a kit for use in the present disclosure comprises: (1) an mRNA encoding a site-directed endonuclease, and (2) reagents for reconstitution and/or dilution of (1). In some embodiments, a kit for use in the present disclosure comprises: (1) a recombinant expression vector encoding a site-directed endonuclease, and (2) reagents for reconstitution and/or dilution of (1). In some embodiments, a kit for use in the present disclosure comprises: (1) a site-directed endonuclease or a nucleic acid encoding a site-directed endonuclease formulated as an LNP, and (2) reagents for reconstitution and/or dilution of (1).

In some embodiments, a kit for use in the present disclosure comprises: (1) (i) one or more gRNAs, (ii) an mRNA comprising a nucleotide sequence encoding a site-directed endonuclease, and (2) reagents for reconstitution and/or dilution of (i) and (ii).

In some embodiments, a kit for use in the present disclosure comprises: (1) (i) one or more gRNAs, (ii) a site-directed endonuclease polypeptide, and (2) reagents for reconstitution and/or dilution of (i) and (ii).

In some embodiments, a kit for use in the present disclosure comprises: (1) a recombinant expression vector comprising a nucleotide sequence encoding one or more gRNAs, and (2) a reagent for reconstitution and/or dilution of the recombinant expression vector(s).

In some embodiments, a kit for use in the present disclosure comprises: (1) a nucleotide sequence encoding a site-directed endonuclease, and (2) a reagent for reconstitution and/or dilution of the recombinant expression vector(s).

In some embodiments, a kit for use in the present disclosure comprises: (1) a recombinant expression vector comprising (i) a nucleotide sequence encoding one or more gRNAs (ii) nucleotide sequence encoding a site-directed endonuclease, and (2) a reagent for reconstitution and/or dilution of the recombinant expression vector(s).

Components of a kit can be in separate containers, or combined in a single container.

Any kit described above can further comprise one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the site-directed endonuclease, or improve the specificity of targeting.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

### Definitions

It must be noted that, as used in the specification and the appended claims, the singular forms “a,” “an” and “the” include plural referents unless the context clearly dictates otherwise. Further, unless otherwise required by context, singular terms shall include pluralities and plural terms shall include the singular.

As used herein, the term “about” (alternatively “approximately”) will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “base pair” refers to two nucleobases on opposite complementary polynucleotide strands, or regions of the same strand, that interact via the formation of specific hydrogen bonds. As used herein, the term “Watson-Crick base pairing”, used interchangeably with “complementary base pairing”, refers to a set of base pairing rules, wherein a purine always binds with a pyrimidine such that the nucleobase adenine (A) forms a complementary base pair with thymine (T) and guanine (G) forms a complementary base pair with cytosine (C) in DNA molecules. In RNA molecules, thymine is replaced by uracil (U), which, similar to thymine (T), forms a complementary base pair with adenine (A). The complementary base pairs are bound together by hydrogen bonds and the number of hydrogen bonds differs between base pairs. As in known in the art, guanine (G)-cytosine (C) base pairs are bound by three (3) hydrogen bonds and adenine (A)-thymine (T) or uracil (U) base pairs are bound by two (2) hydrogen bonds.

As used herein, the term “codon” refers to a sequence of three nucleotides that together form a unit of genetic code in a DNA or RNA molecule. A codon is operationally defined by the initial nucleotide from which translation starts and sets the frame for a run of successive nucleotide triplets, which is known as an “open reading frame” (ORF). For example, the string GGGAAACCC, if read from the first position, contains the codons GGG, AAA, and CCC; if read from the second position, it contains the codons GGA and AAC; and if read from the third position, GAA and ACC. Thus, every nucleic sequence read in its 5′→3′ direction comprises three reading frames, each producing a possibly distinct amino acid sequence (in the given example, Gly-Lys-Pro, Gly-Asn, or Glu-Thr, respectively). DNA is double-stranded defining six possible reading frames, three in the forward orientation on one strand and three reverse on the opposite strand. Open reading frames encoding polypeptides are typically defined by a start codon, usually the first AUG codon in the sequence.

The term “induces a mutation” refers to an incorporation of an alteration by a gene-editing system described herein that results in a change of one or more nucleotides in a genomic DNA molecule such that expression of the genomic DNA is altered in a desired manner. In some embodiments, the induction of a mutation is for therapeutic purposes or results in a therapeutic effect (e.g., modulation of FAAH expression and/or activity).

As used herein, the term “complementary” or “complementarity” refers to a relationship between the sequence of nucleotides comprising two polynucleotide strands, or regions of the same polynucleotide strand, and the formation of a duplex comprising the strands or regions, wherein the extent of consecutive base pairing between the two strands or regions is sufficient for the generation of a duplex structure. It is known that adenine (A) forms specific hydrogen bonds, or “base pairs”, with thymine (T) or uracil (U). Similarly, it is known that a cytosine (C) base pairs with guanine (G). It is also known that non-canonical nucleobases (e.g., inosine) can hydrogen bond with natural bases. A sequence of nucleotides comprising a first strand of a polynucleotide, or a region, portion or fragment thereof, is said to be “sufficiently complementary” to a sequence of nucleotides comprising a second strand of the same or a different nucleic acid, or a region, portion, or fragment thereof, if, when the first and second strands are arranged in an antiparallel fashion, the extent of base pairing between the two strands maintains the duplex structure under the conditions in which the duplex structure is used (e.g., physiological conditions in a cell). It should be understood that complementary strands or regions of polynucleotides can include some base pairs that are non-complementary. Complementarity may be “partial,” in which only some of the nucleobases comprising the polynucleotide are matched according to base pairing rules. Or, there may be “complete” or “total” complementarity between the nucleic acids. Although the degree of complementarity between polynucleotide strands or regions has significant effects on the efficiency and strength of hybridization between the strands or regions, it is not required for two complementary polynucleotides to base pair at every nucleotide position. In some embodiments, a first polynucleotide is 100% or “fully” complementary to a second polynucleotide and thus forms a base pair at every nucleotide position. In some embodiments, a first polynucleotide is not 100% complementary (e.g., is 90%, or 80% or 70% complementary) and contains mismatched nucleotides at one or more nucleotide positions. While perfect complementarity is often desired, some embodiments can include one or more but preferably 6, 5, 4, 3, 2, or 1 mismatches.

As used herein, the term “contacting” means establishing a physical connection between two or more entities. For example, contacting a cell with an agent (e.g., a nucleic acid molecule, a system, a lipid nanoparticle composition, or pharmaceutical composition of the disclosure) means that the cell and the agent are made to share a physical connection. Methods of contacting cells with external entities both in vivo, in vitro, and ex vivo are well known in the biological arts. In exemplary embodiments of the disclosure, the step of contacting a mammalian cell with a composition (e.g a nucleic acid molecule, a system, a lipid nanoparticle composition, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a nucleic acid molecule, a system, a lipid nanoparticle composition, or pharmaceutical composition of the disclosure) and a cell may be contacted, for example, by adding the composition to the culture medium of the cell and may involve or result in transfection. Moreover, more than one cell (e.g., a population of cells) may be contacted by an agent described herein.

As used herein, the term “culture” can be used interchangeably with the terms “culturing”, “grow”, “growing”, “maintain”, “maintaining”, “expand”, “expanding” when referring to a cell culture or the process of culturing. The term refers to a cell (e.g., a primary cell) that is maintained outside its normal environment (e.g., a tissue in a living organism) under controlled conditions. Cultured cells are treated in a manner that enables survival. Culturing conditions can be modified to alter cell growth, homeostasis, differentiation, division, or a combination thereof in a controlled and reproducible manner. The term does not imply that all cells in the culture survive, grow, or divide as some may die, enter a state of quiescence, or enter a state of senescence. Cells are typically cultured in media, which can be changed during the course of the culture. Components can be added to the media or environmental factors (e.g., temperature, humidity, atmospheric gas levels) to promote cell survival, growth, homeostasis, division, or a combination thereof.

As used herein the term, “double-strand break” (DSB) refers to a DNA lesion generated when the two complementary strands of a DNA molecule are broken or cleaved, resulting in two free DNA ends or termini DSBs may occur via exposure to environmental insults (e.g., irradiation, chemical agents, or UV light) or generated deliberately (e.g., via a system comprising a site-directed endonuclease) and for a defined biological purpose (e.g., to induce a mutation in a genomic DNA molecule).

As used herein, the term “effective dose” or “effective dosage” is defined as an amount sufficient to achieve or at least partially achieve the desired effect.

As used herein, the term “genome editing”, “gene-editing” and “genomic editing” are used interchangeably, and generally refer to the process of editing or changing the nucleotide sequence of a genome, preferably in a precise or predetermined manner Examples of methods of genome editing described herein include methods of using site-directed endonucleases to cut genomic DNA at a precise target location or sequence within a genome, thereby creating a DNA break (e.g., a DSB) within the target sequence, and repairing the DNA break such that the nucleotide sequence of the repaired genome has been changed at or near the site of the DNA break.

Double-strand DNA breaks (DSBs) can be and regularly are repaired by natural, endogenous cellular processes such as homology-directed repair (HDR) and non-homologous end-joining (NHEJ) (see e.g., Cox et al., (2015) Nature Medicine 21(2):121-131).

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment.

As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule (e.g., a wild-type gene allele).

As used herein, the term “intron” refers to any nucleotide sequence within a gene that is removed by RNA splicing mechanisms during maturation of the final RNA product (e.g., an mRNA). An intron refers to both the DNA sequence within a gene and the corresponding sequence in a RNA transcript (e.g., a pre-mRNA). Sequences that are joined together in the final mature RNA after RNA splicing are “exons”. As used herein, the term “intronic sequence” refers to a nucleotide sequence comprising an intron or a portion of an intron. Introns are found in the genes of most eukaryotic organisms and can be located in a wide range of genes, including those that generate proteins, ribosomal RNA (rRNA), and transfer RNA (tRNA). When proteins are generated from intron-containing genes, RNA splicing takes place as part of the RNA processing pathway that follows transcription and precedes translation.

As used herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

As used herein, an “mRNA” refers to a messenger ribonucleic acid. An mRNA may be naturally or non-naturally occurring or synthetic. For example, an mRNA may include modified and/or non-naturally occurring components such as one or more nucleobases, nucleosides, nucleotides, or linkers. An mRNA may include a cap structure, a 5′ transcript leader, a 5′ untranslated region, an initiator codon, an open reading frame, a stop codon, a chain terminating nucleoside, a stem-loop, a hairpin, a polyA sequence, a polyadenylation signal, and/or one or more cis-regulatory elements. An mRNA may have a nucleotide sequence encoding a polypeptide. Translation of an mRNA, for example, in vivo translation of an mRNA inside a mammalian cell, may produce a polypeptide. Traditionally, the basic components of a natural mRNA molecule include at least a coding region, a 5′-untranslated region (5′-UTR), a 3′UTR, a 5′ cap and a polyA sequence.

As used herein, the term “naturally occurring” as applied to an object refers to the fact that an object can be found in nature. For example, a polypeptide or polynucleotide sequence (e.g., a splice site), or components thereof such as amino acids or nucleotides, that is present in an organism (including viruses) that can be isolated from a source in nature and which has not been intentionally modified by man in the laboratory is naturally occurring.

As used herein, the term “nucleic acid” refers to deoxyribonucleotides or ribonucleotides and polymers or oligomers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Polymers of nucleotides are referred to as “polynucleotides”.

As used herein, a nucleic acid, or fragment or portion thereof, such as a polynucleotide or oligonucleotide is “operably linked” when it is placed into a functional relationship with another nucleic acid sequence, or fragment or portion thereof.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As used herein, the term “percent identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website. The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “site-directed endonuclease” refers to a nuclease for use with a CRISPR/Cas system (e.g., Cas9) that recognizes a specific target sequence in a DNA molecule (e.g., a genomic DNA molecule) and generates a DNA break (e.g., a DSB) within the DNA molecule at, near or within the target sequence, when combined with a gRNA molecule comprising a spacer sequence corresponding to the target sequence. After creation of the DNA break, the cellular DNA repair machinery is co-opted to repair the DNA break, thereby resulting in a mutation proximal the target sequence in the DNA molecule. The site-directed endonuclease refers to the nuclease in polypeptide form. In some embodiments, the site-directed endonuclease is encoded by a nucleic acid molecule (e.g., mRNA). In some embodiments, the site-directed endonuclease is encoded by a recombinant expression vector (e.g., AAV).

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions.

### EQUIVALENTS AND SCOPE

Those skilled in the art will recognize, or be able to ascertain using no more than routine experimentation, many equivalents to the specific embodiments, described herein. The scope of the present disclosure is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein.

It is also noted that the term “comprising” is intended to be open and permits but does not require the inclusion of additional elements or steps. When the term “comprising” is used herein, the term “consisting of” is thus also encompassed and disclosed

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

### Example 1: In Silico Identification of gRNA Target Sequences in the FAAH Coding Sequence

To develop a CRISPR/Cas9 system targeting the FAAH coding sequence, the human FAAH gene was evaluated for candidate guide RNA (gRNA) target sequences. Specifically, an in silico algorithm based on the CCTop algorithm (see, e.g., Stemmer, M. et al (2015) PLoS ONE 10(4):e0124633) was used to identify gRNA target sequences immediately upstream a PAM for S. pyogenes Cas9 (SpCas9), S. lugdunensis Cas9 (SluCas9), or S. aureus Cas9 (SaCas9) in the FAAH coding sequence.

The region of the FAAH coding sequence evaluated for potential target sequences encompassed either exons 1-2 or exons 1-4, as introducing a mutation (e.g., frameshift mutation) in an exon proximal to the start codon was expected to increase the likelihood of a functional knock down (e.g., by inhibiting FAAH expression and/or producing a dysfunctional protein product). Chromosomal location of FAAH genomic regions are identified in Table 2.

TABLE 2
Chromosomal location of regions of FAAH

| Region of FAAH | Chromosome Location* |
|---|---|
| FAAH gene with regulatory elements | Chr1: 46,392,317-46,415,848 |
| FAAH 5′UTR | Chr1: 46,394,317-46,394,348 |
| FAAH coding sequence | Chr1: 46,394,349-46,413,575 |
| Exon 1 | Chr1: 46,394,317-46,394,543 |
| Exon 2 | Chr1: 46,402,091-46,402,204 |
| Exon 3 | Chr1: 46,405,014-46,405,148 |
| Exon 4 | Chr1: 46,405,372-46,405,505 |
| FAAH 3′UTR | Chr1: 46,413,576-46,413,845 |

*According to human reference genome Hg38

An approximately 4 kb region (i.e., 4193 bp for exon 1 and 4115 bp for exon2) from 2 kb upstream to 2 kb downstream of exon 1 and exon 2 of the FAAH coding sequence (i.e., exon 1 chr1:46,392,351-46,396,543; exon 2 chr1:46,400,090-46,404,204 of Hg38) was evaluated to identify gRNA target sequences for use with SpCas9, i.e., target sequences with the pattern N₂₀NGG (N=A,G,C,T; SEQ ID NO: 1282) using the CCTop algorithm (Stemmer et al, 2015 PLOS ONE 10:e0124633).

Likewise, the same region was evaluated to identify gRNA target sequences for use with SluCas9, i.e., target sequences with the pattern N₂₀NNGG (N=A,G,C,T; SEQ ID NO: 1283) using the CCTop algorithm.

The same region was evaluated to identify gRNA target sequences for use with SaCas9, i.e., target sequences with the pattern N₂₁NNGRRT (N=A,G,C,T; R=A,G; SEQ ID NO: 1284) using the CCTop algorithm.

The analysis identified approximately 1586 gRNA target sequences upstream SpCas9 PAM (NGG), approximately 1586 gRNA target sequences upstream SluCas9 PAM (NNGG), and approximately 241 gRNA target sequences upstream SaCas9 PAM (NNGRRT).

Subsequently, spacer sequences corresponding to the gRNA target sequences for SpCas9, SluCas9, and SaCas9 were filtered using the information on off-target sites generated by the CCTop algorithm. Specifically, spacers were filtered to remove any that had one or more perfect matches to a different target site in the human genome (Hg38). The spacers were also filtered based upon prediction of off-target sites with up to 4 mismatches in the human genome (Hg38). Spacers were removed that were predicted to have either (i) one or more off-target sites with one mismatch; or (ii) three or more off-target sites with two mismatches. Moreover, spacers were selected for target sequences having a minor allele frequency of less than or equal to 0.001 in the human population and an exonic or 5′ upstream sequence annotation in the human genome (see, e.g., Aken, et al (2016), The Enxembl gene annotation system, Database, Volume 2016, baw093). Finally, spacers were removed if the target sequence contained a homopolymer (i.e., consecutive sequence of five or more identical nucleotides, e.g., “AAAAA”, “CCCCC”, “GGGGG”, “TTTTT”). The spacer sequences for SpCas9 and SluCas9 gRNAs were further filtered to identify those with 100% homology to target sequences in the FAAH gene of cynomolgus monkey/macaque/Macaca fascicularis (i.e., suitable for use in pre-clinical studies in a non-human primate animal model).

Additionally, an approximately 200 bp region encompassing exon 4 (i.e., chr1: 46,405,341-46,405,540 of Hg38) was evaluated to identify gRNA target sequences for use with SaCas9, i.e., target sequences with the pattern N₂₁NNGRRT (N=A,G,C,T; R=A,G) using the CCTop algorithm. This analysis identified 9 additional target sequences upstream an SaCas9 PAM that reside within or adjacent the exon 4 coding region.

The analysis provided (i) 34 spacer sequences for SpCas9 (Table 3; target sequences identified by SEQ ID NOs: 1-34; spacer sequences identified by SEQ ID NOs: 35-68); (ii) 40 spacer sequences for SluCas9 (Table 4; target sequences identified by SEQ ID NOs: 69-108; spacer sequences identified by SEQ ID NOs: 109-148) The FAAH target sequence for SluCas9 gRNA spacers was extended to 22 nucleotides post-analysis; and (iii) 16 spacer sequences for SaCas9 gRNAs (Table 5; target sequences identified by SEQ ID NOs: 149-164; spacer sequences identified by SEQ ID NOs: 165-180).

Certain target sequence were identified that were located in FAAH intronic regions that were either upstream or downstream of FAAH exonic regions. These include SpCh1, SpCh2, SpCh3, SpCh4, SpCh5, SpCh6, SpCh22, and SpCh23 shown in Table 3; SluCh1, SluCh2, SluCh3, SluCh4, SluCh5, SluCh6, SluCh25, and SluCh26 shown in Table 4; and SpCh1, SaCh2, SaCh3, SaCh5, SaCh6, SaCh9, and SaCh16 shown in Table 5.

TABLE 3
Target Sequences for SpCas9 gRNAs in the FAAH Coding Sequence

| | | SEQ | | | SEQ |
|---|---|---|---|---|---|
| | Target Sequence | ID | Cut site | | ID |
| Name | PAM in bold underline | NO | Location* | Spacer Sequence | NO |
| SpCh1 | AAACCCGGACTGGATCAGCCGGG | 1 | 46394259 | AAACCCGGACUGGAUCAGCC | 35 |
| SpCh2 | AAAACCCGGACTGGATCAGCCGG | 2 | 46394260 | AAAACCCGGACUGGAUCAGC | 36 |
| SpCh3 | CTGATCCAGTCCGGGTTTTGCGG | 3 | 46394274 | CUGAUCCAGUCCGGGUUUUG | 37 |
| SpCh4 | ATCCAGTCCGGGTTTTGCGGCGG | 4 | 46394277 | AUCCAGUCCGGGUUUUGCGG | 38 |
| SpCh5 | CTCCGCCGCAAAACCCGGACTGG | 5 | 46394269 | CUCCGCCGCAAAACCCGGAC | 39 |
| SpCh6 | TCCGGGTTTTGCGGCGGAGCGGG | 6 | 46394283 | UCCGGGUUUUGCGGCGGAGC | 40 |
| SpCh7 | TGGCGCCTCCGGGGTCGCCCTGG | 7 | 46394397 | UGGCGCCUCCGGGGUCGCCC | 41 |
| SpCh8 | GCAGGCCAGGGCGACCCCGGAGG | 8 | 46394392 | GCAGGCCAGGGCGACCCCGG | 42 |
| SpCh9 | CGCCCTGGCCTGCTGCTTCGTGG | 9 | 46394412 | CGCCCUGGCCUGCUGCUUCG | 43 |
| SpCh10 | CGCCACGAAGCAGCAGGCCAGGG | 10 | 46394404 | CGCCACGAAGCAGCAGGCCA | 44 |
| SpCh11 | CCGCCACGAAGCAGCAGGCCAGG | 11 | 46394405 | CCGCCACGAAGCAGCAGGCC | 45 |
| SpCh12 | CCTGGCCTGCTGCTTCGTGGCGG | 12 | 46394415 | CCUGGCCUGCUGCUUCGUGG | 46 |
| SpCh13 | GGCCTGCTGCTTCGTGGCGGCGG | 13 | 46394418 | GGCCUGCUGCUUCGUGGCGG | 47 |
| SpCh14 | GGCCGCCGCCACGAAGCAGCAGG | 14 | 46394410 | GGCCGCCGCCACGAAGCAGC | 48 |
| SpCh15 | CTGCTTCGTGGCGGCGGCCGTGG | 15 | 46394424 | CUGCUUCGUGGCGGCGGCCG | 49 |
| SpCh16 | GCCGTGGCCCTGCGCTGGTCCGG | 16 | 46394440 | GCCGUGGCCCUGCGCUGGUC | 50 |
| SpCh17 | CCGTGGCCCTGCGCTGGTCCGGG | 17 | 46394441 | CCGUGGCCCUGCGCUGGUCC | 51 |
| SpCh18 | CCCGGACCAGCGCAGGGCCACGG | 18 | 46394431 | CCCGGACCAGCGCAGGGCCA | 52 |
| SpCh19 | CCGGCGCCCGGACCAGCGCAGGG | 19 | 46394437 | CCGGCGCCCGGACCAGCGCA | 53 |
| SpCh20 | CCCTGCGCTGGTCCGGGCGCCGG | 20 | 46394447 | CCCUGCGCUGGUCCGGGCGC | 54 |
| SpCh21 | GGCGCAGCGCTTCCGGCTCCAGG | 21 | 46394538 | GGCGCAGCGCUUCCGGCUCC | 55 |
| SpCh22 | GTCCAGGTCTGGGTTCTGTGGGG | 22 | 46402089 | GUCCAGGUCUGGGUUCUGUG | 56 |
| SpCh23 | AGTCCAGGTCTGGGTTCTGTGGG | 23 | 46402090 | AGUCCAGGUCUGGGUUCUGU | 57 |
| SpCh24 | GCGCCTCTGAGTCCAGGTCTGGG | 24 | 46402099 | GCGCCUCUGAGUCCAGGUCU | 58 |
| SpCh25 | AGCGCCTCTGAGTCCAGGTCTGG | 25 | 46402100 | AGCGCCUCUGAGUCCAGGUC | 59 |
| SpCh26 | CTAGCAGCGCCTCTGAGTCCAGG | 26 | 46402105 | CUAGCAGCGCCUCUGAGUCC | 60 |
| SpCh27 | CTTCTGCACCAGCTGAGGCAGGG | 27 | 46402134 | CUUCUGCACCAGCUGAGGCA | 61 |
| SpCh28 | TGTAACTTCTGCACCAGCTGAGG | 28 | 46402139 | UGUAACUUCUGCACCAGCUG | 62 |
| SpCh29 | GGTGAAGAGCACGGCCTCAGGGG | 29 | 46402176 | GGUGAAGAGCACGGCCUCAG | 63 |
| SpCh30 | GGCCGTGCTCTTCACCTATGTGG | 30 | 46402193 | GGCCGUGCUCUUCACCUAUG | 64 |
| SpCh31 | GCCGTGCTCTTCACCTATGTGGG | 31 | 46402194 | GCCGUGCUCUUCACCUAUGU | 65 |
| SpCh32 | TCCCACATAGGTGAAGAGCACGG | 32 | 46402185 | UCCCACAUAGGUGAAGAGCA | 66 |
| SpCh33 | GCTCTTCACCTATGTGGGAAAGG | 33 | 46402199 | GCUCUUCACCUAUGUGGGAA | 67 |
| SpCh34 | TGGCCTTACCTTTCCCACATAGG | 34 | 46402197 | UGGCCUUACCUUUCCCACAU | 68 |

*chromosomal location of guide cut-site in chromosome 1 of human genome Hg38

TABLE 4
Target Sequences for SluCas9 gRNAs in the FAAH Coding Sequence

| | | SEQ | | | SEQ |
|---|---|---|---|---|---|
| | Target Sequence | ID | Cut site | | ID |
| Name | PAM in bold underline | NO | Location* | Spacer Sequence | NO |
| SluChl | GCAAAACCCGGACTGGATCAGCCGGG | 69 | 46394260 | GCAAAACCCGGACUGGAUCAGC | 109 |
| SluCh2 | CGCAAAACCCGGACTGGATCAGCCGG | 70 | 46394261 | CGCAAAACCCGGACUGGAUCAG | 110 |
| SluCh3 | CGGCTGATCCAGTCCGGGTTTTGCGG | 71 | 46394273 | CGGCUGAUCCAGUCCGGGUUUU | 111 |
| SluCh4 | CTGATCCAGTCCGGGTTTTGCGGCGG | 72 | 46394276 | CUGAUCCAGUCCGGGUUUUGCG | 112 |
| SluCh5 | CCGCTCCGCCGCAAAACCCGGACTGG | 73 | 46394270 | CCGCUCCGCCGCAAAACCCGGA | 113 |
| SluCh6 | CAGTCCGGGTTTTGCGGCGGAGCGGG | 74 | 46394282 | CAGUCCGGGUUUUGCGGCGGAG | 114 |
| SluCh7 | GCCTGGCGCCTCCGGGGTCGCCCTGG | 75 | 46394396 | GCCUGGCGCCUCCGGGGUCGCC | 115 |
| SluCh8 | GCCAGGGCGACCCCGGAGGCGCCAGG | 76 | 46394386 | GCCAGGGCGACCCCGGAGGCGC | 116 |
| SluCh9 | GCAGCAGGCCAGGGCGACCCCGGAGG | 77 | 46394393 | GCAGCAGGCCAGGGCGACCCCG | 117 |
| SluCh10 | GAAGCAGCAGGCCAGGGCGACCCCGG | 78 | 46394396 | GAAGCAGCAGGCCAGGGCGACC | 118 |
| SluCh11 | GGTCGCCCTGGCCTGCTGCTTCGTGG | 79 | 46394411 | GGUCGCCCUGGCCUGCUGCUUC | 119 |
| SluCh12 | CGCCCTGGCCTGCTGCTTCGTGGCGG | 80 | 46394414 | CGCCCUGGCCUGCUGCUUCGUG | 120 |
| SluCh13 | CGCCGCCACGAAGCAGCAGGCCAGGG | 81 | 46394405 | CGCCGCCACGAAGCAGCAGGCC | 121 |
| SluCh14 | CCGCCGCCACGAAGCAGCAGGCCAGG | 82 | 46394406 | CCGCCGCCACGAAGCAGCAGGC | 122 |
| SluCh15 | CCTGGCCTGCTGCTTCGTGGCGGCGG | 83 | 46394417 | CCUGGCCUGCUGCUUCGUGGCG | 123 |
| SluCh16 | CACGGCCGCCGCCACGAAGCAGCAGG | 84 | 46394411 | CACGGCCGCCGCCACGAAGCAG | 124 |
| SluCh17 | CTGCTGCTTCGTGGCGGCGGCCGTGG | 85 | 46394423 | CUGCUGCUUCGUGGCGGCGGCC | 125 |
| SluCh18 | TGGCGGCGGCCGTGGCCCTGCGCTGG | 86 | 46394434 | UGGCGGCGGCCGUGGCCCUGCG | 126 |
| SluCh19 | GCGGCCGTGGCCCTGCGCTGGTCCGG | 87 | 46394439 | GCGGCCGUGGCCCUGCGCUGGU | 127 |
| SluCh20 | CGGCCGTGGCCCTGCGCTGGTCCGGG | 88 | 46394440 | CGGCCGUGGCCCUGCGCUGGUC | 128 |
| SluCh21 | GCGCCCGGACCAGCGCAGGGCCACGG | 89 | 46394432 | GCGCCCGGACCAGCGCAGGGCC | 129 |
| SluCh22 | TGGCCCTGCGCTGGTCCGGGCGCCGG | 90 | 46394446 | UGGCCCUGCGCUGGUCCGGGCG | 130 |
| SluCh23 | CCGCTCGCTGCCTCTGTCGCGCCCGG | 91 | 46394481 | CCGCUCGCUGCCUCUGUCGCGC | 131 |
| SluCh24 | GGCGGCGCAGCGCTTCCGGCTCCAGG | 92 | 46394537 | GGCGGCGCAGCGCUUCCGGCUC | 132 |
| SluCh23 | TGAGTCCAGGTCTGGGTTCTGTGGGG | 93 | 46402090 | UGAGUCCAGGUCUGGGUUCUGU | 133 |
| SluCh26 | CTGAGTCCAGGTCTGGGTTCTGTGGG | 94 | 46402091 | CUGAGUCCAGGUCUGGGUUCUG | 134 |
| SluCh27 | TCTGAGTCCAGGTCTGGGTTCTGTGG | 95 | 46402092 | UCUGAGUCCAGGUCUGGGUUCU | 135 |
| SluCh28 | ACAGAACCCAGACCTGGACTCAGAGG | 96 | 46402105 | ACAGAACCCAGACCUGGACUCA | 136 |
| SluCh29 | GCAGCGCCTCTGAGTCCAGGTCTGGG | 97 | 46402100 | GCAGCGCCUCUGAGUCCAGGUC | 137 |
| SluCh30 | AGCAGCGCCTCTGAGTCCAGGTCTGG | 98 | 46402101 | AGCAGCGCCUCUGAGUCCAGGU | 138 |
| SluCh31 | GGGCTAGCAGCGCCTCTGAGTCCAGG | 99 | 46402106 | GGGCUAGCAGCGCCUCUGAGUC | 139 |
| SluCh32 | AACTTCTGCACCAGCTGAGGCAGGGG | 100 | 46402134 | AACUUCUGCACCAGCUGAGGCA | 140 |
| SluCh33 | GTAACTTCTGCACCAGCTGAGGCAGG | 101 | 46402136 | GUAACUUCUGCACCAGCUGAGG | 141 |
| SluCh34 | CTGTGTAACTTCTGCACCAGCTGAGG | 102 | 46402140 | CUGUGUAACUUCUGCACCAGCU | 142 |
| SluCh33 | ATAGGTGAAGAGCACGGCCTCAGGGG | 103 | 46402177 | AUAGGUGAAGAGCACGGCCUCA | 143 |
| SluCh36 | ACATAGGTGAAGAGCACGGCCTCAGG | 104 | 46402179 | ACAUAGGUGAAGAGCACGGCCU | 144 |
| SluCh37 | TGAGGCCGTGCTCTTCACCTATGTGG | 105 | 46402192 | UGAGGCCGUGCUCUUCACCUAU | 145 |
| SluCh38 | GAGGCCGTGCTCTTCACCTATGTGGG | 106 | 46402193 | GAGGCCGUGCUCUUCACCUAUG | 146 |
| SluCh39 | CTTTCCCACATAGGTGAAGAGCACGG | 107 | 46402186 | CUUUCCCACAUAGGUGAAGAGC | 147 |
| SluCh40 | CGTGCTCTTCACCTATGTGGGAAAGG | 108 | 46402198 | CGUGCUCUUCACCUAUGUGGGA | 148 |

*chromosomal location of guide cut-site in chromosome 1 of human genome Hg38

TABLE 5
Target Sequences for SaCas9 gRNAs in the FAAH Coding Sequence

| | | SEQ | | | SEQ |
|---|---|---|---|---|---|
| | Target Sequence | ID | Cut site | | ID |
| Name | PAM in bold underline | NO | Location* | Spacer Sequence | NO |
| SaCh1 | TGGGATCCCGGCTGATCCAGTCCGGGT | 149 | 46394264 | UGGGAUCCCGGCUGAUCCAGU | 165 |
| SaCh2 | CAAAACCCGGACTGGATCAGCCGGGAT | 150 | 46394260 | CAAAACCCGGACUGGAUCAGC | 166 |
| SaCh3 | CGCTCCGCCGCAAAACCCGGACTGGAT | 151 | 46394270 | CGCUCCGCCGCAAAACCCGGA | 167 |
| SaCh4 | GGCCGCGCTGCCTGGCGCCTCCGGGGT | 152 | 46394386 | GGCCGCGCUGCCUGGCGCCUC | 168 |
| SaCh5 | CAGGTGACTGCCGGAGCGTAGTGGGAT | 153 | 46394558 | CAGGUGACUGCCGGAGCGUAG | 169 |
| SaCh6 | TGGGTTCTGTGGGGAACAAACTCGGAT | 154 | 46402079 | UGGGUUCUGUGGGGAACAAAC | 170 |
| SaCh7 | GCAGCGCCTCTGAGTCCAGGTCTGGGT | 155 | 46402101 | GCAGCGCCUCUGAGUCCAGGU | 171 |
| SaCh8 | GGGGCAGGGCTAGCAGCGCCTCTGAGT | 156 | 46402113 | GGGGCAGGGCUAGCAGCGCCU | 172 |
| SaCh9 | TGGAGTCCTGGCCCTGGGAGGAGGGAT | 157 | 46405367 | UGGAGUCCUGGCCCUGGGAGG | 173 |
| SaCh10 | GACTCCACGCTGGGCTTGAGCCTGAAT | 158 | 46405395 | GACUCCACGCUGGGCUUGAGC | 174 |
| SaCh11 | CATTCAGGCTCAAGCCCAGCGTGGAGT | 159 | 46405388 | CAUUCAGGCUCAAGCCCAGCG | 175 |
| SaCh12 | GCTGGGCTTGAGCCTGAATGAAGGGGT | 160 | 46405403 | GCUGGGCUUGAGCCUGAAUGA | 176 |
| SaCh13 | GCCTGAATGAAGGGGTGCCGGCGGAGT | 161 | 46405414 | GCCUGAAUGAAGGGGUGCCGG | 177 |
| SaCh14 | GTGGTGCATGTGCTGAAGCTGCAGGGT | 162 | 46405452 | GUGGUGCAUGUGCUGAAGCUG | 178 |
| SaCh15 | GTTCCACAGTCCATGTTCAGGTTGGGT | 163 | 46405503 | GUUCCACAGUCCAUGUUCAGG | 179 |
| SaCh16 | GTCCATGTTCAGGTTGGGTCTTGGGGT | 164 | 46405511 | GUCCAUGUUCAGGUUGGGUCU | 180 |

*chromosomal location of guide cut-site in chromosome 1 of human genome Hg38

### Example 2: Evaluation of In Vitro Gene Editing and Functional Activity of gRNA/SpCas9 Targeting the FAAH Coding Sequence

Analysis of editing using sgRNA/Cas9 was performed by measuring the frequency of small insertions and deletions (INDELs) induced in the FAAH coding sequence using complexes of SpCas9 sgRNA prepared with the spacers identified in Example 1 and SpCas9 polypeptide.

Specifically, SpCas9 sgRNA were prepared with the spacers identified in Table 3 (SpCh1-SpCh34; SEQ ID NOs: 35-68) inserted into the sgRNA backbone identified by SEQ ID NO: 1267 and shown in Table 6. The SpCas9 sgRNA sequences were chemically synthesized by a commercial vendor.

TABLE 6
Sequences of SpCas9 sgRNA

| | | SEQ |
|---|---|---|
| | | ID |
| Name | sgRNA Sequence (spacer in bold) | NO |
| Sp | mN*mN*mN*NNNNNNNNNNNNNNNNNGUUUU | 1267 |
| sgRNA | AGAGCUAGAAAUAGCAAGUUAAAAUAAGGCU | |
| | AGUCCGUUAUCAACUUGAAAAAGUGGCACCG | |
| | AGUCGGUmG*mC*mU* | |

mN*: 2′-O-methyl 3′phosphorothioate

The SpCas9 sgRNA were individually evaluated as complexes with SpCas9 protein for inducing INDELs at predicted cut sites in the FAAH coding sequence. Editing efficiency was measured in MCF7 cells. Briefly, 1×10⁵ MCF7 cells were suspended in SE solution (Lonza) and electroporated with 0.5 μg SpCas9 sgRNA and 0.5 μg SpCas9 protein (SEQ ID NO: 1268) using the 4D-nucleofector X unit (Lonza) CM-113 program. Following electroporation, the cells were incubated for 72 hours. Thereafter, genomic DNA was extracted and purified using a Quick DNA Kit (Zymo #D3011).

The frequency of INDELs induced at predicted cut sites in the genomic DNA was evaluated by TIDE analysis (see, e.g., Brinkman, et al (2014) NUCLEIC ACIDS RESEARCH 42:e168). Specifically, primers flanking the target site of each SpCas9 sgRNA were used in a PCR reaction with 2 μL (40-70 ng) of genomic DNA to amplify a region 1 of 955 bp and region 2 of 759 bp, flanking exon 1 and exon 2 respectively, surrounding the predicted cut site of each sgRNA. The primers used for amplification corresponding to each SpCas9 sgRNA are identified in Table 7. The PCR product was purified using AMPure XP PCR Purification (Beckman Coulter #A63881) and Sanger sequencing (Genewiz) was performed using the sequencing primers identified in Table 7. The sequence data was analyzed using Tsunami software to determine the frequency of INDELs at the predicted cut site for each sgRNA/SpCas9 complex.

The guides were categorized based on cleavage efficiency as measured by total frequency of INDELs introduced at the predicted cut site. As shown in Table 8, guides with low cleavage efficiency (total frequency of INDELs less than 15%), moderate cleavage efficiency (total frequency of INDELs 15-25%), and high cleavage efficiency (total frequency of INDELs greater than 25%) are indicated.

TABLE 7
TIDE Primer Sequences for Analysis of INDEL Frequency at
Cut Site Corresponding to SpCas9 sgRNAs

| | | SEQ | | SEQ | Sequencing | SEQ |
|---|---|---|---|---|---|---|
| sgRNA | PCR primer 1 | ID NO | PCR primer 2 | ID NO | primer | ID NO |
| SpCh1 | TCTAACAGCTGGCAT | 1291 | AAGCTCTCCAGATCC | 1325 | GGGCGCAGTCTTCAG | 1359 |
| | GTCTG | | CCTTG | | CATT | |
| SpCh2 | TCTAACAGCTGGCAT | 1292 | AAGCTCTCCAGATCC | 1326 | GGGCGCAGTCTTCAG | 1360 |
| | GTCTG | | CCTTG | | CATT | |
| SpCh3 | TCTAACAGCTGGCAT | 1293 | AAGCTCTCCAGATCC | 1327 | GGGCGCAGTCTTCAG | 1361 |
| | GTCTG | | CCTTG | | CATT | |
| SpCh4 | TCTAACAGCTGGCAT | 1294 | AAGCTCTCCAGATCC | 1328 | GGGCGCAGTCTTCAG | 1362 |
| | GTCTG | | CCTTG | | CATT | |
| SpCh5 | TCTAACAGCTGGCAT | 1295 | AAGCTCTCCAGATCC | 1329 | GGGCGCAGTCTTCAG | 1363 |
| | GTCTG | | CCTTG | | CATT | |
| SpCh6 | TCTAACAGCTGGCAT | 1296 | AAGCTCTCCAGATCC | 1330 | GGGCGCAGTCTTCAG | 1364 |
| | GTCTG | | CCTTG | | CATT | |
| SpCh7 | TCTAACAGCTGGCAT | 1297 | AAGCTCTCCAGATCC | 1331 | GGGCGCAGTCTTCAG | 1365 |
| | GTCTG | | CCTTG | | CATT | |
| SpCh8 | TCTAACAGCTGGCAT | 1298 | AAGCTCTCCAGATCC | 1332 | GGGCGCAGTCTTCAG | 1366 |
| | GTCTG | | CCTTG | | CATT | |
| SpCh9 | TCTAACAGCTGGCAT | 1299 | AAGCTCTCCAGATCC | 1333 | GGGCGCAGTCTTCAG | 1367 |
| | GTCTG | | CCTTG | | CATT | |
| SpCh10 | TCTAACAGCTGGCAT | 1300 | AAGCTCTCCAGATCC | 1334 | GGGCGCAGTCTTCAG | 1368 |
| | GTCTG | | CCTTG | | CATT | |
| SpCh11 | TCTAACAGCTGGCAT | 1301 | AAGCTCTCCAGATCC | 1335 | GTCCTCAACCCCTGG | 1369 |
| | GTCTG | | CCTTG | | CATCC | |
| SpCh12 | TCTAACAGCTGGCAT | 1302 | AAGCTCTCCAGATCC | 1336 | GTCCTCAACCCCTGG | 1370 |
| | GTCTG | | CCTTG | | CATCC | |
| SpCh13 | TCTAACAGCTGGCAT | 1303 | AAGCTCTCCAGATCC | 1337 | GTCCTCAACCCCTGG | 1371 |
| | GTCTG | | CCTTG | | CATCC | |
| SpCh14 | TCTAACAGCTGGCAT | 1304 | AAGCTCTCCAGATCC | 1338 | GTCCTCAACCCCTGG | 1372 |
| | GTCTG | | CCTTG | | CATCC | |
| SpCh15 | TCTAACAGCTGGCAT | 1305 | AAGCTCTCCAGATCC | 1339 | GTCCTCAACCCCTGG | 1373 |
| | GTCTG | | CCTTG | | CATCC | |
| SpCh16 | TCTAACAGCTGGCAT | 1306 | AAGCTCTCCAGATCC | 1340 | GTCCTCAACCCCTGG | 1374 |
| | GTCTG | | CCTTG | | CATCC | |
| SpCh17 | TCTAACAGCTGGCAT | 1307 | AAGCTCTCCAGATCC | 1341 | GTCCTCAACCCCTGG | 1375 |
| | GTCTG | | CCTTG | | CATCC | |
| SpCh18 | TCTAACAGCTGGCAT | 1308 | AAGCTCTCCAGATCC | 1342 | GTCCTCAACCCCTGG | 1376 |
| | GTCTG | | CCTTG | | CATCC | |
| SpCh19 | TCTAACAGCTGGCAT | 1309 | AAGCTCTCCAGATCC | 1343 | GTCCTCAACCCCTGG | 1377 |
| | GTCTG | | CCTTG | | CATCC | |
| SpCh20 | TCTAACAGCTGGCAT | 1310 | AAGCTCTCCAGATCC | 1344 | GTCCTCAACCCCTGG | 1378 |
| | GTCTG | | CCTTG | | CATCC | |
| SpCh21 | TCTAACAGCTGGCAT | 1311 | AAGCTCTCCAGATCC | 1345 | GTCCTCAACCCCTGG | 1379 |
| | GTCTG | | CCTTG | | CATCC | |
| SpCh22 | CATCAGTCTGGAGCT | 1312 | AGACCAGACTTGTTG | 1346 | AGCATGTGCCTGTAG | 1380 |
| | AGGCA | | CCCAA | | TTC | |
| SpCh23 | CATCAGTCTGGAGCT | 1313 | AGACCAGACTTGTTG | 1347 | AGCATGTGCCTGTAG | 1381 |
| | AGGCA | | CCCAA | | TTC | |
| SpCh24 | CATCAGTCTGGAGCT | 1314 | AGACCAGACTTGTTG | 1348 | AGCATGTGCCTGTAG | 1382 |
| | AGGCA | | CCCAA | | TTC | |
| SpCh25 | CATCAGTCTGGAGCT | 1315 | AGACCAGACTTGTTG | 1349 | AGCATGTGCCTGTAG | 1383 |
| | AGGCA | | CCCAA | | TTC | |
| SpCh26 | CATCAGTCTGGAGCT | 1316 | AGACCAGACTTGTTG | 1350 | AGCATGTGCCTGTAG | 1384 |
| | AGGCA | | CCCAA | | TTC | |
| SpCh27 | CATCAGTCTGGAGCT | 1317 | AGACCAGACTTGTTG | 1351 | AGCATGTGCCTGTAG | 1385 |
| | AGGCA | | CCCAA | | TTC | |
| SpCh28 | CATCAGTCTGGAGCT | 1318 | AGACCAGACTTGTTG | 1352 | AGCATGTGCCTGTAG | 1386 |
| | AGGCA | | CCCAA | | TTC | |
| SpCh29 | CATCAGTCTGGAGCT | 1319 | AGACCAGACTTGTTG | 1353 | AGCATGTGCCTGTAG | 1387 |
| | AGGCA | | CCCAA | | TTC | |
| SpCh30 | CATCAGTCTGGAGCT | 1320 | AGACCAGACTTGTTG | 1354 | AGCATGTGCCTGTAG | 1388 |
| | AGGCA | | CCCAA | | TTC | |
| SpCh31 | CATCAGTCTGGAGCT | 1321 | AGACCAGACTTGTTG | 1355 | AGCATGTGCCTGTAG | 1389 |
| | AGGCA | | CCCAA | | TTC | |
| SpCh32 | CATCAGTCTGGAGCT | 1322 | AGACCAGACTTGTTG | 1356 | AGCATGTGCCTGTAG | 1390 |
| | AGGCA | | CCCAA | | TTC | |
| SpCh33 | CATCAGTCTGGAGCT | 1323 | AGACCAGACTTGTTG | 1357 | AGCATGTGCCTGTAG | 1391 |
| | AGGCA | | CCCAA | | TTC | |
| SpCh34 | CATCAGTCTGGAGCT | 1324 | AGACCAGACTTGTTG | 1358 | AGCATGTGCCTGTAG | 1392 |
| | AGGCA | | CCCAA | | TTC | |

TABLE 8
SpCas9 sgRNAs Categorized Based on Cleavage Efficiency

| Total | |
|---|---|
| INDEL % | Guides |
| <15% | SpCh1, SpCh2, SpCh15 |
| 15%-25% | SpCh4, SpCh5, SpCh7, SpCh14, SpCh20 |
| >25% | SpCh3, SpCh6, SpCh8, SpCh9, SpCh10, SpCh11, SpCh12, |
| | SpCh13, SpCh16, SpCh17, SpCh18, SpCh19, SpCh21, |
| | SpCh22, SpCh23, SpCh24, SpCh25, SpCh26, SpCh27, |
| | SpCh28, SpCh29, SpCh30, SpCh31, SpCh32, SpCh33, |
| | SpCh34 |

A subset of SpCas9 sgRNAs were selected for subsequent evaluation, including measurement of INDEL frequency at the predicted cut site, measurement of FAAH mRNA levels, and measurement of FAAH polypeptide levels in cells edited the sgRNA/SpCas9 complex. This subset included the sgRNAs that are identified in Table 9, which includes SpCh8, SpCh9, SpCh26, SpCh29, SpCh30, SpCh31, SpCh32, and SpCh34 having cut locations within FAAH exon 1 or exon 2, and SpCh22 and SpCh23 having cut locations outside of FAAH exon 1 or exon 2.

Briefly, 3×10⁵ MCF7 cells were electroporated with 1.5 μg SpCas9 sgRNA and 1.5 μg SpCas9 protein as described above. The cells were harvested and extracted for genomic DNA for INDEL quantification by TIDE analysis as described above. The overall INDEL frequency at the predicted cut site of each sgRNA is provided in Table 9. The INDELs resulting in an in-frame mutation (i.e., ±3 nt, ±6 nt, ±9 nt, etc.) were removed to provide the percentage of INDELs expected to produce a frameshift mutation (i.e., ±1 nt, ±2 nt, ±4 nt, etc), is also shown in Table 9. The sgRNA were ranked according to frequency of INDELs that cause a frameshift mutation, as shown in FIG. 1A. The sgRNAs having cut sites outside the exon 1 or exon 2 regions of FAAH are shown by asterisk. As a frameshift mutation for these guides is not applicable, the value represented by “frameshift INDELs” refers to the frequency of total INDELs minus the frequency of INDELs that are divisible by 3 (e.g., ±3 nt, ±6 nt, ±9 nt, etc).

The overall frequency of INDELs exceeding 90% for each sgRNA evaluated. Additionally, most sgRNAs with cut locations within FAAH exons resulted in a frequency INDELs introducing a frameshift mutation that exceeded 80%. The SpCh30 sgRNA induced the highest frequency of INDELs of the SpCas9 sgRNAs cutting within a FAAH exon (98.1% total, 95% introducing a frameshift mutation).

Edited MCF7 cells were also harvested for RNA extraction to determine FAAH mRNA levels using a quantitative PCR (qPCR) assay. Specifically, RNA extraction was performed using a Quick-RNA 96 Kit (Zymo Research, #R1052). RNA concentration was measured by DropSense (Trinean) and 250 ng RNA was used for reverse transcription using a QuantiTect Reverse Transcription kit (Qiagen #205311) to prepare cDNA. Subsequently, 40 ng of cDNA was used for qPCR to measure FAAH mRNA levels. For qPCR quantification, TaqMan Gene Expression Master Mix (ThermoFisher #4369016) was combined with the reagents below. TBP mRNA levels were used as qPCR internal controls.

| | Forward primer: |
|---|---|
| (SEQ ID NO: 1273) | |
| | TGATATCGGAGGCAGCATCC; |
| | Reverse primer: |
| (SEQ ID NO: 1274) | |
| | CTTCAGGCCACTCTTGCTGA; |
| | and |
| | Probe: |
| (SEQ ID NO: 1275) | |
| | CTTCCCCTCCTCCTTCTGC. |

FAAH mRNA levels were quantified as a fold change between an edited sample and an untreated control sample subjected to electroporation without CRISPR/Cas9 components. Fold change was calculated using the 2\hat{ }(−ddCt) method and is provided for each sgRNA in Table 9. The sgRNA were further ranked by FAAH mRNA level following editing, as shown in FIG. 1B. Most sgRNA achieved at least a 50% reduction in FAAH mRNA levels, with SpCh31 sgRNA producing the greatest reduction.

Edited MCF7 cells were also harvested for total protein extraction to quantify FAAH protein levels by Simple Wes. Protein extraction was performed using RIPA lysis and extraction buffer (ThermoFisher #89900). Subsequently, 1-3 μg of protein was loaded onto Simple Wes and analyzed using a mouse anti-FAAH1 antibody (Abcam #ab54615; 1:25 dilution) and an anti-mouse secondary antibody (Abcam #ab97040) for detection of FAAH protein and a rabbit anti-GAPDH mAb 14C10 (CST #2118S; 1:25 dilution) in antibody diluent (ProteinSimple) with NIR anti-rabbit secondary antibody (ProteinSimple #043-819) for detection of GAPDH as an internal control protein.

The relative expression level of FAAH protein was compared to GAPDH as internal control. The relative expression level of FAAH protein was then normalized for samples treated with sgRNA/SpCas9 to a PBS control sample that was not subjected to electroporation. Normalized FAAH protein levels following editing are provided in Table 9. The sgRNA were further ranked based on the FAAH protein level, as shown in FIG. 1C. Several of the sgRNAs evaluated, including SpCh9, SpCh23, SpCh32, SpCh8, SpCh22, and SpCh26, resulted in a reduction of FAAH protein expression of 30% or more. Notably, sgRNAs with cut sites outside exon 1 or 2 (e.g., SpCh22 and SpCh23) resulted in a substantial reduction in FAAH mRNA and protein levels.

TABLE 9
Quantification of Editing Efficiency and Functional Activity
of SpCas9 sgRNAs Targeting FAAH Coding Sequence

| sgRNA | Indel (%) | | FAAH mRNA | FAAH protein |
|---|---|---|---|---|
| Name | Total | Frameshift* | (fold change) | (FAAH:GAPDH) |
| SpCh22 | 99.6 | 98.6 | 0.870335 | 0.645349 |
| SpCh30 | 98.1 | 95 | 0.593378 | 0.7750086 |
| SpCh32 | 98.6 | 94 | 0.287005 | 0.598545394 |
| SpCh9 | 96.3 | 93.2 | 0.3941 | 0.584547936 |
| SpCh29 | 99.4 | 91.8 | 0.33686 | 0.835150453 |
| SpCh34 | 95.5 | 91.7 | 0.43471 | 0.983583045 |
| SpCh31 | 97.1 | 91 | 0.263267 | 1.10133581 |
| SpCh23 | 96.1 | 81.3 | 0.61771 | 0.5913049 |
| SpCh8 | 93.5 | 81.1 | 0.351606 | 0.603549182 |
| SpCh26 | 93.4 | 63.8 | 0.723949 | 0.667934285 |

*Frameshift INDEL % refers to INDELs expected to result in a frameshift mutation in the FAAH coding sequence (i.e., ±1 nt, ±2 nt, ±4 nt). The sgRNAs with values in underline have cut sites outside exon 1 or exon 2 of FAAH, wherein frameshift mutations are not applicable. Thus, Frameshift INDEL % refers to frequency of total INDELs minus frequency of INDELs that are ±3 nt, ±6 nt, ±9 nt, etc.

### Example 3: Evaluation of In Vitro Gene Editing and Functional Activity of gRNA/SluCas9 Targeting the FAAH Coding Sequence

Frequency of INDELs induced at predicted cut sites in the FAAH coding sequence was also evaluated following in vitro treatment with complexes of SluCas9 protein and sgRNA that were prepared with spacers identified in Example 1.

Specifically, SluCas9 sgRNA were prepared with the spacers identified in Table 4 (SluCh1-SluCh40; SEQ ID NOs: 109-148) inserted into a sgRNA backbone identified by SEQ ID NO: 1269 and shown in Table 10. The SluCas9 sgRNA sequences were chemically synthesized by a commercial vendor.

TABLE 10
Sequence of SluCas9 sgRNA

| | | SEQ |
|---|---|---|
| | sgRNA Sequence | ID |
| Name | (spacer in bold) | NO |
| SluCas9 | mN*mN*mN*NNNNNNNNNNNNNNNNNNN | 1269 |
| sgRNA | GUUUUAGUACUCUGGAAACAGAAUCUAC | |
| | UGAAACAAGACAAUAUGUCGUGUUUAUC | |
| | CCAUCAAUUUAUUGGUGGmG*mA*mU* | |

mN*: 2′-O-methyl 3′phosphorothioate

The SluCas9 sgRNA were individually evaluated as complexes with SluCas9 protein for inducing INDELs at predicted cut sites in the FAAH coding sequence. Editing efficiency was measured in MCF7 cells. Briefly, 1×10⁵ MCF7 cells were electroporated with 0.5 μg sgRNA and 0.4 μg SluCas9 protein (SEQ ID NO: 1270) and incubated for 72 hours. Cells were harvested for genomic DNA extraction, followed by TIDE analysis as described in Example 2. TIDE PCR and sequencing primers corresponding to each SluCas9 sgRNA are identified in Table 11.

The guides were categorized based on cleavage efficiency as measured by total frequency of INDELs introduced at the predicted cut site. As shown in Table 12, guides with low cleavage efficiency (total frequency of INDELs less than 15%), moderate cleavage efficiency (total frequency of INDELs 15-25%), and high cleavage efficiency (total frequency of INDELs greater than 25%) are indicated.

TABLE II
TIDE Primer Sequences for Analysis of INDEL Frequency
at Cut Site Corresponding to SluCas9 sgRNAs

| | | SEQ | | SEQ | | SEQ |
|---|---|---|---|---|---|---|
| | | ID | | ID | Sequencing | ID |
| sgRNA | PCR primer 1 | NO | PCR primer 2 | NO | primer | NO |
| SluCh1 | TCTAACAGCTGGC | 1393 | AAGCTCTCCAGAT | 1433 | GGGCGCAGTCTTC | 1473 |
| | ATGTCTG | | CCCCTTG | | AGCATT | |
| SluCh2 | TCTAACAGCTGGC | 1394 | AAGCTCTCCAGAT | 1434 | GGGCGCAGTCTTC | 1474 |
| | ATGTCTG | | CCCCTTG | | AGCATT | |
| SluCh3 | TCTAACAGCTGGC | 1395 | AAGCTCTCCAGAT | 1435 | GGGCGCAGTCTTC | 1475 |
| | ATGTCTG | | CCCCTTG | | AGCATT | |
| SluCh4 | TCTAACAGCTGGC | 1396 | AAGCTCTCCAGAT | 1436 | GGGCGCAGTCTTC | 1476 |
| | ATGTCTG | | CCCCTTG | | AGCATT | |
| SluCh5 | TCTAACAGCTGGC | 1397 | AAGCTCTCCAGAT | 1437 | GGGCGCAGTCTTC | 1477 |
| | ATGTCTG | | CCCCTTG | | AGCATT | |
| SluCh6 | TCTAACAGCTGGC | 1398 | AAGCTCTCCAGAT | 1438 | GGGCGCAGTCTTC | 1478 |
| | ATGTCTG | | CCCCTTG | | AGCATT | |
| SluCh7 | TCTAACAGCTGGC | 1399 | AAGCTCTCCAGAT | 1439 | GGGCGCAGTCTTC | 1479 |
| | ATGTCTG | | CCCCTTG | | AGCATT | |
| SluCh8 | TCTAACAGCTGGC | 1400 | AAGCTCTCCAGAT | 1440 | GGGCGCAGTCTTC | 1480 |
| | ATGTCTG | | CCCCTTG | | AGCATT | |
| SluCh9 | TCTAACAGCTGGC | 1401 | AAGCTCTCCAGAT | 1441 | GGGCGCAGTCTTC | 1481 |
| | ATGTCTG | | CCCCTTG | | AGCATT | |
| SluCh10 | TCTAACAGCTGGC | 1402 | AAGCTCTCCAGAT | 1442 | GGGCGCAGTCTTC | 1482 |
| | ATGTCTG | | CCCCTTG | | AGCATT | |
| SluCh11 | TCTAACAGCTGGC | 1403 | AAGCTCTCCAGAT | 1443 | GTCCTCAACCCCT | 1483 |
| | ATGTCTG | | CCCCTTG | | GGCATCC | |
| SluCh12 | TCTAACAGCTGGC | 1404 | AAGCTCTCCAGAT | 1444 | GTCCTCAACCCCT | 1484 |
| | ATGTCTG | | CCCCTTG | | GGCATCC | |
| SluCh13 | TCTAACAGCTGGC | 1405 | AAGCTCTCCAGAT | 1445 | GTCCTCAACCCCT | 1485 |
| | ATGTCTG | | CCCCTTG | | GGCATCC | |
| SluCh14 | TCTAACAGCTGGC | 1406 | AAGCTCTCCAGAT | 1446 | GTCCTCAACCCCT | 1486 |
| | ATGTCTG | | CCCCTTG | | GGCATCC | |
| SluCh15 | TCTAACAGCTGGC | 1407 | AAGCTCTCCAGAT | 1447 | GTCCTCAACCCCT | 1487 |
| | ATGTCTG | | CCCCTTG | | GGCATCC | |
| SluCh16 | TCTAACAGCTGGC | 1408 | AAGCTCTCCAGAT | 1448 | GTCCTCAACCCCT | 1488 |
| | ATGTCTG | | CCCCTTG | | GGCATCC | |
| SluCh17 | TCTAACAGCTGGC | 1409 | AAGCTCTCCAGAT | 1449 | GTCCTCAACCCCT | 1489 |
| | ATGTCTG | | CCCCTTG | | GGCATCC | |
| SluCh18 | TCTAACAGCTGGC | 1410 | AAGCTCTCCAGAT | 1450 | GTCCTCAACCCCT | 1490 |
| | ATGTCTG | | CCCCTTG | | GGCATCC | |
| SluCh19 | TCTAACAGCTGGC | 1411 | AAGCTCTCCAGAT | 1451 | GTCCTCAACCCCT | 1491 |
| | ATGTCTG | | CCCCTTG | | GGCATCC | |
| SluCh20 | TCTAACAGCTGGC | 1412 | AAGCTCTCCAGAT | 1452 | GTCCTCAACCCCT | 1492 |
| | ATGTCTG | | CCCCTTG | | GGCATCC | |
| SluCh21 | TCTAACAGCTGGC | 1413 | AAGCTCTCCAGAT | 1453 | GTCCTCAACCCCT | 1493 |
| | ATGTCTG | | CCCCTTG | | GGCATCC | |
| SluCh22 | TCTAACAGCTGGC | 1414 | AAGCTCTCCAGAT | 1454 | GTCCTCAACCCCT | 1494 |
| | ATGTCTG | | CCCCTTG | | GGCATCC | |
| SluCh23 | TCTAACAGCTGGC | 1415 | AAGCTCTCCAGAT | 1455 | GTCCTCAACCCCT | 1495 |
| | ATGTCTG | | CCCCTTG | | GGCATCC | |
| SluCh24 | TCTAACAGCTGGC | 1416 | AAGCTCTCCAGAT | 1456 | GTCCTCAACCCCT | 1496 |
| | ATGTCTG | | CCCCTTG | | GGCATCC | |
| SluCh25 | CATCAGTCTGGAG | 1417 | AGACCAGACTTGT | 1457 | AGCATGTGCCTGT | 1497 |
| | CTAGGCA | | TGCCCAA | | AGTTC | |
| SluCh26 | CATCAGTCTGGAG | 1418 | AGACCAGACTTGT | 1458 | AGCATGTGCCTGT | 1498 |
| | CTAGGCA | | TGCCCAA | | AGTTC | |
| SluCh27 | CATCAGTCTGGAG | 1419 | AGACCAGACTTGT | 1459 | AGCATGTGCCTGT | 1499 |
| | CTAGGCA | | TGCCCAA | | AGTTC | |
| SluCh28 | CATCAGTCTGGAG | 1420 | AGACCAGACTTGT | 1460 | AGCATGTGCCTGT | 1500 |
| | CTAGGCA | | TGCCCAA | | AGTTC | |
| SluCh29 | CATCAGTCTGGAG | 1421 | AGACCAGACTTGT | 1461 | AGCATGTGCCTGT | 1501 |
| | CTAGGCA | | TGCCCAA | | AGTTC | |
| SluCh30 | CATCAGTCTGGAG | 1422 | AGACCAGACTTGT | 1462 | AGCATGTGCCTGT | 1502 |
| | CTAGGCA | | TGCCCAA | | AGTTC | |
| SluCh31 | CATCAGTCTGGAG | 1423 | AGACCAGACTTGT | 1463 | AGCATGTGCCTGT | 1503 |
| | CTAGGCA | | TGCCCAA | | AGTTC | |
| SluCh32 | CATCAGTCTGGAG | 1424 | AGACCAGACTTGT | 1464 | AGCATGTGCCTGT | 1504 |
| | CTAGGCA | | TGCCCAA | | AGTTC | |
| SluCh33 | CATCAGTCTGGAG | 1425 | AGACCAGACTTGT | 1465 | AGCATGTGCCTGT | 1505 |
| | CTAGGCA | | TGCCCAA | | AGTTC | |
| SluCh34 | CATCAGTCTGGAG | 1426 | AGACCAGACTTGT | 1466 | AGCATGTGCCTGT | 1506 |
| | CTAGGCA | | TGCCCAA | | AGTTC | |
| SluCh35 | CATCAGTCTGGAG | 1427 | AGACCAGACTTGT | 1467 | AGCATGTGCCTGT | 1507 |
| | CTAGGCA | | TGCCCAA | | AGTTC | |
| SluCh36 | CATCAGTCTGGAG | 1428 | AGACCAGACTTGT | 1468 | AGCATGTGCCTGT | 1508 |
| | CTAGGCA | | TGCCCAA | | AGTTC | |
| SluCh37 | CATCAGTCTGGAG | 1429 | AGACCAGACTTGT | 1469 | AGCATGTGCCTGT | 1509 |
| | CTAGGCA | | TGCCCAA | | AGTTC | |
| SluCh38 | CATCAGTCTGGAG | 1430 | AGACCAGACTTGT | 1470 | AGCATGTGCCTGT | 1510 |
| | CTAGGCA | | TGCCCAA | | AGTTC | |
| SluCh39 | CATCAGTCTGGAG | 1431 | AGACCAGACTTGT | 1471 | AGCATGTGCCTGT | 1511 |
| | CTAGGCA | | TGCCCAA | | AGTTC | |
| SluCh40 | CATCAGTCTGGAG | 1432 | AGACCAGACTTGT | 1472 | AGCATGTGCCTGT | 1512 |
| | CTAGGCA | | TGCCCAA | | AGTTC | |

TABLE 12
SluCas9 sgRNAs Categorized Based on Cleavage Efficiency

| Total INDEL % | Guides |
|---|---|
| <15% | SluCh3, SluCh5, SluCh6, SluCh7, SluCh12, SluCh13, SluCh14, SluCh15, |
| | SluCh16, SluCh17, SluCh18, SluCh19, SluCh23, SluCh26, SluCh29, |
| | SluCh30, SluCh31, SluCh33, SluCh37, SluCh38, SluCh40 |
| 15%-25% | SluCh1, SluCh2, SluCh10, SluCh21, SluCh22, SluCh24, SluCh34 |
| >25% | SluCh4, SluCh8, SluCh9, SluCh11, SluCh20, SluCh25, SluCh27, SluCh28, |
| | SluCh32, SluCh35, SluCh36, SluCh39 |

A subset of the SluCas9 sgRNAs were selected for subsequent evaluation, including measurement of INDEL frequency, FAAH mRNA levels, and FAAH protein levels in edited cells. The sgRNA evaluated are identified in Table 13, which includes SluCh8, SluCh9, SluCh11, SluCh20, SluCh27, SluCh28, SluCh32, and SluCh39 having cut locations within exon 1 or 2 of FAAH, and SluCh4 and SluCh25 having cut locations outside exon 1 or 2 of FAAH. Briefly, 3×10⁵ MCF7 cells were electroporated with 1.5 μg sgRNA and 1 μg SluCas9 protein, incubated for 72 hours. Cells were harvested for extraction of genomic DNA for use in INDEL quantification by TIDE analysis, for extraction of RNA for quantification of FAAH mRNA by qPCR, and for extraction of protein for quantification of FAAH protein by Simple Wes, each as described in Example 2.

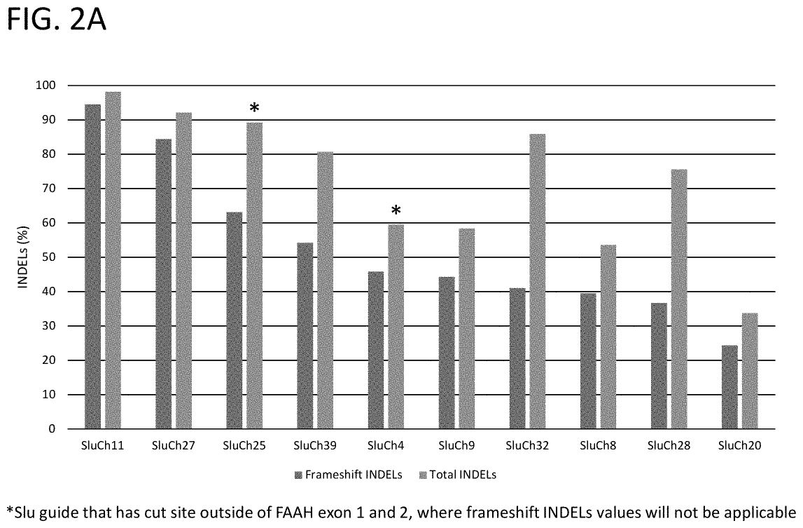

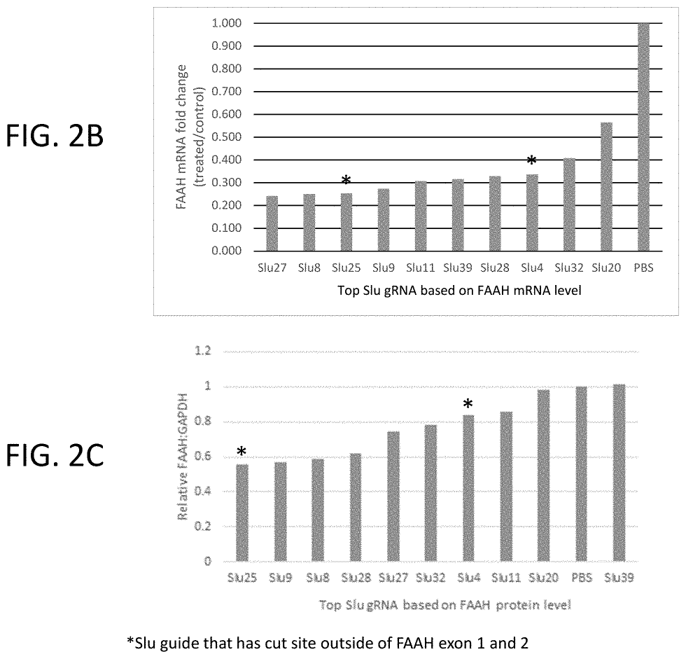

Quantification of overall INDEL frequency, as well as frequency of INDELs resulting in a frameshift mutation, is identified in Table 13. As shown in FIG. 2A, the sgRNA are further ranked based on frequency of INDELs expected to result in a frameshift mutation. The top sgRNAs that cut within an exon (SluCh11, SluCh27, and SluCh39) resulting in a frequency of INDELs resulting in a frameshift mutation that exceeded 50%.

Also provided in Table 13 are FAAH mRNA levels as measured by qPCR, provided as fold-change in cells electroporated with SluCas9/sgRNA complexes compared to control cells electroporated in PBS only. As shown in FIG. 2B, the sgRNA are further ranked based upon reduction of FAAH mRNA expression levels, with most of the sgRNAs resulting in a 60% or higher reduction in mRNA expression level.

The expression level of FAAH protein measured by Simple Wes was normalized to expression level of the internal control protein GAPDH. The relative expression level of FAAH protein was then normalized for edited samples relative to a PBS control sample that was not subjected to electroporation (see Table 13). As shown in FIG. 2C, the sgRNA are further ranked based upon reduction of FAAH protein levels, with the top four sgRNAs reducing FAAH protein levels by approximately 40%.

TABLE 13
Quantification of Editing Efficiency and Functional Activity
of SluCas9 sgRNAs Targeting FAAH Coding Sequence

| | Indel (%) | | FAAH mRNA (fold | FAAH protein |
|---|---|---|---|---|
| sgRNA ID | Total | Frameshift* | change) | (FAAH:GAPDH) |
| SluCh11 | 98.2 | 94.5 | 0.307527 | 0.857667575 |
| SluCh27 | 92.1 | 84.4 | 0.242103 | 0.745333219 |
| SluCh25 | 89.2 | 63.1 | 0.253054 | 0.553892166 |
| SluCh39 | 80.7 | 54.2 | 0.316017 | 1.01355642 |
| SluCh4 | 59.5 | 45.8 | 0.336368 | 0.838423787 |
| SluCh9 | 58.4 | 44.3 | 0.27299 | 0.571584882 |
| SluCh32 | 85.9 | 41 | 0.40806 | 0.782063469 |
| SluCh8 | 53.6 | 39.5 | 0.25047 | 0.587178518 |
| SluCh28 | 75.6 | 36.7 | 0.328629 | 0.622313139 |
| SluCh20 | 33.7 | 24.3 | 0.564625 | 0.980897116 |

*Frameshift INDEL % refers to INDELs expected to result in a frameshift mutation in the FAAH coding sequence (i.e., ±1 nt, ±2 nt, ±4 nt). The sgRNAs with values in underline have cut sites outside exon 1 or exon 2 of FAAH, wherein frameshift mutations are not applicable. Thus, Frameshift INDEL% refers to frequency of total INDELs minus frequency of INDELs that are a multiple of 3 (e.g., ±3 nt, ±6 nt, ±9 nt, etc.)

### Example 4: Evaluation of In Vitro Gene Editing and Functional Activity of gRNA/SaCas9 Targeting the FAAH Coding Sequence

Frequency of INDELs induced at predicted cut sites in the FAAH coding sequences was determined following in vitro treatment with SaCas9 protein and sgRNA prepared with spacers identified in Example 1.

Specifically, SaCas9 sgRNA were prepared with the spacers identified in Table 5 (SaCh1-SaCh16; SEQ ID NOs: 165-180) inserted into the sgRNA backbone identified by SEQ ID NO: 1271. Sequence of the SaCas9 sgRNA backbone is identified in Table 14. The SaCas9 sgRNA sequences were chemically synthesized by a commercial vendor.

TABLE 14
Sequences of SaCas9 sgRNA Targeting
FAAH Coding Sequence

| | | SEQ |
|---|---|---|
| | sgRNA Sequence | ID |
| Name | (Spacer in Bold) | NO |
| SaCh1 | mN*mN*mN*NNNNNNNNNNNNNNNNNNG | 1271 |
| sgRNA | UUUUAGUACUCUGGAAACAGAAUCUACU | |
| | AAAACAAGGCAAAAUGCCGUGUUUAUCU | |
| | CGUCAACUUGUUGGCGAmG*mA*mU* | |

mN*: 2′-O-methyl 3′phosphorothioate

The sgRNA were individually evaluated as complexes with SaCas9 protein for inducing INDELs at predicted cut sites in the FAAH coding sequence and for expression of FAAH mRNA. Briefly, 1×10⁵ MCF7 cells were electroporated with 3 μg sgRNA and 3 μg SaCas9 protein (SEQ ID NO: 1272) and incubated for 72 hours. The cells were then harvested for INDEL quantification by TIDE analysis or for FAAH mRNA expression by qPCR as described in Example 2.

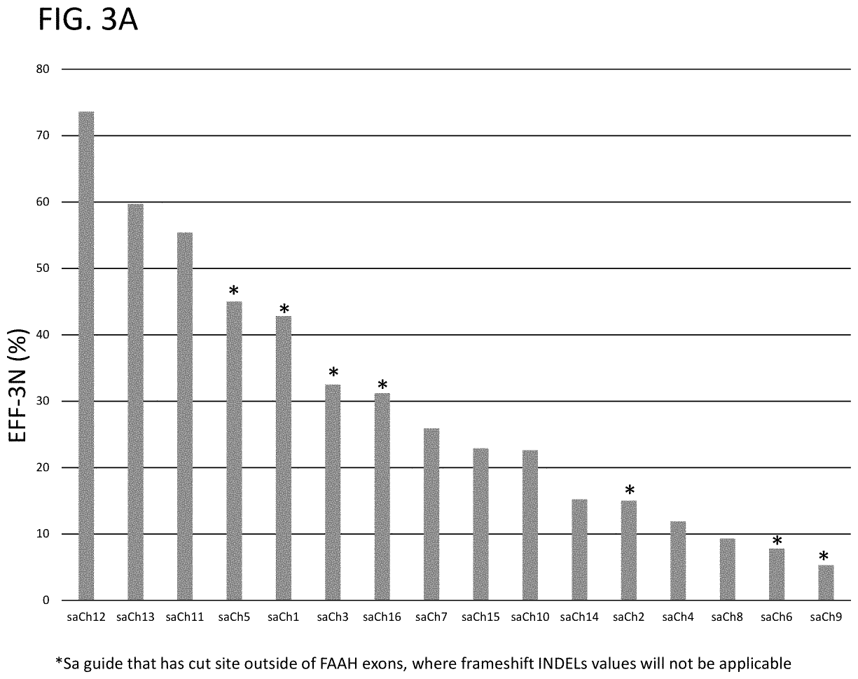

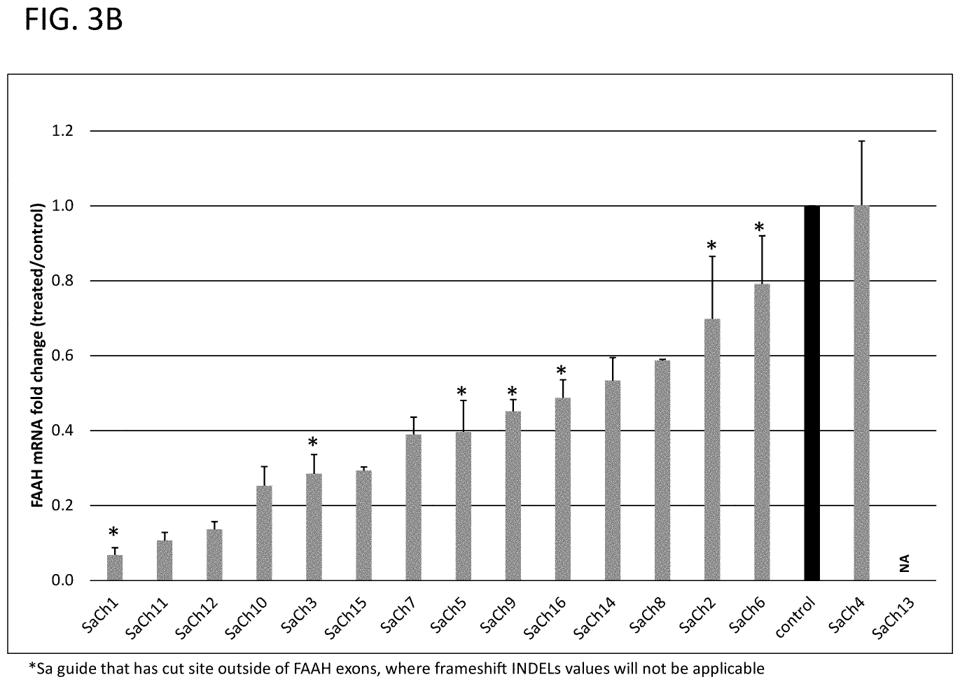

For INDEL quantification, genomic DNA was extracted and 1 μL (30-50 ng) of genomic DNA was used for PCR amplification of regions containing predicted cut sites. The purified PCR products were then sequenced using Sanger sequencing, and cutting efficiency was analyzed by Tsunami. The PCR and sequencing primers corresponding to each sgRNA are identified in Table 15. Quantification of overall INDEL frequency, as well as frequency of INDELs introducing a frameshift mutation, are identified for each sgRNA in Table 16. As shown in FIG. 3A, the sgRNA are further ranked based upon frequency of INDELs expected to disrupt the FAAH ORF through a frameshift mutation, with the top 3 sgRNA having a frequency exceeding 50%.

Quantification of FAAH mRNA levels by qPCR is provided in Table 16 as fold change for edited cells relative to control cells electroporated with SaCas9 protein only. Fold change was calculated by the 2\hat{ }(−ddCt) method. As shown in FIG. 3B, the sgRNA are further ranked based upon reduction of FAAH mRNA expression levels, with most sgRNAs resulting in a reduction of FAAH mRNA levels by 40% or more.

TABLE 15
TIDE Primer Sequences for Analysis of INDEL Frequency
at Cut Site Corresponding to SaCas9 sgRNAs

| | | SEQ | | SEQ | | SEQ | | SEQ | | SEQ |
|---|---|---|---|---|---|---|---|---|---|---|
| | PCR | ID | PCR | ID | Sequencing | ID | Sequencing | ID | Sequencing | ID |
| sgRNA | primer 1 | NO | primer 2 | NO | primer 1 | NO | primer 2 | NO | primer 3 | NO |
| SaCh 1 | TCTAACA | 1513 | AAGCTCT | 1529 | TCTAACA | 1545 | AAGCTCT | 1561 | CACTACG | 1577 |
| | GCTGGCA | | CCAGATC | | GCTGGCA | | CCAGATC | | CTCCGGC | |
| | TGTCTG | | CCCTTG | | TGTCTG | | CCCTTG | | AGTCACC | |
| SaCh 2 | TCTAACA | 1514 | AAGCTCT | 1530 | TCTAACA | 1546 | AAGCTCT | 1562 | CACTACG | 1578 |
| | GCTGGCA | | CCAGATC | | GCTGGCA | | CCAGATC | | CTCCGGC | |
| | TGTCTG | | CCCTTG | | TGTCTG | | CCCTTG | | AGTCACC | |
| SaCh 3 | TCTAACA | 1515 | AAGCTCT | 1531 | TCTAACA | 1547 | AAGCTCT | 1563 | CACTACG | 1579 |
| | GCTGGCA | | CCAGATC | | GCTGGCA | | CCAGATC | | CTCCGGC | |
| | TGTCTG | | CCCTTG | | TGTCTG | | CCCTTG | | AGTCACC | |
| SaCh 4 | TCTAACA | 1516 | AAGCTCT | 1532 | TCTAACA | 1548 | AAGCTCT | 1564 | CACTACG | 1580 |
| | GCTGGCA | | CCAGATC | | GCTGGCA | | CCAGATC | | CTCCGGC | |
| | TGTCTG | | CCCTTG | | TGTCTG | | CCCTTG | | AGTCACC | |
| SaCh 5 | TCTAACA | 1517 | AAGCTCT | 1533 | TCTAACA | 1549 | AAGCTCT | 1565 | CACTACG | 1581 |
| | GCTGGCA | | CCAGATC | | GCTGGCA | | CCAGATC | | CTCCGGC | |
| | TGTCTG | | CCCTTG | | TGTCTG | | CCCTTG | | AGTCACC | |
| SaCh 6 | CATCAGT | 1518 | AGACCAG | 1534 | CATCAGT | 1550 | AGACCAG | 1566 | AGACCAG | 1566 |
| | CTGGAGC | | ACTTGTT | | CTGGAGC | | ACTTGTT | | ACTTGTT | |
| | TAGGCA | | GCCCAA | | TAGGCA | | GCCCAA | | GCCCAA | |
| SaCh 7 | CATCAGT | 1519 | AGACCAG | 1535 | CATCAGT | 1551 | AGACCAG | 1567 | AGACCAG | 1567 |
| | CTGGAGC | | ACTTGTT | | CTGGAGC | | ACTTGTT | | ACTTGTT | |
| | TAGGCA | | GCCCAA | | TAGGCA | | GCCCAA | | GCCCAA | |
| SaCh 8 | CATCAGT | 1520 | AGACCAG | 1536 | CATCAGT | 1552 | AGACCAG | 1568 | AGACCAG | 1568 |
| | CTGGAGC | | ACTTGTT | | CTGGAGC | | ACTTGTT | | ACTTGTT | |
| | TAGGCA | | GCCCAA | | TAGGCA | | GCCCAA | | GCCCAA | |
| SaCh 9 | GACCAAC | 1521 | TCTGAAC | 1537 | GACCAAC | 1553 | TCTGAAC | 1569 | ACCTACA | 1582 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 10 | GACCAAC | 1522 | TCTGAAC | 1538 | GACCAAC | 1554 | TCTGAAC | 1570 | ACCTACA | 1583 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 11 | GACCAAC | 1523 | TCTGAAC | 1539 | GACCAAC | 1555 | TCTGAAC | 1571 | ACCTACA | 1584 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 12 | GACCAAC | 1524 | TCTGAAC | 1540 | GACCAAC | 1556 | TCTGAAC | 1572 | ACCTACA | 1585 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 13 | GACCAAC | 1525 | TCTGAAC | 1541 | GACCAAC | 1557 | TCTGAAC | 1573 | ACCTACA | 1586 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 14 | GACCAAC | 1526 | TCTGAAC | 1542 | GACCAAC | 1558 | TCTGAAC | 1574 | ACCTACA | 1587 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 15 | GACCAAC | 1527 | TCTGAAC | 1543 | GACCAAC | 1559 | TCTGAAC | 1575 | ACCTACA | 1588 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |
| SaCh 16 | GACCAAC | 1528 | TCTGAAC | 1544 | GACCAAC | 1560 | TCTGAAC | 1576 | ACCTACA | 1589 |
| | TGTGTGA | | ACTCACC | | TGTGTGA | | ACTCACC | | AGGTATG | |
| | CCTCCT | | GCTTTG | | CCTCCT | | GCTTTG | | CTCTGC | |

TABLE 16
Quantification of Editing Efficiency and Functional Activity
of SaCas9 sgRNAs Targeting FAAH Coding Sequence

| | | | fold | fold |
|---|---|---|---|---|
| Name | Total Indel % | Eff-3N* | change1** | change2** |
| SaCh1 | 53.9 | 42.8 | 0.082 | 0.054 |
| SaCh2 | 20.1 | 15 | 0.816 | 0.580 |
| SaCh3 | 51.2 | 32.5 | 0.321 | 0.249 |
| SaCh4 | 15.1 | 11.9 | 1.123 | 0.881 |
| SaCh5 | 54.5 | 45 | 0.456 | 0.337 |
| SaCh6 | 9.4 | 7.8 | 0.882 | 0.700 |
| SaCh7 | 86.3 | 25.9 | 0.357 | 0.422 |
| SaCh8 | 15.9 | 9.3 | 0.585 | 0.589 |
| SaCh9 | 7.4 | 5.3 | 0.429 | 0.474 |
| SaCh10 | 46.7 | 22.6 | 0.217 | 0.289 |
| SaCh11 | 65.1 | 55.4 | 0.092 | 0.122 |
| SaCh12 | 78.4 | 73.6 | 0.122 | 0.151 |
| SaCh13 | 67 | 59.7 | | |
| SaCh14 | 42.4 | 15.2 | 0.577 | 0.490 |
| SaCh15 | 53.2 | 22.9 | 0.286 | 0.300 |
| SaCh16 | 41.9 | 31.2 | 0.522 | 0.454 |

*EFF-3N = total frequency of INDELs minus frequency of in-frame INDELs (e.g., ±3 nt, ±6 nt, ±9 nt, etc). The sgRNAs with values in underline have cut sites outside exon 1, exon 2, or exon 4 of FAAH, wherein frameshift mutations are not applicable.
**control = 1.00

### Example 5: In Silico Identification of gRNA Target Sequences for Inducing a Microdeletion in FAAH-OUT

It was investigated whether use of a CRISPR/Cas9 genome editing system to induce a microdeletion in FAAH-OUT would result in decreased levels of FAAH expression.

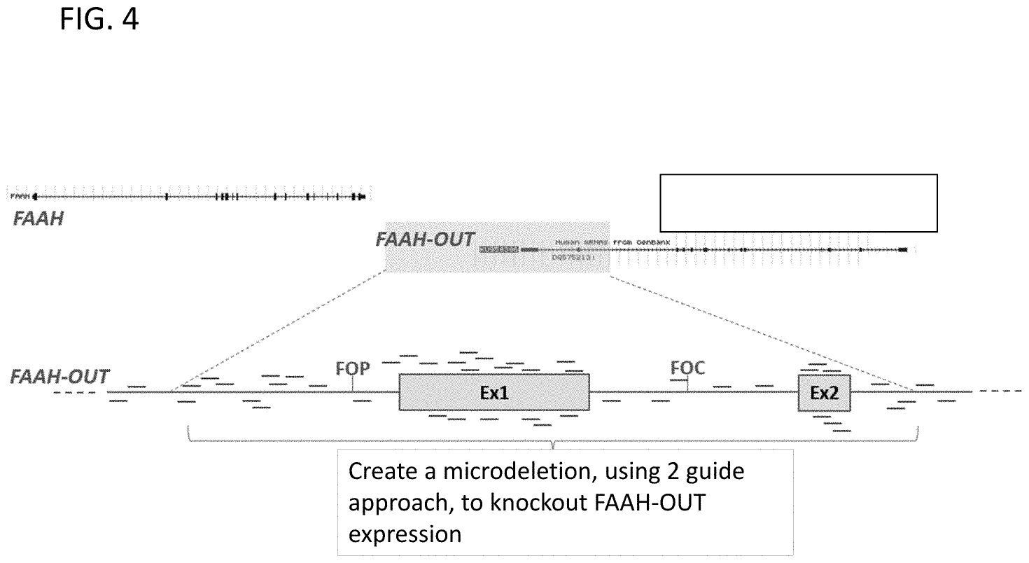

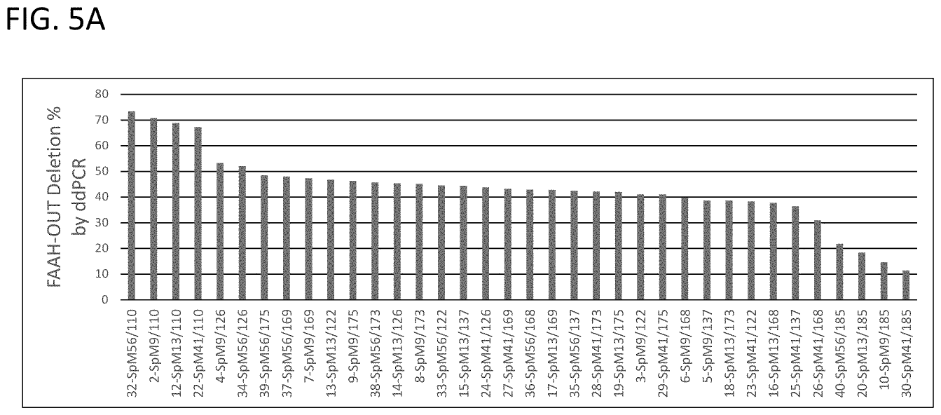

The 5′ end of the PT microdeletion is approximately 4.7 kb downstream the FAAH 3′ UTR, and is schematically depicted in FIG. 4. The microdeletion removes regulatory elements, including FOP and FOC. The DNaseI hypersensitivity cluster is targeted by the known gRNA “FOP1”, and the conserved region is targeted by the known gRNA “FOC1” (see, e.g., Mikaeli, et al (2019) bioRxiv, 633396). Approximately location of these elements are depicted in the schematic provide by FIG. 4. and further identified in Table 17.

TABLE 17
Chromosomal location of regions of FAAH-OUT

| Region of FAAH-OUT | Chromosome Location* |
|---|---|
| FOP | chr1: 46,422,536 (±200 bp)-46,422-695 (±200 bp) |
| FOP1 target sequence | Chr1: 46,422,643-46,422,663 |
| FOC | Chr1: 46,424,520 (±200 bp)-46,425,325 (±200 bp) |
| FOC1 target sequence | Chr1: 46,424,886-46,424,906 |
| Exon 1 | Chr1: 46,422,994-46,424,020 |
| Exon 2 | chr1: 46,426,339-46,426,460 |
| Exon 3 | chr1: 46,432,135-46,432,248 |
| PT microdeletion | Chr1: 46,418,743 (±600 bp)-46,426,873 (±600 bp) |

*According to human reference genome Hg38

Accordingly, a dual gRNA approach was developed to induce a microdeletion to remove regulatory elements, intronic elements, and/or coding sequence of FAAH-OUT, such as those removed by the PT microdeletion. In this approach, a first gRNA is combined with a second gRNA and Cas9 to induce two DSBs that result in a microdeletion. The first gRNA produces a DSB at an upstream target sequence in FAAH-OUT, and the second gRNA produces a DSB at a downstream target sequence in FAAH-OUT. Suitable regions for the target sequence of the first gRNA include a sequence upstream or within FOP. Suitable regions for the target sequence of the second gRNA include a sequence within or downstream FOC. As used herein, the first gRNA is referred to as the “left gRNA”, and the second gRNA is referred to as the “right gRNA”.

Thus, FAAH-OUT was evaluated for candidate gRNA target sequences using the CCTop algorithm based upon prediction of off-target sites with up to 4 mismatches in the human genome (Hg38). The region of FAAH-OUT evaluated for potential target sequences encompassed the PT microdeletion. A region extending from approximately 1 kb upstream the PT microdeletion (i.e., approximately 1 k upstream chr1:46,418,743 of Hg38) to approximately 1 kb downstream the PT microdeletion (i.e., approximately 1 kb downstream chr1:46,426,873 of Hg38) was evaluated for target sequences, as depicted by the schematic in FIG. 4. Specifically, the region was evaluated for 20 bp target sequences immediately upstream an SpCas9 PAM (pattern: N₂₀NGG (N=A,G,C,T); SEQ ID NO: 1282); 20 bp target sequences immediately upstream a SluCas9 PAM (pattern: N₂₀NNGG (N=A,G,C,T); SEQ ID NO: 1283); and 21 bp target sequences immediately upstream a SaCas9 PAM (pattern: N₂₁NNGRRT (N=A,G,C,T; R=A,G); SEQ ID NO: 1284).

The analysis identified approximately 2756 gRNA target sequences upstream SpCas9 PAM (NGG), approximately 2202 gRNA target sequences upstream SluCas9 PAM (NNGG), and approximately 470 gRNA target sequences upstream SaCas9 PAM (NNGRRT).

Subsequently, spacer sequences corresponding to the gRNA target sequences for SpCas9, SluCas9, and SaCas9 were filtered using the CCTop algorithm. Specifically, spacers were filtered to remove any that had one or more perfect matches to a different target site in the human genome (Hg38). Spacers were removed that were predicted to have either (i) one or more off-target sites with one mismatch; or (ii) three or more off-target sites with two mismatches. Moreover, spacers were selected for target sequences having a minor allele frequency of less than or equal to 0.001 in the human population. Finally, spacers were removed if the target sequence contained a homopolymer (i.e., consecutive sequence of five or more identical nucleotides, e.g., “AAAAA”, “CCCCC”, “GGGGG”, “TTTTT”). For SluCas9 and SpCas9 spacer sequences, certain spacers were removed that corresponded to difficult to sequence regions. SluCas9 and SpCas9 spacer sequences were selected for target sequences outside of the central FOP1-FOC1 region (chr1: 46,422,693-46,424,836). Also for SluCas9 and SpCas9 spacer sequences, CCTop score filters were applied to further eliminate spacer sequence with Raw CCTop score greater than −500 (SluCas9 spacers) and Raw CCTop score greater than −600 (SpCas9 spacers).

Based on this analysis, 185 spacer sequences for SpCas9 (Table 18; target sequences identified by SEQ ID NOs: 181-365; spacer sequences identified by SEQ ID NOs: 366-350, 186 spacer sequences for SluCas9 (Table 19; target sequences identified by SEQ ID NOs: 551-736; spacer sequences identified by SEQ ID NOs: 737-922); and 172 spacer sequences for SaCas9 (Table 20; target sequences identified by SEQ ID NOs: 923-1094; spacer sequences identified by SEQ ID NOs: 1095-1266) were identified. Target sequences identified upstream a SluCas9 PAM were extended to include 22 bp.

TABLE 18
Target and Spacer Sequences for SpCas9 gRNAs in FAAH-OUT

| | | SEQ | | SEQ | |
|---|---|---|---|---|---|
| | Target Sequence | ID | | ID | Cut site |
| Name | PAM in bold underline | NO | Spacer Sequence | NO | location* |
| SpM1 | TTGTAGCATTATCACTCTCTGAG | 181 | UUGUAGCAUUAUCACUCUCU | 366 | 46418017 |
| SpM2 | CAGAGAGTGATAATGCTACAAAG | 182 | CAGAGAGUGAUAAUGCUACA | 367 | 46418005 |
| SpM3 | ACTATGAGCCATCTACTTTCTGG | 183 | ACUAUGAGCCAUCUACUUUC | 368 | 46418398 |
| SpM4 | CTATGAGCCATCTACTTTCTGGG | 184 | CUAUGAGCCAUCUACUUUCU | 369 | 46418399 |
| SpM5 | TGAAGTGCCCAGAAAGTAGATGG | 185 | UGAAGUGCCCAGAAAGUAGA | 370 | 46418396 |
| SpM6 | AGGGTTCACAGAGGATTAAATGG | 186 | AGGGUUCACAGAGGAUUAAA | 371 | 46418427 |
| SpM7 | ATCCTCTGTGAACCCTATGATGG | 187 | AUCCUCUGUGAACCCUAUGA | 372 | 46418444 |
| SpM8 | TTCTGGCCATCGTACTCACTGGG | 188 | UUCUGGCCAUCGUACUCACU | 373 | 46418590 |
| SpM9 | CTTCTGGCCATCGTACTCACTGG | 189 | CUUCUGGCCAUCGUACUCAC | 374 | 46418591 |
| SpM10 | GATTTGTGCTCTCACTCTTCTGG | 190 | GAUUUGUGCUCUCACUCUUC | 375 | 46418607 |
| SpM11 | GGCAGTAGCCACCAGCACACTGG | 191 | GGCAGUAGCCACCAGCACAC | 376 | 46418657 |
| SpM12 | AGATGTCCAGTCTGGAGCCCAGG | 192 | AGAUGUCCAGUCUGGAGCCC | 377 | 46419046 |
| SpM13 | ATTGTTGGAGATGTCCAGTCTGG | 193 | AUUGUUGGAGAUGUCCAGUC | 378 | 46419054 |
| SpM14 | ATCTCCAACAATCTTTCACATGG | 194 | AUCUCCAACAAUCUUUCACA | 379 | 46419075 |
| SpM15 | ACTGCCATGTGAAAGATTGTTGG | 195 | ACUGCCAUGUGAAAGAUUGU | 380 | 46419069 |
| SpM16 | CAATCTTTCACATGGCAGTTAGG | 196 | CAAUCUUUCACAUGGCAGUU | 381 | 46419083 |
| SpM17 | CATAACAAGGCTTCTGGACTTGG | 197 | CAUAACAAGGCUUCUGGACU | 382 | 46419131 |
| SpM18 | GCAGGTCATAACAAGGCTTCTGG | 198 | GCAGGUCAUAACAAGGCUUC | 383 | 46419137 |
| SpM19 | AAGCCTTGTTATGACCTGCAAGG | 199 | AAGCCUUGUUAUGACCUGCA | 384 | 46419151 |
| SpM20 | TGGCCTTGCAGGTCATAACAAGG | 200 | UGGCCUUGCAGGUCAUAACA | 385 | 46419144 |
| SpM21 | CAGGTTAATCATGGCCTTGCAGG | 201 | CAGGUUAAUCAUGGCCUUGC | 386 | 46419155 |
| SpM22 | CTGCAGGGTCAGGTTAATCATGG | 202 | CUGCAGGGUCAGGUUAAUCA | 387 | 46419164 |
| SpM23 | GATTAACCTGACCCTGCAGCTGG | 203 | GAUUAACCUGACCCUGCAGC | 388 | 46419178 |
| SpM24 | GTGCTCACAGCCCGGCCACGTGG | 204 | GUGCUCACAGCCCGGCCACG | 389 | 46419232 |
| SpM25 | AGCCCGGCCACGTGGCCTGCAGG | 205 | AGCCCGGCCACGUGGCCUGC | 390 | 46419240 |
| SpM26 | TACCTGCAGGCCACGTGGCCGGG | 206 | UACCUGCAGGCCACGUGGCC | 391 | 46419232 |
| SpM27 | TTACCTGCAGGCCACGTGGCCGG | 207 | UUACCUGCAGGCCACGUGGC | 392 | 46419233 |
| SpM28 | AACATTACCTGCAGGCCACGTGG | 208 | AACAUUACCUGCAGGCCACG | 393 | 46419237 |
| SpM29 | GTGTTCTGAACATTACCTGCAGG | 209 | GUGUUCUGAACAUUACCUGC | 394 | 46419245 |
| SpM30 | CTCTTGGTGCTTGCTGTGCCTGG | 210 | CUCUUGGUGCUUGCUGUGCC | 395 | 46419307 |
| SpM31 | TGTGCCTGGAGTGTTGTTCCTGG | 211 | UGUGCCUGGAGUGUUGUUCC | 396 | 46419321 |
| SpM32 | GTGCCTGGAGTGTTGTTCCTGGG | 212 | GUGCCUGGAGUGUUGUUCCU | 397 | 46419322 |
| SpM33 | ACGAGTAGGTGGTCTTTAGGTGG | 213 | ACGAGUAGGUGGUCUUUAGG | 398 | 46419339 |
| SpM34 | AAAGACCACCTACTCGTCCAAGG | 214 | AAAGACCACCUACUCGUCCA | 399 | 46419355 |
| SpM35 | TGGACGAGTAGGTGGTCTTTAGG | 215 | UGGACGAGUAGGUGGUCUUU | 400 | 46419342 |
| SpM36 | CACCTACTCGTCCAAGGTGAGGG | 216 | CACCUACUCGUCCAAGGUGA | 401 | 46419361 |
| SpM37 | CCTCACCTTGGACGAGTAGGTGG | 217 | CCUCACCUUGGACGAGUAGG | 402 | 46419350 |
| SpM38 | TGCCCTCACCTTGGACGAGTAGG | 218 | UGCCCUCACCUUGGACGAGU | 403 | 46419353 |
| SpM39 | TCAACACAGCCTGACAGAGTTGG | 219 | UCAACACAGCCUGACAGAGU | 404 | 46419385 |
| SpM40 | ACCCAACATCTGTTAGGCTGTGG | 220 | ACCCAACAUCUGUUAGGCUG | 405 | 46419474 |
| SpM41 | TCCACAGCCTAACAGATGTTGGG | 221 | UCCACAGCCUAACAGAUGUU | 406 | 46419465 |
| SpM42 | TGTAGGTCACGCCCTTTCCTTGG | 222 | UGUAGGUCACGCCCUUUCCU | 407 | 46420652 |
| SpM43 | AACTAAAGATACATCTGGCTGGG | 223 | AACUAAAGAUACAUCUGGCU | 408 | 46420712 |
| SpM44 | GAACTAAAGATACATCTGGCTGG | 224 | GAACUAAAGAUACAUCUGGC | 409 | 46420713 |
| SpM45 | ATGTGAACTAAAGATACATCTGG | 225 | AUGUGAACUAAAGAUACAUC | 410 | 46420717 |
| SpM46 | TTTCTCTGGCTGGGCTTAGCTGG | 226 | UUUCUCUGGCUGGGCUUAGC | 411 | 46420749 |
| SpM47 | TTCTCTGGCTGGGCTTAGCTGGG | 227 | UUCUCUGGCUGGGCUUAGCU | 412 | 46420750 |
| SpM48 | TATGTGTAGGAAACTTGGGAGGG | 228 | UAUGUGUAGGAAACUUGGGA | 413 | 46420763 |
| SpM49 | CTATGTGTAGGAAACTTGGGAGG | 229 | CUAUGUGUAGGAAACUUGGG | 414 | 46420764 |
| SpM50 | AATCTATGTGTAGGAAACTTGGG | 230 | AAUCUAUGUGUAGGAAACUU | 415 | 46420767 |
| SpM51 | AAGGTGAAGCCACTTGGGATCGG | 231 | AAGGUGAAGCCACUUGGGAU | 416 | 46420827 |
| SpM52 | TCCATTCAACAAGCCTTCTCTGG | 232 | UCCAUUCAACAAGCCUUCUC | 417 | 46420888 |
| SpM53 | AGGCTTGTTGAATGGAGCAATGG | 233 | AGGCUUGUUGAAUGGAGCAA | 418 | 46420905 |
| SpM54 | GGCTTGTTGAATGGAGCAATGGG | 234 | GGCUUGUUGAAUGGAGCAAU | 419 | 46420906 |
| SpM55 | GCAATGGGTGACTTGTTATTAGG | 235 | GCAAUGGGUGACUUGUUAUU | 420 | 46420921 |
| SpM56 | CAATGGGTGACTTGTTATTAGGG | 236 | CAAUGGGUGACUUGUUAUUA | 421 | 46420922 |
| SpM57 | GCAAAGGGTCAGGGACTGATTGG | 237 | GCAAAGGGUCAGGGACUGAU | 422 | 46420991 |
| SpM58 | ATGGGCACTAATCAAGATCATGG | 238 | AUGGGCACUAAUCAAGAUCA | 423 | 46421322 |
| SpM59 | GATCTTGATTAGTGCCCATGAGG | 239 | GAUCUUGAUUAGUGCCCAUG | 424 | 46421336 |
| SpM60 | TGGGAATTATTTGTCCTCATGGG | 240 | UGGGAAUUAUUUGUCCUCAU | 425 | 46421340 |
| SpM61 | CTGGGAATTATTTGTCCTCATGG | 241 | CUGGGAAUUAUUUGUCCUCA | 426 | 46421341 |
| SpM62 | CTATAGGTTGAATGTAGACTGGG | 242 | CUAUAGGUUGAAUGUAGACU | 427 | 46421359 |
| SpM63 | GCTATAGGTTGAATGTAGACTGG | 243 | GCUAUAGGUUGAAUGUAGAC | 428 | 46421360 |
| SpM64 | TTCAACCTATAGCTTTCTCCTGG | 244 | UUCAACCUAUAGCUUUCUCC | 429 | 46421380 |
| SpM65 | ACAGACCAGGAGAAAGCTATAGG | 245 | ACAGACCAGGAGAAAGCUAU | 430 | 46421375 |
| SpM66 | TAGATGAGGATCTACAGACCAGG | 246 | UAGAUGAGGAUCUACAGACC | 431 | 46421388 |
| SpM67 | ATCCAACATCCGTTAGGCTGTGG | 247 | AUCCAACAUCCGUUAGGCUG | 432 | 46421525 |
| SpM68 | GGTTCAACTCCACAGCCTAACGG | 248 | GGUUCAACUCCACAGCCUAA | 433 | 46421524 |
| SpM69 | GGTTGCTCTCTGAACAACAATGG | 249 | GGUUGCUCUCUGAACAACAA | 434 | 46421625 |
| SpM70 | GCTCTCTGAACAACAATGGAGGG | 250 | GCUCUCUGAACAACAAUGGA | 435 | 46421629 |
| SpM71 | GGTTACCCTGAACATACTGTGGG | 251 | GGUUACCCUGAACAUACUGU | 436 | 46421693 |
| SpM72 | TGGTTACCCTGAACATACTGTGG | 252 | UGGUUACCCUGAACAUACUG | 437 | 46421694 |
| SpM73 | AGGCAGGGACTATTTCTGATTGG | 253 | AGGCAGGGACUAUUUCUGAU | 438 | 46421714 |
| SpM74 | TACATTTGATGTCTGTTTCCTGG | 254 | UACAUUUGAUGUCUGUUUCC | 439 | 46421756 |
| SpM75 | GTTTATTCTCATAATACCCAGGG | 255 | GUUUAUUCUCAUAAUACCCA | 440 | 46421822 |
| SpM76 | GGTTTATTCTCATAATACCCAGG | 256 | GGUUUAUUCUCAUAAUACCC | 441 | 46421823 |
| SpM77 | GGAATGAGTGTGTTTCAAGGAGG | 257 | GGAAUGAGUGUGUUUCAAGG | 442 | 46421960 |
| SpM78 | ATGCTGCTGATTTACTCTTGAGG | 258 | AUGCUGCUGAUUUACUCUUG | 443 | 46422002 |
| SpM79 | TTTACTCTTGAGGAGATCACTGG | 259 | UUUACUCUUGAGGAGAUCAC | 444 | 46422012 |
| SpM80 | TTACTCTTGAGGAGATCACTGGG | 260 | UUACUCUUGAGGAGAUCACU | 445 | 46422013 |
| SpM81 | TTAGGGAGGGTGTAAATCTGAGG | 261 | UUAGGGAGGGUGUAAAUCUG | 446 | 46422156 |
| SpM82 | TAGGGAGGGTGTAAATCTGAGGG | 262 | UAGGGAGGGUGUAAAUCUGA | 447 | 46422157 |
| SpM83 | GAGCTAGCAGCAACGCACAGAGG | 263 | GAGCUAGCAGCAACGCACAG | 448 | 46422238 |
| SpM84 | AGCTAGCAGCAACGCACAGAGGG | 264 | AGCUAGCAGCAACGCACAGA | 449 | 46422239 |
| SpM85 | ATGAGGTATGTGGTAACGGAAGG | 265 | AUGAGGUAUGUGGUAACGGA | 450 | 46422624 |
| SpM86 | TGAGGTATGTGGTAACGGAAGGG | 266 | UGAGGUAUGUGGUAACGGAA | 451 | 46422625 |
| SpM87 | GTAACGGAAGGGTGTAACCCAGG | 267 | GUAACGGAAGGGUGUAACCC | 452 | 46422636 |
| SpM88 | AGGCCTAGAGTGCTGTGCCGTGG | 268 | AGGCCUAGAGUGCUGUGCCG | 453 | 46422675 |
| SpM89 | GGCCTAGAGTGCTGTGCCGTGGG | 269 | GGCCUAGAGUGCUGUGCCGU | 454 | 46422676 |
| SpM90 | ATCCCACGGCACAGCACTCTAGG | 270 | AUCCCACGGCACAGCACUCU | 455 | 46422668 |
| SpM91 | GAGTGCTGTGCCGTGGGATGTGG | 271 | GAGUGCUGUGCCGUGGGAUG | 456 | 46422682 |
| SpM92 | CTGTGCCGTGGGATGTGGTGCGG | 272 | CUGUGCCGUGGGAUGUGGUG | 457 | 46422687 |
| SpM93 | TGTGCCGTGGGATGTGGTGCGGG | 273 | UGUGCCGUGGGAUGUGGUGC | 458 | 46422688 |
| SpM94 | GTCACCCGCACCACATCCCACGG | 274 | GUCACCCGCACCACAUCCCA | 459 | 46422682 |
| SpM95 | GATGTGGTGCGGGTGACAAGTGG | 275 | GAUGUGGUGCGGGUGACAAG | 460 | 46422698 |
| SpM96 | GGTGCGGGTGACAAGTGGCCTGG | 276 | GGUGCGGGUGACAAGUGGCC | 461 | 46422703 |
| SpM97 | GTGCGGGTGACAAGTGGCCTGGG | 277 | GUGCGGGUGACAAGUGGCCU | 462 | 46422704 |
| SpM98 | GGAGTTCATGAAGGTGGAGTGGG | 278 | GGAGUUCAUGAAGGUGGAGU | 463 | 46422752 |
| SpM99 | GTTACAGAGTGGGCAACTTCAGG | 279 | GUUACAGAGUGGGCAACUUC | 464 | 46422855 |
| SpM100 | TTACAGAGTGGGCAACTTCAGGG | 280 | UUACAGAGUGGGCAACUUCA | 465 | 46422856 |
| SpM101 | AGACAAACATAGACTGAGCCTGG | 281 | AGACAAACAUAGACUGAGCC | 466 | 46424709 |
| SpM102 | GACAAACATAGACTGAGCCTGGG | 282 | GACAAACAUAGACUGAGCCU | 467 | 46424710 |
| SpM103 | GACGGGTTGTCACATCCTCCAGG | 283 | GACGGGUUGUCACAUCCUCC | 468 | 46424736 |
| SpM104 | AGGATGTGACAACCCGTCTCTGG | 284 | AGGAUGUGACAACCCGUCUC | 469 | 46424751 |
| SpM105 | GGATGTGACAACCCGTCTCTGGG | 285 | GGAUGUGACAACCCGUCUCU | 470 | 46424752 |
| SpM106 | GGGATGGGCTCATGGTCTCTCGG | 286 | GGGAUGGGCUCAUGGUCUCU | 471 | 46424801 |
| SpM107 | TGATGATGGTGGACTCAGTCTGG | 287 | UGAUGAUGGUGGACUCAGUC | 472 | 46424840 |
| SpM108 | GATGATGGTGGACTCAGTCTGGG | 288 | GAUGAUGGUGGACUCAGUCU | 473 | 46424841 |
| SpM109 | GTGGACTCAGTCTGGGAGCCCGG | 289 | GUGGACUCAGUCUGGGAGCC | 474 | 46424848 |
| SpM110 | GACTCAGTCTGGGAGCCCGGAGG | 290 | GACUCAGUCUGGGAGCCCGG | 475 | 46424851 |
| SpM111 | AGTCTGGGAGCCCGGAGGTAGGG | 291 | AGUCUGGGAGCCCGGAGGUA | 476 | 46424856 |
| SpM112 | GAGGTGCTGTTCCCATGCTTTGG | 292 | GAGGUGCUGUUCCCAUGCUU | 477 | 46424895 |
| SpM113 | AAGCATGGGAACAGCACCTCAGG | 293 | AAGCAUGGGAACAGCACCUC | 478 | 46424882 |
| SpM114 | ACTCAGGAACTCCAAAGCATGGG | 294 | ACUCAGGAACUCCAAAGCAU | 479 | 46424896 |
| SpM115 | GGGCCATCAATCACCATCCAGGG | 295 | GGGCCAUCAAUCACCAUCCA | 480 | 46424932 |
| SpM116 | TCCTCCTCATCAACCAGGGAGGG | 296 | UCCUCCUCAUCAACCAGGGA | 481 | 46425063 |
| SpM117 | TTCCTCCTCATCAACCAGGGAGG | 297 | UUCCUCCUCAUCAACCAGGG | 482 | 46425064 |
| SpM118 | GACTTCCTCCTCATCAACCAGGG | 298 | GACUUCCUCCUCAUCAACCA | 483 | 46425067 |
| SpM119 | TGGTTGATGAGGAGGAAGTCTGG | 299 | UGGUUGAUGAGGAGGAAGUC | 484 | 46425080 |
| SpM120 | AGACTTCCTCCTCATCAACCAGG | 300 | AGACUUCCUCCUCAUCAACC | 485 | 46425068 |
| SpM121 | GGTTGATGAGGAGGAAGTCTGGG | 301 | GGUUGAUGAGGAGGAAGUCU | 486 | 46425081 |
| SpM122 | AGGAGGAAGTCTGGGCTAATGGG | 302 | AGGAGGAAGUCUGGGCUAAU | 487 | 46425089 |
| SpM123 | TCTGGGCTAATGGGTTGCAGTGG | 303 | UCUGGGCUAAUGGGUUGCAG | 488 | 46425098 |
| SpM124 | ACCCACCACCGCACACAGATGGG | 304 | ACCCACCACCGCACACAGAU | 489 | 46425152 |
| SpM125 | TACCCACCACCGCACACAGATGG | 305 | UACCCACCACCGCACACAGA | 490 | 46425153 |
| SpM126 | GGGTATAGCTTCCTTTACTGCGG | 306 | GGGUAUAGCUUCCUUUACUG | 491 | 46425181 |
| SpM127 | TGCTTTCTTGTGCCTCCTGCTGG | 307 | UGCUUUCUUGUGCCUCCUGC | 492 | 46425213 |
| SpM128 | GCTGGCATTTCATTGTGTTGTGG | 308 | GCUGGCAUUUCAUUGUGUUG | 493 | 46425231 |
| SpM129 | GTTGTGGTTGGTTGTGTGTCTGG | 309 | GUUGUGGUUGGUUGUGUGUC | 494 | 46425247 |
| SpM130 | TGGCTGTGTGGTTATGTGCCTGG | 310 | UGGCUGUGUGGUUAUGUGCC | 495 | 46425272 |
| SpM131 | TGTGTGGTTATGTGCCTGGCTGG | 311 | UGUGUGGUUAUGUGCCUGGC | 496 | 46425276 |
| SpM132 | GTGTGCATGTGTTGGGTTATTGG | 312 | GUGUGCAUGUGUUGGGUUAU | 497 | 46425299 |
| SpM133 | GCATGTGTTGGGTTATTGGTTGG | 313 | GCAUGUGUUGGGUUAUUGGU | 498 | 46425303 |
| SpM134 | TGTGTACATCTAGCTATGTGTGG | 314 | UGUGUACAUCUAGCUAUGUG | 499 | 46425328 |
| SpM135 | CTAGCTATGTGTGGCTGGTGTGG | 315 | CUAGCUAUGUGUGGCUGGUG | 500 | 46425337 |
| SpM136 | TAGCTATGTGTGGCTGGTGTGGG | 316 | UAGCUAUGUGUGGCUGGUGU | 501 | 46425338 |
| SpM137 | CTGGTGTGGGTCTGAATGTCTGG | 317 | CUGGUGUGGGUCUGAAUGUC | 502 | 46425351 |
| SpM138 | CAGCTGGTTTGGTATGTGTCTGG | 318 | CAGCUGGUUUGGUAUGUGUC | 503 | 46425416 |
| SpM139 | AGCTGGTTTGGTATGTGTCTGGG | 319 | AGCUGGUUUGGUAUGUGUCU | 504 | 46425417 |
| SpM140 | TTGGTATGTGTCTGGGCATCTGG | 320 | UUGGUAUGUGUCUGGGCAUC | 505 | 46425424 |
| SpM141 | GCATCTGGTTGGTGAACATGTGG | 321 | GCAUCUGGUUGGUGAACAUG | 506 | 46425439 |
| SpM142 | TTGGTGAACATGTGGATGTCTGG | 322 | UUGGUGAACAUGUGGAUGUC | 507 | 46425447 |
| SpM143 | TGGTGAACATGTGGATGTCTGGG | 323 | UGGUGAACAUGUGGAUGUCU | 508 | 46425448 |
| SpM144 | CATGTGGATGTCTGGGCTGTTGG | 324 | CAUGUGGAUGUCUGGGCUGU | 509 | 46425455 |
| SpM145 | ATGTGGATGTCTGGGCTGTTGGG | 325 | AUGUGGAUGUCUGGGCUGUU | 510 | 46425456 |
| SpM146 | GGATGTCTGGGCTGTTGGGCTGG | 326 | GGAUGUCUGGGCUGUUGGGC | 511 | 46425460 |
| SpM147 | GATGTCTGGGCTGTTGGGCTGGG | 327 | GAUGUCUGGGCUGUUGGGCU | 512 | 46425461 |
| SpM148 | GTATATGTCTGGATGGCTGGAGG | 328 | GUAUAUGUCUGGAUGGCUGG | 513 | 46425496 |
| SpM149 | GTGTCTCCAGCCTCCCATTGTGG | 329 | GUGUCUCCAGCCUCCCAUUG | 514 | 46425552 |
| SpM150 | CAGCCTCCCATTGTGGTTTCAGG | 330 | CAGCCUCCCAUUGUGGUUUC | 515 | 46425559 |
| SpM151 | CATTGTGGTTTCAGGCTTCTTGG | 331 | CAUUGUGGUUUCAGGCUUCU | 516 | 46425567 |
| SpM152 | ACAGACCTGTATAGCTTGTTGGG | 332 | ACAGACCUGUAUAGCUUGUU | 517 | 46425601 |
| SpM153 | GACAGACCTGTATAGCTTGTTGG | 333 | GACAGACCUGUAUAGCUUGU | 518 | 46425602 |
| SpM154 | ACATGACTGAGAAGGTGCCCAGG | 334 | ACAUGACUGAGAAGGUGCCC | 519 | 46425624 |
| SpM155 | AGTGACAACCTCGAGACCTCAGG | 335 | AGUGACAACCUCGAGACCUC | 520 | 46425672 |
| SpM156 | GGTGGACACCTGAGGTCTCGAGG | 336 | GGUGGACACCUGAGGUCUCG | 521 | 46425670 |
| SpM157 | AGGTGTCCACCTTTATGTCCCGG | 337 | AGGUGUCCACCUUUAUGUCC | 522 | 46425692 |
| SpM158 | GGTGTCCACCTTTATGTCCCGGG | 338 | GGUGUCCACCUUUAUGUCCC | 523 | 46425693 |
| SpM159 | ATTTGTTTGCTGAGCCTGTGAGG | 339 | AUUUGUUUGCUGAGCCUGUG | 524 | 46425735 |
| SpM160 | AAGACCTGGAGAAATTCCCTGGG | 340 | AAGACCUGGAGAAAUUCCCU | 525 | 46425785 |
| SpM161 | CAAGACCTGGAGAAATTCCCTGG | 341 | CAAGACCUGGAGAAAUUCCC | 526 | 46425786 |
| SpM162 | TTTAGCACAAGTGTGAGTCAGGG | 342 | UUUAGCACAAGUGUGAGUCA | 527 | 46425857 |
| SpM163 | TCACCAGTTCTGTGGGCATCTGG | 343 | UCACCAGUUCUGUGGGCAUC | 528 | 46425963 |
| SpM164 | AGGAGGGTGGCTGGTCTGTCTGG | 344 | AGGAGGGUGGCUGGUCUGUC | 529 | 46426002 |
| SpM165 | AGCTCACTCACCACCCGTCTGGG | 345 | AGCUCACUCACCACCCGUCU | 530 | 46426038 |
| SpM166 | CAGCTCACTCACCACCCGTCTGG | 346 | CAGCUCACUCACCACCCGUC | 531 | 46426039 |
| SpM167 | CTGAACCTCATGGCACCTGTAGG | 347 | CUGAACCUCAUGGCACCUGU | 532 | 46426069 |
| SpM168 | GAAACGAGAAAGGCAGTACCAGG | 348 | GAAACGAGAAAGGCAGUACC | 533 | 46426107 |
| SpM169 | AAACGAGAAAGGCAGTACCAGGG | 349 | AAACGAGAAAGGCAGUACCA | 534 | 46426108 |
| SpM170 | CGAGAAAGGCAGTACCAGGGAGG | 350 | CGAGAAAGGCAGUACCAGGG | 535 | 46426111 |
| SpM171 | ACAGAAACACTGCCTCATCTGGG | 351 | ACAGAAACACUGCCUCAUCU | 536 | 46426131 |
| SpM172 | ATAATATTCCTAGGACCCATTGG | 352 | AUAAUAUUCCUAGGACCCAU | 537 | 46426178 |
| SpM173 | TAATATTCCTAGGACCCATTGGG | 353 | UAAUAUUCCUAGGACCCAUU | 538 | 46426179 |
| SpM174 | CCTAGGACCCATTGGGTAAATGG | 354 | CCUAGGACCCAUUGGGUAAA | 539 | 46426186 |
| SpM175 | CCATTTACCCAATGGGTCCTAGG | 355 | CCAUUUACCCAAUGGGUCCU | 540 | 46426176 |
| SpM176 | AGCTGGTCCATTTACCCAATGGG | 356 | AGCUGGUCCAUUUACCCAAU | 541 | 46426183 |
| SpM177 | CAGCTGGTCCATTTACCCAATGG | 357 | CAGCUGGUCCAUUUACCCAA | 542 | 46426184 |
| SpM178 | GTAAATGGACCAGCTGCTCATGG | 358 | GUAAAUGGACCAGCUGCUCA | 543 | 46426201 |
| SpM179 | ATGGACCAGCTGCTCATGGCTGG | 359 | AUGGACCAGCUGCUCAUGGC | 544 | 46426205 |
| SpM180 | TGCTCAAGCTACTCATGGCCAGG | 360 | UGCUCAAGCUACUCAUGGCC | 545 | 46426231 |
| SpM181 | TAATTAGAAGTTGTCTAGCATGG | 361 | UAAUUAGAAGUUGUCUAGCA | 546 | 46427784 |
| SpM182 | TGTGGCTTCTGTTGTTGGGCTGG | 362 | UGUGGCUUCUGUUGUUGGGC | 547 | 46427817 |
| SpM183 | GTGTAAGTGTTTGCTGGGTTTGG | 363 | GUGUAAGUGUUUGCUGGGUU | 548 | 46427961 |
| SpM184 | ATTTAAGTGTAAGTGTTTGCTGG | 364 | AUUUAAGUGUAAGUGUUUGC | 549 | 46427967 |
| SpM185 | TAAATGTTTACAGTGGTGCCTGG | 365 | UAAAUGUUUACAGUGGUGCC | 550 | 46428074 |

*chromosomal location of guide cut-site in chromosome 1 of human genome Hg38

TABLE 19
Target and Spacer Sequences for SluCas9 gRNAs in FAAH-OUT

| | | SEQ | | SEQ | |
|---|---|---|---|---|---|
| | Target Sequence | ID | | ID | Cut site |
| Name | PAM in bold underline | NO | Spacer Sequence | NO | location * |
| SluM1 | CCTACTATGAGCCATCTACTTTCTGG | 551 | c | 737 | 46418397 |
| SluM2 | CTACTATGAGCCATCTACTTTCTGGG | 552 | CUACUAUGAGCCAUCUACUUUC | 738 | 46418398 |
| SluM3 | CTGTGAAGTGCCCAGAAAGTAGATGG | 533 | CUGUGAAGUGCCCAGAAAGUAG | 739 | 46418397 |
| SluM4 | CATAGGGTTCACAGAGGATTAAATGG | 554 | CAUAGGGUUCACAGAGGAUUAA | 740 | 46418428 |
| SluM5 | TTAATCCTCTGTGAACCCTATGATGG | 555 | UUAAUCCUCUGUGAACCCUAUG | 741 | 46418443 |
| SluM6 | TAATCCTCTGTGAACCCTATGATGGG | 556 | UAAUCCUCUGUGAACCCUAUGA | 742 | 46418444 |
| SluM7 | CCAGGGTCCCACAGCTAGAAGTTGGG | 337 | CCAGGGUCCCACAGCUAGAAGU | 743 | 46418510 |
| SluM8 | ACTCTTCTGGCCATCGTACTCACTGG | 558 | ACUCUUCUGGCCAUCGUACUCA | 744 | 46418592 |
| SluM9 | GCTGATTTGTGCTCTCACTCTTCTGG | 339 | GCUGAUUUGUGCUCUCACUCUU | 745 | 46418608 |
| SluM10 | GGCAGTAGCCACCAGCACACTGGTGG | 560 | GGCAGUAGCCACCAGCACACUG | 746 | 46418655 |
| Slumll | ACAGGCAGTAGCCACCAGCACACTGG | 561 | ACAGGCAGUAGCCACCAGCACA | 747 | 46418658 |
| SluM12 | CCTCGCTTCCCTGGGCTCCAGACTGG | 562 | CCUCGCUUCCCUGGGCUCCAGA | 748 | 46419049 |
| SluM13 | CCAGTCTGGAGCCCAGGGAAGCGAGG | 563 | CCAGUCUGGAGCCCAGGGAAGC | 749 | 46419038 |
| SluM14 | GGAGATGTCCAGTCTGGAGCCCAGGG | 564 | GGAGAUGUCCAGUCUGGAGCCC | 750 | 46419046 |
| SluM15 | TGGAGATGTCCAGTCTGGAGCCCAGG | 565 | UGGAGAUGUCCAGUCUGGAGCC | 751 | 46419047 |
| SluM16 | AAGATTGTTGGAGATGTCCAGTCTGG | 566 | AAGAUUGUUGGAGAUGUCCAGU | 752 | 46419055 |
| SluM17 | GACATCTCCAACAATCTTTCACATGG | 567 | GACAUCUCCAACAAUCUUUCAC | 753 | 46419074 |
| SluM18 | CAACAATCTTTCACATGGCAGTTAGG | 368 | CAACAAUCUUUCACAUGGCAGU | 754 | 46419082 |
| SluM19 | CTAACTGCCATGTGAAAGATTGTTGG | 569 | CUAACUGCCAUGUGAAAGAUUG | 755 | 46419070 |
| SluM20 | GGTCATAACAAGGCTTCTGGACTTGG | 370 | GGUCAUAACAAGGCUUCUGGAC | 756 | 46419132 |
| SluM21 | CAGAAGCCTTGTTATGACCTGCAAGG | 571 | CAGAAGCCUUGUUAUGACCUGC | 757 | 46419150 |
| SluM22 | CTTGCAGGTCATAACAAGGCTTCTGG | 572 | CUUGCAGGUCAUAACAAGGCUU | 758 | 46419138 |
| SluM23 | TCATGGCCTTGCAGGTCATAACAAGG | 373 | UCAUGGCCUUGCAGGUCAUAAC | 759 | 46419145 |
| SluM24 | GGTCAGGTTAATCATGGCCTTGCAGG | 574 | GGUCAGGUUAAUCAUGGCCUUG | 760 | 46419156 |
| SluM25 | CATGATTAACCTGACCCTGCAGCTGG | 373 | CAUGAUUAACCUGACCCUGCAG | 761 | 46419177 |
| SluM26 | CAGCTGCAGGGTCAGGTTAATCATGG | 576 | CAGCUGCAGGGUCAGGUUAAUC | 762 | 46419165 |
| SluM27 | GCCCTTCCTCAGTGCTCACAGCCCGG | 377 | GCCCUUCCUCAGUGCUCACAGC | 763 | 46419223 |
| SluM28 | GCCGGGCTGTGAGCACTGAGGAAGGG | 378 | GCCGGGCUGUGAGCACUGAGGA | 764 | 46419213 |
| SluM29 | ACGTGGCCGGGCTGTGAGCACTGAGG | 379 | ACGUGGCCGGGCUGUGAGCACU | 765 | 46419218 |
| SluM30 | TCAGTGCTCACAGCCCGGCCACGTGG | 380 | UCAGUGCUCACAGCCCGGCCAC | 766 | 46419231 |
| SluM31 | CACAGCCCGGCCACGTGGCCTGCAGG | 381 | CACAGCCCGGCCACGUGGCCUG | 767 | 46419239 |
| SluM32 | CATTACCTGCAGGCCACGTGGCCGGG | 382 | CAUUACCUGCAGGCCACGUGGC | 768 | 46419233 |
| SluM33 | ACATTACCTGCAGGCCACGTGGCCGG | 383 | ACAUUACCUGCAGGCCACGUGG | 769 | 46419234 |
| SluM34 | CTGAACATTACCTGCAGGCCACGTGG | 384 | CUGAACAUUACCUGCAGGCCAC | 770 | 46419238 |
| SluM35 | TCGGTGTTCTGAACATTACCTGCAGG | 383 | UCGGUGUUCUGAACAUUACCUG | 771 | 46419246 |
| SluM36 | GCACAGCAAGCACCAAGAGCAAAGGG | 386 | GCACAGCAAGCACCAAGAGCAA | 772 | 46419291 |
| SluM37 | GGCACAGCAAGCACCAAGAGCAAAGG | 387 | GGCACAGCAAGCACCAAGAGCA | 773 | 46419292 |
| SluM38 | TTGCTCTTGGTGCTTGCTGTGCCTGG | 388 | UUGCUCUUGGUGCUUGCUGUGC | 774 | 46419306 |
| SluM39 | TGCTGTGCCTGGAGTGTTGTTCCTGG | 389 | UGCUGUGCCUGGAGUGUUGUUC | 775 | 46419320 |
| SluM40 | GCTGTGCCTGGAGTGTTGTTCCTGGG | 390 | GCUGUGCCUGGAGUGUUGUUCC | 776 | 46419321 |
| SluM41 | TGGACGAGTAGGTGGTCTTTAGGTGG | 591 | UGGACGAGUAGGUGGUCUUUAG | 777 | 46419340 |
| SluM42 | CCTAAAGACCACCTACTCGTCCAAGG | 592 | CCUAAAGACCACCUACUCGUCC | 778 | 46419354 |
| SluM43 | CCTTGGACGAGTAGGTGGTCTTTAGG | 393 | CCUUGGACGAGUAGGUGGUCUU | 779 | 46419343 |
| SluM44 | AGACCACCTACTCGTCCAAGGTGAGG | 594 | AGACCACCUACUCGUCCAAGGU | 780 | 46419359 |
| SluM45 | GACCACCTACTCGTCCAAGGTGAGGG | 393 | GACCACCUACUCGUCCAAGGUG | 781 | 46419360 |
| SluM46 | TGCCCTCACCTTGGACGAGTAGGTGG | 596 | UGCCCUCACCUUGGACGAGUAG | 782 | 46419351 |
| SluM47 | AATTGCCCTCACCTTGGACGAGTAGG | 397 | AAUUGCCCUCACCUUGGACGAG | 783 | 46419354 |
| SluM48 | CTTTCAACACAGCCTGACAGAGTTGG | 598 | CUUUCAACACAGCCUGACAGAG | 784 | 46419386 |
| SluM49 | ATTAATAGAACCCAACATCTGTTAGG | 599 | AUUAAUAGAACCCAACAUCUGU | 785 | 46419467 |
| SluM50 | AGAACCCAACATCTGTTAGGCTGTGG | 600 | AGAACCCAACAUCUGUUAGGCU | 786 | 46419473 |
| SluM51 | AACTCCACAGCCTAACAGATGTTGGG | 601 | AACUCCACAGCCUAACAGAUGU | 787 | 46419466 |
| SluM52 | AGTTGAATACCCATTATCTCATTTGG | 602 | AGUUGAAUACCCAUUAUCUCAU | 788 | 46419499 |
| SluM53 | TAGTTAGGGAGGGTGTAAATCTGAGG | 603 | UAGUUAGGGAGGGUGUAAAUCU | 789 | 46422155 |
| SluM54 | AGTTAGGGAGGGTGTAAATCTGAGGG | 604 | AGUUAGGGAGGGUGUAAAUCUG | 790 | 46422156 |
| SluM55 | GACTTAGCATGTTAAATGCTGCTAGG | 605 | GACUUAGCAUGUUAAAUGCUGC | 791 | 46422211 |
| SluM56 | AAAGAGCTAGCAGCAACGCACAGAGG | 606 | AAAGAGCUAGCAGCAACGCACA | 792 | 46422237 |
| SluM57 | AAGAGCTAGCAGCAACGCACAGAGGG | 607 | AAGAGCUAGCAGCAACGCACAG | 793 | 46422238 |
| SluM58 | GGGGGAAGTCAAGGTAGGAATGGAGG | 608 | GGGGGAAGUCAAGGUAGGAAUG | 794 | 46422262 |
| SluM59 | GGGGAAGTCAAGGTAGGAATGGAGGG | 609 | GGGGAAGUCAAGGUAGGAAUGG | 795 | 46422263 |
| SluM60 | CTCTTCAGTGAAGAAATGATGGCAGG | 610 | CUCUUCAGUGAAGAAAUGAUGG | 796 | 46422283 |
| SluM61 | GTATCTCTTCAGTGAAGAAATGATGG | 611 | GUAUCUCUUCAGUGAAGAAAUG | 797 | 46422287 |
| SluM62 | AAAATGAGGTATGTGGTAACGGAAGG | 612 | AAAAUGAGGUAUGUGGUAACGG | 798 | 46422623 |
| SluM63 | AAATGAGGTATGTGGTAACGGAAGGG | 613 | AAAUGAGGUAUGUGGUAACGGA | 799 | 46422624 |
| SluM64 | GTGGTAACGGAAGGGTGTAACCCAGG | 614 | GUGGUAACGGAAGGGUGUAACC | 800 | 46422635 |
| SluM65 | ACGAGGCCTAGAGTGCTGTGCCGTGG | 615 | ACGAGGCCUAGAGUGCUGUGCC | 801 | 46422674 |
| SluM66 | CGAGGCCTAGAGTGCTGTGCCGTGGG | 616 | CGAGGCCUAGAGUGCUGUGCCG | 802 | 46422675 |
| SluM67 | CTAGAGTGCTGTGCCGTGGGATGTGG | 617 | CUAGAGUGCUGUGCCGUGGGAU | 803 | 46422681 |
| SluM68 | CACATCCCACGGCACAGCACTCTAGG | 618 | CACAUCCCACGGCACAGCACUC | 804 | 46422669 |
| SluM69 | GTGCTGTGCCGTGGGATGTGGTGCGG | 619 | GUGCUGUGCCGUGGGAUGUGGU | 805 | 46422686 |
| SluM70 | TGCTGTGCCGTGGGATGTGGTGCGGG | 620 | UGCUGUGCCGUGGGAUGUGGUG | 806 | 46422687 |
| SluM71 | CTTGTCACCCGCACCACATCCCACGG | 621 | CUUGUCACCCGCACCACAUCCC | 807 | 46422683 |
| SluM72 | TGGGATGTGGTGCGGGTGACAAGTGG | 622 | UGGGAUGUGGUGCGGGUGACAA | 808 | 46422697 |
| SluM73 | TGTGGTGCGGGTGACAAGTGGCCTGG | 623 | UGUGGUGCGGGUGACAAGUGGC | 809 | 46422702 |
| SluM74 | GTGGTGCGGGTGACAAGTGGCCTGGG | 624 | GUGGUGCGGGUGACAAGUGGCC | 810 | 46422703 |
| SluM75 | ACTCAGTCTGGGAGCCCGGAGGTAGG | 625 | ACUCAGUCUGGGAGCCCGGAGG | 811 | 46424854 |
| SluM76 | CTCAGTCTGGGAGCCCGGAGGTAGGG | 626 | CUCAGUCUGGGAGCCCGGAGGU | 812 | 46424855 |
| SluM77 | CCTGAGGTGCTGTTCCCATGCTTTGG | 627 | CCUGAGGUGCUGUUCCCAUGCU | 813 | 46424894 |
| SluM78 | CCAAAGCATGGGAACAGCACCTCAGG | 628 | CCAAAGCAUGGGAACAGCACCU | 814 | 46424883 |
| SluM79 | GACACTCAGGAACTCCAAAGCATGGG | 629 | GACACUCAGGAACUCCAAAGCA | 813 | 46424897 |
| SluM80 | GGACACTCAGGAACTCCAAAGCATGG | 630 | GGACACUCAGGAACUCCAAAGC | 816 | 46424898 |
| SluM81 | CTCAGACAGAGAAGCTGCCCATTGGG | 631 | CUCAGACAGAGAAGCUGCCCAU | 817 | 46424954 |
| SluM82 | ACTCAGACAGAGAAGCTGCCCATTGG | 632 | ACUCAGACAGAGAAGCUGCCCA | 818 | 46424955 |
| SluM83 | GAGTATGTGTATTAATATTAATTAGG | 633 | GAGUAUGUGUAUUAAUAUUAAU | 819 | 46425005 |
| SluM84 | ACTTCCTCCTCATCAACCAGGGAGGG | 634 | ACUUCCUCCUCAUCAACCAGGG | 820 | 46425064 |
| SluM85 | GACTTCCTCCTCATCAACCAGGGAGG | 635 | GACUUCCUCCUCAUCAACCAGG | 821 | 46425065 |
| SluM86 | CCCTGGTTGATGAGGAGGAAGTCTGG | 636 | CCCUGGUUGAUGAGGAGGAAGU | 822 | 46425079 |
| SluM87 | CCTGGTTGATGAGGAGGAAGTCTGGG | 637 | CCUGGUUGAUGAGGAGGAAGUC | 823 | 46425080 |
| SluM88 | CCAGACTTCCTCCTCATCAACCAGGG | 638 | CCAGACUUCCUCCUCAUCAACC | 824 | 46425068 |
| SluM89 | CCCAGACTTCCTCCTCATCAACCAGG | 639 | CCCAGACUUCCUCCUCAUCAAC | 825 | 46425069 |
| Slum90 | ATGAGGAGGAAGTCTGGGCTAATGGG | 640 | AUGAGGAGGAAGUCUGGGCUAA | 826 | 46425088 |
| SluM91 | AAGTCTGGGCTAATGGGTTGCAGTGG | 641 | AAGUCUGGGCUAAUGGGUUGCA | 827 | 46425097 |
| SluM92 | TATACCCACCACCGCACACAGATGGG | 642 | UAUACCCACCACCGCACACAGA | 828 | 46425153 |
| SluM93 | CTATACCCACCACCGCACACAGATGG | 643 | CUAUACCCACCACCGCACACAG | 829 | 46425154 |
| SluM94 | GGTGGGTATAGCTTCCTTTACTGCGG | 644 | GGUGGGUAUAGCUUCCUUUACU | 830 | 46425180 |
| SluM95 | GCCTGCTTTCTTGTGCCTCCTGCTGG | 645 | GCCUGCUUUCUUGUGCCUCCUG | 831 | 46425212 |
| SluM96 | CCTGCTGGCATTTCATTGTGTTGTGG | 646 | CCUGCUGGCAUUUCAUUGUGUU | 832 | 46425230 |
| SluM97 | CCACAACACAATGAAATGCCAGCAGG | 647 | CCACAACACAAUGAAAUGCCAG | 833 | 46425219 |
| SluM98 | CTGGCATTTCATTGTGTTGTGGTTGG | 648 | CUGGCAUUUCAUUGUGUUGUGG | 834 | 46425234 |
| SluM99 | TGTGGTTGGTTGTGTGTCTGGTCTGG | 649 | UGUGGUUGGUUGUGUGUCUGGU | 835 | 46425251 |
| SluM100 | GTTGTGTGTCTGGTCTGGCTGTGTGG | 650 | GUUGUGUGUCUGGUCUGGCUGU | 836 | 46425259 |
| SluM101 | GTCTGGCTGTGTGGTTATGTGCCTGG | 651 | GUCUGGCUGUGUGGUUAUGUGC | 837 | 46425271 |
| SluM102 | GGCTGTGTGGTTATGTGCCTGGCTGG | 652 | GGCUGUGUGGUUAUGUGCCUGG | 838 | 46425275 |
| SluM103 | GCTGTGTGGTTATGTGCCTGGCTGGG | 653 | GCUGUGUGGUUAUGUGCCUGGC | 839 | 46425276 |
| SluM104 | TGCCTGGCTGGGTGTGCATGTGTTGG | 654 | UGCCUGGCUGGGUGUGCAUGUG | 840 | 46425290 |
| SluM105 | ACCCAACACATGCACACCCAGCCAGG | 655 | ACCCAACACAUGCACACCCAGC | 841 | 46423281 |
| SluM106 | TGGGTGTGCATGTGTTGGGTTATTGG | 656 | UGGGUGUGCAUGUGUUGGGUUA | 842 | 46425298 |
| SluM107 | TGTGCATGTGTTGGGTTATTGGTTGG | 657 | UGUGCAUGUGUUGGGUUAUUGG | 843 | 46425302 |
| SluM108 | GAGTGTGTACATCTAGCTATGTGTGG | 638 | GAGUGUGUACAUCUAGCUAUGU | 844 | 46425327 |
| SluM109 | GTGTACATCTAGCTATGTGTGGCTGG | 659 | GUGUACAUCUAGCUAuGUGUGG | 845 | 46425331 |
| SluMl10 | CATCTAGCTATGTGTGGCTGGTGTGG | 660 | CAUCUAGCUAUGUGUGGCUGGU | 846 | 46425336 |
| SluM111 | ATCTAGCTATGTGTGGCTGGTGTGGG | 661 | AUCUAGCUAUGUGUGGCUGGUG | 847 | 46425337 |
| SluM112 | TGGCTGGTGTGGGTCTGAATGTCTGG | 662 | UGGCUGGUGUGGGUCUGAAUGU | 848 | 46425350 |
| SluM113 | GTCTGGTAGAGAGTGTTTGTGTGTGG | 663 | GUCUGGUAGAGAGUGUUUGUGU | 849 | 46425370 |
| SluM114 | GTGTGGTTGTGTGTCTGATGTGTGGG | 664 | GUGUGGUUGUGUGUCUGAUGUG | 850 | 46425390 |
| SluM115 | GGGCAGCTGGTTTGGTATGTGTCTGG | 665 | GGGCAGCUGGUUUGGUAUGUGU | 831 | 46423415 |
| SluM116 | GGCAGCTGGTTTGGTATGTGTCTGGG | 666 | GGCAGCUGGUUUGGUAUGUGUC | 852 | 46423416 |
| SluM117 | GGTTTGGTATGTGTCTGGGCATCTGG | 667 | GGUUUGGUAUGUGUCUGGGCAU | 853 | 46425423 |
| SluMl18 | TGGTATGTGTCTGGGCATCTGGTTGG | 668 | UGGUAUGUGUCUGGGCAUCUGG | 854 | 46425427 |
| SluM119 | TGGGCATCTGGTTGGTGAACATGTGG | 669 | UGGGCAUCUGGUUGGUGAACAU | 833 | 46425438 |
| SluM120 | TGGTTGGTGAACATGTGGATGTCTGG | 670 | UGGUUGGUGAACAUGUGGAUGU | 856 | 46425446 |
| SluM121 | GGTTGGTGAACATGTGGATGTCTGGG | 671 | GGUUGGUGAACAUGUGGAUGUC | 857 | 46425447 |
| SluM122 | GAACATGTGGATGTCTGGGCTGTTGG | 672 | GAACAUGUGGAUGUCUGGGCUG | 858 | 46425454 |
| SluM123 | AACATGTGGATGTCTGGGCTGTTGGG | 673 | AACAUGUGGAUGUCUGGGCUGU | 859 | 46425455 |
| SluM124 | TGTGGATGTCTGGGCTGTTGGGCTGG | 674 | UGUGGAUGUCUGGGCUGUUGGG | 860 | 46425459 |
| SluM125 | GTGGATGTCTGGGCTGTTGGGCTGGG | 675 | GUGGAUGUCUGGGCUGUUGGGC | 861 | 46425460 |
| SluM126 | GGGGTATATGTCTGGATGGCTGGAGG | 676 | GGGGUAUAUGUCUGGAUGGCUG | 862 | 46425495 |
| SluM127 | CTGGATGGCTGGAGGAGTGGAAGAGG | 677 | CUGGAUGGCUGGAGGAGUGGAA | 863 | 46425506 |
| SluM128 | ATGGTGTCTCCAGCCTCCCATTGTGG | 678 | AUGGUGUCUCCAGCCUCCCAUU | 864 | 46425551 |
| SluM129 | CTCCAGCCTCCCATTGTGGTTTCAGG | 679 | CUCCAGCCUCCCAUUGUGGUUU | 865 | 46425558 |
| SluM130 | AGCCTGAAACCACAATGGGAGGCTGG | 680 | AGCCUGAAACCACAAUGGGAGG | 866 | 46425549 |
| SluM131 | AAGAAGCCTGAAACCACAATGGGAGG | 681 | AAGAAGCCUGAAACCACAAUGG | 867 | 46425553 |
| SluM132 | TCCCATTGTGGTTTCAGGCTTCTTGG | 682 | UCCCAUUGUGGUUUCAGGCUUC | 868 | 46425566 |
| SluM133 | GCCAAGAAGCCTGAAACCACAATGGG | 683 | GCCAAGAAGCCUGAAACCACAA | 869 | 46425556 |
| SluM134 | AGCCAAGAAGCCTGAAACCACAATGG | 684 | AGCCAAGAAGCCUGAAACCACA | 870 | 46425557 |
| SluM135 | CAACAAGCTATACAGGTCTGTCCTGG | 683 | CAACAAGCUAUACAGGUCUGUC | 871 | 46425615 |
| SluM136 | AGGACAGACCTGTATAGCTTGTTGGG | 686 | AGGACAGACCUGUAUAGCUUGU | 872 | 46425602 |
| SluM137 | CAGGACAGACCTGTATAGCTTGTTGG | 687 | CAGGACAGACCUGUAUAGCUUG | 873 | 46425603 |
| SluM138 | GTGACATGACTGAGAAGGTGCCCAGG | 688 | GUGACAUGACUGAGAAGGUGCC | 874 | 46425625 |
| SluM139 | CGAGGTTGTCACTGGCAGAGAGAGGG | 689 | CGAGGUUGUCACUGGCAGAGAG | 875 | 46425650 |
| SluM140 | TCGAGGTTGTCACTGGCAGAGAGAGG | 690 | UCGAGGUUGUCACUGGCAGAGA | 876 | 46425651 |
| SluM141 | GCCAGTGACAACCTCGAGACCTCAGG | 691 | GCCAGUGACAACCUCGAGACCU | 877 | 46423671 |
| SluM142 | ACCTGAGGTCTCGAGGTTGTCACTGG | 692 | ACCUGAGGUCUCGAGGUUGUCA | 878 | 46425661 |
| SluM143 | AAAGGTGGACACCTGAGGTCTCGAGG | 693 | AAAGGUGGACACCUGAGGUCUC | 879 | 46423671 |
| SluM144 | CTCAGGTGTCCACCTTTATGTCCCGG | 694 | CUCAGGUGUCCACCUUUAUGUC | 880 | 46425691 |
| SluM145 | TCAGGTGTCCACCTTTATGTCCCGGG | 695 | UCAGGUGUCCACCUUUAUGUCC | 881 | 46425692 |
| SluM146 | CGGGACATAAAGGTGGACACCTGAGG | 696 | CGGGACAUAAAGGUGGACACCU | 882 | 46425679 |
| SluM147 | GAATTTGTTTGCTGAGCCTGTGAGGG | 697 | GAAUUUGUUUGCUGAGCCUGUG | 883 | 46425735 |
| SluM148 | TGAATTTGTTTGCTGAGCCTGTGAGG | 698 | UGAAUUUGUUUGCUGAGCCUGU | 884 | 46425736 |
| SluM149 | AAAATTTCCCAGGGAATTTCTCCAGG | 699 | AAAAUUUCCCAGGGAAUUUCUC | 885 | 46425790 |
| SluM150 | GGCAAGACCTGGAGAAATTCCCTGGG | 700 | GGCAAGACCUGGAGAAAUUCCC | 886 | 46425786 |
| SluM151 | GGGCAAGACCTGGAGAAATTCCCTGG | 701 | GGGCAAGACCUGGAGAAAUUCC | 887 | 46425787 |
| Slum152 | AGAGCTCAGCACAGGGCAAGACCTGG | 702 | AGAGCUCAGCACAGGGCAAGAC | 888 | 46425800 |
| SluM153 | AGGTCTTGCCCTGTGCTGAGCTCTGG | 703 | AGGUCUUGCCCUGUGCUGAGCU | 889 | 46425813 |
| SluM154 | GGTCTTGCCCTGTGCTGAGCTCTGGG | 704 | GGUCUUGCCCUGUGCUGAGCUC | 890 | 46423814 |
| SluM155 | AGATGCCCACAGAACTGGTGACTTGG | 703 | AGAUGCCCACAGAACUGGUGAC | 891 | 46425977 |
| SluM156 | AAGTCACCAGTTCTGTGGGCATCTGG | 706 | AAGUCACCAGUUCUGUGGGCAU | 892 | 46425964 |
| SluM157 | GGCAGGAGGGTGGCTGGTCTGTCTGG | 707 | GGCAGGAGGGUGGCUGGUCUGU | 893 | 46426001 |
| SluM158 | GAGGGTGGCTGGTCTGTCTGGAGAGG | 708 | GAGGGUGGCUGGUCUGUCUGGA | 894 | 46426006 |
| SluM159 | GGTCTGTCTGGAGAGGATCATGTTGG | 709 | GGUCUGUCUGGAGAGGAUCAUG | 893 | 46426016 |
| SluM160 | TTCAGCTCACTCACCACCCGTCTGGG | 710 | UUCAGCUCACUCACCACCCGUC | 896 | 46426039 |
| SluM161 | GTTCAGCTCACTCACCACCCGTCTGG | 711 | GUUCAGCUCACUCACCACCCGU | 897 | 46426040 |
| SluM162 | GGTGGTGAGTGAGCTGAACCTCATGG | 712 | GGUGGUGAGUGAGCUGAACCUC | 898 | 46426058 |
| SluMl63 | GAGCTGAACCTCATGGCACCTGTAGG | 713 | GAGCUGAACCUCAUGGCACCUG | 899 | 46426068 |
| SluMl64 | GGAGAAACGAGAAAGGCAGTACCAGG | 714 | GGAGAAACGAGAAAGGCAGUAC | 900 | 46426106 |
| SluMl65 | GAGAAACGAGAAAGGCAGTACCAGGG | 715 | GAGAAACGAGAAAGGCAGUACC | 901 | 46426107 |
| SluMl66 | AAACGAGAAAGGCAGTACCAGGGAGG | 716 | AAACGAGAAAGGCAGUACCAGG | 902 | 46426110 |
| SluMl67 | AACGAGAAAGGCAGTACCAGGGAGGG | 717 | AACGAGAAAGGCAGUACCAGGG | 903 | 46426111 |
| SluM168 | TCTACAGAAACACTGCCTCATCTGGG | 718 | UCUACAGAAACACUGCCUCAUC | 904 | 46426132 |
| SluMl69 | TTCTACAGAAACACTGCCTCATCTGG | 719 | UUCUACAGAAACACUGCCUCAU | 905 | 46426133 |
| SluMl70 | AAAATAATATTCCTAGGACCCATTGG | 720 | AAAAUAAUAUUCCUAGGACCCA | 906 | 46426177 |
| SluMl71 | AAATAATATTCCTAGGACCCATTGGG | 721 | AAAUAAUAUUCCUAGGACCCAU | 907 | 46426178 |
| SluMl72 | ATTCCTAGGACCCATTGGGTAAATGG | 722 | AUUCCUAGGACCCAUUGGGUAA | 908 | 46426185 |
| SluM173 | GGTCCATTTACCCAATGGGTCCTAGG | 723 | GGUCCAUUUACCCAAUGGGUCC | 909 | 46426177 |
| SluMl74 | AGCAGCTGGTCCATTTACCCAATGGG | 724 | AGCAGCUGGUCCAUUUACCCAA | 910 | 46426184 |
| SluMl75 | GAGCAGCTGGTCCATTTACCCAATGG | 725 | GAGCAGCUGGUCCAUUUACCCA | 911 | 46426185 |
| SluMl76 | TGGGTAAATGGACCAGCTGCTCATGG | 726 | UGGGUAAAUGGACCAGCUGCUC | 912 | 46426200 |
| SluMl77 | TAAATGGACCAGCTGCTCATGGCTGG | 727 | UAAAUGGACCAGCUGCUCAUGG | 913 | 46426204 |
| SluMl78 | CACTAATTAGAAGTTGTCTAGCATGG | 728 | CACUAAUUAGAAGUUGUCUAGC | 914 | 46427783 |
| SluM179 | GCTAAGAGTGTGGCTTCTGTTGTTGG | 729 | GCUAAGAGUGUGGCUUCUGUUG | 915 | 46427811 |
| SluM180 | CTAAGAGTGTGGCTTCTGTTGTTGGG | 730 | CUAAGAGUGUGGCUUCUGUUGU | 916 | 46427812 |
| SluM181 | GAGTGTGGCTTCTGTTGTTGGGCTGG | 731 | GAGUGUGGCUUCUGUUGUUGGG | 917 | 46427816 |
| SluM182 | GTGTAAGTGTTTGCTGGGTTTGGTGG | 732 | GUGUAAGUGUUUGCUGGGUUUG | 918 | 46427959 |
| SluMl83 | TAAGTGTAAGTGTTTGCTGGGTTTGG | 733 | UAAGUGUAAGUGUUUGCUGGGU | 919 | 46427962 |
| SluMl84 | TCATTTAAGTGTAAGTGTTTGCTGGG | 734 | UCAUUUAAGUGUAAGUGUUUGC | 920 | 46427967 |
| SluMl85 | GTCATTTAAGTGTAAGTGTTTGCTGG | 733 | GUCAUUUAAGUGUAAGUGUUUG | 921 | 46427968 |
| SluMl86 | TAATAAATGTTTACAGTGGTGCCTGG | 736 | UAAUAAAUGUUUACAGUGGUGC | 922 | 46428073 |

* chromosomal location of guide cut-site in chromosome 1 of human genome Hg38

TABLE 20
Target and Spacer Sequences for SaCas9 gRNAs in FAAH-OUT

| | | SEQ | | SEQ | |
|---|---|---|---|---|---|
| | Target Sequence | ID | | ID | Cut site |
| Name | PAM in bold underline | NO | Spacer Sequence | NO | location* |
| saM1 | TCATCACTTGTTCTTGGCTTAGAGGAT | 923 | UCAUCACUUGUUCUUGGCUUA | 1095 | 46418107 |
| saM2 | AGGATGGTGCTCCACAAATTCTGGGAT | 924 | AGGAUGGUGCUCCACAAAUUC | 1096 | 46418129 |
| saM3 | CACAGCCACACTTTATCATCCCAGAAT | 925 | CACAGCCACACUUUAUCAUCC | 1097 | 46418138 |
| saM4 | GTGAGGGGCCAAGGCACTGTGCTGGGT | 926 | GUGAGGGGCCAAGGCACUGUG | 1098 | 46418172 |
| saM5 | CTCGTGGAGCTCACATTCTGGAGGGAT | 927 | CUCGUGGAGCUCACAUUCUGG | 1099 | 46418236 |
| saM6 | CTCACATTCTGGAGGGATTTGTTGAAT | 928 | CUCACAUUCUGGAGGGAUUUG | 1100 | 46418245 |
| saM7 | GACCTCTAAATATTTAATGTCTGGAGT | 929 | GACCUCUAAAUAUUUAAUGUC | 1101 | 46418306 |
| saM8 | GGGAATTCTAGACCACATTTACTGAGT | 930 | GGGAAUUCUAGACCACAUUUA | 1102 | 46418368 |
| saM9 | AGGTACTCAGTAAATGTGGTCTAGAAT | 931 | AGGUACUCAGUAAAUGUGGUC | 1103 | 46418364 |
| saM10 | TTCTGTTGATGCCAAGCCCCAGTGAGT | 932 | UUCUGUUGAUGCCAAGCCCCA | 1104 | 46418584 |
| saM11 | CCCAGTGAGTACGATGGCCAGAAGAGT | 933 | cCCAGUGAGUACGAUGGCCAG | 1105 | 46418601 |
| saM12 | TCATGGCCTTTCCCCTTCTCACCGGGT | 934 | UCAUGGCCUUUCCCCUUCUCA | 1106 | 46418696 |
| saM13 | AGGCTTCTGGACTTGGCACAAGTGAGT | 933 | AGGCUUCUGGACUUGGCACAA | 1107 | 46419123 |
| saM14 | CTGCAGGTAATGTTCAGAACACCGAGT | 936 | CUGCAGGUAAUGUUCAGAACA | 1108 | 46419257 |
| saM15 | CTGTGCCTGGAGTGTTGTTCCTGGGGT | 937 | CUGUGCCUGGAGUGUUGUUCC | 1109 | 46419321 |
| saM16 | AGCTCTTTCAACACAGCCTGACAGAGT | 938 | AGCUCUUUCAACACAGCCUGA | 1110 | 46419391 |
| saM17 | AACCCAACATCTGTTAGGCTGTGGAGT | 939 | AACCCAACAUCUGUUAGGCUG | 1111 | 46419474 |
| saM18 | AACATCTGTTAGGCTGTGGAGTTGAAT | 940 | AACAUCUGUUAGGCUGUGGAG | 1112 | 46419479 |
| saM19 | GCCCACTTTCCAAATGAGATAATGGGT | 941 | GCCCACUUUCCAAAUGAGAUA | 1113 | 46419498 |
| saM20 | CCTGGAGTCCCAGCTATACTCGGGAGT | 942 | CCUGGAGUCCCAGCUAUACUC | 1114 | 46419968 |
| saM21 | ACTTGTTGAAGGCTGATCATTATGGGT | 943 | ACUUGUUGAAGGCUGAUCAUU | 1115 | 46420211 |
| saM22 | AGTAAGTTTAGAGTTGAGCGTGTGGGT | 944 | AGUAAGUUUAGAGUUGAGCGU | 1116 | 46420247 |
| saM23 | AGCAGAAACAGCCAGAGCTCTTGGGAT | 943 | AGCAGAAACAGCCAGAGCUCU | 1117 | 46420537 |
| saM24 | AGAGGTCCAGTGCTCAGATTTGTGGAT | 946 | AGAGGUCCAGUGCUCAGAUUU | 1118 | 46420584 |
| saM25 | ATCCAAGTCACCCATAAACCTATGGAT | 947 | AUCCAAGUCACCCAUAAACCU | 1119 | 46420627 |
| saM26 | ATCCATAGGTTTATGGGTGACTTGGAT | 948 | AUCCAUAGGUUUAUGGGUGAC | 1120 | 46420619 |
| saM27 | CCTTAACAGACAGACACATAACCGAGT | 949 | CCUUAACAGACAGACACAUAA | 1121 | 46420687 |
| saM28 | TCGGTTATGTGTCTGTCTGTTAAGGGT | 950 | UCGGUUAUGUGUCUGUCUGUU | 1122 | 46420677 |
| saM29 | CAAGTTTCCTACACATAGATTTGGGGT | 951 | CAAGUUUCCUACACAUAGAUU | 1123 | 46420780 |
| saM30 | CCTCAGGAAGGTGAAGCCACTTGGGAT | 952 | CCUCAGGAAGGUGAAGCCACU | 1124 | 46420821 |
| saM31 | CAGCATGGTGCCTGGTACTCAGTGGGT | 933 | CAGCAUGGUGCCUGGUACUCA | 1125 | 46420870 |
| saM32 | CACTGAGTACCAGGCACCATGCTGGGT | 954 | CACUGAGUACCAGGCACCAUG | 1126 | 46420859 |
| saM33 | TGGGTACCCAGAGAAGGCTTGTTGAAT | 933 | UGGGUACCCAGAGAAGGCUUG | 1127 | 46420892 |
| saM34 | CAAGCCTTCTCTGGGTACCCACTGAGT | 956 | CAAGCCUUCUCUGGGUACCCA | 1128 | 46420878 |
| saM35 | GCTCCATTCAACAAGCCTTCTCTGGGT | 957 | GCUCCAUUCAACAAGCCUUCU | 1129 | 46420889 |
| saM36 | GAAGGCTTGTTGAATGGAGCAATGGGT | 958 | GAAGGCUUGUUGAAUGGAGCA | 1130 | 46420904 |
| saM37 | AAGTAATCAGAATGGACCAAAATGGGT | 959 | AAGUAAUCAGAAUGGACCAAA | 1131 | 46420939 |
| saM38 | ACAATAAATGTGAAAGGAGCAAAGGGT | 960 | ACAAUAAAUGUGAAAGGAGCA | 1132 | 46421008 |
| saM39 | GCTATAGGTTGAATGTAGACTGGGAAT | 961 | GCUAUAGGUUGAAUGUAGACU | 1133 | 46421359 |
| saM40 | AGACCAGGAGAAAGCTATAGGTTGAAT | 962 | AGACCAGGAGAAAGCUAUAGG | 1134 | 46421372 |
| saM41 | AAAGCAGGGACTGTGTCTTAATAGAAT | 963 | AAAGCAGGGACUGUGUCUUAA | 1135 | 46421301 |
| saM42 | AATCCAACATCCGTTAGGCTGTGGAGT | 964 | AAUCCAACAUCCGUUAGGCUG | 1136 | 46421325 |
| saM43 | TGGGTTCAACTCCACAGCCTAACGGAT | 965 | UGGGUUCAACUCCACAGCCUA | 1137 | 46421325 |
| saM44 | GCCCATTTTACAAAGGAGATAATGGGT | 966 | GCCCAUUUUACAAAGGAGAUA | 1138 | 46421347 |
| saM45 | TTGTTCAGAGAGCAACCCTCTCTGAAT | 967 | UUGUUCAGAGAGCAACCCUCU | 1139 | 46421608 |
| saM46 | GTTGCTCTCTGAACAACAATGGAGGGT | 968 | GUUGCUCUCUGAACAACAAUG | 1140 | 46421627 |
| saM47 | TTTTATTGCATGGATGGGAGGATGAAT | 969 | UUUUAUUGCAUGGAUGGGAGG | 1141 | 46421656 |
| saM48 | CCAATCAGAAATAGTCCCTGCCTGAAT | 970 | CCAAUCAGAAAUAGUCCCUGC | 1142 | 46421723 |
| saM49 | CCAGGAAACAGACATCAAATGTAGAAT | 971 | CCAGGAAACAGACAUCAAAUG | 1143 | 46421767 |
| saM50 | CATTTGATGTCTGTTTCCTGGTAGAAT | 972 | CAUUUGAUGUCUGUUUCCUGG | 1144 | 46421753 |
| saM51 | AGCAGAAGCCCTGGGTATTATGAGAAT | 973 | AGCAGAAGCCCUGGGUAUUAU | 1145 | 46421825 |
| saM52 | CACACATACACACATCTCAGGCTGGAT | 974 | CACACAUACACACAUCUCAGG | 1146 | 46421861 |
| saM53 | ACTGCATTGTCACCTGAGCCAAGGAAT | 973 | ACUGCAUUGUCACCUGAGCCA | 1147 | 46421939 |
| saM54 | CATTGTCACCTGAGCCAAGGAATGAGT | 976 | CAUUGUCACCUGAGCCAAGGA | 1148 | 46421943 |
| saM55 | AAGGAATGAGTGTGTTTCAAGGAGGGT | 977 | AAGGAAUGAGUGUGUUUCAAG | 1149 | 46421959 |
| saM56 | CAAGAGTAAATCAGCAGCATTTAGAGT | 978 | CAAGAGUAAAUCAGCAGCAUU | 1150 | 46421988 |
| saM57 | ATTTACTCTTGAGGAGATCACTGGGAT | 979 | AUUUACUCUUGAGGAGAUCAC | 1131 | 46422012 |
| saM58 | TATCATCCCAGTGATCTCCTCAAGAGT | 980 | UAUCAUCCCAGUGAUCUCCUC | 1132 | 46422008 |
| saM59 | ATAGGCACAAGCCAGACTTTGTTGGGT | 981 | AUAGGCACAAGCCAGACUUUG | 1153 | 46422042 |
| saM60 | TTAGGGAGGGTGTAAATCTGAGGGAAT | 982 | UUAGGGAGGGUGUAAAUCUGA | 1134 | 46422137 |
| saM61 | TTCTAGGGAGGGAGGCAGGAGGTGAGT | 983 | UUCUAGGGAGGGAGGCAGGAG | 1155 | 46422546 |
| saM62 | AAATGAGGTATGTGGTAACGGAAGGGT | 984 | AAAUGAGGUAUGUGGUAACGG | 1156 | 46422623 |
| saM63 | GCCTCGTGACCTCACCTTTTCCTGGGT | 983 | GCCUCGUGACCUCACCUUUUC | 1157 | 46422645 |
| saM64 | AAAAGGTGAGGTCACGAGGCCTAGAGT | 986 | AAAAGGUGAGGUCACGAGGCC | 1158 | 46422660 |
| saM65 | GAGGCCTAGAGTGCTGTGCCGTGGGAT | 987 | GAGGCCUAGAGUGCUGUGCCG | 1159 | 46422675 |
| saM66 | TGCTGTGCCGTGGGATGTGGTGCGGGT | 988 | UGCUGUGCCGUGGGAUGUGGU | 1160 | 46422686 |
| saM67 | GAGAGCTGGAGTTCATGAAGGTGGAGT | 989 | GAGAGCUGGAGUUCAUGAAGG | 1161 | 46422746 |
| saM68 | CTGGAGGCCCCAGGGAGATGACAGAGT | 990 | CUGGAGGCCCCAGGGAGAUGA | 1162 | 46422829 |
| saM69 | ATGCGAGGTCAGCAGTTGACTAAGGGT | 991 | AUGCGAGGUCAGCAGUUGACU | 1163 | 46422874 |
| saM70 | TAATGTTGAGTCTGAGTACCCGAGGGT | 992 | UAAUGUUGAGUCUGAGUACCC | 1164 | 46422913 |
| saM71 | TCAGACTCAACATTACCAACCATGAAT | 993 | UCAGACUCAACAUUACCAACC | 1165 | 46422933 |
| saM72 | TCATGGTTGGTAATGTTGAGTCTGAGT | 994 | UCAUGGUUGGUAAUGUUGAGU | 1166 | 46422923 |
| saM73 | CTGAATTCATGGTTGGTAATGTTGAGT | 993 | CUGAAUUCAUGGUUGGUAAUG | 1167 | 46422929 |
| saM74 | GCAGGTTAGGGTGGGAAGGAACTGAAT | 996 | GCAGGUUAGGGUGGGAAGGAA | 1168 | 46422950 |
| saM75 | GAAGCCAGGGCAGCAGCAGGTTAGGGT | 997 | GAAGCCAGGGCAGCAGCAGGU | 1169 | 46422965 |
| saM76 | ACTGCAAGTCACTGTACCCAGGAGAAT | 998 | ACUGCAAGUCACUGUACCCAG | 1170 | 46423015 |
| saM77 | GATGGAGGCCCCTGAGGAGAGCAGAAT | 999 | GAUGGAGGCCCCUGAGGAGAG | 1171 | 46423079 |
| saM78 | CAACTCCCCTCCACCAGCAGGGAGAGT | 1000 | CAACUCCCCUCCACCAGCAGG | 1172 | 46423197 |
| saM79 | TGACTCTCCCTGCTGGTGGAGGGGAGT | 1001 | UGACUCUCCCUGCUGGUGGAG | 1173 | 46423191 |
| saM80 | TAGACTGGGGAGGCTGGAACCCCGGAT | 1002 | UAGACUGGGGAGGCUGGAACC | 1174 | 46423227 |
| saM81 | AGCAAGGCCATGACTCTGATCCGGGGT | 1003 | AGCAAGGCCAUGACUCUGAUC | 1175 | 46423237 |
| saM82 | ATCAGAGTCATGGCCTTGCTTTGGAGT | 1004 | AUCAGAGUCAUGGCCUUGCUU | 1176 | 46423252 |
| saM83 | ATGTCCTGGCATCATCTCCCCTGGGGT | 1005 | AUGUCCUGGCAUCAUCUCCCC | 1177 | 46423312 |
| saM84 | CCTGGCATCATCTCCCCTGGGGTGGAT | 1006 | CCUGGCAUCAUCUCCCCUGGG | 1178 | 46423316 |
| saM85 | ACTGAACAGGCCATGTTTGCCTAGAGT | 1007 | ACUGAACAGGCCAUGUUUGCC | 1179 | 46423404 |
| saM86 | TGCGGAACAAAGGAGCTTTGGGAGAAT | 1008 | UGCGGAACAAAGGAGCUUUGG | 1180 | 46423461 |
| saM87 | TCCTGGGGTTCCCATTGCCTTCAGGAT | 1009 | UCCUGGGGUUCCCAUUGCCUU | 1181 | 46423478 |
| saM88 | AGTTATGGATGGGGTTGCTCCTGGGGT | 1010 | AGUUAUGGAUGGGGUUGCUCC | 1182 | 46423496 |
| saM89 | GCAACCCCATCCATAACTCCTGAGGGT | 1011 | GCAACCCCAUCCAUAACUCCU | 1183 | 46423513 |
| saM90 | TGGAGTAGGGAATCTGAGGGGTGGGAT | 1012 | UGGAGUAGGGAAUCUGAGGGG | 1184 | 46423540 |
| saM91 | GGGTGGGATCTGAGAGGTAGGATGGGT | 1013 | GGGUGGGAUCUGAGAGGUAGG | 1185 | 46423558 |
| saM92 | GATCTGAGAGGTAGGATGGGTGGGAGT | 1014 | GAUCUGAGAGGUAGGAUGGGU | 1186 | 46423564 |
| saM93 | GAGAGGTAGGATGGGTGGGAGTAGGAT | 1015 | GAGAGGUAGGAUGGGUGGGAG | 1187 | 46423569 |
| saM94 | CCTCTGCATGGCCCGGGAGATAGGGGT | 1016 | CCUCUGCAUGGCCCGGGAGAU | 1188 | 46423668 |
| saM95 | TTTGTCGCCAGGAAGTCCAGATGGGGT | 1017 | UUUGUCGCCAGGAAGUCCAGA | 1189 | 46423695 |
| saM96 | CACCCAGGGCCCGAGATGTGCGTGGGT | 1018 | CACCCAGGGCCCGAGAUGUGC | 1190 | 46423751 |
| saM97 | CACCCACGCACATCTCGGGCCCTGGGT | 1019 | CACCCACGCACAUCUCGGGCC | 1191 | 46423744 |
| saM98 | ATACTCCTGAGGAAACAGCAGCTGGAT | 1020 | AUACUCCUGAGGAAACAGCAG | 1192 | 46423800 |
| saM99 | GAATCCAGCTGCTGTTTCCTCAGGAGT | 1021 | GAAUCCAGCUGCUGUUUCCUC | 1193 | 46423794 |
| saM100 | CTTAGGTATTGCACGACCTGTGTGAAT | 1022 | CUUAGGUAUUGCACGACCUGU | 1194 | 46423817 |
| saM101 | CCAGCAGAAGTAGCATCATCAGGGAGT | 1023 | CCAGCAGAAGUAGCAUCAUCA | 1195 | 46423860 |
| saM102 | CTCCCTCCACTTCTGGGCCCTGGGGAT | 1024 | CUCCCUCCACUUCUGGGCCCU | 1196 | 46423955 |
| saM103 | GCCCAGTGACTCCGGCAGCAGGTGAGT | 1025 | GCCCAGUGACUCCGGCAGCAG | 1197 | 46424017 |
| saM104 | TCCGGCAGCAGGTGAGTCGACACGGGT | 1026 | UCCGGCAGCAGGUGAGUCGAC | 1198 | 46424027 |
| saM105 | CCGTGTCGACTCACCTGCTGCCGGAGT | 1027 | CCGUGUCGACUCACCUGCUGC | 1199 | 46424017 |
| saM106 | CCCAACAGGAGGACTACACCTCAGGAT | 1028 | CCCAACAGGAGGACUACACCU | 1200 | 46424093 |
| saM107 | CCTGAGGTGTAGTCCTCCTGTTGGGAT | 1029 | CCUGAGGUGUAGUCCUCCUGU | 1201 | 46424083 |
| saM108 | ATCTCCAGGGTCTCAAAGGCCGGGGAT | 1030 | AUCUCCAGGGUCUCAAAGGCC | 1202 | 46424224 |
| saM109 | CAAGTGCTGGGAGAACAGAGAAAGAAT | 1031 | CAAGUGCUGGGAGAACAGAGA | 1203 | 46424263 |
| saM110 | GTAACATGGGAGGTGCCCACTTAGGGT | 1032 | GUAACAUGGGAGGUGCCCACU | 1204 | 46424310 |
| saM111 | GGTAGGAGGGGACAGGTAGACTAGGGT | 1033 | GGUAGGAGGGGACAGGUAGAC | 1205 | 46424424 |
| saM112 | GCAAGTGTTTTCAGAGCTGAATGGGGT | 1034 | GCAAGUGUUUUCAGAGCUGAA | 1206 | 46424454 |
| saM113 | GCTCAGCAAGTGTTTTCAGAGCTGAAT | 1035 | GCUCAGCAAGUGUUUUCAGAG | 1207 | 46424459 |
| saM114 | GAGACAAACATAGACTGAGCCTGGGAT | 1036 | GAGACAAACAUAGACUGAGCC | 1208 | 46424709 |
| saM115 | TGGGATTTGCTGTGTGGCCTGGAGGAT | 1037 | UGGGAUUUGCUGUGUGGCCUG | 1209 | 46424730 |
| saM116 | TGGGGAGTCCAAGGCCCAGAGACGGGT | 1038 | UGGGGAGUCCAAGGCCCAGAG | 1210 | 46424755 |
| saM117 | GGGGATGGGCTCATGGTCTCTCGGGGT | 1039 | GGGGAUGGGCUCAUGGUCUCU | 1211 | 46424801 |
| saM118 | CCCTACCTCCGGGCTCCCAGACTGAGT | 1040 | CCCUACCUCCGGGCUCCCAGA | 1212 | 46424845 |
| saM119 | TGAGGTGCTGTTCCCATGCTTTGGAGT | 1041 | UGAGGUGCUGUUCCCAUGCUU | 1213 | 46424895 |
| saM120 | TGTTCCCATGCTTTGGAGTTCCTGAGT | 1042 | UGUUCCCAUGCUUUGGAGUUC | 1214 | 46424903 |
| saM121 | CTGAGTGTCCTCTGCTGTCCCCTGGAT | 1043 | CUGAGUGUCCUCUGCUGUCCC | 1215 | 46424924 |
| saM122 | CCCAATGGGCAGCTTCTCTGTCTGAGT | 1044 | CCCAAUGGGCAGCUUCUCUGU | 1216 | 46424964 |
| saM123 | CTGTCTGAGTTGCTGCAGTTGCTGAGT | 1045 | CUGUCUGAGUUGCUGCAGUUG | 1217 | 46424981 |
| saM124 | ATGAGGAGGAAGTCTGGGCTAATGGGT | 1046 | AUGAGGAGGAAGUCUGGGCUA | 1218 | 46425087 |
| saM125 | CTGGGCTAATGGGTTGCAGTGGTGAAT | 1047 | CUGGGCUAAUGGGUUGCAGUG | 1219 | 46425100 |
| saM126 | AGGCCCCATCTGTGTGCGGTGGTGGGT | 1048 | AGGCCCCAUCUGUGUGCGGUG | 1220 | 46425159 |
| saM127 | GCTGTGTGGTTATGTGCCTGGCTGGGT | 1049 | GCUGUGUGGUUAUGUGCCUGG | 1221 | 46425275 |
| saM128 | GCCTGGCTGGGTGTGCATGTGTTGGGT | 1050 | GCCUGGCUGGGUGUGCAUGUG | 1222 | 46425290 |
| saM129 | TGCATGTGTTGGGTTATTGGTTGGAGT | 1051 | UGCAUGUGUUGGGUUAUUGGU | 1223 | 46425303 |
| saM130 | ATCTAGCTATGTGTGGCTGGTGTGGGT | 1052 | AUCUAGCUAUGUGUGGCUGGU | 1224 | 46425336 |
| saM131 | CTATGTGTGGCTGGTGTGGGTCTGAAT | 1053 | CUAUGUGUGGCUGGUGUGGGU | 1225 | 46425342 |
| saM132 | TGTGGGTCTGAATGTCTGGTAGAGAGT | 1054 | UGUGGGUCUGAAUGUCUGGUA | 1226 | 46425356 |
| saM133 | GGGCATCTGGTTGGTGAACATGTGGAT | 1055 | GGGCAUCUGGUUGGUGAACAU | 1227 | 46425438 |
| saM134 | GGTATATGTCTGGATGGCTGGAGGAGT | 1056 | GGUAUAUGUCUGGAUGGCUGG | 1228 | 46425496 |
| saM135 | CTGGAGGAGTGGAAGAGGTTTTGGGGT | 1057 | CUGGAGGAGUGGAAGAGGUUU | 1229 | 46425513 |
| saM136 | CAATGGGAGGCTGGAGACACCATGAGT | 1058 | CAAUGGGAGGCUGGAGACACC | 1230 | 46425538 |
| saM137 | CGAGGTTGTCACTGGCAGAGAGAGGGT | 1059 | CGAGGUUGUCACUGGCAGAGA | 1231 | 46425651 |
| saM138 | GAATTTGTTTGCTGAGCCTGTGAGGGT | 1060 | GAAUUUGUUUGCUGAGCCUGU | 1232 | 46425736 |
| saM139 | CAGCAAACAAATTCAATTCTGCTGAGT | 1061 | CAGCAAACAAAUUCAAUUCUG | 1233 | 46425757 |
| saM140 | ATTTTACTAAACTCAGCAGAATTGAAT | 1062 | AUUUUACUAAACUCAGCAGAA | 1234 | 46425759 |
| saM141 | GGGAAATTTTACTAAACTCAGCAGAAT | 1063 | GGGAAAUUUUACUAAACUCAG | 1235 | 46425764 |
| saM142 | TGAGTTTAGTAAAATTTCCCAGGGAAT | 1064 | UGAGUUUAGUAAAAUUUCCCA | 1236 | 46425779 |
| saM143 | GGGGACACATTCATTATAAAGATGAAT | 1065 | GGGGACACAUUCAUUAUAAAG | 1237 | 46425836 |
| saM144 | GGGGTCAGATTCATCTTTATAATGAAT | 1066 | GGGGUCAGAUUCAUCUUUAUA | 1238 | 46425836 |
| saM145 | CTATGCAGGGCAGCAGCACGGCAGAGT | 1067 | CUAUGCAGGGCAGCAGCACGG | 1239 | 46425938 |
| saM146 | ACAGAACTGGTGACTTGGCAGGAGGGT | 1068 | ACAGAACUGGUGACUUGGCAG | 1240 | 46425984 |
| saM147 | AGGGTGGCTGGTCTGTCTGGAGAGGAT | 1069 | AGGGUGGCUGGUCUGUCUGGA | 1241 | 46426006 |
| saM148 | GGAGGGCTCCCCAGACGGGTGGTGAGT | 1070 | GGAGGGCUCCCCAGACGGGUG | 1242 | 46426040 |
| saM149 | AAATAATATTCCTAGGACCCATTGGGT | 1071 | AAAUAAUAUUCCUAGGACCCA | 1243 | 46426177 |
| saM150 | TCCATTTACCCAATGGGTCCTAGGAAT | 1072 | UCCAUUUACCCAAUGGGUCCU | 1244 | 46426176 |
| saM151 | AGCAGCTGGTCCATTTACCCAATGGGT | 1073 | AGCAGCUGGUCCAUUUACCCA | 1245 | 46426185 |
| saM152 | GGTGAGCAGAGCTTCCTGGCCATGAGT | 1074 | GGUGAGCAGAGCUUCCUGGCC | 1246 | 46426234 |
| saM153 | CTAAACATTTAACCACCACATTGGAAT | 1075 | CUAAACAUUUAACCACCACAU | 1247 | 46426314 |
| saM154 | AACTAGGCTGGAGGCAGCACCCTGAGT | 1076 | AACUAGGCUGGAGGCAGCACC | 1248 | 46426694 |
| saM155 | GCACCCTGAGTACAGAGAAGGCTGGAT | 1077 | GCACCCUGAGUACAGAGAAGG | 1249 | 46426710 |
| saM156 | ACATCCAGCCTTCTCTGTACTCAGGGT | 1078 | ACAUCCAGCCUUCUCUGUACU | 1250 | 46426704 |
| saM157 | CTGTGGGATGGAGCTGGAGGGAAGGGT | 1079 | CUGUGGGAUGGAGCUGGAGGG | 1251 | 46426747 |
| saM158 | CAAAAGATGATAGCCACATCACAGGAT | 1080 | CAAAAGAUGAUAGCCACAUCA | 1252 | 46427494 |
| saM159 | TTCAAAGCGCTCCTGATACATTGGAGT | 1081 | UUCAAAGCGCUCCUGAUACAU | 1253 | 46427545 |
| saM160 | GTCACTTGCAGTCTGATTAAGGAGAGT | 1082 | GUCACUUGCAGUCUGAUUAAG | 1254 | 46427578 |
| saM161 | TTAGTGATATTGTTCCGTGGGTGGAGT | 1083 | UUAGUGAUAUUGUUCCGUGGG | 1255 | 46427618 |
| saM162 | TATTAGAAAAGCTAGAAAATTGTGAGT | 1084 | UAUUAGAAAAGCUAGAAAAUU | 1256 | 46427672 |
| saM163 | TTAAGTAATAAACATTGTTATTAGAAT | 1085 | UUAAGUAAUAAACAUUGUUAU | 1257 | 46427747 |
| saM164 | TTCTGTTGTTGGGCTGGCTGTTTGAAT | 1086 | UUCUGUUGUUGGGCUGGCUGU | 1258 | 46427824 |
| saM165 | TCATTTAAGTGTAAGTGTTTGCTGGGT | 1087 | UCAUUUAAGUGUAAGUGUUUG | 1259 | 46427968 |
| saM166 | AGAGATGAAGAAGTTAAGATACAGAGT | 1088 | AGAGAUGAAGAAGUUAAGAUA | 1260 | 46427997 |
| saM167 | TGTAAACATTTATTAACTTGTTTGAGT | 1089 | UGUAAACAUUUAUUAACUUGU | 1261 | 46428052 |
| saM168 | CTGGCAACACAGTACCCTGTAGAGGAT | 1090 | CUGGCAACACAGUACCCUGUA | 1262 | 46428291 |
| saM169 | AGGGAAACAGTGAATCCTCTACAGGGT | 1091 | AGGGAAACAGUGAAUCCUCUA | 1263 | 46428296 |
| saM170 | CACAAAATCACAAGGGAAACAGTGAAT | 1092 | CACAAAAUCACAAGGGAAACA | 1264 | 46428308 |
| saM171 | TTGCTGGCACTGTCCAGTATCGAGAAT | 1093 | UUGCUGGCACUGUCCAGUAUC | 1265 | 46428361 |
| saM172 | CTGTCCAGTATCGAGAATCAAGAGAGT | 1094 | CUGUCCAGUAUCGAGAAUCAA | 1266 | 46428370 |

* chromosomal location of guide cut-site in chromosome 1 of human enome Hg38

### Example 6: Evaluation of In Vitro Gene Editing of SpCas9 gRNA Targeting FAAH-OUT

Frequency of INDELs induced at predicted cut sites in FAAH-OUT was evaluated following in vitro treatment with complexes of SpCas9 protein and sgRNA with spacers for SpCas9 as identified in Example 5.

Specifically, SpCas9 sgRNA were prepared with spacers shown in Table 18 (SpM1-SpM185; SEQ ID NOs: 366-550) inserted into a sgRNA backbone identified by SEQ ID NO: 1267. The SpCas9 sgRNA sequences were chemically synthesized and modified by a commercial vendor.

The sgRNA were individually evaluated as complexes with SpCas9 protein for inducing INDELs at predicted cut sites in FAAH-OUT. Editing efficiency was measured in MCF7 cells. Briefly, 1×10⁵ MCF7 cells were electroporated with 0.5 μg sgRNA and 0.5 μg SpCas9 protein (SEQ ID NO: 1268), then incubated for 48-72 hours. Genomic DNA was extracted as described in Example 2, and 1 μL (30-50 ng) of genomic DNA was used for PCR amplification of regions containing predicted cut sites. The purified PCR products were then sequenced using Sanger sequencing, and cutting efficiency was analyzed by Tsunami TIDE PCR and sequencing primers corresponding to each SpCas9 sgRNA are identified in Table 21.

The guides were categorized based on cleavage efficiency as measured by INDELs introduced at the predicted cut site. As shown in Table 22, guides without detectable cleavage efficiency (frequency of INDELs not detectable above threshold of the assay), with low cleavage efficiency (total frequency of INDELs less than 15%), moderate cleavage efficiency (total frequency of INDELs 15-25%), and high cleavage efficiency (total frequency of INDELs greater than 25%) are indicated.

TABLE 21
TIDE Analysis of SpCas9 gRNAs

| | | SEQ | | SEQ | | SEQ | | SEQ |
|---|---|---|---|---|---|---|---|---|
| | PCR | ID | PCR | ID | TIDE | ID | TIDE | ID |
| sgRNA | forward | NO | reverse | NO | seq1 | NO | seq2 | NO |
| SpM1 | ccctgcccc | 1590 | ttgagcg | | CGCCCTG | 1960 | CGGTTCC | 2141 |
| | ttgttactt | | tgtgggt | | CCCCTTG | | AAGCCCC | |
| | tc | | ttcaag | | TTACTT | | CAACTT | |
| SpM2 | ccctgcccc | 1591 | ttgagcg | 1776 | CGCCCTG | 1961 | CGGTTCC | 2142 |
| | ttgttactt | | tgtgggt | | CCCCTTG | | AAGCCCC | |
| | tc | | ttcaag | | TTACTT | | CAACTT | |
| SpM3 | ccctgcccc | 1592 | ttgagcg | 1777 | NA | | NA | |
| | ttgttactt | | tgtgggt | | | | | |
| | tc | | ttcaag | | | | | |
| SpM4 | ccctgcccc | 1593 | ttgagcg | 1778 | NA | | NA | |
| | ttgttactt | | tgtgggt | | | | | |
| | tc | | ttcaag | | | | | |
| SpM5 | ccctgcccc | 1594 | ttgagcg | 1779 | NA | | NA | |
| | ttgttactt | | tgtgggt | | | | | |
| | tc | | ttcaag | | | | | |
| SpM6 | ccctgcccc | 1595 | ttgagcg | 1780 | NA | | NA | |
| | ttgttactt | | tgtgggt | | | | | |
| | tc | | ttcaag | | | | | |
| SpM7 | ccctgcccc | 1596 | ttgagcg | 1781 | CTGGGTG | 1962 | CAAAAAG | 2143 |
| | ttgttactt | | tgtgggt | | CTGGCAG | | CTGTGGC | |
| | tc | | ttcaag | | TGACAA | | AGGCCG | |
| SpM8 | ccctgcccc | 1597 | ttgagcg | 1782 | AAAGAAG | 1963 | GGCTTAG | 2144 |
| | ttgttactt | | tgtgggt | | CTGTGGC | | AGGATGG | |
| | tc | | ttcaag | | AGTGGA | | TGCTCC | |
| SpM9 | ccctgcccc | 1598 | ttgagcg | 1783 | AAAGAAG | 1964 | GGCTTAG | 2145 |
| | ttgttactt | | tgtgggt | | CTGTGGC | | AGGATGG | |
| | tc | | ttcaag | | AGTGGA | | TGCTCC | |
| SpM10 | ccctgcccc | 1599 | ttgagcg | 1784 | ACAGAAG | 1965 | AAGCGAG | 2146 |
| | ttgttactt | | tgtgggt | | GGGGACA | | GCAAAAA | |
| | tc | | ttcaag | | GAGAGT | | GCTGTG | |
| SpM11 | ccctgcccc | 1600 | ttgagcg | 1785 | TTCTGGG | 1966 | CAGCTGC | 2147 |
| | ttgttactt | | tgtgggt | | CACTTCA | | AGGGTCA | |
| | tc | | ttcaag | | CAGTCA | | GGTTAA | |
| SpM12 | ccctgcccc | 1601 | ttgagcg | 1786 | CTGTGAG | 1967 | CCACAGC | 2148 |
| | ttgttactt | | tgtgggt | | CACTGAG | | TAGAAGT | |
| | tc | | ttcaag | | GAAGGG | | TGGGGG | |
| SpM13 | ccctgcccc | 1602 | ttgagcg | 1787 | CTGTGAG | 1968 | CCACAGC | 2149 |
| | ttgttactt | | tgtgggt | | CACTGAG | | TAGAAGT | |
| | tc | | ttcaag | | GAAGGG | | TGGGGG | |
| SpM14 | ccctgcccc | 1603 | ttgagcg | 1788 | CTGTGAG | 1969 | CCACAGC | 2150 |
| | ttgttactt | | tgtgggt | | CACTGAG | | TAGAAGT | |
| | tc | | ttcaag | | GAAGGG | | TGGGGG | |
| SpM15 | ccctgcccc | 1604 | ttgagcg | 1789 | CTGTGAG | 1970 | CCACAGC | 2151 |
| | ttgttactt | | tgtgggt | | CACTGAG | | TAGAAGT | |
| | tc | | ttcaag | | GAAGGG | | TGGGGG | |
| SpM16 | ccctgcccc | 1605 | ttgagcg | 1790 | CTGTGAG | 1971 | AGCACAA | 2152 |
| | ttgttactt | | tgtgggt | | CACTGAG | | ATCAGCC | |
| | tc | | ttcaag | | GAAGGG | | TCCTCC | |
| SpM17 | ccctgcccc | 1606 | ttgagcg | 1791 | ACACAGC | 1972 | GCACAAA | 2153 |
| | ttgttactt | | tgtgggt | | CTGACAG | | TCAGCCT | |
| | tc | | ttcaag | | AGTTGG | | CCTCCT | |
| SpM18 | ccctgcccc | 1607 | ttgagcg | 1792 | ACACAGC | 1973 | GCACAAA | 2154 |
| | ttgttactt | | tgtgggt | | CTGACAG | | TCAGCCT | |
| | tc | | ttcaag | | AGTTGG | | CCTCCT | |
| SpM19 | ccctgcccc | 1608 | ttgagcg | 1793 | ACACAGC | 1974 | GCACAAA | 2155 |
| | ttgttactt | | tgtgggt | | CTGACAG | | TCAGCCT | |
| | tc | | ttcaag | | AGTTGG | | CCTCCT | |
| SpM20 | ccctgcccc | 1609 | ttgagcg | 1794 | ACACAGC | 1975 | GCACAAA | 2156 |
| | ttgttactt | | tgtgggt | | CTGACAG | | TCAGCCT | |
| | tc | | ttcaag | | AGTTGG | | CCTCCT | |
| SpM21 | ccctgcccc | 1610 | ttgagcg | 1795 | ACACAGC | 1976 | GCACAAA | 2157 |
| | ttgttactt | | tgtgggt | | CTGACAG | | TCAGCCT | |
| | tc | | ttcaag | | AGTTGG | | CCTCCT | |
| SpM22 | ccctgcccc | 1611 | ttgagcg | 1796 | ACACAGC | 1977 | GCACAAA | 2158 |
| | ttgttactt | | tgtgggt | | CTGACAG | | TCAGCCT | |
| | tc | | ttcaag | | AGTTGG | | CCTCCT | |
| SpM23 | ccctgcccc | 1612 | ttgagcg | 1797 | ACACAGC | 1978 | GCACAAA | 2159 |
| | ttgttactt | | tgtgggt | | CTGACAG | | TCAGCCT | |
| | tc | | ttcaag | | AGTTGG | | CCTCCT | |
| SpM24 | ccctgcccc | 1613 | ttgagcg | 1798 | ACACAGC | 1979 | ACCTCTC | 2160 |
| | ttgttactt | | tgtgggt | | CTGACAG | | TGACCAC | |
| | tc | | ttcaag | | AGTTGG | | CAGTGT | |
| SpM25 | ccctgcccc | 1614 | ttgagcg | 1799 | TTGCTTT | 1980 | ACTGCCT | 2161 |
| | ttgttactt | | tgtgggt | | TGACCAC | | GTTTTCA | |
| | tc | | ttcaag | | GTGCAG | | TGGCCT | |
| SpM26 | ccctgcccc | 1615 | ttgagcg | 1800 | ACACAGC | 1981 | ACCTCTC | 2162 |
| | ttgttactt | | tgtgggt | | CTGACAG | | TGACCAC | |
| | tc | | ttcaag | | AGTTGG | | CAGTGT | |
| SpM27 | ccctgcccc | 1616 | ttgagcg | 1801 | ACACAGC | 1982 | ACCTCTC | 2163 |
| | ttgttactt | | tgtgggt | | CTGACAG | | TGACCAC | |
| | tc | | ttcaag | | AGTTGG | | CAGTGT | |
| SpM28 | ccctgcccc | 1617 | ttgagcg | 1802 | ACACAGC | 1983 | ACCTCTC | 2164 |
| | ttgttactt | | tgtgggt | | CTGACAG | | TGACCAC | |
| | tc | | ttcaag | | AGTTGG | | CAGTGT | |
| SpM29 | ccctgcccc | 1618 | ttgagcg | 1803 | TTGCTTT | 1984 | ACTGCCT | 2165 |
| | ttgttactt | | tgtgggt | | TGACCAC | | GTTTTCA | |
| | tc | | ttcaag | | GTGCAG | | TGGCCT | |
| SpM30 | ccctgcccc | 1619 | ttgagcg | 1804 | GCTCCAG | 1985 | GCAGAGG | 2166 |
| | ttgttactt | | tgtgggt | | ACTGGAC | | AAGACGC | |
| | tc | | ttcaag | | ATCTCCA | | CATCTCA | |
| | | | | | AC | | AA | |
| SpM31 | ccctgcccc | 1620 | ttgagcg | 1805 | GCTCCAG | 1986 | GCAGAGG | 2167 |
| | ttgttactt | | tgtgggt | | ACTGGAC | | AAGACGC | |
| | tc | | ttcaag | | ATCTCCA | | CATCTCA | |
| | | | | | AC | | AA | |
| SpM32 | ccctgcccc | 1621 | ttgagcg | 1806 | GCTCCAG | 1987 | GCAGAGG | 2168 |
| | ttgttactt | | tgtgggt | | ACTGGAC | | AAGACGC | |
| | tc | | ttcaag | | ATCTCCA | | CATCTCA | |
| | | | | | AC | | AA | |
| SpM33 | ccctgcccc | 1622 | ttgagcg | 1807 | GCCATGA | 1988 | GCAGAGG | 2169 |
| | ttgttactt | | tgtgggt | | TTAACCT | | AAGACGC | |
| | tc | | ttcaag | | GACCCTG | | CATCTCA | |
| | | | | | CA | | AA | |
| SpM34 | ccctgcccc | 1623 | ttgagcg | 1808 | GCCATGA | 1989 | GCAGAGG | 2170 |
| | ttgttactt | | tgtgggt | | TTAACCT | | AAGACGC | |
| | tc | | ttcaag | | GACCCTG | | CATCTCA | |
| | | | | | CA | | AA | |
| SpM35 | ccctgcccc | 1624 | ttgagcg | 1809 | GCCATGA | 1990 | GCAGAGG | 2171 |
| | ttgttactt | | tgtgggt | | TTAACCT | | AAGACGC | |
| | tc | | ttcaag | | GACCCTG | | CATCTCA | |
| | | | | | CA | | AA | |
| SpM36 | ccctgcccc | 1625 | ttgagcg | 1810 | GCCATGA | 1991 | GCAGAGG | 2172 |
| | ttgttactt | | tgtgggt | | TTAACCT | | AAGACGC | |
| | tc | | ttcaag | | GACCCTG | | CATCTCA | |
| | | | | | CA | | AA | |
| SpM37 | ccctgcccc | 1626 | ttgagcg | 1811 | GCCATGA | 1992 | GCAGAGG | 2173 |
| | ttgttactt | | tgtgggt | | TTAACCT | | AAGACGC | |
| | tc | | ttcaag | | GACCCTG | | CATCTCA | |
| | | | | | CA | | AA | |
| SpM38 | ccctgcccc | 1627 | ttgagcg | 1812 | GCCATGA | 1993 | GCAGAGG | 2174 |
| | ttgttactt | | tgtgggt | | TTAACCT | | AAGACGC | |
| | tc | | ttcaag | | GACCCTG | | CATCTCA | |
| | | | | | CA | | AA | |
| SpM39 | ccctgcccc | 1628 | ttgagcg | 1813 | GCCATGA | 1994 | GCAGAGG | 2175 |
| | ttgttactt | | tgtgggt | | TTAACCT | | AAGACGC | |
| | tc | | ttcaag | | GACCCTG | | CATCTCA | |
| | | | | | CA | | AA | |
| SpM40 | ccctgcccc | 1629 | ttgagcg | 1814 | GCAGAGG | 1995 | GCTCCAG | 2176 |
| | ttgttactt | | tgtgggt | | AAGACGC | | ACTGGAC | |
| | tc | | ttcaag | | CATCTCA | | ATCTCCA | |
| | | | | | AA | | AC | |
| SpM41 | ccctgcccc | 1630 | ttgagcg | 1815 | GCAGAGG | 1996 | GCTCCAG | 2177 |
| | ttgttactt | | tgtgggt | | AAGACGC | | ACTGGAC | |
| | tc | | ttcaag | | CATCTCA | | ATCTCCA | |
| | | | | | AA | | AC | |
| SpM42 | ctcatttgg | 1631 | tcacctt | 1816 | TGTGAAA | 1997 | AGCTACC | 2178 |
| | aaagtgggc | | tcactca | | GGAGCAA | | GTGTCTG | |
| | att | | ctcccc | | AGGGTCA | | GCCCTAT | |
| | | | | | GG | | TA | |
| SpM43 | ctcatttgg | 1632 | tcacctt | 1817 | TGGGTGC | 1998 | TGAAACC | 2179 |
| | aaagtgggc | | tcactca | | TGAGCAT | | CACACGC | |
| | att | | ctcccc | | ACACAG | | TCAACT | |
| SpM44 | ctcatttgg | 1633 | tcacctt | 1818 | TGGGTGC | 1999 | TGAAACC | 2180 |
| | aaagtgggc | | tcactca | | TGAGCAT | | CACACGC | |
| | att | | ctcccc | | ACACAG | | TCAACT | |
| SpM45 | ctcatttgg | 1634 | tcacctt | 1819 | TGGGTGC | 2000 | TGAAACC | 2181 |
| | aaagtgggc | | tcactca | | TGAGCAT | | CACACGC | |
| | att | | ctcccc | | ACACAG | | TCAACT | |
| SpM46 | ctcatttgg | 1635 | tcacctt | 1820 | AAAGGAG | 2001 | TGAAACC | 2182 |
| | aaagtgggc | | tcactca | | CAAAGGG | | CACACGC | |
| | att | | ctcccc | | TCAGGG | | TCAACT | |
| SpM47 | ctcatttgg | 1636 | tcacctt | 1821 | AAAGGAG | 2002 | TGAAACC | 2183 |
| | aaagtgggc | | tcactca | | CAAAGGG | | CACACGC | |
| | att | | ctcccc | | TCAGGG | | TCAACT | |
| SpM48 | ctcatttgg | 1637 | tcacctt | 1822 | AAAGGAG | 2003 | TGAAACC | 2184 |
| | aaagtgggc | | tcactca | | CAAAGGG | | CACACGC | |
| | att | | ctcccc | | TCAGGG | | TCAACT | |
| SpM49 | ctcatttgg | 1638 | tcacctt | 1823 | AAAGGAG | 2004 | TGAAACC | 2185 |
| | aaagtgggc | | tcactca | | CAAAGGG | | CACACGC | |
| | att | | ctcccc | | TCAGGG | | TCAACT | |
| SpM50 | ctcatttgg | 1639 | tcacctt | 1824 | AAAGGAG | 2005 | TGAAACC | 2186 |
| | aaagtgggc | | tcactca | | CAAAGGG | | CACACGC | |
| | att | | ctcccc | | TCAGGG | | TCAACT | |
| SpM51 | ctcatttgg | 1640 | tcacctt | 1825 | AAAGGAG | 2006 | CATTCTT | 2187 |
| | aaagtgggc | | tcactca | | CAAAGGG | | CGGACAC | |
| | att | | ctcccc | | TCAGGG | | CAGCCT | |
| SpM52 | ctcatttgg | 1641 | tcacctt | 1826 | GGCGTGA | 2007 | ACAGCCT | 2188 |
| | aaagtgggc | | tcactca | | CCTACAC | | GAGAGAG | |
| | att | | ctcccc | | CCTTAAC | | ATGAAGG | |
| | | | | | AG | | AGT | |
| SpM53 | ctcatttgg | 1642 | tcacctt | 1827 | TTTCTCT | 2008 | CCCTGCT | 2189 |
| | aaagtgggc | | tcactca | | GGCTGGG | | TTCTACC | |
| | att | | ctcccc | | CTTAGC | | AAGTGC | |
| SpM54 | ctcatttgg | 1643 | tcacctt | 1828 | TTTCTCT | 2009 | CCCTGCT | 2190 |
| | aaagtgggc | | tcactca | | GGCTGGG | | TTCTACC | |
| | att | | ctcccc | | CTTAGC | | AAGTGC | |
| SpM55 | ctcatttgg | 1644 | tcacctt | 1829 | TTTCTCT | 2010 | CCCTGCT | 2191 |
| | aaagtgggc | | tcactca | | GGCTGGG | | TTCTACC | |
| | att | | ctcccc | | CTTAGC | | AAGTGC | |
| SpM56 | ctcatttgg | 1645 | tcacctt | 1830 | TTTCTCT | 2011 | CCCTGCT | 2192 |
| | aaagtgggc | | tcactca | | GGCTGGG | | TTCTACC | |
| | att | | ctcccc | | CTTAGC | | AAGTGC | |
| SpM57 | ctcatttgg | 1646 | tcacctt | 1831 | GGCCCTC | 2012 | ATGGGTT | 2193 |
| | aaagtgggc | | tcactca | | CCAAGTT | | CAACTCC | |
| | att | | ctcccc | | TCCTAC | | ACAGCC | |
| SpM58 | ctcatttgg | 1647 | tcacctt | 1832 | ATGGGTT | 2013 | GGCCCTC | 2194 |
| | aaagtgggc | | tcactca | | CAACTCC | | CCAAGTT | |
| | att | | ctcccc | | ACAGCC | | TCCTAC | |
| SpM59 | ctcatttgg | 1648 | tcacctt | 1833 | ATGGGTT | 2014 | GGCCCTC | 2195 |
| | aaagtgggc | | tcactca | | CAACTCC | | CCAAGTT | |
| | att | | ctcccc | | ACAGCC | | TCCTAC | |
| SpM60 | ctcatttgg | 1649 | tcacctt | 1834 | ATGGGTT | 2015 | GGCCCTC | 2196 |
| | aaagtgggc | | tcactca | | CAACTCC | | CCAAGTT | |
| | att | | ctcccc | | ACAGCC | | TCCTAC | |
| SpM61 | ctcatttgg | 1650 | tcacctt | 1835 | ATGGGTT | 2016 | GGCCCTC | 2197 |
| | aaagtgggc | | tcactca | | CAACTCC | | CCAAGTT | |
| | att | | ctcccc | | ACAGCC | | TCCTAC | |
| SpM62 | ctcatttgg | 1651 | tcacctt | 1836 | ATGGGTT | 2017 | TGTGTAT | 2198 |
| | aaagtgggc | | tcactca | | CAACTCC | | GCTCAGC | |
| | att | | ctcccc | | ACAGCC | | ACCCAG | |
| SpM63 | ctcatttgg | 1652 | tcacctt | 1837 | ATGGGTT | 2018 | TGTGTAT | 2199 |
| | aaagtgggc | | tcactca | | CAACTCC | | GCTCAGC | |
| | att | | ctcccc | | ACAGCC | | ACCCAG | |
| SpM64 | ctcatttgg | 1653 | tcacctt | 1838 | ATGGGTT | 2019 | TGTGTAT | 2200 |
| | aaagtgggc | | tcactca | | CAACTCC | | GCTCAGC | |
| | att | | ctcccc | | ACAGCC | | ACCCAG | |
| SpM65 | ctcatttgg | 1654 | tcacctt | 1839 | ATGGGTT | 2020 | TGTGTAT | 2201 |
| | aaagtgggc | | tcactca | | CAACTCC | | GCTCAGC | |
| | att | | ctcccc | | ACAGCC | | ACCCAG | |
| SpM66 | ctcatttgg | 1655 | tcacctt | 1840 | ATGGGTT | 2021 | TGTGTAT | 2202 |
| | aaagtgggc | | tcactca | | CAACTCC | | GCTCAGC | |
| | att | | ctcccc | | ACAGCC | | ACCCAG | |
| SpM67 | ctcatttgg | 1656 | tcacctt | 1841 | TGGAAGC | 2022 | CCCTGAC | 2203 |
| | aaagtgggc | | tcactca | | TCCATTC | | CCTTTGC | |
| | att | | ctcccc | | AGGCAG | | TCCTTT | |
| SpM68 | ctcatttgg | 1657 | tcacctt | 1842 | TGGAAGC | 2023 | CCCTGAC | 2204 |
| | aaagtgggc | | tcactca | | TCCATTC | | CCTTTGC | |
| | att | | ctcccc | | AGGCAG | | TCCTTT | |
| SpM69 | ctcatttgg | 1658 | tcacctt | 1843 | TGCACTT | 2024 | AGTTTTC | 2205 |
| | aaagtgggc | | tcactca | | GGTAGAA | | TACACGG | |
| | att | | ctcccc | | AGCAGGG | | GCTGCCT | |
| | | | | | AC | | TT | |
| SpM70 | ctcatttgg | 1659 | tcacctt | 1844 | TGCACTT | 2025 | ACGTCTT | 2206 |
| | aaagtgggc | | tcactca | | GGTAGAA | | TTGTCCG | |
| | att | | ctcccc | | AGCAGGG | | CTTCCTG | |
| | | | | | AC | | AA | |
| SpM71 | ctcatttgg | 1660 | tcacctt | 1845 | GGCTGTG | 2026 | GCGTTGC | 2207 |
| | aaagtgggc | | tcactca | | GAGTTGA | | TGCTAGC | |
| | att | | ctcccc | | ACCCAT | | TCTTTC | |
| SpM72 | ctcatttgg | 1661 | tcacctt | 1846 | GGCTGTG | 2027 | GCGTTGC | 2208 |
| | aaagtgggc | | tcactca | | GAGTTGA | | TGCTAGC | |
| | att | | ctcccc | | ACCCAT | | TCTTTC | |
| SpM73 | ctcatttgg | 1662 | tcacctt | 1847 | GGCTGTG | 2028 | GCGTTGC | 2209 |
| | aaagtgggc | | tcactca | | GAGTTGA | | TGCTAGC | |
| | att | | ctcccc | | ACCCAT | | TCTTTC | |
| SpM74 | gaccctttg | 1663 | gctcctt | 1848 | GGCTGTG | 2029 | GCGTTGC | 2210 |
| | ctcctttca | | tgttccg | | GAGTTGA | | TGCTAGC | |
| | ca | | cataag | | ACCCAT | | TCTTTC | |
| SpM75 | gaccctttg | 1664 | gctcctt | 1849 | GGCTGTG | 2030 | TGAGCCC | 2211 |
| | ctcctttca | | tgttccg | | GAGTTGA | | TCCATTC | |
| | ca | | cataag | | ACCCAT | | CTACCT | |
| SpM76 | gaccctttg | 1665 | gctcctt | 1850 | GGCTGTG | 2031 | TGAGCCC | 2212 |
| | ctcctttca | | tgttccg | | GAGTTGA | | TCCATTC | |
| | ca | | cataag | | ACCCAT | | CTACCT | |
| SpM77 | gaccctttg | 1666 | gctcctt | 1851 | GCGTTGC | 2032 | GCACTTG | 2213 |
| | ctcctttca | | tgttccg | | TGCTAGC | | GTAGAAA | |
| | ca | | cataag | | TCTTTC | | GCAGGG | |
| SpM78 | gaccctttg | 1667 | gctcctt | 1852 | GCGTTGC | 2033 | GGCTGTG | 2214 |
| | ctcctttca | | tgttccg | | TGCTAGC | | GAGTTGA | |
| | ca | | cataag | | TCTTTC | | ACCCAT | |
| SpM79 | gaccctttg | 1668 | gctcctt | 1853 | GCGTTGC | 2034 | GGCTGTG | 2215 |
| | ctcctttca | | tgttccg | | TGCTAGC | | GAGTTGA | |
| | ca | | cataag | | TCTTTC | | ACCCAT | |
| SpM80 | gaccctttg | 1669 | gctcctt | 1854 | GCGTTGC | 2035 | GGCTGTG | 2216 |
| | ctcctttca | | tgttccg | | TGCTAGC | | GAGTTGA | |
| | ca | | cataag | | TCTTTC | | ACCCAT | |
| SpM81 | gaccctttg | 1670 | gctcctt | 1855 | TGGGGGA | 2036 | TCCCTCC | 2217 |
| | ctcctttca | | tgttccg | | GCATAGA | | ATTCATC | |
| | ca | | cataag | | CCTTGT | | CTCCCA | |
| SpM82 | gaccctttg | 1671 | gctcctt | 1856 | TGGGGGA | 2037 | TCCCTCC | 2218 |
| | ctcctttca | | tgttccg | | GCATAGA | | ATTCATC | |
| | ca | | cataag | | CCTTGT | | CTCCCA | |
| SpM83 | gaccctttg | 1672 | gctcctt | 1857 | TGGGGGA | 2038 | TCCCTCC | 2219 |
| | ctcctttca | | tgttccg | | GCATAGA | | ATTCATC | |
| | ca | | cataag | | CCTTGT | | CTCCCA | |
| SpM84 | gaccctttg | 1673 | gctcctt | 1858 | TGGGGGA | 2039 | TCCCTCC | 2220 |
| | ctcctttca | | tgttccg | | GCATAGA | | ATTCATC | |
| | ca | | cataag | | CCTTGT | | CTCCCA | |
| SpM85 | gaccctttg | 1674 | gctcctt | 1859 | CCTGAAG | 2040 | AGGAAGC | 2221 |
| | ctcctttca | | tgttccg | | TTGCCCA | | GGACAAA | |
| | ca | | cataag | | CTCTGT | | AGACGT | |
| SpM86 | gaccctttg | 1675 | gctcctt | 1860 | CCTGAAG | 2041 | AGGAAGC | 2222 |
| | ctcctttca | | tgttccg | | TTGCCCA | | GGACAAA | |
| | ca | | cataag | | CTCTGT | | AGACGT | |
| SpM87 | gaccctttg | 1676 | gctcctt | 1861 | CCTGAAG | 2042 | AGGAAGC | 2223 |
| | ctcctttca | | tgttccg | | TTGCCCA | | GGACAAA | |
| | ca | | cataag | | CTCTGT | | AGACGT | |
| SpM88 | gaccctttg | 1677 | gctcctt | 1862 | CCTGAAG | 2043 | AGGAAGC | 2224 |
| | ctcctttca | | tgttccg | | TTGCCCA | | GGACAAA | |
| | ca | | cataag | | CTCTGT | | AGACGT | |
| SpM89 | gaccctttg | 1678 | gctcctt | 1863 | CCTGAAG | 2044 | AGGAAGC | 2225 |
| | ctcctttca | | tgttccg | | TTGCCCA | | GGACAAA | |
| | ca | | cataag | | CTCTGT | | AGACGT | |
| SpM90 | gaccctttg | 1679 | gctcctt | 1864 | CCTGAAG | 2045 | AGGAAGC | 2226 |
| | ctcctttca | | tgttccg | | TTGCCCA | | GGACAAA | |
| | ca | | cataag | | CTCTGT | | AGACGT | |
| SpM91 | gaccctttg | 1680 | gctcctt | 1865 | CCTGAAG | 2046 | AGGAAGC | 2227 |
| | ctcctttca | | tgttccg | | TTGCCCA | | GGACAAA | |
| | ca | | cataag | | CTCTGT | | AGACGT | |
| SpM92 | gaccctttg | 1681 | gctcctt | 1866 | TGAGTCT | 2047 | GAAAGAG | 2228 |
| | ctcctttca | | tgttccg | | GAGTACC | | CTAGCAG | |
| | ca | | cataag | | CGAGGG | | CAACGC | |
| SpM93 | gaccctttg | 1682 | gctcctt | 1867 | TGAGTCT | 2048 | GAAAGAG | 2229 |
| | ctcctttca | | tgttccg | | GAGTACC | | CTAGCAG | |
| | ca | | cataag | | CGAGGG | | CAACGC | |
| SpM94 | gaccctttg | 1683 | gctcctt | 1868 | CCTGAAG | 2049 | AGGAAGC | 2230 |
| | ctcctttca | | tgttccg | | TTGCCCA | | GGACAAA | |
| | ca | | cataag | | CTCTGT | | AGACGT | |
| SpM95 | gaccctttg | 1684 | gctcctt | 1869 | TGAGTCT | 2050 | GAAAGAG | 2231 |
| | ctcctttca | | tgttccg | | GAGTACC | | CTAGCAG | |
| | ca | | cataag | | CGAGGG | | CAACGC | |
| SpM96 | gaccctttg | 1685 | gctcctt | 1870 | TGAGTCT | 2051 | GAAAGAG | 2232 |
| | ctcctttca | | tgttccg | | GAGTACC | | CTAGCAG | |
| | ca | | cataag | | CGAGGG | | CAACGC | |
| SpM97 | gaccctttg | 1686 | gctcctt | 1871 | TGAGTCT | 2052 | GAAAGAG | 2233 |
| | ctcctttca | | tgttccg | | GAGTACC | | CTAGCAG | |
| | ca | | cataag | | CGAGGG | | CAACGC | |
| SpM98 | gaccctttg | 1687 | gctcctt | 1872 | TGAGTCT | 2053 | GAAAGAG | 2234 |
| | ctcctttca | | tgttccg | | GAGTACC | | CTAGCAG | |
| | ca | | cataag | | CGAGGG | | CAACGC | |
| SpM99 | gaccctttg | 1688 | gctcctt | 1873 | TGGAAAA | 2054 | ACAAGGT | 2235 |
| | ctcctttca | | tgttccg | | GTGGTGC | | CTATGCT | |
| | ca | | cataag | | AGAGGG | | CCCCCA | |
| SpM100 | gaccctttg | 1689 | gctcctt | 1874 | TGGAAAA | 2055 | ACAAGGT | 2236 |
| | ctcctttca | | tgttccg | | GTGGTGC | | CTATGCT | |
| | ca | | cataag | | AGAGGG | | CCCCCA | |
| SpM101 | gagagotgg | 1690 | catgttc | 1875 | GGGCCAT | 2056 | CCAGCTC | 2237 |
| | agttcatga | | accaacc | | CAATCAC | | TGTGTGT | |
| | agg | | agatgc | | CATCCA | | GGTTGT | |
| SpM102 | gagagotgg | 1691 | catgttc | 1876 | GGGCCAT | 2057 | CCAGCTC | 2238 |
| | agttcatga | | accaacc | | CAATCAC | | TGTGTGT | |
| | agg | | agatgc | | CATCCA | | GGTTGT | |
| SpM103 | gagagctgg | 1692 | catgttc | 1877 | GGGCCAT | 2058 | CCAGCTC | 2239 |
| | agttcatga | | accaacc | | CAATCAC | | TGTGTGT | |
| | agg | | agatgc | | CATCCA | | GGTTGT | |
| SpM104 | gagagotgg | 1693 | catgttc | 1878 | CAGCAAC | 2059 | GCACGTG | 2240 |
| | agttcatga | | accaacc | | TGCAGCA | | GCTCAGT | |
| | agg | | agatgc | | ACTCAG | | AACATG | |
| SpM105 | gagagotgg | 1694 | catgttc | 1879 | CAGCAAC | 2060 | ACGTGGC | 2241 |
| | agttcatga | | accaacc | | TGCAGCA | | TCAGTAA | |
| | agg | | agatgc | | ACTCAG | | CATGGG | |
| SpM106 | gagagotgg | 1695 | catgttc | 1880 | CAGCAAC | 2061 | ACGTGGC | 2242 |
| | agttcatga | | accaacc | | TGCAGCA | | TCAGTAA | |
| | agg | | agatgc | | ACTCAG | | CATGGG | |
| SpM107 | gagagotgg | 1696 | catgttc | 1881 | CAGCAAC | 2062 | ACGTGGC | 2243 |
| | agttcatga | | accaacc | | TGCAGCA | | TCAGTAA | |
| | agg | | agatgc | | ACTCAG | | CATGGG | |
| SpM108 | gagagatgg | 1697 | catgttc | 1882 | CAGCAAC | 2063 | ACGTGGC | 2244 |
| | agttcatga | | accaacc | | TGCAGCA | | TCAGTAA | |
| | agg | | agatgc | | ACTCAG | | CATGGG | |
| SpM109 | gagagatgg | 1698 | catgttc | 1883 | CAGCAAC | 2064 | ACGTGGC | 2245 |
| | agttcatga | | accaacc | | TGCAGCA | | TCAGTAA | |
| | agg | | agatgc | | ACTCAG | | CATGGG | |
| SpM110 | gagagctgg | 1699 | catgttc | 1884 | CAGCAAC | 2065 | ACGTGGC | 2246 |
| | agttcatga | | accaacc | | TGCAGCA | | TCAGTAA | |
| | agg | | agatgc | | ACTCAG | | CATGGG | |
| SpM111 | gagagctgg | 1700 | catgttc | 1885 | GCAACCC | 2066 | GAGCACT | 2247 |
| | agttcatga | | accaacc | | ATTAGCC | | CCAAAAT | |
| | agg | | agatgc | | CAGACT | | CCCCCA | |
| SpM112 | gagagctgg | 1701 | catgttc | 1886 | GCAACCC | 2067 | GAGCACT | 2248 |
| | agttcatga | | accaacc | | ATTAGCC | | CCAAAAT | |
| | agg | | agatgc | | CAGACT | | CCCCCA | |
| SpM113 | gagagctgg | 1702 | catgttc | 1887 | GCAACCC | 2068 | GAGCACT | 2249 |
| | agttcatga | | accaacc | | ATTAGCC | | CCAAAAT | |
| | agg | | agatgc | | CAGACT | | CCCCCA | |
| SpM114 | gagagctgg | 1703 | catgttc | 1888 | GCAACCC | 2069 | GAGCACT | 2250 |
| | agttcatga | | accaacc | | ATTAGCC | | CCAAAAT | |
| | agg | | agatgc | | CAGACT | | CCCCCA | |
| SpM115 | gagagctgg | 1704 | catgttc | 1889 | GCAACCC | 2070 | GAGCACT | 2251 |
| | agttcatga | | accaacc | | ATTAGCC | | CCAAAAT | |
| | agg | | agatgc | | CAGACT | | CCCCCA | |
| SpM116 | gagagctgg | 1705 | catgttc | 1890 | AGCCAGA | 2071 | CAGGAAG | 2252 |
| | agttcatga | | accaacc | | CCAGACA | | CTGCAGG | |
| | agg | | agatgc | | CACAAC | | TCTTCA | |
| SpM117 | gagagctgg | 1706 | catgttc | 1891 | AGCCAGA | 2072 | CAGGAAG | 2253 |
| | agttcatga | | accaacc | | CCAGACA | | CTGCAGG | |
| | agg | | agatgc | | CACAAC | | TCTTCA | |
| SpM118 | gagagctgg | 1707 | catgttc | 1892 | AGCCAGA | 2073 | CAGGAAG | 2254 |
| | agttcatga | | accaacc | | CCAGACA | | CTGCAGG | |
| | agg | | agatgc | | CACAAC | | TCTTCA | |
| SpM119 | gagagctgg | 1708 | catgttc | 1893 | AGCCAGA | 2074 | CAGGAAG | 2255 |
| | agttcatga | | accaacc | | CCAGACA | | CTGCAGG | |
| | agg | | agatgc | | CACAAC | | TCTTCA | |
| SpM120 | gagagctgg | 1709 | catgttc | 1894 | AGCCAGA | 2075 | CAGGAAG | 2256 |
| | agttcatga | | accaacc | | CCAGACA | | CTGCAGG | |
| | agg | | agatgc | | CACAAC | | TCTTCA | |
| SpM121 | gagagctgg | 1710 | catgttc | 1895 | AGCCAGA | 2076 | CAGGAAG | 2257 |
| | agttcatga | | accaacc | | CCAGACA | | CTGCAGG | |
| | agg | | agatgc | | CACAAC | | TCTTCA | |
| SpM122 | gagagctgg | 1711 | catgttc | 1896 | AGCCAGA | 2077 | CAGGAAG | 2258 |
| | agttcatga | | accaacc | | CCAGACA | | CTGCAGG | |
| | agg | | agatgc | | CACAAC | | TCTTCA | |
| SpM123 | gagagctgg | 1712 | catgttc | 1897 | AGCCAGA | 2078 | CAGGAAG | 2259 |
| | agttcatga | | accaacc | | CCAGACA | | CTGCAGG | |
| | agg | | agatgc | | CACAAC | | TCTTCA | |
| SpM124 | ggtgctgtt | 1713 | caggagc | 1898 | CAGCCCA | 2079 | GGGAAGG | 2260 |
| | cccatgctt | | gctttga | | GACATCC | | AGGGACA | |
| | tg | | aagaca | | ACATGT | | TGGAGA | |
| SpM125 | ggtgctgtt | 1714 | caggagc | 1899 | CAGCCCA | 2080 | GGGAAGG | 2261 |
| | cccatgctt | | gctttga | | GACATCC | | AGGGACA | |
| | tg | | aagaca | | ACATGT | | TGGAGA | |
| SpM126 | ggtgctgtt | 1715 | caggagc | 1900 | CAGCCCA | 2081 | GACTGAG | 2262 |
| | cccatgctt | | gctttga | | GACATCC | | CCTGGGA | |
| | tg | | aagaca | | ACATGT | | TTTGCT | |
| SpM127 | ggtgctgtt | 1716 | caggagc | 1901 | CAGCCCA | 2082 | GACTGAG | 2263 |
| | cccatgctt | | gctttga | | GACATCC | | CCTGGGA | |
| | tg | | aagaca | | ACATGT | | TTTGCT | |
| SpM128 | ggtgctgtt | 1717 | caggagc | 1902 | CAGCCCA | 2083 | GACTGAG | 2264 |
| | cccatgctt | | gctttga | | GACATCC | | CCTGGGA | |
| | tg | | aagaca | | ACATGT | | TTTGCT | |
| SpM129 | ggtgctgtt | 1718 | caggagc | 1903 | CAGCCCA | 2084 | GACTGAG | 2265 |
| | cccatgctt | | gctttga | | GACATCC | | CCTGGGA | |
| | tg | | aagaca | | ACATGT | | TTTGCT | |
| SpM130 | ggtgctgtt | 1719 | caggagc | 1904 | CAGCCCA | 2085 | GATGGGC | 2266 |
| | cccatgctt | | gctttga | | GACATCC | | TCATGGT | |
| | tg | | aagaca | | ACATGT | | CTCTCG | |
| SpM131 | ggtgctgtt | 1720 | caggagc | 1905 | CAGCCCA | 2086 | GATGGGC | 2267 |
| | cccatgctt | | gctttga | | GACATCC | | TCATGGT | |
| | tg | | aagaca | | ACATGT | | CTCTCG | |
| SpM132 | ggtgctgtt | 1721 | caggagc | 1906 | CAGCCCA | 2087 | GATGGGC | 2268 |
| | cccatgctt | | gctttga | | GACATCC | | TCATGGT | |
| | tg | | aagaca | | ACATGT | | CTCTCG | |
| SpM133 | ggtgctgtt | 1722 | caggagc | 1907 | CAGCCCA | 2088 | GATGGGC | 2269 |
| | cccatgctt | | gctttga | | GACATCC | | TCATGGT | |
| | tg | | aagaca | | ACATGT | | CTCTCG | |
| SpM134 | ggtgctgtt | 1723 | caggagc | 1908 | CAGCCCA | 2089 | GATGGGC | 2270 |
| | cccatgctt | | gctttga | | GACATCC | | TCATGGT | |
| | tg | | aagaca | | ACATGT | | CTCTCG | |
| SpM135 | ggtgctgtt | 1724 | caggagc | 1909 | CAGCCCA | 2090 | GATGGGC | 2271 |
| | cccatgctt | | gctttga | | GACATCC | | TCATGGT | |
| | tg | | aagaca | | ACATGT | | CTCTCG | |
| SpM136 | ggtgctgtt | 1725 | caggagc | 1910 | ACAGCCC | 2091 | GATGGGC | 2272 |
| | cccatgctt | | gctttga | | AGACATC | | TCATGGT | |
| | tg | | aagaca | | CACATG | | CTCTCG | |
| SpM137 | ggtgctgtt | 1726 | caggagc | 1911 | CAAGGTG | 2092 | GTGCTGT | 2273 |
| | cccatgctt | | gctttga | | CTGAGAG | | TCCCATG | |
| | tg | | aagaca | | CCAAGA | | CTTTGG | |
| SpM138 | ggtgctgtt | 1727 | caggagc | 1912 | CAAGGTG | 2093 | GTGCTGT | 2274 |
| | cccatgctt | | gctttga | | CTGAGAG | | TCCCATG | |
| | tg | | aagaca | | CCAAGA | | CTTTGG | |
| SpM139 | ggtgctgtt | 1728 | caggagc | 1913 | CAAGGTG | 2094 | GTGCTGT | 2275 |
| | cccatgctt | | gctttga | | CTGAGAG | | TCCCATG | |
| | tg | | aagaca | | CCAAGA | | CTTTGG | |
| SpM140 | ggtgctgtt | 1729 | caggagc | 1914 | CAAGGTG | 2095 | GTGCTGT | 2276 |
| | cccatgctt | | gctttga | | CTGAGAG | | TCCCATG | |
| | tg | | aagaca | | CCAAGA | | CTTTGG | |
| SpM141 | ggtgctgtt | 1730 | caggagc | 1915 | CAAGGTG | 2096 | GTGCTGT | 2277 |
| | cccatgctt | | gctttga | | CTGAGAG | | TCCCATG | |
| | tg | | aagaca | | CCAAGA | | CTTTGG | |
| SpM142 | ggtgctgtt | 1731 | caggagc | 1916 | CAAGGTG | 2097 | GTGCTGT | 2278 |
| | cccatgctt | | gctttga | | CTGAGAG | | TCCCATG | |
| | tg | | aagaca | | CCAAGA | | CTTTGG | |
| SpM143 | ggtgctgtt | 1732 | caggagc | 1917 | CAAGGTG | 2098 | GTGCTGT | 2279 |
| | cccatgctt | | gctttga | | CTGAGAG | | TCCCATG | |
| | tg | | aagaca | | CCAAGA | | CTTTGG | |
| SpM144 | ggtgctgtt | 1733 | caggagc | 1918 | CAAGGTG | 2099 | GTGCTGT | 2280 |
| | cccatgctt | | gctttga | | CTGAGAG | | TCCCATG | |
| | tg | | aagaca | | CCAAGA | | CTTTGG | |
| SpM145 | ggtgctgtt | 1734 | caggagc | 1919 | CAAGGTG | 2100 | GTGCTGT | 2281 |
| | cccatgctt | | gctttga | | CTGAGAG | | TCCCATG | |
| | tg | | aagaca | | CCAAGA | | CTTTGG | |
| SpM146 | ggtgctgtt | 1735 | caggagc | 1920 | CAAGGTG | 2101 | GTGCTGT | 2282 |
| | cccatgctt | | gctttga | | CTGAGAG | | TCCCATG | |
| | tg | | aagaca | | CCAAGA | | CTTTGG | |
| SpM147 | ggtgctgtt | 1736 | caggagc | 1921 | CAAGGTG | 2102 | GTGCTGT | 2283 |
| | cccatgctt | | gctttga | | CTGAGAG | | TCCCATG | |
| | tg | | aagaca | | CCAAGA | | CTTTGG | |
| SpM148 | ggtgctgtt | 1737 | caggagc | 1922 | GGTCTCG | 2103 | CTGAGTT | 2284 |
| | cccatgctt | | gctttga | | AGGTTGT | | GCTGCAG | |
| | tg | | aagaca | | CACTGG | | TTGCTG | |
| SpM149 | ggtgctgtt | 1738 | caggagc | 1923 | GGTCTCG | 2104 | AGTCTGG | 2285 |
| | cccatgctt | | gctttga | | AGGTTGT | | GCTAATG | |
| | tg | | aagaca | | CACTGG | | GGTTGC | |
| SpM150 | ggtgctgtt | 1739 | caggagc | 1924 | CCCGGGA | 2105 | AGTCTGG | 2286 |
| | cccatgctt | | gctttga | | CATAAAG | | GCTAATG | |
| | tg | | aagaca | | GTGGAC | | GGTTGC | |
| SpM151 | ggtgctgtt | 1740 | caggagc | 1925 | CCCGGGA | 2106 | AGTCTGG | 2287 |
| | cccatgctt | | gctttga | | CATAAAG | | GCTAATG | |
| | tg | | aagaca | | GTGGAC | | GGTTGC | |
| SpM152 | ggtgctgtt | 1741 | caggagc | 1926 | AGTGTGA | 2107 | AGTCTGG | 2288 |
| | cccatgctt | | gctttga | | GTCAGGG | | GCTAATG | |
| | tg | | aagaca | | GTCAGA | | GGTTGC | |
| SpM153 | ggtgctgtt | 1742 | caggagc | 1927 | AGTGTGA | 2108 | AGTCTGG | 2289 |
| | cccatgctt | | gctttga | | GTCAGGG | | GCTAATG | |
| | tg | | aagaca | | GTCAGA | | GGTTGC | |
| SpM154 | ggtgctgtt | 1743 | caggagc | 1928 | AGTGTGA | 2109 | AGTCTGG | 2290 |
| | cccatgctt | | gctttga | | GTCAGGG | | GCTAATG | |
| | tg | | aagaca | | GTCAGA | | GGTTGC | |
| SpM155 | ggtgctgtt | 1744 | caggagc | 1929 | AGTGTGA | 2110 | AGTCTGG | 2291 |
| | cccatgctt | | gctttga | | GTCAGGG | | GCTAATG | |
| | tg | | aagaca | | GTCAGA | | GGTTGC | |
| SpM156 | ggtgctgtt | 1745 | caggagc | 1930 | AGTGTGA | 2111 | AGTCTGG | 2292 |
| | cccatgctt | | gctttga | | GTCAGGG | | GCTAATG | |
| | tg | | aagaca | | GTCAGA | | GGTTGC | |
| SpM157 | ggtgctgtt | 1746 | caggagc | 1931 | TCACCAG | 2112 | CCTGCTT | 2293 |
| | cccatgctt | | gctttga | | TTCTGTG | | TCTTGTG | |
| | tg | | aagaca | | GGCATC | | CCTCCT | |
| SpM158 | ggtgctgtt | 1747 | caggagc | 1932 | TCACCAG | 2113 | CCTGCTT | 2294 |
| | cccatgctt | | gctttga | | TTCTGTG | | TCTTGTG | |
| | tg | | aagaca | | GGCATC | | CCTCCT | |
| SpM159 | ggtgctgtt | 1748 | caggagc | 1933 | TCACCAG | 2114 | GTTGTGT | 2295 |
| | cccatgctt | | gctttga | | TTCTGTG | | GTCTGGT | |
| | tg | | aagaca | | GGCATC | | CTGGCT | |
| SpM160 | ggtgctgtt | 1749 | caggagc | 1934 | TCACCAG | 2115 | GTTGTGT | 2296 |
| | cccatgctt | | gctttga | | TTCTGTG | | GTCTGGT | |
| | tg | | aagaca | | GGCATC | | CTGGCT | |
| SpM161 | ggtgctgtt | 1750 | caggagc | 1935 | TCACCAG | 2116 | GTTGTGT | 2297 |
| | cccatgctt | | gctttga | | TTCTGTG | | GTCTGGT | |
| | tg | | aagaca | | GGCATC | | CTGGCT | |
| SpM162 | ggtgctgtt | 1751 | caggagc | 1936 | GCCATGA | 2117 | TGTGTGT | 2298 |
| | cccatgctt | | gctttga | | GGTTCAG | | CTGATGT | |
| | tg | | aagaca | | CTCACT | | GTGGGG | |
| SpM163 | ggtgctgtt | 1752 | caggagc | 1937 | AGCAGCT | 2118 | CATGTGG | 2299 |
| | cccatgctt | | gctttga | | GGTCCAT | | ATGTCTG | |
| | tg | | aagaca | | TTACCC | | GGCTGT | |
| SpM164 | ggtgctgtt | 1753 | caggagc | 1938 | AGCAGCT | 2119 | CATGTGG | 2300 |
| | cccatgctt | | gctttga | | GGTCCAT | | ATGTCTG | |
| | tg | | aagaca | | TTACCC | | GGCTGT | |
| SpM165 | ggtgctgtt | 1754 | caggagc | 1939 | AGCAGCT | 2120 | CATGTGG | 2301 |
| | cccatgctt | | gctttga | | GGTCCAT | | ATGTCTG | |
| | tg | | aagaca | | TTACCC | | GGCTGT | |
| SpM166 | ggtgctgtt | 1755 | caggagc | 1940 | AGCAGCT | 2121 | TCTTGGC | 2302 |
| | cccatgctt | | gctttga | | GGTCCAT | | TCTCAGC | |
| | tg | | aagaca | | TTACCC | | ACCTTG | |
| SpM167 | ggtgctgtt | 1756 | caggagc | 1941 | AGCAGCT | 2122 | TCTTGGC | 2303 |
| | cccatgctt | | gctttga | | GGTCCAT | | TCTCAGC | |
| | tg | | aagaca | | TTACCC | | ACCTTG | |
| SpM168 | ggtgctgtt | 1757 | caggagc | 1942 | GGCCATG | 2123 | TCTTGGC | 2304 |
| | cccatgctt | | gctttga | | AGTAGCT | | TCTCAGC | |
| | tg | | aagaca | | TGAGCA | | ACCTTG | |
| SpM169 | ggtgctgtt | 1758 | caggagc | 1943 | GGCCATG | 2124 | TCTTGGC | 2305 |
| | cccatgctt | | gctttga | | AGTAGCT | | TCTCAGC | |
| | tg | | aagaca | | TGAGCA | | ACCTTG | |
| SpM170 | ggtgctgtt | 1759 | caggagc | 1944 | GGCCATG | 2125 | TCTTGGC | 2306 |
| | cccatgctt | | gctttga | | AGTAGCT | | TCTCAGC | |
| | tg | | aagaca | | TGAGCA | | ACCTTG | |
| SpM171 | ggtgctgtt | 1760 | caggagc | 1945 | GAGGTGA | 2126 | TCTTGGC | 2307 |
| | cccatgctt | | gctttga | | GCAGAGC | | TCTCAGC | |
| | tg | | aagaca | | TTCCTG | | ACCTTG | |
| SpM172 | ggtgctgtt | 1761 | caggagc | 1946 | TTAGTTG | 2127 | CTTTATG | 2308 |
| | cccatgctt | | gctttga | | GGCTTGG | | TCCCGGG | |
| | tg | | aagaca | | TGGGAC | | GAGGTG | |
| SpM173 | ggtgctgtt | 1762 | caggagc | 1947 | TTAGTTG | 2128 | CTTTATG | 2309 |
| | cccatgctt | | gctttga | | GGCTTGG | | TCCCGGG | |
| | tg | | aagaca | | TGGGAC | | GAGGTG | |
| SpM174 | ggtgctgtt | 1763 | caggagc | 1948 | TTAGTTG | 2129 | CTTTATG | 2310 |
| | cccatgctt | | gctttga | | GGCTTGG | | TCCCGGG | |
| | tg | | aagaca | | TGGGAC | | GAGGTG | |
| SpM175 | ggtgctgtt | 1764 | caggagc | 1949 | TTAGTTG | 2130 | CTTTATG | 2311 |
| | cccatgctt | | gctttga | | GGCTTGG | | TCCCGGG | |
| | tg | | aagaca | | TGGGAC | | GAGGTG | |
| SpM176 | ggtgctgtt | 1765 | caggagc | 1950 | TTAGTTG | 2131 | CTTTATG | 2312 |
| | cccatgctt | | gctttga | | GGCTTGG | | TCCCGGG | |
| | tg | | aagaca | | TGGGAC | | GAGGTG | |
| SpM177 | ggtgctgtt | 1766 | caggagc | 1951 | TTAGTTG | 2132 | CTTTATG | 2313 |
| | cccatgctt | | gctttga | | GGCTTGG | | TCCCGGG | |
| | tg | | aagaca | | TGGGAC | | GAGGTG | |
| SpM178 | ggtgctgtt | 1767 | caggagc | 1952 | TTAGTTG | 2133 | GTCAAAC | 2314 |
| | cccatgctt | | gctttga | | GGCTTGG | | CCTCACA | |
| | tg | | aagaca | | TGGGAC | | GGCTCA | |
| SpM179 | ggtgctgtt | 1768 | caggagc | 1953 | TTAGTTG | 2134 | GTCAAAC | 2315 |
| | cccatgctt | | gctttga | | GGCTTGG | | CCTCACA | |
| | tg | | aagaca | | TGGGAC | | GGCTCA | |
| SpM180 | ggtgctgtt | 1769 | caggagc | 1954 | TTAGTTG | 2135 | GTCAAAC | 2316 |
| | cccatgctt | | gctttga | | GGCTTGG | | CCTCACA | |
| | tg | | aagaca | | TGGGAC | | GGCTCA | |
| SpM181 | ctggaggga | 1770 | cccagtc | 1955 | ATTGTTC | 2136 | CAGTGCC | 2317 |
| | agggttagc | | agccaca | | CGTGGGT | | AGCAAGA | |
| | tc | | aaatca | | GGAGTC | | CTAGCT | |
| SpM182 | ctggaggga | 1771 | cccagtc | 1956 | ATTGTTC | 2137 | CAGTGCC | 2318 |
| | agggttagc | | agccaca | | CGTGGGT | | AGCAAGA | |
| | tc | | aaatca | | GGAGTC | | CTAGCT | |
| SpM183 | ctggaggga | 1772 | cccagtc | 1957 | TGTTTGC | 2138 | TGCGCCT | 2319 |
| | agggttagc | | agccaca | | TGTGTAC | | GGCTAAT | |
| | tc | | aaatca | | CAGGCA | | TTGTTG | |
| SpM184 | ctggaggga | 1773 | cccagtc | 1958 | TGTTTGC | 2139 | TGCGCCT | 2320 |
| | agggttagc | | agccaca | | TGTGTAC | | GGCTAAT | |
| | tc | | aaatca | | CAGGCA | | TTGTTG | |
| SpM185 | ctggaggga | 1774 | cccagtc | 1959 | CAGGTGA | 2140 | TCCCTGT | 2321 |
| | agggttagc | | agccaca | | TTTTGCC | | CTTTCAA | |
| | tc | | aaatca | | CAACCG | | AGCGCT | |

TABLE 22
SpCas9 sgRNAs Categorized Based on Cleavage Efficiency

| Total INDEL % | Guides |
|---|---|
| Not detectable | SpM2, SpM3, SpM4, SpM5, SpM6, SpM8, SpM14, SpM28, SpM64, |
| above assay | SpM69, SpM70, SpM83, SpM84, SpM102, SpM105, SpM116, SpM129, |
| threshold of | SpM132, SpM133, SpM134, SpM135, SpM136, SpM141, SpM143, |
| detection | SpM145, SpM146, SpM147, SpM149, SpM150, SpM151, SpM152, |
| | SpM174, SpM176 |
| <15% | SpM1, SpM7, SpM10, SpM11, SpM12, SpM15, SpM17, SpM18, |
| | SpM19, SpM20, SpM21, SpM22, SpM23, SpM25, SpM26, SpM27, |
| | SpM29, SpM30, SpM31, SpM32, SpM33, SpM34, SpM35, SpM36, |
| | SpM37, SpM38, SpM39, SpM42, SpM43, SpM44, SpM45, SpM46, |
| | SpM47, SpM48, SpM49, SpM50, SpM51, SpM52, SpM54, SpM57, |
| | SpM58, SpM59, SpM61, SpM62, SpM63, SpM65, SpM66, SpM67, |
| | SpM68, SpM71, SpM72, SpM73, SpM74, SpM75, SpM76, SpM77, |
| | SpM78, SpM81, SpM82, SpM85, SpM86, SpM87, SpM88, SpM89, |
| | SpM90, SpM91, SpM92, SpM94, SpM95, SpM96, SpM97, SpM98 |
| | SpM101, SpM103, SpM104, SpM106, SpM107, SpM108, SpM109, |
| | SpM111, SpM114, SpM117, SpM119, SpM120, SpM121, SpM123, |
| | SpM125, SpM127, SpM128, SpM131, SpM142, SpM144, SpM148, |
| | SpM153, SpM159, SpM163, SpM166, SpM180, SpM182, SpM183, |
| | SpM184 |
| 15%-25% | SpM24, SpM53, SpM55, SpM79, SpM93, SpM99, SpM112, SpM130, |
| | SpM138, SpM140, SpM157, SpM158, SpM162, SpM165, SpM170, |
| | SpM181 |
| >25% | SpM9, SpM13, SpM16, SpM40, SpM41, SpM56, SpM60, SpM80, |
| | SpM100, SpM110, SpM113, SpM115, SpM118, SpM122, SpM124, |
| | SpM126, SpM137, SpM139, SpM154, SpM155, SpM156, SpM160, |
| | SpM161, SpM164, SpM167, SpM168, SpM169, SpM171, SpM172, |
| | SpM173, SpM175, SpM177, SpM178, SpM179, SpM185 |

A subset of the SpCas9 sgRNAs was selected for inducing a microdeletion in FAAH-OUT. Specifically, 4 left guides (SpM9, SpM13, SpM41, and SpM56) and 9 right guides (SpM110, SpM122, SpM126, SpM137, SpM168, SpM169, SpM173, SpM175, and SpM185) with high overall INDEL frequency were selected and re-evaluated for editing efficiency. The selected SpCas9 sgRNAs and corresponding frequency of INDELs at predicted cut-sites are identified in Table 23.

TABLE 23
Left and Right SpCas9 sgRNAs Targeting FAAH-OUT

| sgRNA Name | Indel % | L/R* |
|---|---|---|
| SpM41 | 82.7 | L |
| SpM169 | 82.7 | R |
| SpM110 | 75.4 | R |
| SpM185 | 75.3 | R |
| SpM173 | 70 | R |
| SpM126 | 68.5 | R |
| SpM9 | 67.3 | L |
| SpM168 | 66 | R |
| SpM122 | 62.7 | R |
| SpM137 | 61.75 | R |
| SpM56 | 60.2 | L |
| SpM13 | 56.9 | L |
| SpM175 | 53.7 | R |

*denotes Left (L) or Right (R) gRNA

Combinations of SpCas9 sgRNAs identified in Table 23 were evaluated for inducing a microdeletion in FAAH-OUT. Specifically, the sgRNA combinations identified in Table 24 were evaluated. Briefly, 0.3×10⁶MCF7 cells were electroporated with a left and right sgRNA (0.8 μg per each) and 1.5 μg SpCas9 protein (1.5 ug) for 48-72 hours. Cells were harvested for genomic DNA extraction, which was eluted in 30 ul DNA elution buffer (TE0.1). DNA concentration was measured by Dropsense (Trinean). 1 ul genomic DNA (˜30-60 ng) was used for droplet digital PCR (ddPCR) using the Bio-Rad QX200 ddPCR System (Bio-Rad, ddPCR™ Supermix for Probes (No dUTP) #1863024) to measure the genome deletion induced by the sgRNA pairs. A region of FAAH-OUT within the PT microdeletion (i.e., proximal to FOC) was amplified using the following primers:

(SEQ ID NO: 1276)
forward primer: CATAGACTGAGCCTGGGATTTG;

reverse primer: CAAAGCATGGGAACAGCACC (SEQ ID NO: 1277); and detected using

probe: AGGATGTGACAACCCGTCTC (SEQ ID NO: 1278). Primers corresponding to a genomic region outside the PT microdeletion (i.e., approximately 300 nt upstream FAAH) were used as a sample reference control:

reference forward primer:
(SEQ ID NO: 1279)
CCCAGTGACTAGTGTTCAGC;
reference reverse primer:
(SEQ ID NO: 1280)
CTTTCGCTCGACATCCACTG;

and detected using

reference probe:
(SEQ ID NO: 1281)
CTGGATCAGGAGCACAGTAGAC.

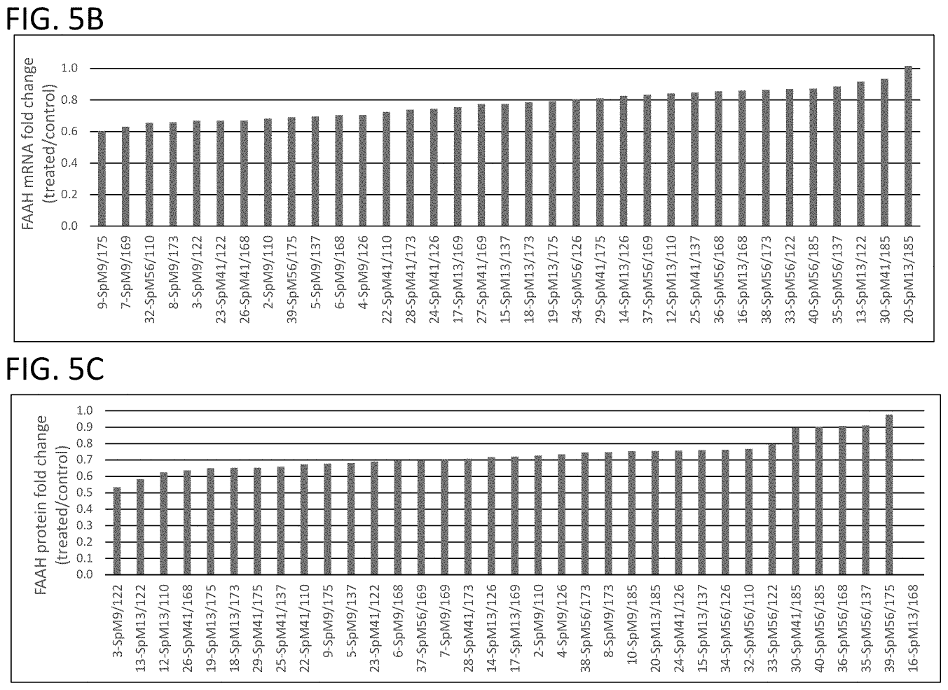

Deletion within FAAH-OUT was quantified based on the number of target sequence (TS) reads in the PT microdeletion relative to reference sequence (RS) reads outside the PT microdeletion, with % deletion equivalent to 100×(1-TS/RS). As shown in FIG. 5A, the majority of sgRNA pairs evaluated resulted in frequency of genomic deletion within FAAH-OUT that exceeded 40%. Quantification of deletion for each sgRNA combination is provided in Table 24.

The combinations of sgRNAs were further evaluated for effect on FAAH mRNA and protein expression. Briefly, MCF7 cells were electroporated with the combination sgRNAs as described above. Following 48-72 hours, the cells were harvested. Either RNA was extracted for quantification of FAAH mRNA by qPCR, or protein was extracted for quantification of FAAH protein by Simple Wes, each as described in Example 2.

As shown in FIG. 5B, the FAAH mRNA levels in treated cells, measured as fold change relative to control cells electroporated with SpCas9 only using the 2\hat{ }(−ddCt) method, were reduced by 20% or more for most of the sgRNA combinations tested. Quantification of fold change is provided in Table 24.

As shown in FIG. 5C, the FAAH protein levels were also evaluated, with FAAH-protein normalized to GAPDH levels then calculated as fold change for treated cells relative to PBS control cells. FAAH protein levels were significantly reduced for most of the sgRNA combinations tested. Quantification of fold change in FAAH protein between treated and control samples is provided in Table 24.

TABLE 24
SpCas9 Left and Right sgRNAs Targeting FAAH-OUT

| | | FAAH mRNA | FAAH protein |
|---|---|---|---|
| gRNA pair ID | Deletion (%) | (fold change) | (fold change) |
| 2-SpM9/110 | 70.78 | 0.68144 | 0.7274 |
| 3-SpM9/122 | 41.05 | 0.66836 | 0.5333 |
| 4-SpM9/126 | 53.23 | 0.70454 | 0.7339 |
| 5-SpM9/137 | 38.68 | 0.69484 | 0.6805 |
| 6-SpM9/168 | 39.67 | 0.70398 | 0.6969 |
| 7-SpM9/169 | 47.35 | 0.63096 | 0.7057 |
| 8-SpM9/173 | 45.13 | 0.65793 | 0.7473 |
| 9-SpM9/175 | 46.26 | 0.60404 | 0.6781 |
| 10-SpM9/185 | 14.65 | 0.60327 | 0.7537 |
| 12-SpM13/110 | 68.81 | 0.84196 | 0.624 |
| 13-SpM13/122 | 46.77 | 0.9163 | 0.5825 |
| 14-SpM13/126 | 45.36 | 0.82587 | 0.7175 |
| 15-SpM13/137 | 44.41 | 0.77515 | 0.7603 |
| 16-SpM13/168 | 37.74 | 0.85882 | NA |
| 17-SpM13/169 | 42.8 | 0.75423 | 0.7203 |
| 18-SpM13/173 | 38.66 | 0.78534 | 0.6519 |
| 19-SpM13/175 | 42 | 0.79203 | 0.6502 |
| 20-SpM13/185 | 18.36 | 1.01604 | 0.7542 |
| 22-SpM41/110 | 67.21 | 0.72344 | 0.6741 |
| 23-SpM41/122 | 38.32 | 0.66861 | 0.6911 |
| 24-SpM41/126 | 43.78 | 0.74379 | 0.7566 |
| 25-SpM41/137 | 36.41 | 0.84673 | 0.6581 |
| 26-SpM41/168 | 30.96 | 0.66892 | 0.6365 |
| 27-SpM41/169 | 43.24 | 0.77428 | 0.5119 |
| 28-SpM41/173 | 42.16 | 0.73828 | 0.7072 |
| 29-SpM41/175 | 41.04 | 0.81009 | 0.6525 |
| 30-SpM41/185 | 11.44 | 0.93321 | 0.8963 |
| 32-SpM56/110 | 73.41 | 0.65477 | 0.7675 |
| 33-SpM56/122 | 44.5 | 0.86968 | 0.7977 |
| 34-SpM56/126 | 52.01 | 0.80454 | 0.7626 |
| 35-SpM56/137 | 42.5 | 0.88465 | 0.91 |
| 36-SpM56/168 | 42.86 | 0.85496 | 0.9078 |
| 37-SpM56/169 | 47.97 | 0.83236 | 0.6972 |
| 38-SpM56/173 | 45.67 | 0.8633 | 0.7459 |
| 39-SpM56/175 | 48.48 | 0.69011 | 0.9765 |
| 40-SpM56/185 | 21.83 | 0.8715 | 0.9026 |

### Example 7: Evaluation of In Vitro Gene Editing of SluCas9 gRNA Targeting FAAH-OUT

Frequency of INDELs induced at predicted cut sites in FAAH-OUT was evaluated following in vitro treatment with complexes of SluCas9 protein and sgRNA with spacers for SluCas9 as identified in Example 5.

Specifically, SluCas9 sgRNA were prepared with spacers shown in Table 19 (SluM1-SluM186; SEQ ID NOs: 737-922) inserted into a sgRNA backbone identified by SEQ ID NO: 1269. The SluCas9 sgRNA sequences were chemically synthesized by a commercial vendor (Agilent).

The sgRNA were individually evaluated as complexes with SluCas9 protein for inducing INDELs at predicted cut sites in FAAH-OUT. Editing efficiency was measured in MCF7 cells. Briefly, 1×10⁵ MCF7 cells were electroporated with 0.7 μg sgRNA and 0.5 μg SluCas9 protein (SEQ ID NO: 1270), then incubated for 48-72 hours. Genomic DNA was extracted as described in Example 2, and 1 μL (30-50 ng) of genomic DNA was used for PCR amplification of regions containing predicted cut sites. The purified PCR products were then sequenced using Sanger sequencing, and cutting efficiency was analyzed by Tsunami TIDE PCR and sequencing primers corresponding to each SluCas9 sgRNA are identified in Table 25.

The guides were categorized based on cleavage efficiency as measured by INDELs introduced at the predicted cut site. As shown in Table 26, guides without detectable cleavage efficiency (frequency of INDELs not detectable above threshold of the assay), with low cleavage efficiency (total frequency of INDELs less than 15%), moderate cleavage efficiency (total frequency of INDELs 15-25%), and high cleavage efficiency (total frequency of INDELs greater than 25%) are indicated.

TABLE 25
TIDE Analysis of SluCas9 gRNAs

| | | SEQ | | SEQ | | SEQ | | SEQ |
|---|---|---|---|---|---|---|---|---|
| | | ID | | ID | | ID | | ID |
| sgRNA | PCR forward | NO | PCR reverse | NO | TIDE seq1 | NO | TIDE seq2 | NO |
| SluM1 | ccctgccccuguactuc | 2322 | ttgagcgtgtg | 2508 | GGAGGAGGCTGAT | 2694 | CAGGATCTTGG | 2880 |
| | | | ggtttcaag | | TTGTGCT | | CTCACTGCA | |
| SluM2 | ccctgccccuguactuc | 2323 | ttgagcgtgtg | 2509 | GGAGGAGGCTGAT | 2695 | CAGGATCTTGG | 2881 |
| | | | ggtttcaag | | TTGTGCT | | CTCACTGCA | |
| SluM3 | ccctgccccttgttactt | 2324 | ttgagcgtgtg | 2510 | GGAGGAGGCTGAT | 2696 | CAGGATCTTGG | 2882 |
| | tc | | ggtttcaag | | TTGTGCT | | CTCACTGCA | |
| SluM4 | ccctgccccttgttactt | 2325 | ttgagcgtgtg | 2511 | GGAGGAGGCTGAT | 2697 | CAGGATCTTGG | 2883 |
| | tc | | ggtttcaag | | TTGTGCT | | CTCACTGCA | |
| SluM5 | ccctgccccttgttactt | 2326 | ttgagcgtgtg | 2512 | GGTGCTGGCAGTGA | 2698 | AAGCGAGGCA | 2884 |
| | tc | | ggtttcaag | | CAAATG | | AAAAGCTGTG | |
| SluM6 | ccctgccccttgttactt | 2327 | ttgagcgtgtg | 2513 | GGTGCTGGCAGTGA | 2699 | AAGCGAGGCA | 2885 |
| | tc | | ggtttcaag | | CAAATG | | AAAAGCTGTG | |
| SluM7 | ccctgccccttgttactt | 2328 | ttgagcgtgtg | 2514 | GGAGGAGGCTGAT | 2700 | CCTGGCCACCC | 2886 |
| | tc | | ggtttcaag | | TTGTGCT | | TTTGTTCTT | |
| SluM8 | ccctgccccttgttactt | 2329 | ttgagcgtgtg | 2515 | AAAGAAGCTGTGG | 2701 | GGCTTAGAGGA | 2887 |
| | tc | | ggtttcaag | | CAGTGGA | | TGGTGCTCC | |
| SluM9 | ccctgccccttgttactt | 2330 | ttgagcgtgtg | 2516 | ACAGAAGGGGGAC | 2702 | AAGCGAGGCA | 2888 |
| | tc | | ggtttcaag | | AGAGAGT | | AAAAGCTGTG | |
| SluM10 | ccctgccccttgttactt | 2331 | ttgagcgtgtg | 2517 | TTCTGGGCACTTCA | 2703 | CAGCTGCAGGG | 2889 |
| | tc | | ggatcaag | | CAGTCA | | TCAGGTTAA | |
| sluMll | ccctgccccttgttactt | 2332 | ttgagcgtgtg | 2518 | TTCTGGGCACTTCA | 2704 | CAGCTGCAGGG | 2890 |
| | tc | | ggatcaag | | CAGTCA | | TCAGGTTAA | |
| SluM12 | ccctgccccttgttactt | 2333 | ttgagcgtgtg | 2519 | CTGTGAGCACTGAG | 2705 | CCACAGCTAGA | 2891 |
| | tc | | ggatcaag | | GAAGGG | | AGTTGGGGG | |
| SluM13 | ccctgccccttgttactt | 2334 | ttgagcgtgtg | 2520 | CTGTGAGCACTGAG | 2706 | CCACAGCTAGA | 2892 |
| | tc | | ggatcaag | | GAAGGG | | AGTTGGGGG | |
| SluM14 | ccctgccccttgttactt | 2335 | ttgagcgtgtg | 2521 | CTGTGAGCACTGAG | 2707 | CCACAGCTAGA | 2893 |
| | tc | | ggatcaag | | GAAGGG | | AGTTGGGGG | |
| SluM15 | ccctgccccttgttactt | 2336 | ttgagcgtgtg | 2522 | CTGTGAGCACTGAG | 2708 | CCACAGCTAGA | 2894 |
| | tc | | ggatcaag | | GAAGGG | | AGTTGGGGG | |
| SluM16 | ccctgccccttgttactt | 2337 | ttgagcgtgtg | 2523 | CTGTGAGCACTGAG | 2709 | CCACAGCTAGA | 2895 |
| | tc | | ggatcaag | | GAAGGG | | AGTTGGGGG | |
| SluM17 | ccctgccccttgttactt | 2338 | ttgagcgtgtg | 2524 | CTGTGAGCACTGAG | 2710 | CCACAGCTAGA | 2896 |
| | tc | | ggatcaag | | GAAGGG | | AGTTGGGGG | |
| SluM18 | ccctgccccttgttactt | 2339 | ttgagcgtgtg | 2525 | CTGTGAGCACTGAG | 2711 | CCACAGCTAGA | 2897 |
| | tc | | ggatcaag | | GAAGGG | | AGTTGGGGG | |
| SluM19 | ccctgccccttgttactt | 2340 | ttgagcgtgtg | 2526 | CTGTGAGCACTGAG | 2712 | CCACAGCTAGA | 2898 |
| | tc | | ggatcaag | | GAAGGG | | AGTTGGGGG | |
| SluM20 | ccctgccccttgttactt | 2341 | ttgagcgtgtg | 2527 | ACACAGCCTGACA | 2713 | GCACAAATCAG | 2899 |
| | tc | | ggatcaag | | GAGTTGG | | CCTCCTCCT | |
| SluM21 | ccctgccccttgttactt | 2342 | ttgagcgtgtg | 2528 | ACACAGCCTGACA | 2714 | GCACAAATCAG | 2900 |
| | tc | | ggatcaag | | GAGTTGG | | CCTCCTCCT | |
| SluM22 | ccctgccccttgttactt | 2343 | ttgagcgtgtg | 2529 | ACACAGCCTGACA | 2715 | GCACAAATCAG | 2901 |
| | tc | | ggatcaag | | GAGTTGG | | CCTCCTCCT | |
| SluM23 | ccctgccccttgttactt | 2344 | ttgagcgtgtg | 2530 | ACACAGCCTGACA | 2716 | GCACAAATCAG | 2902 |
| | tc | | ggatcaag | | GAGTTGG | | CCTCCTCCT | |
| SluM24 | ccctgccccttgttactt | 2345 | ttgagcgtgtg | 2531 | ACACAGCCTGACA | 2717 | GCACAAATCAG | 2903 |
| | tc | | ggatcaag | | GAGTTGG | | CCTCCTCCT | |
| SluM25 | ccctgccccttgttactt | 2346 | ttgagcgtgtg | 2532 | ACACAGCCTGACA | 2718 | GCACAAATCAG | 2904 |
| | tc | | ggatcaag | | GAGTTGG | | CCTCCTCCT | |
| SluM26 | ccctgccccttgttactt | 2347 | ttgagcgtgtg | 2533 | ACACAGCCTGACA | 2719 | GCACAAATCAG | 2905 |
| | tc | | ggatcaag | | GAGTTGG | | CCTCCTCCT | |
| SluM27 | ccctgccccttgttactt | 2348 | ttgagcgtgtg | 2534 | ACACAGCCTGACA | 2720 | ACCTCTCTGAC | 2906 |
| | tc | | ggatcaag | | GAGTTGG | | CACCAGTGT | |
| SluM28 | ccctgccccttgttactt | 2349 | ttgagcgtgtg | 2535 | ACACAGCCTGACA | 2721 | GCACAAATCAG | 2907 |
| | tc | | ggatcaag | | GAGTTGG | | CCTCCTCCT | |
| SluM29 | ccctgccccttgttactt | 2350 | ttgagcgtgtg | 2536 | ACACAGCCTGACA | 2722 | ACCTCTCTGAC | 2908 |
| | tc | | ggatcaag | | GAGTTGG | | CACCAGTGT | |
| SluM30 | ccctgccccttgttactt | 2351 | ttgagcgtgtg | 2537 | ACACAGCCTGACA | 2723 | ACCTCTCTGAC | 2909 |
| | tc | | ggatcaag | | GAGTTGG | | CACCAGTGT | |
| SluM31 | ccctgccccttgttactt | 2352 | ttgagcgtgtg | 2538 | TTGCTTTTGACCAC | 2724 | ACTGCCTGTTT | 2910 |
| | tc | | ggatcaag | | GTGCAG | | TCATGGCCT | |
| SluM32 | ccctgccccttgttactt | 2353 | ttgagcgtgtg | 2539 | ACACAGCCTGACA | 2725 | ACCTCTCTGAC | 2911 |
| | tc | | ggatcaag | | GAGTTGG | | CACCAGTGT | |
| SluM33 | ccctgccccttgttactt | 2354 | ttgagcgtgtg | 2540 | ACACAGCCTGACA | 2726 | ACCTCTCTGAC | 2912 |
| | tc | | ggatcaag | | GAGTTGG | | CACCAGTGT | |
| SluM34 | ccctgccccttgttactt | 2355 | ttgagcgtgtg | 2541 | TTGCTTTTGACCAC | 2727 | ACTGCCTGTTT | 2913 |
| | tc | | ggatcaag | | GTGCAG | | TCATGGCCT | |
| SluM35 | ccctgccccttgttactt | 2356 | ttgagcgtgtg | 2542 | TTGCTTTTGACCAC | 2728 | ACTGCCTGTTT | 2914 |
| | tc | | ggatcaag | | GTGCAG | | TCATGGCCT | |
| SluM36 | ccctgccccttgttactt | 2357 | ttgagcgtgtg | 2543 | TTGCTTTTGACCAC | 2729 | TCCACTGCCAC | 2915 |
| | tc | | ggatcaag | | GTGCAG | | AGCTTCTTT | |
| SluM37 | ccctgccccttgttactt | 2358 | ttgagcgtgtg | 2544 | TTGCTTTTGACCAC | 2730 | TCCACTGCCAC | 2916 |
| | tc | | ggatcaag | | GTGCAG | | AGCTTCTTT | |
| SluM38 | ccctgccccttgttactt | 2359 | ttgagcgtgtg | 2545 | CACAGCTTTTTGCC | 2731 | GCAGAGGAAG | 2917 |
| | tc | | ggatcaag | | TCGCTT | | ACGCCATCTC | |
| SluM39 | ccctgccccttgttactt | 2360 | ttgagcgtgtg | 2546 | CACAGCTTTTTGCC | 2732 | GCAGAGGAAG | 2918 |
| | tc | | ggatcaag | | TCGCTT | | ACGCCATCTC | |
| SluM40 | ccctgccccttgttactt | 2361 | ttgagcgtgtg | 2547 | CACAGCTTTTTGCC | 2733 | GCAGAGGAAG | 2919 |
| | tc | | ggatcaag | | TCGCTT | | ACGCCATCTC | |
| SluM41 | ccctgccccttgttactt | 2362 | ttgagcgtgtg | 2548 | CACAGCTTTTTGCC | 2734 | GCAGAGGAAG | 2920 |
| | tc | | ggatcaag | | TCGCTT | | ACGCCATCTC | |
| SluM42 | ccctgccccttgttactt | 2363 | ttgagcgtgtg | 2549 | CACAGCTTTTTGCC | 2735 | GCAGAGGAAG | 2921 |
| | tc | | ggatcaag | | TCGCTT | | ACGCCATCTC | |
| SluM43 | ccctgccccttgttactt | 2364 | ttgagcgtgtg | 2550 | CACAGCTTTTTGCC | 2736 | GCAGAGGAAG | 2922 |
| | tc | | ggatcaag | | TCGCTT | | ACGCCATCTC | |
| SluM44 | ccctgccccttgttactt | 2365 | ttgagcgtgtg | 2551 | TTAACCTGACCCTG | 2737 | GCAGAGGAAG | 2923 |
| | tc | | ggatcaag | | CAGCTG | | ACGCCATCTC | |
| SluM45 | ccctgccccttgttactt | 2366 | ttgagcgtgtg | 2552 | TTAACCTGACCCTG | 2738 | GCAGAGGAAG | 2924 |
| | tc | | ggatcaag | | CAGCTG | | ACGCCATCTC | |
| SluM46 | ccctgccccttgttactt | 2367 | ttgagcgtgtg | 2553 | CACAGCTTTTTGCC | 2739 | GCAGAGGAAG | 2925 |
| | tc | | ggatcaag | | TCGCTT | | ACGCCATCTC | |
| SluM47 | ccctgccccttgttactt | 2368 | ttgagcgtgtg | 2554 | CACAGCTTTTTGCC | 2740 | GCAGAGGAAG | 2926 |
| | tc | | ggatcaag | | TCGCTT | | ACGCCATCTC | |
| SluM48 | ccctgccccttgttactt | 2369 | ttgagcgtgtg | 2555 | CACAGCTTTTTGCC | 2741 | GCAGAGGAAG | 2927 |
| | tc | | ggatcaag | | TCGCTT | | ACGCCATCTC | |
| SluM49 | ccctgccccttgttactt | 2370 | ttgagcgtgtg | 2556 | CAAAACATAGCCG | 2742 | CACAGCTTTTT | 2928 |
| | tc | | ggatcaag | | GGCACAG | | GCCTCGCTT | |
| SluM50 | ccctgccccttgttactt | 2371 | ttgagcgtgtg | 2557 | CAAAACATAGCCG | 2743 | CACAGCTTTTT | 2929 |
| | tc | | ggatcaag | | GGCACAG | | GCCTCGCTT | |
| SluM51 | ccctgccccttgttactt | 2372 | ttgagcgtgtg | 2558 | CAAAACATAGCCG | 2744 | CACAGCTTTTT | 2930 |
| | tc | | ggatcaag | | GGCACAG | | GCCTCGCTT | |
| SluM52 | ccctgccccttgttactt | 2373 | ttgagcgtgtg | 2559 | CAAAACATAGCCG | 2745 | CACAGCTTTTT | 2931 |
| | tc | | ggatcaag | | GGCACAG | | GCCTCGCTT | |
| SluM53 | gaccctttgctcctttca | 2374 | gctcctttgtt | 2560 | TGGGGGAGCATAG | 2746 | TCCCTCCATTC | 2932 |
| | ca | | ccgcataag | | ACCTTGT | | ATCCTCCCA | |
| SluM54 | gaccctttgctcctttca | 2375 | gctcctttgtt | 2561 | TGGGGGAGCATAG | 2747 | TCCCTCCATTC | 2933 |
| | ca | | ccgcataag | | ACCTTGT | | ATCCTCCCA | |
| SluM55 | gaccctttgctcctttca | 2376 | gctcctttgtt | 2562 | TGGGGGAGCATAG | 2748 | TCCCTCCATTC | 2934 |
| | ca | | ccgcataag | | ACCTTGT | | ATCCTCCCA | |
| SluM56 | gaccctttgctcctttca | 2377 | gctcctttgtt | 2563 | TGGGGGAGCATAG | 2749 | TCCCTCCATTC | 2935 |
| | ca | | ccgcataag | | ACCTTGT | | ATCCTCCCA | |
| SluM57 | gaccctttgctcctttca | 2378 | gctcctttgtt | 2564 | TGGGGGAGCATAG | 2750 | TCCCTCCATTC | 2936 |
| | ca | | ccgcataag | | ACCTTGT | | ATCCTCCCA | |
| SluM58 | gaccctttgctcctttca | 2379 | gctcctttgtt | 2565 | TTGGGCGGATCAAT | 2751 | CCTGCCTGAAT | 2937 |
| | ca | | ccgcataag | | TGAGCT | | GGAGCTTCC | |
| SluM59 | gaccctttgctcctttca | 2380 | gctcctttgtt | 2566 | TTGGGCGGATCAAT | 2752 | CCTGCCTGAAT | 2938 |
| | ca | | ccgcataag | | TGAGCT | | GGAGCTTCC | |
| SluM60 | gaccctttgctcctttca | 2381 | gctcctttgtt | 2567 | TTGGGCGGATCAAT | 2753 | GTGCAATCAAG | 2939 |
| | ca | | ccgcataag | | TGAGCT | | CAGAAGCCC | |
| SluM61 | gaccctttgctcctttca | 2382 | gctcctttgtt | 2568 | TTGGGCGGATCAAT | 2754 | GTGCAATCAAG | 2940 |
| | ca | | ccgcataag | | TGAGCT | | CAGAAGCCC | |
| SluM62 | gaccctttgctcctttca | 2383 | gctcctttgtt | 2569 | CCTGAAGTTGCCCA | 2755 | AGGAAGCGGA | 2941 |
| | ca | | ccgcataag | | CTCTGT | | CAAAAGACGT | |
| SluM63 | gaccctttgctcctttca | 2384 | gctcctttgtt | 2570 | CCTGAAGTTGCCCA | 2756 | AGGAAGCGGA | 2942 |
| | ca | | ccgcataag | | CTCTGT | | CAAAAGACGT | |
| SluM64 | gaccctttgctcctttca | 2385 | gctcctttgtt | 2571 | CCTGAAGTTGCCCA | 2757 | AGGAAGCGGA | 2943 |
| | ca | | ccgcataag | | CTCTGT | | CAAAAGACGT | |
| SluM65 | gaccctttgctcctttca | 2386 | gctcctttgtt | 2572 | CCTGAAGTTGCCCA | 2758 | AGGAAGCGGA | 2944 |
| | ca | | ccgcataag | | CTCTGT | | CAAAAGACGT | |
| SluM66 | gaccctttgctcctttca | 2387 | gctcctttgtt | 2573 | CCTGAAGTTGCCCA | 2759 | AGGAAGCGGA | 2945 |
| | ca | | ccgcataag | | CTCTGT | | CAAAAGACGT | |
| SluM67 | gaccctttgctcctttca | 2388 | gctcctttgtt | 2574 | CCTGAAGTTGCCCA | 2760 | AGGAAGCGGA | 2946 |
| | ca | | ccgcataag | | CTCTGT | | CAAAAGACGT | |
| SluM68 | gaccctttgctcctttca | 2389 | gctcctttgtt | 2575 | CCTGAAGTTGCCCA | 2761 | AGGAAGCGGA | 2947 |
| | ca | | ccgcataag | | CTCTGT | | CAAAAGACGT | |
| SluM69 | gaccctttgctcctttca | 2390 | gctcctttgtt | 2576 | CCTGAAGTTGCCCA | 2762 | AGGAAGCGGA | 2948 |
| | ca | | ccgcataag | | CTCTGT | | CAAAAGACGT | |
| SluM70 | gaccctttgctcctttca | 2391 | gctcctttgtt | 2577 | TGAGTCTGAGTACC | 2763 | GAAAGAGCTA | 2949 |
| | ca | | ccgcataag | | CGAGGG | | GCAGCAACGC | |
| SluM71 | gaccctttgctcctttca | 2392 | gctcctttgtt | 2578 | CCTGAAGTTGCCCA | 2764 | AGGAAGCGGA | 2950 |
| | ca | | ccgcataag | | CTCTGT | | CAAAAGACGT | |
| SluM72 | gaccctttgctcctttca | 2393 | gctcctttgtt | 2579 | TGAGTCTGAGTACC | 2765 | GAAAGAGCTA | 2951 |
| | ca | | ccgcataag | | CGAGGG | | GCAGCAACGC | |
| SluM73 | gaccctttgctcctttca | 2394 | gctcctttgtt | 2580 | TGAGTCTGAGTACC | 2766 | GAAAGAGCTA | 2952 |
| | ca | | ccgcataag | | CGAGGG | | GCAGCAACGC | |
| SluM74 | gaccctttgctcctttca | 2395 | gctcctttgtt | 2581 | TGAGTCTGAGTACC | 2767 | GAAAGAGCTA | 2953 |
| | ca | | ccgcataag | | CGAGGG | | GCAGCAACGC | |
| SluM75 | gagagaggagacatgaag | 2396 | catgttcacca | 2582 | CAGCAACTGCAGC | 2768 | ACGTGGCTCAG | 2954 |
| | g | | accagatgc | | AACTCAG | | TAACATGGG | |
| SluM76 | gagagaggagacatgaag | 2397 | catgttcacca | 2583 | GCAACCCATTAGCC | 2769 | TGAGCACTCCA | 2955 |
| | g | | accagatgc | | CAGACT | | AAATCCCCC | |
| SluM77 | gagagaggagacatgaag | 2398 | catgttcacca | 2584 | GCAACCCATTAGCC | 2770 | GAGCACTCCAA | 2956 |
| | g | | accagatgc | | CAGACT | | AATCCCCCA | |
| SluM78 | gagagaggagacatgaag | 2399 | catgttcacca | 2585 | GCAACCCATTAGCC | 2771 | GAGCACTCCAA | 2957 |
| | g | | accagatgc | | CAGACT | | AATCCCCCA | |
| SluM79 | gagagaggagacatgaag | 2400 | catgttcacca | 2586 | GCAACCCATTAGCC | 2772 | GAGCACTCCAA | 2958 |
| | g | | accagatgc | | CAGACT | | AATCCCCCA | |
| SluM80 | gagagaggagacatgaag | 2401 | catgttcacca | 2587 | GCAACCCATTAGCC | 2773 | GAGCACTCCAA | 2959 |
| | g | | accagatgc | | CAGACT | | AATCCCCCA | |
| SluM81 | gagagaggagacatgaag | 2402 | catgttcacca | 2588 | GCAACCCATTAGCC | 2774 | GAGCACTCCAA | 2960 |
| | g | | accagatgc | | CAGACT | | AATCCCCCA | |
| SluM82 | gagagaggagacatgaag | 2403 | catgttcacca | 2589 | GCAACCCATTAGCC | 2775 | GAGCACTCCAA | 2961 |
| | g | | accagatgc | | CAGACT | | AATCCCCCA | |
| SluM83 | gagagaggagacatgaag | 2404 | catgttcacca | 2590 | GGAAGCTATACCCA | 2776 | CAGGAAGCTGC | 2962 |
| | g | | accagatgc | | CCACCG | | AGGTCTTCA | |
| SluM84 | gagagaggagacatgaag | 2405 | catgttcacca | 2591 | AGCCAGACCAGAC | 2777 | CAGGAAGCTGC | 2963 |
| | g | | accagatgc | | ACACAAC | | AGGTCTTCA | |
| SluM85 | gagagaggagacatgaag | 2406 | catgttcacca | 2592 | AGCCAGACCAGAC | 2778 | CAGGAAGCTGC | 2964 |
| | g | | accagatgc | | ACACAAC | | AGGTCTTCA | |
| SluM86 | gagagaggagacatgaag | 2407 | catgttcacca | 2593 | AGCCAGACCAGAC | 2779 | CAGGAAGCTGC | 2965 |
| | g | | accagatgc | | ACACAAC | | AGGTCTTCA | |
| SluM87 | gagagaggagacatgaag | 2408 | catgttcacca | 2594 | AGCCAGACCAGAC | 2780 | CAGGAAGCTGC | 2966 |
| | g | | accagatgc | | ACACAAC | | AGGTCTTCA | |
| SluM88 | gagagaggagacatgaag | 2409 | catgttcacca | 2595 | AGCCAGACCAGAC | 2781 | CAGGAAGCTGC | 2967 |
| | g | | accagatgc | | ACACAAC | | AGGTCTTCA | |
| SluM89 | gagagaggagacatgaag | 2410 | catgttcacca | 2596 | AGCCAGACCAGAC | 2782 | CAGGAAGCTGC | 2968 |
| | g | | accagatgc | | ACACAAC | | AGGTCTTCA | |
| SluM90 | gagagaggagacatgaag | 2411 | catgttcacca | 2597 | AGCCAGACCAGAC | 2783 | CAGGAAGCTGC | 2969 |
| | g | | accagatgc | | ACACAAC | | AGGTCTTCA | |
| SluM91 | gagagaggagacatgaag | 2412 | catgttcacca | 2598 | AGCCAGACCAGAC | 2784 | CAGGAAGCTGC | 2970 |
| | g | | accagatgc | | ACACAAC | | AGGTCTTCA | |
| SluM92 | ggtgagttcccatgattg | 2413 | caggagcgatt | 2599 | CAGCCCAGACATCC | 2785 | GGGAAGGAGG | 2971 |
| | | | gaaagaca | | ACATGT | | GACATGGAGA | |
| SluM93 | ggtgagttcccatgattg | 2414 | caggagcgatt | 2600 | CAGCCCAGACATCC | 2786 | GGGAAGGAGG | 2972 |
| | | | gaaagaca | | ACATGT | | GACATGGAGA | |
| SluM94 | ggtgagttcccatgattg | 2415 | caggagcgatt | 2601 | CAGCCCAGACATCC | 2787 | GACTGAGCCTG | 2973 |
| | | | gaaagaca | | ACATGT | | GGATTTGCT | |
| SluM95 | ggtgagttcccatgattg | 2416 | caggagcgatt | 2602 | CAGCCCAGACATCC | 2788 | GACTGAGCCTG | 2974 |
| | | | gaaagaca | | ACATGT | | GGATTTGCT | |
| SluM96 | ggtgagttcccatgattg | 2417 | caggagcgatt | 2603 | CAGCCCAGACATCC | 2789 | GACTGAGCCTG | 2975 |
| | | | gaaaagac | | ACATGT | | GGATTTGCT | |
| SluM97 | ggtgagttcccatgattg | 2418 | caggagcgatt | 2604 | CAGCCCAGACATCC | 2790 | GACTGAGCCTG | 2976 |
| | | | gaaaagac | | ACATGT | | GGATTTGCT | |
| SluM98 | ggtgagttcccatgattg | 2419 | caggagcgatt | 2605 | CAGCCCAGACATCC | 2791 | GACTGAGCCTG | 2977 |
| | | | gaaagaca | | ACATGT | | GGATTTGCT | |
| SluM99 | ggtgagttcccatgattg | 2420 | caggagcgatt | 2606 | CAGCCCAGACATCC | 2792 | GACTGAGCCTG | 2978 |
| | | | gaaagaca | | ACATGT | | GGATTTGCT | |
| sluM100 | ggtgagttcccatgattg | 2421 | caggagcgatt | 2607 | CAGCCCAGACATCC | 2793 | GATGGGCTCAT | 2979 |
| | | | gaaagaca | | ACATGT | | GGTCTCTCG | |
| slum101 | ggtgagttcccatgattg | 2422 | caggagcgatt | 2608 | CAGCCCAGACATCC | 2794 | GATGGGCTCAT | 2980 |
| | | | gaaagaca | | ACATGT | | GGTCTCTCG | |
| SluM102 | ggtgagttcccatgattg | 2423 | caggagcgatt | 2609 | CAGCCCAGACATCC | 2795 | GATGGGCTCAT | 2981 |
| | | | gaaagaca | | ACATGT | | GGTCTCTCG | |
| SluM103 | ggtgagttcccatgattg | 2424 | caggagcgatt | 2610 | CAGCCCAGACATCC | 2796 | GATGGGCTCAT | 2982 |
| | | | gaaagaca | | ACATGT | | GGTCTCTCG | |
| SluM104 | ggtgagttcccatgattg | 2425 | caggagcgatt | 2611 | CAGCCCAGACATCC | 2797 | GATGGGCTCAT | 2983 |
| | | | gaaagaca | | ACATGT | | GGTCTCTCG | |
| SluM105 | ggtgagttcccatgattg | 2426 | caggagcgatt | 2612 | CAGCCCAGACATCC | 2798 | GATGGGCTCAT | 2984 |
| | | | gaaagaca | | ACATGT | | GGTCTCTCG | |
| SluM106 | ggtgagttcccatgattg | 2427 | caggagcgatt | 2613 | CAGCCCAGACATCC | 2799 | GATGGGCTCAT | 2985 |
| | | | gaaagaca | | ACATGT | | GGTCTCTCG | |
| SluM107 | ggtgagttcccatgattg | 2428 | caggagcgatt | 2614 | CAGCCCAGACATCC | 2800 | GATGGGCTCAT | 2986 |
| | | | gaaagaca | | ACATGT | | GGTCTCTCG | |
| SluM108 | ggtgagttcccatgattg | 2429 | caggagcgatt | 2615 | CAGCCCAGACATCC | 2801 | GATGGGCTCAT | 2987 |
| | | | gaaagaca | | ACATGT | | GGTCTCTCG | |
| SluM109 | ggtgagttcccatgattg | 2430 | caggagcgatt | 2616 | CAGCCCAGACATCC | 2802 | GATGGGCTCAT | 2988 |
| | | | gaaagaca | | ACATGT | | GGTCTCTCG | |
| slum110 | ggtgagttcccatgattg | 2431 | caggagcgatt | 2617 | CAGCCCAGACATCC | 2803 | GATGGGCTCAT | 2989 |
| | | | gaaagaca | | ACATGT | | GGTCTCTCG | |
| slum111 | ggtgagttcccatgattg | 2432 | caggagcgatt | 2618 | CAGCCCAGACATCC | 2804 | GATGGGCTCAT | 2990 |
| | | | gaaagaca | | ACATGT | | GGTCTCTCG | |
| SluM112 | ggtgagttcccatgattg | 2433 | caggagcgatt | 2619 | CAAGGTGCTGAGA | 2805 | GGTGCTGTTCC | 2991 |
| | | | gaaagaca | | GCCAAGA | | CATGCTTTG | |
| SluM113 | ggtgagttcccatgattg | 2434 | caggagcgatt | 2620 | CAAGGTGCTGAGA | 2806 | GTGCTGTTCCC | 2992 |
| | | | gaaagaca | | GCCAAGA | | ATGCTTTGG | |
| SluM114 | ggtgagttcccatgattg | 2435 | caggagcgatt | 2621 | CAAGGTGCTGAGA | 2807 | GTGCTGTTCCC | 2993 |
| | | | gaaagaca | | GCCAAGA | | ATGCTTTGG | |
| SluM115 | ggtgagttcccatgattg | 2436 | caggagcgatt | 2622 | CAAGGTGCTGAGA | 2808 | GTGCTGTTCCC | 2994 |
| | | | gaaagaca | | GCCAAGA | | ATGCTTTGG | |
| SluM116 | ggtgagttcccatgattg | 2437 | caggagcgatt | 2623 | CAAGGTGCTGAGA | 2809 | GTGCTGTTCCC | 2995 |
| | | | gaaagaca | | GCCAAGA | | ATGCTTTGG | |
| SluM117 | ggtgagttcccatgattg | 2438 | caggagcgatt | 2624 | CAAGGTGCTGAGA | 2810 | GTGCTGTTCCC | 2996 |
| | | | gaaagaca | | GCCAAGA | | ATGCTTTGG | |
| slutm118 | ggtgagttcccatgattg | 2439 | caggagcgatt | 2625 | CAAGGTGCTGAGA | 2811 | GTGCTGTTCCC | 2997 |
| | | | gaaagaca | | GCCAAGA | | ATGCTTTGG | |
| SluM119 | ggtgagttcccatgattg | 2440 | caggagcgatt | 2626 | CAAGGTGCTGAGA | 2812 | GTGCTGTTCCC | 2998 |
| | | | gaaagaca | | GCCAAGA | | ATGCTTTGG | |
| SluM120 | ggtgagttcccatgattg | 2441 | caggagcgatt | 2627 | CAAGGTGCTGAGA | 2813 | GTGCTGTTCCC | 2999 |
| | | | gaaagaca | | GCCAAGA | | ATGCTTTGG | |
| SluM121 | ggtgagttcccatgattg | 2442 | caggagcgatt | 2628 | CAAGGTGCTGAGA | 2814 | GTGCTGTTCCC | 3000 |
| | | | gaaagaca | | GCCAAGA | | ATGCTTTGG | |
| SluM122 | ggtgagttcccatgattg | 2443 | caggagcgatt | 2629 | CAAGGTGCTGAGA | 2815 | GTGCTGTTCCC | 3001 |
| | | | gaaagaca | | GCCAAGA | | ATGCTTTGG | |
| SluM123 | ggtgagttcccatgattg | 2444 | caggagcgatt | 2630 | CAAGGTGCTGAGA | 2816 | GTGCTGTTCCC | 3002 |
| | | | gaaagaca | | GCCAAGA | | ATGCTTTGG | |
| SluM124 | ggtgagttcccatgattg | 2445 | caggagcgatt | 2631 | CAAGGTGCTGAGA | 2817 | GTGCTGTTCCC | 3003 |
| | | | gaaagaca | | GCCAAGA | | ATGCTTTGG | |
| SluM125 | ggtgagttcccatgattg | 2446 | caggagcgatt | 2632 | CAAGGTGCTGAGA | 2818 | GTGCTGTTCCC | 3004 |
| | | | gaaagaca | | GCCAAGA | | ATGCTTTGG | |
| SluM126 | ggtgagttcccatgattg | 2447 | caggagcgatt | 2633 | GGTCTCGAGGTTGT | 2819 | CTGAGTTGCTG | 3005 |
| | | | gaaagaca | | CACTGG | | CAGTTGCTG | |
| SluM127 | ggtgagttcccatgattg | 2448 | caggagcgatt | 2634 | GGTCTCGAGGTTGT | 2820 | CTGAGTTGCTG | 3006 |
| | | | gaaagaca | | CACTGG | | CAGTTGCTG | |
| SluM128 | ggtgagttcccatgattg | 2449 | caggagcgatt | 2635 | GGTCTCGAGGTTGT | 2821 | AGTCTGGGCTA | 3007 |
| | | | gaaagaca | | CACTGG | | ATGGGTTGC | |
| SluM129 | ggtgagttcccatgattg | 2450 | caggagcgatt | 2636 | CCCGGGACATAAA | 2822 | AGTCTGGGCTA | 3008 |
| | | | gaaagaca | | GGTGGAC | | ATGGGTTGC | |
| SluM130 | ggtgagttcccatgattg | 2451 | caggagcgatt | 2637 | GGTCTCGAGGTTGT | 2823 | AGTCTGGGCTA | 3009 |
| | | | gaaagaca | | CACTGG | | ATGGGTTGC | |
| SluM131 | ggtgagttcccatgattg | 2452 | caggagcgatt | 2638 | GGTCTCGAGGTTGT | 2824 | AGTCTGGGCTA | 3010 |
| | | | gaaagaca | | CACTGG | | ATGGGTTGC | |
| SluM132 | ggtgagttcccatgattg | 2453 | caggagcgatt | 2639 | CCCGGGACATAAA | 2825 | AGTCTGGGCTA | 3011 |
| | | | gaaagaca | | GGTGGAC | | ATGGGTTGC | |
| SluM133 | ggtgagttcccatgattg | 2454 | caggagcgatt | 2640 | CCCGGGACATAAA | 2826 | AGTCTGGGCTA | 3012 |
| | | | gaaagaca | | GGTGGAC | | ATGGGTTGC | |
| SluM134 | ggtgagttcccatgattg | 2455 | caggagcgatt | 2641 | CCCGGGACATAAA | 2827 | AGTCTGGGCTA | 3013 |
| | | | gaaagaca | | GGTGGAC | | ATGGGTTGC | |
| SluM135 | ggtgagttcccatgattg | 2456 | caggagcgatt | 2642 | AGTGTGAGTCAGG | 2828 | AGTCTGGGCTA | 3014 |
| | | | gaaagaca | | GGTCAGA | | ATGGGTTGC | |
| SluM136 | ggtgagttcccatgattg | 2457 | caggagcgatt | 2643 | AGTGTGAGTCAGG | 2829 | AGTCTGGGCTA | 3015 |
| | | | gaaagaca | | GGTCAGA | | ATGGGTTGC | |
| SluM137 | ggtgagttcccatgattg | 2458 | caggagcgatt | 2644 | AGTGTGAGTCAGG | 2830 | AGTCTGGGCTA | 3016 |
| | | | gaaagaca | | GGTCAGA | | ATGGGTTGC | |
| SluM138 | ggtgagttcccatgattg | 2459 | caggagcgatt | 2645 | AGTGTGAGTCAGG | 2831 | AGTCTGGGCTA | 3017 |
| | | | gaaagaca | | GGTCAGA | | ATGGGTTGC | |
| SluM139 | ggtgagttcccatgattg | 2460 | caggagcgatt | 2646 | AGTGTGAGTCAGG | 2832 | AGTCTGGGCTA | 3018 |
| | | | gaaagaca | | GGTCAGA | | ATGGGTTGC | |
| SluM140 | ggtgagttcccatgatgt | 2461 | caggagcgatt | 2647 | AGTGTGAGTCAGG | 2833 | AGTCTGGGCTA | 3019 |
| | | | gaaagaca | | GGTCAGA | | ATGGGTTGC | |
| SluM141 | ggtgagttcccatgattg | 2462 | caggagcgatt | 2648 | AGTGTGAGTCAGG | 2834 | AGTCTGGGCTA | 3020 |
| | | | gaaagaca | | GGTCAGA | | ATGGGTTGC | |
| SluM142 | ggtgagttcccatgattg | 2463 | caggagcgatt | 2649 | AGTGTGAGTCAGG | 2835 | AGTCTGGGCTA | 3021 |
| | | | gaaagaca | | GGTCAGA | | ATGGGTTGC | |
| SluM143 | ggtgagttcccatgattg | 2464 | caggagcgatt | 2650 | AGTGTGAGTCAGG | 2836 | AGTCTGGGCTA | 3022 |
| | | | gaaagaca | | GGTCAGA | | ATGGGTTGC | |
| SluM144 | ggtgagttcccatgattg | 2465 | caggagcgatt | 2651 | TCACCAGTTCTGTG | 2837 | CCTGCTTTCTT | 3023 |
| | | | gaaagaca | | GGCATC | | GTGCCTCCT | |
| SluM145 | ggtgagttcccatgattg | 2466 | caggagcgatt | 2652 | TCACCAGTTCTGTG | 2838 | CCTGCTTTCTT | 3024 |
| | | | gaaagaca | | GGCATC | | GTGCCTCCT | |
| SluM146 | ggtgctgacccatgattg | 2467 | caggagcgcttt | 2653 | AGTGTGAGTCAGG | 2839 | AGTCTGGGCTA | 3025 |
| | | | gaaagaca | | GGTCAGA | | ATGGGTTGC | |
| SluM147 | ggtgctgacccatgattg | 2468 | caggagcgcttt | 2654 | TCACCAGTTCTGTG | 2840 | GTTGTGTGTCT | 3026 |
| | | | gaaagaca | | GGCATC | | GGTCTGGCT | |
| SluM148 | ggtgctgacccatgattg | 2469 | caggagcgcttt | 2655 | TCACCAGTTCTGTG | 2841 | GTTGTGTGTCT | 3027 |
| | | | gaaagaca | | GGCATC | | GGTCTGGCT | |
| SluM149 | ggtgctgacccatgattg | 2470 | caggagcgcttt | 2656 | TCACCAGTTCTGTG | 2842 | GTTGTGTGTCT | 3028 |
| | | | gaaagaca | | GGCATC | | GGTCTGGCT | |
| SluM150 | ggtgctgacccatgattg | 2471 | caggagcgcttt | 2657 | TCACCAGTTCTGTG | 2843 | GTTGTGTGTCT | 3029 |
| | | | gaaagaca | | GGCATC | | GGTCTGGCT | |
| SluM151 | ggtgctgacccatgattg | 2472 | caggagcgcttt | 2658 | TCACCAGTTCTGTG | 2844 | GTTGTGTGTCT | 3030 |
| | | | gaaagaca | | GGCATC | | GGTCTGGCT | |
| SluM152 | ggtgctgacccatgattg | 2473 | caggagcgcttt | 2659 | TCACCAGTTCTGTG | 2845 | GTTGTGTGTCT | 3031 |
| | | | gaaagaca | | GGCATC | | GGTCTGGCT | |
| SluM153 | ggtgctgacccatgattg | 2474 | caggagcgcttt | 2660 | TCACCAGTTCTGTG | 2846 | GTTGTGTGTCT | 3032 |
| | | | gaaagaca | | GGCATC | | GGTCTGGCT | |
| SluM154 | ggtgctgacccatgattg | 2475 | caggagcgcttt | 2661 | TCACCAGTTCTGTG | 2847 | GTTGTGTGTCT | 3033 |
| | | | gaaagaca | | GGCATC | | GGTCTGGCT | |
| SluM155 | ggtgctgacccatgattg | 2476 | caggagcgcttt | 2662 | AGCAGCTGGTCCAT | 2848 | CATGTGGATGT | 3034 |
| | | | gaaagaca | | TTACCC | | CTGGGCTGT | |
| SluM156 | ggtgctgacccatgattg | 2477 | caggagcgcttt | 2663 | AGCAGCTGGTCCAT | 2849 | CATGTGGATGT | 3035 |
| | | | gaaagaca | | TTACCC | | CTGGGCTGT | |
| SluM157 | ggtgctgacccatgattg | 2478 | caggagcgcttt | 2664 | AGCAGCTGGTCCAT | 2850 | CATGTGGATGT | 3036 |
| | | | gaaagaca | | TTACCC | | CTGGGCTGT | |
| SluM158 | ggtgctgacccatgattg | 2479 | caggagcgcttt | 2665 | AGCAGCTGGTCCAT | 2851 | CATGTGGATGT | 3037 |
| | | | gaaagaca | | TTACCC | | CTGGGCTGT | |
| SluM159 | ggtgctgacccatgattg | 2480 | caggagcgcttt | 2666 | AGCAGCTGGTCCAT | 2852 | CATGTGGATGT | 3038 |
| | | | gaaagaca | | TTACCC | | CTGGGCTGT | |
| SluM160 | ggtgctgacccatgattg | 2481 | caggagcgcttt | 2667 | AGCAGCTGGTCCAT | 2853 | TCTTGGCTCTC | 3039 |
| | | | gaaagaca | | TTACCC | | AGCACCTTG | |
| SluM161 | ggtgctgacccatgattg | 2482 | caggagcgcttt | 2668 | AGCAGCTGGTCCAT | 2854 | TCTTGGCTCTC | 3040 |
| | | | gaaagaca | | TTACCC | | AGCACCTTG | |
| SluM162 | ggtgctgacccatgattg | 2483 | caggagcgcttt | 2669 | AGCAGCTGGTCCAT | 2855 | TCTTGGCTCTC | 3041 |
| | | | gaaagaca | | TTACCC | | AGCACCTTG | |
| SluM163 | ggtgctgacccatgattg | 2484 | caggagcgcttt | 2670 | AGCAGCTGGTCCAT | 2856 | TCTTGGCTCTC | 3042 |
| | | | gaaagaca | | TTACCC | | AGCACCTTG | |
| SluM164 | ggtgctgacccatgattg | 2485 | caggagcgcttt | 2671 | GGCCATGAGTAGCT | 2857 | TCTTGGCTCTC | 3043 |
| | | | gaaagaca | | TGAGCA | | AGCACCTTG | |
| SluM165 | ggtgctgacccatgattg | 2486 | caggagcgcttt | 2672 | GGCCATGAGTAGCT | 2858 | TCTTGGCTCTC | 3044 |
| | | | gaaagaca | | TGAGCA | | AGCACCTTG | |
| SluM166 | ggtgctgacccatgattg | 2487 | caggagcgcttt | 2673 | GGCCATGAGTAGCT | 2859 | TCTTGGCTCTC | 3045 |
| | | | gaaagaca | | TGAGCA | | AGCACCTTG | |
| SluM167 | ggtgctgacccatgattg | 2488 | caggagcgcttt | 2674 | GGCCATGAGTAGCT | 2860 | TCTTGGCTCTC | 3046 |
| | | | gaaagaca | | TGAGCA | | AGCACCTTG | |
| SluM168 | ggtgctgacccatgattg | 2489 | caggagcgcttt | 2675 | GAGGTGAGCAGAG | 2861 | TCTTGGCTCTC | 3047 |
| | | | gaaagaca | | CTICCTG | | AGCACCTTG | |
| SluM169 | ggtgctgacccatgattg | 2490 | caggagcgcttt | 2676 | GAGGTGAGCAGAG | 2862 | TCTTGGCTCTC | 3048 |
| | | | gaaagaca | | CTICCTG | | AGCACCTTG | |
| SluM170 | ggtgctgacccatgattg | 2491 | caggagcgcttt | 2677 | TTAGTTGGGCTTGG | 2863 | CTTTATGTCCC | 3049 |
| | | | gaaagaca | | TGGGAC | | GGGGAGGTG | |
| SluM171 | ggtgctgacccatgattg | 2492 | caggagcgcttt | 2678 | TTAGTTGGGCTTGG | 2864 | CTTTATGTCCC | 3050 |
| | | | gaaagaca | | TGGGAC | | GGGGAGGTG | |
| SluM172 | ggtgctgacccatgattg | 2493 | caggagcgcttt | 2679 | TTAGTTGGGCTTGG | 2865 | CTTTATGTCCC | 3051 |
| | | | gaaagaca | | TGGGAC | | GGGGAGGTG | |
| SluM173 | ggtgctgacccatgattg | 2494 | caggagcgcttt | 2680 | TTAGTTGGGCTTGG | 2866 | CTTTATGTCCC | 3052 |
| | | | gaaagaca | | TGGGAC | | GGGGAGGTG | |
| SluM174 | ggtgctgacccatgattg | 2495 | caggagcgcttt | 2681 | TTAGTTGGGCTTGG | 2867 | CTTTATGTCCC | 3053 |
| | | | gaaagaca | | TGGGAC | | GGGGAGGTG | |
| SluM175 | ggtgctgacccatgattg | 2496 | caggagcgcttt | 2682 | TTAGTTGGGCTTGG | 2868 | CTTTATGTCCC | 3054 |
| | | | gaaagaca | | TGGGAC | | GGGGAGGTG | |
| SluM176 | ggtgctgacccatgattg | 2497 | caggagcgcttt | 2683 | TTAGTTGGGCTTGG | 2869 | GTCAAACCCTC | 3055 |
| | | | gaaagaca | | TGGGAC | | ACAGGCTCA | |
| SluM177 | ggtgctgacccatgattg | 2498 | caggagcgcttt | 2684 | TTAGTTGGGCTTGG | 2870 | GTCAAACCCTC | 3056 |
| | | | gaaagaca | | TGGGAC | | ACAGGCTCA | |
| SluM178 | ctggagggaagggttag | 2499 | cccagtcagcca | 2685 | ATTGTTCCGTGGGT | 2871 | CAGTGCCAGCA | 3057 |
| | ctc | | caaaatca | | GGAGTC | | AGACTAGCT | |
| SluM179 | ctggagggaagggttag | 2500 | cccagtcagcca | 2686 | ATTGTTCCGTGGGT | 2872 | CAGTGCCAGCA | 3058 |
| | ctc | | caaaatca | | GGAGTC | | AGACTAGCT | |
| SluM180 | ctggagggaagggttag | 2501 | cccagtcagcca | 2687 | ATTGTTCCGTGGGT | 2873 | CAGTGCCAGCA | 3059 |
| | ctc | | caaaatca | | GGAGTC | | AGACTAGCT | |
| SluM181 | ctggagggaagggttag | 2502 | cccagtcagcca | 2688 | ATTGTTCCGTGGGT | 2874 | CAGTGCCAGCA | 3060 |
| | ctc | | caaaatca | | GGAGTC | | AGACTAGCT | |
| SluM182 | ctggagggaagggttag | 2503 | cccagtcagcca | 2689 | TGTTTGCTGTGTAC | 2875 | TGCGCCTGGCT | 3061 |
| | ctc | | caaaatca | | CAGGCA | | AATTTGTTG | |
| SluM183 | ctggagggaagggttag | 2504 | cccagtcagcca | 2690 | TGTTTGCTGTGTAC | 2876 | TGCGCCTGGCT | 3062 |
| | ctc | | caaaatca | | CAGGCA | | AATTTGTTG | |
| SluM184 | ctggagggaagggttag | 2505 | cccagtcagcca | 2691 | TGTTTGCTGTGTAC | 2877 | TGCGCCTGGCT | 3063 |
| | ctc | | caaaatca | | CAGGCA | | AATTTGTTG | |
| SluM185 | ctggagggaagggttag | 2506 | cccagtcagcca | 2692 | TGTTTGCTGTGTAC | 2878 | TGCGCCTGGCT | 3064 |
| | ctc | | caaaatca | | CAGGCA | | AATTTGTTG | |
| SluM186 | ctggagggaagggttag | 2507 | cccagtcagcca | 2693 | CAGGTGATTTTGCC | 2879 | TCCCTGTCTTT | 3065 |
| | ctc | | caaaatca | | CAACCG | | CAAAGCGCT | |

TABLE 26
SluCas9 sgRNAs Categorized Based on Cleavage Efficiency

| Total INDEL % | Guides |
|---|---|
| Not detectable above | SluM8, SluM56, SluM57, SluM85, SluM106, SluM107, SluM108, SluM109, |
| assay threshold of | SluM110, SluM111, SluM114, SluM119, SluM121, SluM122, SluM123, |
| detection | SluM124, SluM125, SluM130, SluM132, SluM136, SluM178 |
| <15% | SluM1, SluM2, SluM3, SluM4, SluM5, SluM6, SluM7, SluM9, SluM10, |
| | SluM12, SluM17, SluM18, SluM19, SluM21, SluM22, SluM24, SluM25, |
| | SluM26, SluM27, SluM30, SluM31, SluM32, SluM33, SluM34, SluM35, |
| | SluM36, SluM37, SluM38, SluM40, SluM41, SluM42, SluM43, SluM44, |
| | SluM45, SluM46, SluM47, SluM48, SluM49, SluM51, SluM52, SluM53, |
| | SluM55, SluM58, SluM59, SluM60, SluM61, SluM62, SluM66, SluM68, |
| | SluM70, SluM72, SluM73, SluM74, SluM75, SluM76, SluM77, SluM81, |
| | SluM82, SluM83, SluM84, SluM86, SluM88, SluM89, SluM90, SluM92, |
| | SluM93, SluM96, SluM97, SluM98, SluM99, SluM100, SluM102, SluM117, |
| | SluM118, SluM128, SluM131, SluM133, SluM134, SluM140, SluM144, |
| | SluM145, SluM147, SluM148, SluM149, SluM154, SluM156, SluM158, |
| | SluM166, SluM167, SluM168, SluM169, SluM177, SluM179, SluM180, |
| | SluM181, SluM183, SluM184 |
| 15%-25% | SluM15, SluM54, SluM69, SluM87, SluM91, SluM101, SluM112, SluM135, |
| | SluM138, SluM139, SluM141, SluM143, SluM146, SluM151, SluM157, |
| | SluM161, SluM170, SluM171, SluM174, SluM175, SluM182, SluM185, |
| | SluM186 |
| >25% | SluM11, SluM13, SluM14, SluM16, SluM20, SluM23, SluM28, SluM29, |
| | SluM39, SluM50, SluM63, SluM64, SluM65, SluM67, SluM71, SluM78, |
| | SluM79, SluM80, SluM94, SluM95, SluM103, SluM104, SluM105, SluM113, |
| | SluM115, SluM116, SluM120, SluM126, SluM127, SluM129, SluM137, |
| | SluM142, SluM150, SluM152, SluM153, SluM155, SluM159, SluM160, |
| | SluM162, SluM163, SluM164, SluM165, SluM172, SluM173, SluM176 |

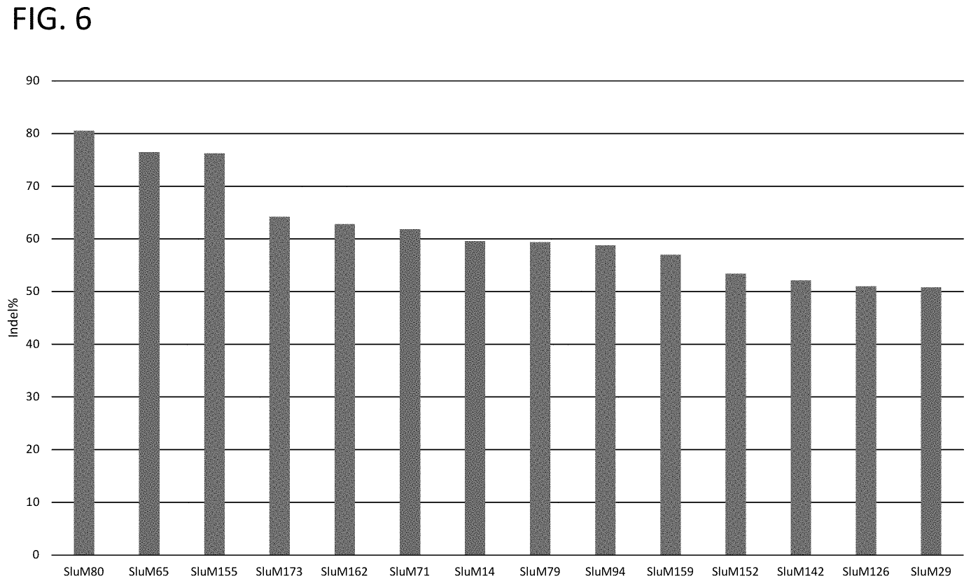

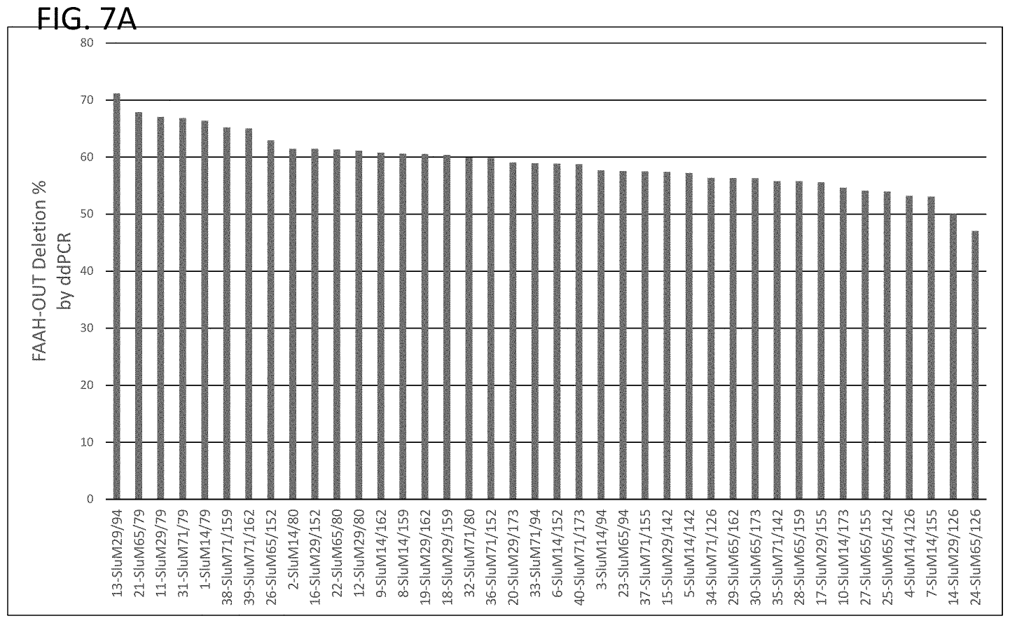

A subset of the SluCas9 sgRNAs was selected for inducing a microdeletion in FAAH-OUT. Specifically, 4 SluCas9 sgRNAs with high overall INDEL frequency and target sites upstream the FOP target sequence were selected as left gRNAs (SluM14, SluM29, SluM65, SluM71); and 10 SluCas9 sgRNAs with high overall INDEL frequency and target sites downstream the FOC target sequence were selected as right gRNAs (SluM79, SluM80, SluM94, SluM126, SluM142, SluM152, SluM155, SluM159, SluM162, SluM173). As shown in FIG. 6, the selected guides are ranked according to overall INDEL frequency at predicted cut sites. The selected SluCas9 sgRNAs and corresponding frequency of INDELs at predicted cut-sites is further identified in Table 27. The 4 left guides and 10 right guides were combined as 40 gRNA pairs to evaluate for inducing a microdeletion in FAAH-OUT. The selected SluCas9 gRNA pairs are identified in Table 28.

TABLE 27
Left and Right SluCas9 sgRNAs Targeting FAAH-OUT

| sgRNA Name | Indel % | L/R* |
|---|---|---|
| SluM14 | 59.55 | L |
| SluM29 | 50.8 | L |
| SluM65 | 76.3 | L |
| SluM71 | 61.85 | L |
| SluM79 | 59.4 | R |
| SluM80 | 80.55 | R |
| SluM94 | 58.8 | R |
| SluM126 | 51 | R |
| SluM142 | 52.15 | R |
| SluM152 | 53.4 | R |
| SluM155 | 76.25 | R |
| SluM159 | 57 | R |
| SluM162 | 62.8 | R |
| SluM173 | 64.2 | R |

*denotes Left (L) or Right (R) gRNA

Combinations of SluCas9 sgRNAs identified in Table 28 were evaluated for inducing a microdeletion in FAAH-OUT. Briefly, 0.3×10⁶ MCF7 cells were electroporated with a left and right sgRNA (1μg per each) and 1.5 μg SluCas9 protein. The cells were incubated 48-72 hours following electroporation, then harvested. Either genomic DNA was extracted for quantification of a genomic deletion in FAAH-OUT by ddPCR as described in Example 6, RNA was extracted for quantification of FAAH mRNA by qPCR as described in Example 2, or protein was extracted for quantification of FAAH protein by Simple Wes as described in Example 2.

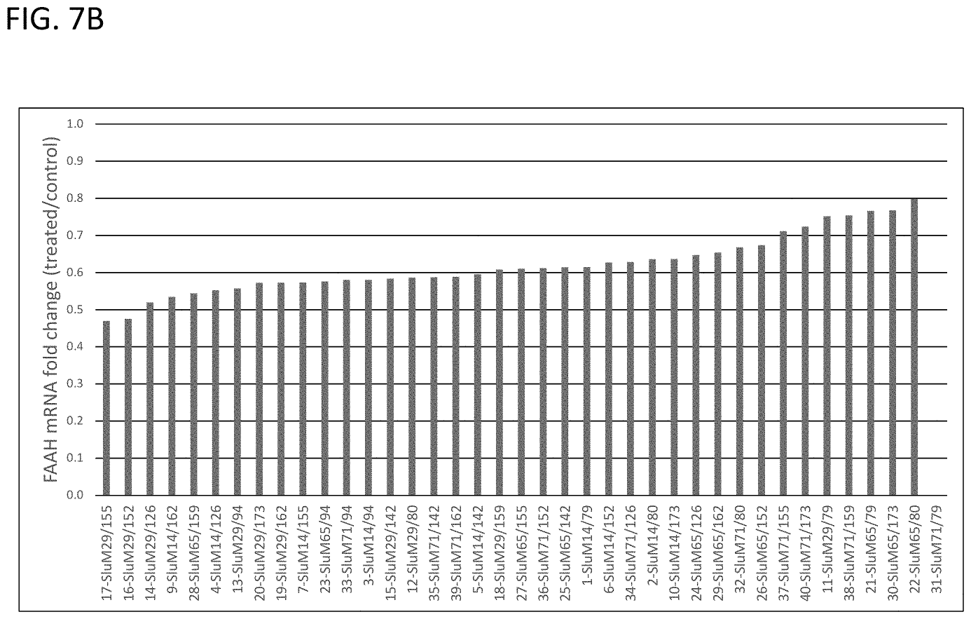

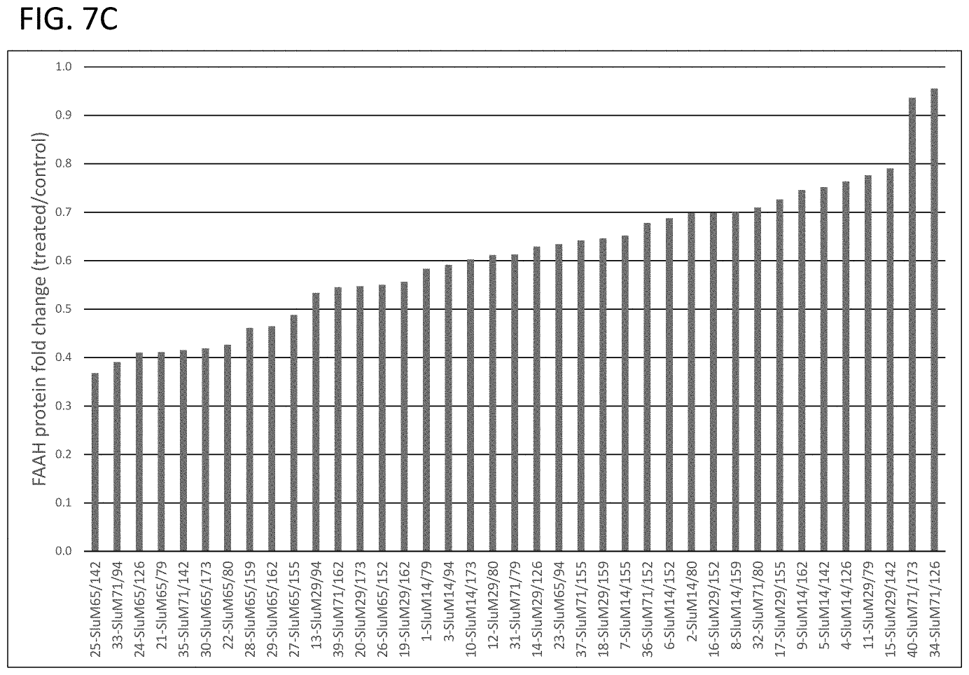

As shown in FIG. 7A, the majority of sgRNA pairs evaluated resulted in a frequency of deletion of FAAH-OUT that exceeded 40%. Quantification of deletion for each sgRNA combination is provided in Table 28.

As shown in FIG. 7B, the FAAH mRNA levels in edited cells, measured as fold change relative to control cells electroporated with SpCas9 only using the 2\hat{ }(−ddCt) method, were reduced by 20% or more for all of the sgRNA combinations tested. Quantification of fold change is provided in Table 28.

As shown in FIG. 7C, the FAAH protein levels were also evaluated, with FAAH-protein normalized to GAPDH levels then calculated as fold change for treated cells relative to PBS control cells. FAAH protein levels were significantly reduced for most of the sgRNA combinations tested. Quantification of fold change in FAAH protein between treated and control samples is provided in Table 28.

TABLE 28
Left and Right SluCas9 sgRNAs Targeting FAAH-OUT

| | | FAAH mRNA | FAAH protein |
|---|---|---|---|
| gRNA pair ID | Deletion (%) | (fold change) | (fold change) |
| 1-SluM14/79 | 66.38 | 0.61472 | 0.5831 |
| 2-SluM14/80 | 61.46 | 0.63591 | 0.6989 |
| 3-SluM14/94 | 57.68 | 0.58022 | 0.5914 |
| 4-SluM14/126 | 53.21 | 0.55215 | 0.7636 |
| 5-SluM14/142 | 57.21 | 0.595 | 0.7514 |
| 6-SluM14/152 | 58.85 | 0.62672 | 0.6875 |
| 7-SluM14/155 | 53.08 | 0.57283 | 0.6517 |
| 8-SluM14/159 | 60.61 | 0.37462 | 0.7002 |
| 9-SluM14/162 | 60.77 | 0.53404 | 0.7458 |
| 10-SluM14/173 | 54.65 | 0.63636 | 0.6026 |
| 11-SluM29/79 | 67.03 | 0.75165 | 0.7761 |
| 12-SluM29/80 | 61.13 | 0.58613 | 0.6117 |
| 13-SluM29/94 | 71.15 | 0.55706 | 0.5334 |
| 14-SluM29/126 | 50.03 | 0.51897 | 0.6288 |
| 15-SluM29/142 | 57.4 | 0.58343 | 0.7904 |
| 16-SluM29/152 | 61.44 | 0.4752 | 0.699 |
| 17-SluM29/155 | 55.6 | 0.46952 | 0.7262 |
| 18-SluM29/159 | 60.39 | 0.60811 | 0.646 |
| 19-SluM29/162 | 60.52 | 0.57229 | 0.5562 |
| 20-SluM29/173 | 59.07 | 0.57218 | 0.5474 |
| 21-SluM65/79 | 67.86 | 0.76598 | 0.4112 |
| 22-SluM65/80 | 61.34 | 0.79858 | 0.4264 |
| 23-SluM65/94 | 57.56 | 0.57588 | 0.634 |
| 24-SluM65/126 | 47.06 | 0.64705 | 0.4103 |
| 25-SluM65/142 | 53.96 | 0.61421 | 0.3679 |
| 26-SluM65/152 | 62.94 | 0.67345 | 0.5503 |
| 27-SluM65/155 | 54.1 | 0.61046 | 0.488 |
| 28-SluM65/159 | 55.79 | 0.54364 | 0.4612 |
| 29-SluM65/162 | 56.32 | 0.65383 | 0.4643 |
| 30-SluM65/173 | 56.3 | 0.7675 | 0.4188 |
| 31-SluM71/79 | 66.84 | NA | 0.6128 |
| 32-SluM71/80 | 59.9 | 0.66776 | 0.7097 |
| 33-SluM71/94 | 58.9 | 0.58014 | 0.3905 |
| 34-SluM71/126 | 56.35 | 0.62803 | 0.9551 |
| 35-SluM71/142 | 55.79 | 0.58714 | 0.4151 |
| 36-SluM71/152 | 59.86 | 0.61155 | 0.6777 |
| 37-SluM71/155 | 57.49 | 0.71143 | 0.6415 |
| 38-SluM71/159 | 65.19 | 0.75368 | 0.342 |
| 39-SluM71/162 | 65.03 | 0.58835 | 0.5449 |
| 40-SluM71/173 | 58.75 | 0.72358 | 0.9359 |

### Example 8: Evaluation of In Vitro Gene Editing of SaCas9 gRNA Targeting FAAH-OUT

Frequency of INDELs induced at predicted cut sites in FAAH-OUT was evaluated following in vitro treatment with complexes of SluCas9 protein and sgRNA with spacers for SaCas9 as identified in Example 5.

Specifically, SaCas9 sgRNA were prepared with spacers shown in Table 20 (SaM1-SaM172; SEQ ID NOs: 1095-1266) inserted into a sgRNA backbone identified by SEQ ID NO: 1271. The SaCas9 sgRNA were provided as sequences that were chemically synthesized and modified by a commercial vendor.

The SaCas9 sgRNA were evaluated for gene-editing of FAAH-OUT in SaCas9-inducible HEK293T cells. The cells were induced to express SaCas9 by treatment with doxycycline at a concentration of 1 μg/mL for 24 hours prior to transfection. The transfection was mediated by Lipofectamine MessengerMax (ThermoFisher #LMRNA008) with SaCas9 sgRNA (200 ng gRNA in 50 k cells per 96-well) for 48-72 hours, and was performed in two biological duplicates. The cells were harvested and genomic DNA was extracted using a Quick DNA Kit—96 (Zymo #D3011). Following DNA quantification, a 1 μl volume containing 30-50 ng of genomic DNA was used for PCR amplification of regions containing predicted cut sites using Q5 Hot Start High Fidelity 2× Master Mix (New England BioLabs #M0494s). The PCR product was purified by AMPure XP PCR Purification (Beckman Coulter #A63881) then sequenced (Genewiz). TIDE PCR and sequencing primers are listed in Table 29.

The guides were categorized based on cleavage efficiency as measured by INDELs introduced at the predicted cut site. As shown in Table 30, guides without detectable cleavage efficiency (frequency of INDELs not detectable above threshold of the assay), with low cleavage efficiency (total frequency of INDELs less than 15%), moderate cleavage efficiency (total frequency of INDELs 15-25%), and high cleavage efficiency (total frequency of INDELs greater than 25%) are indicated.

TABLE 29
TIDE Analysis of SaCas9 gRNAs

| gRNA | PCR | SEQ | PCR | SEQ | | SEQ | | SEQ |
|---|---|---|---|---|---|---|---|---|
| ID NO | forward | ID NO | reverse | ID NO | TIDE seq1 | ID NO | TIDE seq2 | ID NO |
| saM1 | ccctgcccc | 3066 | ttgagcgtg | 3238 | CAGGATCTT | 3410 | GGAGGAGGC | 3582 |
| | ttgttactt | | tgggtttca | | GGCTCACTG | | TGATTTGTG | |
| | tc | | ag | | CA | | CT | |
| saM2 | ccctgcccc | 3067 | ttgagcgtg | 3239 | CAGGATCTT | 3411 | GGAGGAGGC | 3583 |
| | ttgttactt | | tgggtttca | | GGCTCACTG | | TGATTTGTG | |
| | tc | | ag | | CA | | CT | |
| saM3 | ccctgcccc | 3068 | ttgagcgtg | 3240 | CAGGATCTT | 3412 | GGAGGAGGC | 3584 |
| | ttgttactt | | tgggtttca | | GGCTCACTG | | TGATTTGTG | |
| | tc | | ag | | CA | | CT | |
| saM4 | ccctgcccc | 3069 | ttgagcgtg | 3241 | CAGGATCTT | 3413 | GGAGGAGGC | 3585 |
| | ttgttactt | | tgggtttca | | GGCTCACTG | | TGATTTGTG | |
| | tc | | ag | | CA | | CT | |
| saM5 | ccctgcccc | 3070 | ttgagcgtg | 3242 | GGAGGAGGC | 3414 | CAGGATCTT | 3586 |
| | ttgttactt | | tgggtttca | | TGATTTGTG | | GGCTCACTG | |
| | tc | | ag | | CT | | CA | |
| saM6 | ccctgcccc | 3071 | ttgagcgtg | 3243 | GGAGGAGGC | 3415 | CAGGATCTT | 3587 |
| | ttgttactt | | tgggtttca | | TGATTTGTG | | GGCTCACTG | |
| | tc | | ag | | CT | | CA | |
| saM7 | ccctgcccc | 3072 | ttgagcgtg | 3244 | GGCTTAGAG | 3416 | GGAGGAGGC | 3588 |
| | ttgttactt | | tgggtttca | | GATGGTGCT | | TGATTTGTG | |
| | tc | | ag | | CC | | CT | |
| saM8 | ccctgcccc | 3073 | ttgagcgtg | 3245 | GGTGCTGGC | 3417 | AAAGAAGCT | 3589 |
| | ttgttactt | | tgggtttca | | AGTGACAAA | | GTGGCAGTG | |
| | tc | | ag | | TG | | GA | |
| saM9 | ccctgcccc | 3074 | ttgagcgtg | 3246 | GGTGCTGGC | 3418 | AAAGAAGCT | 3590 |
| | ttgttactt | | tgggtttca | | AGTGACAAA | | GTGGCAGTG | |
| | tc | | ag | | TG | | GA | |
| saM10 | ccctgcccc | 3075 | ttgagcgtg | 3247 | GGTGCTGGC | 3419 | AAGCGAGGC | 3591 |
| | ttgttactt | | tgggtttca | | AGTGACAAA | | AAAAAGCTG | |
| | tc | | ag | | TG | | TG | |
| saM11 | ccctgcccc | 3076 | ttgagcgtg | 3248 | GGTGCTGGC | 3420 | AAGCGAGGC | 3592 |
| | ttgttactt | | tgggtttca | | AGTGACAAA | | AAAAAGCTG | |
| | tc | | ag | | TG | | TG | |
| saM12 | ccctgcccc | 3077 | ttgagcgtg | 3249 | AAGCGAGGC | 3421 | GGTGCTGGC | 3593 |
| | ttgttactt | | tgggtttca | | AAAAAGCTG | | AGTGACAAA | |
| | tc | | ag | | TG | | TG | |
| saM13 | ccctgcccc | 3078 | ttgagcgtg | 3250 | TTGCTTTTG | 3422 | GCACAAATC | 3594 |
| | ttgttactt | | tgggtttca | | ACCACGTGC | | AGCCTCCTC | |
| | tc | | ag | | AG | | CT | |
| saM14 | ccctgcccc | 3079 | ttgagcgtg | 3251 | CACAGCTTT | 3423 | CAAAACATA | 3595 |
| | ttgttactt | | tgggtttca | | TTGCCTCGC | | GCCGGGCAC | |
| | tc | | ag | | TT | | AG | |
| saM15 | ccctgcccc | 3080 | ttgagcgtg | 3252 | CACAGCTTT | 3424 | CAAAACATA | 3596 |
| | ttgttactt | | tgggtttca | | TTGCCTCGC | | GCCGGGCAC | |
| | tc | | ag | | TT | | AG | |
| saM16 | ccctgcccc | 3081 | ttgagcgtg | 3253 | CAAAACATA | 3425 | CACAGCTTT | 3597 |
| | ttgttactt | | tgggtttca | | GCCGGGCAC | | TTGCCTCGC | |
| | tc | | ag | | AG | | TT | |
| saM17 | ccctgcccc | 3082 | ttgagcgtg | 3254 | CACAGCTTT | 3426 | CAGCCTGGC | 3598 |
| | ttgttactt | | tgggtttca | | TTGCCTCGC | | CAACATAGT | |
| | tc | | ag | | TT | | GA | |
| saM18 | ccctgcccc | 3083 | ttgagcgtg | 3255 | CACAGCTTT | 3427 | CAGCCTGGC | 3599 |
| | ttgttactt | | tgggtttca | | TTGCCTCGC | | CAACATAGT | |
| | tc | | ag | | TT | | GA | |
| saM19 | ccctgcccc | 3084 | ttgagcgtg | 3256 | CACAGCTTT | 3428 | ATCTCAGCA | 3600 |
| | ttgttactt | | tgggtttca | | TTGCCTCGC | | CTTTGGGAG | |
| | tc | | ag | | TT | | GC | |
| saM20 | ctcatttgg | 3085 | tcacctttc | 3257 | GCACAGTAC | 3429 | TGCACGTGG | 3601 |
| | aaagtgggc | | actcactcc | | ACAGGACTG | | TCAAAAGCA | |
| | att | | cc | | CT | | AG | |
| saM21 | ctcatttgg | 3086 | tcacctttc | 3258 | CTGTGCCCG | 3430 | GTAGGAAAC | 3602 |
| | aaagtgggc | | actcactcc | | GCTATGTTT | | TTGGGAGGG | |
| | att | | cc | | TG | | cc | |
| saM22 | ctcatttgg | 3087 | tcacctttc | 3259 | GTAGGAAAC | 3431 | CTGTGCCCG | 3603 |
| | aaagtgggc | | actcactcc | | TTGGGAGGG | | GCTATGTTT | |
| | att | | cc | | CC | | TG | |
| saM23 | ctcatttgg | 3088 | tcacctttc | 3260 | CATTCTTCG | 3432 | TGGGTGCTG | 3604 |
| | aaagtgggc | | actcactcc | | GACACCAGC | | AGCATACAC | |
| | att | | cc | | CT | | AG | |
| saM24 | ctcatttgg | 3089 | tcacctttc | 3261 | AGCAGTCCT | 3433 | AAAGGAGCA | 3605 |
| | aaagtgggc | | actcactcc | | GTGTACTGT | | AAGGGTCAG | |
| | att | | cc | | GC | | GG | |
| saM25 | ctcatttgg | 3090 | tcacctttc | 3262 | AGCAGTCCT | 3434 | AAAGGAGCA | 3606 |
| | aaagtgggc | | actcactcc | | GTGTACTGT | | AAGGGTCAG | |
| | att | | cc | | GC | | GG | |
| saM26 | ctcatttgg | 3091 | tcacctttc | 3263 | AGCAGTCCT | 3435 | AAAGGAGCA | 3607 |
| | aaagtgggc | | actcactcc | | GTGTACTGT | | AAGGGTCAG | |
| | att | | cc | | GC | | GG | |
| saM27 | ctcatttgg | 3092 | tcacctttc | 3264 | AGCAGTCCT | 3436 | AAAGGAGCA | 3608 |
| | aaagtgggc | | actcactcc | | GTGTACTGT | | AAGGGTCAG | |
| | att | | cc | | GC | | GG | |
| saM28 | ctcatttgg | 3093 | tcacctttc | 3265 | AGCAGTCCT | 3437 | AAAGGAGCA | 3609 |
| | aaagtgggc | | actcactcc | | GTGTACTGT | | AAGGGTCAG | |
| | att | | cc | | GC | | GG | |
| saM29 | ctcatttgg | 3094 | tcacctttc | 3266 | AGCAGTCCT | 3438 | GGAGGCTGA | 3610 |
| | aaagtgggc | | actcactcc | | GTGTACTGT | | GGCAGGAAA | |
| | att | | cc | | GC | | AT | |
| saM30 | ctcatttgg | 3095 | tcacctttc | 3267 | GGAGGCTGA | 3439 | AGCAGTCCT | 3611 |
| | aaagtgggc | | actcactcc | | GGCAGGAAA | | GTGTACTGT | |
| | att | | cc | | AT | | GC | |
| saM31 | ctcatttgg | 3096 | tcacctttc | 3268 | GGTGGATTG | 3440 | AGCAGTCCT | 3612 |
| | aaagtgggc | | actcactcc | | CCTGAGGTC | | GTGTACTGT | |
| | att | | cc | | AA | | GC | |
| saM32 | ctcatttgg | 3097 | tcacctttc | 3269 | GGTGGATTG | 3441 | AGCAGTCCT | 3613 |
| | aaagtgggc | | actcactcc | | CCTGAGGTC | | GTGTACTGT | |
| | att | | cc | | AA | | GC | |
| saM33 | ctcatttgg | 3098 | tcacctttc | 3270 | GGTGGATTG | 3442 | AGCAGTCCT | 3614 |
| | aaagtgggc | | actcactcc | | CCTGAGGTC | | GTGTACTGT | |
| | att | | cc | | AA | | GC | |
| saM34 | ctcatttgg | 3099 | tcacctttc | 3271 | GGTGGATTG | 3443 | AGCAGTCCT | 3615 |
| | aaagtgggc | | actcactcc | | CCTGAGGTC | | GTGTACTGT | |
| | att | | cc | | AA | | GC | |
| saM35 | ctcatttgg | 3100 | tcacctttc | 3272 | GGTGGATTG | 3444 | AGCAGTCCT | 3616 |
| | aaagtgggc | | actcactcc | | CCTGAGGTC | | GTGTACTGT | |
| | att | | cc | | AA | | GC | |
| saM36 | ctcatttgg | 3101 | tcacctttc | 3273 | GGTGGATTG | 3445 | AGCAGTCCT | 3617 |
| | aaagtgggc | | actcactcc | | CCTGAGGTC | | GTGTACTGT | |
| | att | | cc | | AA | | GC | |
| saM37 | ctcatttgg | 3102 | tcacctttc | 3274 | TTTCTCTGG | 3446 | ATGGGTTCA | 3618 |
| | aaagtgggc | | actcactcc | | CTGGGCTTA | | ACTCCACAG | |
| | att | | cc | | GC | | CC | |
| saM38 | ctcatttgg | 3103 | tcacctttc | 3275 | GGCCCTCCC | 3447 | ATGGGTTCA | 3619 |
| | aaagtgggc | | actcactcc | | AAGTTTCCT | | ACTCCACAG | |
| | att | | cc | | AC | | CC | |
| saM39 | ctcatttgg | 3104 | tcacctttc | 3276 | TGGAAGCTC | 3448 | TGTGTATGC | 3620 |
| | aaagtgggc | | actcactcc | | CATTCAGGC | | TCAGCACCC | |
| | att | | cc | | AG | | AG | |
| saM40 | ctcatttgg | 3105 | tcacctttc | 3277 | TGGAAGCTC | 3449 | TGTGTATGC | 3621 |
| | aaagtgggc | | actcactcc | | CATTCAGGC | | TCAGCACCC | |
| | att | | cc | | AG | | AG | |
| saM41 | ctcatttgg | 3106 | tcacctttc | 3278 | CCCTGACCC | 3450 | CTTTCACTC | 3622 |
| | aaagtgggc | | actcactcc | | TTTGCTCCT | | ACTCCCCCA | |
| | att | | cc | | TT | | CC | |
| saM42 | ctcatttgg | 3107 | tcacctttc | 3279 | CCCTGACCC | 3451 | CTTTCACTC | 3623 |
| | aaagtgggc | | actcactcc | | TTTGCTCCT | | ACTCCCCCA | |
| | att | | cc | | TT | | CC | |
| saM43 | ctcatttgg | 3108 | tcacctttc | 3280 | CCCTGACCC | 3452 | CTTTCACTC | 3624 |
| | aaagtgggc | | actcactcc | | TTTGCTCCT | | ACTCCCCCA | |
| | att | | cc | | TT | | CC | |
| saM44 | ctcatttgg | 3109 | tcacctttc | 3281 | CTTTCACTC | 3453 | CCCTGACCC | 3625 |
| | aaagtgggc | | actcactcc | | ACTCCCCCA | | TTTGCTCCT | |
| | att | | cc | | CC | | TT | |
| saM45 | ctcatttgg | 3110 | tcacctttc | 3282 | ATTTTCCTG | 3454 | CTTTCACTC | 3626 |
| | aaagtgggc | | actcactcc | | CCTCAGCCT | | ACTCCCCCA | |
| | att | | cc | | CC | | CC | |
| saM46 | ctcatttgg | 3111 | tcacctttc | 3283 | CTTTCACTC | 3455 | ATTTTCCTG | 3627 |
| | aaagtgggc | | actcactcc | | ACTCCCCCA | | CCTCAGCCT | |
| | att | | cc | | CC | | CC | |
| saM47 | gaccctttg | 3112 | gctcctttg | 3284 | CTTTCACTC | 3456 | ATTTTCCTG | 3628 |
| | ctcctttca | | ttccgcata | | ACTCCCCCA | | CCTCAGCCT | |
| | ca | | ag | | CC | | CC | |
| saM48 | gaccctttg | 3113 | gctcctttg | 3285 | GGCTGTGGA | 3457 | CTTTCACTC | 3629 |
| | ctcctttca | | ttccgcata | | GTTGAACCC | | ACTCCCCCA | |
| | ca | | ag | | AT | | CC | |
| saM49 | gaccctttg | 3114 | gctcctttg | 3286 | GGCTGTGGA | 3458 | CCTTTCACT | 3630 |
| | ctcctttca | | ttccgcata | | GTTGAACCC | | CACTCCCCC | |
| | ca | | ag | | AT | | AC | |
| saM50 | gaccctttg | 3115 | gctcctttg | 3287 | GGCTGTGGA | 3459 | CCTTTCACT | 3631 |
| | ctcctttca | | ttccgcata | | GTTGAACCC | | CACTCCCCC | |
| | ca | | ag | | AT | | AC | |
| saM51 | gaccctttg | 3116 | gctcctttg | 3288 | GGCTGTGGA | 3460 | GCGTTGCTG | 3632 |
| | ctcctttca | | ttccgcata | | GTTGAACCC | | CTAGCTCTT | |
| | ca | | ag | | AT | | TC | |
| saM52 | gaccctttg | 3117 | gctcctttg | 3289 | GGCTGTGGA | 3461 | TTGGGCGGA | 3633 |
| | ctcctttca | | ttccgcata | | GTTGAACCC | | TCAATTGAG | |
| | ca | | ag | | AT | | CT | |
| saM53 | gaccctttg | 3118 | gctcctttg | 3290 | GGCTGTGGA | 3462 | TTGGGCGGA | 3634 |
| | ctcctttca | | ttccgcata | | GTTGAACCC | | TCAATTGAG | |
| | ca | | ag | | AT | | CT | |
| saM54 | gaccctttg | 3119 | gctcctttg | 3291 | GGCTGTGGA | 3463 | TTGGGCGGA | 3635 |
| | ctcctttca | | ttccgcata | | GTTGAACCC | | TCAATTGAG | |
| | ca | | ag | | AT | | CT | |
| saM55 | gaccctttg | 3120 | gctcctttg | 3292 | GGCTGTGGA | 3464 | TTGGGCGGA | 3636 |
| | ctcctttca | | ttccgcata | | GTTGAACCC | | TCAATTGAG | |
| | ca | | ag | | AT | | CT | |
| saM56 | gaccctttg | 3121 | gctcctttg | 3293 | GGCTGTGGA | 3465 | TTGGGCGGA | 3637 |
| | ctcctttca | | ttccgcata | | GTTGAACCC | | TCAATTGAG | |
| | ca | | ag | | AT | | CT | |
| saM57 | gaccctttg | 3122 | gctcctttg | 3294 | GTGCAATCA | 3466 | TTGGGCGGA | 3638 |
| | ctcctttca | | ttccgcata | | AGCAGAAGC | | TCAATTGAG | |
| | ca | | ag | | CC | | CT | |
| saM58 | gaccctttg | 3123 | gctcctttg | 3295 | GTGCAATCA | 3467 | TTGGGCGGA | 3639 |
| | ctcctttca | | ttccgcata | | AGCAGAAGC | | TCAATTGAG | |
| | ca | | ag | | CC | | CT | |
| saM59 | gaccctttg | 3124 | gctcctttg | 3296 | GTGCAATCA | 3468 | TTGGGCGGA | 3640 |
| | ctcctttca | | ttccgcata | | AGCAGAAGC | | TCAATTGAG | |
| | ca | | ag | | CC | | CT | |
| saM60 | gaccctttg | 3125 | gctcctttg | 3297 | TTGGGCGGA | 3469 | GTGCAATCA | 3641 |
| | ctcctttca | | ttccgcata | | TCAATTGAG | | AGCAGAAGC | |
| | ca | | ag | | CT | | cc | |
| saM61 | gaccctttg | 3126 | gctcctttg | 3298 | GGCTGTGGA | 3470 | GGGGGAGTG | 3642 |
| | ctcctttca | | ttccgcata | | GTTGAACCC | | AGTGAAAGG | |
| | ca | | ag | | AT | | TG | |
| saM62 | gaccctttg | 3127 | gctcctttg | 3299 | CAGCAGGTT | 3471 | GGGGGAGTG | 3643 |
| | ctcctttca | | ttccgcata | | AGGGTGGGA | | AGTGAAAGG | |
| | ca | | ag | | AG | | TG | |
| saM63 | gaccctttg | 3128 | gctcctttg | 3300 | AGCTCAATT | 3472 | CAGCAGGTT | 3644 |
| | ctcctttca | | ttccgcata | | GATCCGCCC | | AGGGTGGGA | |
| | ca | | ag | | AA | | AG | |
| saM64 | gaccctttg | 3129 | gctcctttg | 3301 | AGCTCAATT | 3473 | CAGCAGGTT | 3645 |
| | ctcctttca | | ttccgcata | | GATCCGCCC | | AGGGTGGGA | |
| | ca | | ag | | AA | | AG | |
| saM65 | gaccctttg | 3130 | gctcctttg | 3302 | AGCTCAATT | 3474 | TTGACTCCA | 3646 |
| | ctcctttca | | ttccgcata | | GATCCGCCC | | AAGCAAGGC | |
| | ca | | ag | | AA | | CA | |
| saM66 | gaccctttg | 3131 | gctcctttg | 3303 | AGCTCAATT | 3475 | TTGACTCCA | 3647 |
| | ctcctttca | | ttccgcata | | GATCCGCCC | | AAGCAAGGC | |
| | ca | | ag | | AA | | CA | |
| saM67 | gaccctttg | 3132 | gctcctttg | 3304 | AGCTCAATT | 3476 | TTGACTCCA | 3648 |
| | ctcctttca | | ttccgcata | | GATCCGCCC | | AAGCAAGGC | |
| | ca | | ag | | AA | | CA | |
| saM68 | gaccctttg | 3133 | gctcctttg | 3305 | AGCTCAATT | 3477 | AAAGAACAC | 3649 |
| | ctcctttca | | ttccgcata | | GATCCGCCC | | CTGGAGGAG | |
| | ca | | ag | | AA | | CG | |
| saM69 | gaccctttg | 3134 | gctcctttg | 3306 | AGCTCAATT | 3478 | AAAGAACAC | 3650 |
| | ctcctttca | | ttccgcata | | GATCCGCCC | | CTGGAGGAG | |
| | ca | | ag | | AA | | CG | |
| saM70 | gaccctttg | 3135 | gctcctttg | 3307 | AAAGAACAC | 3479 | AGCTCAATT | 3651 |
| | ctcctttca | | ttccgcata | | CTGGAGGAG | | GATCCGCCC | |
| | ca | | ag | | CG | | AA | |
| saM71 | gaccctttg | 3136 | gctcctttg | 3308 | AAAGAACAC | 3480 | AGCTCAATT | 3652 |
| | ctcctttca | | ttccgcata | | CTGGAGGAG | | GATCCGCCC | |
| | ca | | ag | | CG | | AA | |
| saM72 | gaccctttg | 3137 | gctcctttg | 3309 | AAAGAACAC | 3481 | AGCTCAATT | 3653 |
| | ctcctttca | | ttccgcata | | CTGGAGGAG | | GATCCGCCC | |
| | ca | | ag | | CG | | AA | |
| saM73 | gaccctttg | 3138 | gctcctttg | 3310 | AAAGAACAC | 3482 | AGCTCAATT | 3654 |
| | ctcctttca | | ttccgcata | | CTGGAGGAG | | GATCCGCCC | |
| | ca | | ag | | CG | | AA | |
| saM74 | gaccctttg | 3139 | gctcctttg | 3311 | AAAGAACAC | 3483 | AGCTCAATT | 3655 |
| | ctcctttca | | ttccgcata | | CTGGAGGAG | | GATCCGCCC | |
| | ca | | ag | | CG | | AA | |
| saM75 | gaccctttg | 3140 | gctcctttg | 3312 | AAAGAACAC | 3484 | AGCTCAATT | 3656 |
| | ctcctttca | | ttccgcata | | CTGGAGGAG | | GATCCGCCC | |
| | ca | | ag | | CG | | AA | |
| saM76 | gaccctttg | 3141 | gctcctttg | 3313 | AAAGAACAC | 3485 | AGCTCAATT | 3657 |
| | ctcctttca | | ttccgcata | | CTGGAGGAG | | GATCCGCCC | |
| | ca | | ag | | CG | | AA | |
| saM77 | gaccctttg | 3142 | gctcctttg | 3314 | ACAGAGTGG | 3486 | CCCACCCCT | 3658 |
| | ctcctttca | | ttccgcata | | GCAACTTCA | | CAGATTCCC | |
| | ca | | ag | | GG | | TA | |
| saM78 | gagagctgg | 3143 | catgttcac | 3315 | CCCTCGGGT | 3487 | CCCACCCCT | 3659 |
| | agttcatga | | caaccagat | | ACTCAGACT | | CAGATTCCC | |
| | agg | | ag | | CA | | TA | |
| saM79 | gagagctgg | 3144 | catgttcac | 3316 | CCCTCGGGT | 3488 | CCCACCCCT | 3660 |
| | agttcatga | | caaccagat | | ACTCAGACT | | CAGATTCCC | |
| | agg | | gc | | CA | | TA | |
| saM80 | gagagctgg | 3145 | catgttcac | 3317 | CCCTCGGGT | 3489 | CCCACCCCT | 3661 |
| | agttcatga | | caaccagat | | ACTCAGACT | | CAGATTCCC | |
| | agg | | gc | | CA | | TA | |
| saM81 | gagagctgg | 3146 | catgttcac | 3318 | CCCTCGGGT | 3490 | ACTCCCACC | 3662 |
| | agttcatga | | caaccagat | | ACTCAGACT | | CATCCTACC | |
| | agg | | gc | | CA | | TC | |
| saM82 | gagagctgg | 3147 | catgttcac | 3319 | CCCTCGGGT | 3491 | ACTCCCACC | 3663 |
| | agttcatga | | caaccagat | | ACTCAGACT | | CATCCTACC | |
| | agg | | gc | | CA | | TC | |
| saM83 | gagagctgg | 3148 | catgttcac | 3320 | CCCTCGGGT | 3492 | CCTGGCGAC | 3664 |
| | agttcatga | | caaccagat | | ACTCAGACT | | AAAACCCCT | |
| | agg | | gc | | CA | | AT | |
| saM84 | gagagctgg | 3149 | catgttcac | 3321 | CCTGGCGAC | 3493 | CCCTCGGGT | 3665 |
| | agttcatga | | caaccagat | | AAAACCCCT | | ACTCAGACT | |
| | agg | | gc | | AT | | CA | |
| saM85 | gagagctgg | 3150 | catgttcac | 3322 | CAGCTGCTG | 3494 | CCCTCGGGT | 3666 |
| | agttcatga | | caaccagat | | TTTCCTCAG | | ACTCAGACT | |
| | agg | | gc | | GA | | CA | |
| saM86 | gagagctgg | 3151 | catgttcac | 3323 | CAGCTGCTG | 3495 | CCCTCGGGT | 3667 |
| | agttcatga | | caaccagat | | TTTCCTCAG | | ACTCAGACT | |
| | agg | | gc | | GA | | CA | |
| saM87 | gagagctgg | 3152 | catgttcac | 3324 | CAGCTGCTG | 3496 | CCCTCGGGT | 3668 |
| | agttcatga | | caaccagat | | TTTCCTCAG | | ACTCAGACT | |
| | agg | | gc | | GA | | CA | |
| saM88 | gagagctgg | 3153 | catgttcac | 3325 | CGACCTGTG | 3497 | CCCTCGGGT | 3669 |
| | agttcatga | | caaccagat | | TGAATCCAG | | ACTCAGACT | |
| | agg | | gc | | CT | | CA | |
| saM89 | gagagctgg | 3154 | catgttcac | 3326 | CGACCTGTG | 3498 | CCCTCGGGT | 3670 |
| | agttcatga | | caaccagat | | TGAATCCAG | | ACTCAGACT | |
| | agg | | gc | | CT | | CA | |
| saM90 | gagagctgg | 3155 | catgttcac | 3327 | TACCTCCCT | 3499 | CTTCCCACC | 3671 |
| | agttcatga | | caaccagat | | CCACTTCTG | | CTAACCTGC | |
| | agg | | gc | | GG | | TG | |
| saM91 | gagagctgg | 3156 | catgttcac | 3328 | TACCTCCCT | 3500 | CTTCCCACC | 3672 |
| | agttcatga | | caaccagat | | CCACTTCTG | | CTAACCTGC | |
| | agg | | gc | | GG | | TG | |
| saM92 | gagagctgg | 3157 | catgttcac | 3329 | TACCTCCCT | 3501 | CTTCCCACC | 3673 |
| | agttcatga | | caaccagat | | CCACTTCTG | | CTAACCTGC | |
| | agg | | gc | | GG | | TG | |
| saM93 | gagagctgg | 3158 | catgttcac | 3330 | CGCTCCTCC | 3502 | TACCTCCCT | 3674 |
| | agttcatga | | caaccagat | | AGGTGTTCT | | CCACTTCTG | |
| | agg | | gc | | TT | | GG | |
| saM94 | gagagctgg | 3159 | catgttcac | 3331 | CGCTCCTCC | 3503 | TACCTCCCT | 3675 |
| | agttcatga | | caaccagat | | AGGTGTTCT | | CCACTTCTG | |
| | agg | | gc | | TT | | GG | |
| saM95 | gagagctgg | 3160 | catgttcac | 3332 | CCAGGTCCT | 3504 | CGCTCCTCC | 3676 |
| | agttcatga | | caaccagat | | CCATCTTCT | | AGGTGTTCT | |
| | agg | | gc | | GC | | TT | |
| saM96 | gagagctgg | 3161 | catgttcac | 3333 | TAGGGAATC | 3505 | CCCATGTTA | 3677 |
| | agttcatga | | caaccagat | | TGAGGGGTG | | CTGAGCCAC | |
| | agg | | gc | | GG | | GT | |
| saM97 | gagagctgg | 3162 | catgttcac | 3334 | TAGGGAATC | 3506 | CCCATGTTA | 3678 |
| | agttcatga | | caaccagat | | TGAGGGGTG | | CTGAGCCAC | |
| | agg | | gc | | GG | | GT | |
| saM98 | gagagctgg | 3163 | catgttcac | 3335 | TAGGGAATC | 3507 | CCCATGTTA | 3679 |
| | agttcatga | | caaccagat | | TGAGGGGTG | | CTGAGCCAC | |
| | agg | | gc | | GG | | GT | |
| saM99 | gagagctgg | 3164 | catgttcac | 3336 | TAGGGAATC | 3508 | CCCATGTTA | 3680 |
| | agttcatga | | caaccagat | | TGAGGGGTG | | CTGAGCCAC | |
| | agg | | gc | | GG | | GT | |
| saM100 | gagagctgg | 3165 | catgttcac | 3337 | TAGGGAATC | 3509 | CCCATGTTA | 3681 |
| | agttcatga | | caaccagat | | TGAGGGGTG | | CTGAGCCAC | |
| | agg | | gc | | GG | | GT | |
| saM101 | gagagctgg | 3166 | catgttcac | 3338 | TAGGGAATC | 3510 | CCCATGTTA | 3682 |
| | agttcatga | | caaccagat | | TGAGGGGTG | | CTGAGCCAC | |
| | agg | | gc | | GG | | GT | |
| saM102 | gagagctgg | 3167 | catgttcac | 3339 | TAGGGAATC | 3511 | TGAAGACCT | 3683 |
| | agttcatga | | caaccagat | | TGAGGGGTG | | GCAGCTTCC | |
| | agg | | gc | | GG | | TG | |
| saM103 | gagagctgg | 3168 | catgttcac | 3340 | TAGGGAATC | 3512 | TGAAGACCT | 3684 |
| | agttcatga | | caaccagat | | TGAGGGGTG | | GCAGCTTCC | |
| | agg | | gc | | GG | | TG | |
| saM104 | gagagctgg | 3169 | catgttcac | 3341 | TAGGGAATC | 3513 | TGAAGACCT | 3685 |
| | agttcatga | | caaccagat | | TGAGGGGTG | | GCAGCTTCC | |
| | agg | | gc | | GG | | TG | |
| saM105 | gagagctgg | 3170 | catgttcac | 3342 | TAGGGAATC | 3514 | TGAAGACCT | 3686 |
| | agttcatga | | caaccagat | | TGAGGGGTG | | GCAGCTTCC | |
| | agg | | gc | | GG | | TG | |
| saM106 | gagagctgg | 3171 | catgttcac | 3343 | TGAAGACCT | 3515 | TAGGGAATC | 3687 |
| | agttcatga | | caaccagat | | GCAGCTTCC | | TGAGGGGTG | |
| | agg | | gc | | TG | | GG | |
| saM107 | gagagctgg | 3172 | catgttcac | 3344 | TGAAGACCT | 3516 | TAGGGAATC | 3688 |
| | agttcatga | | caaccagat | | GCAGCTTCC | | TGAGGGGTG | |
| | agg | | gc | | TG | | GG | |
| saM108 | gagagctgg | 3173 | catgttcac | 3345 | CTGAGGAAA | 3517 | CGAGAGACC | 3689 |
| | agttcatga | | caaccagat | | CAGCAGCTG | | ATGAGCCCA | |
| | agg | | gc | | GA | | TC | |
| saM109 | gagagctgg | 3174 | catgttcac | 3346 | CTGAGGAAA | 3518 | CGAGAGACC | 3690 |
| | agttcatga | | caaccagat | | CAGCAGCTG | | ATGAGCCCA | |
| | agg | | gc | | GA | | TC | |
| saM110 | gagagctgg | 3175 | catgttcac | 3347 | CTGAGGAAA | 3519 | CGAGAGACC | 3691 |
| | agttcatga | | caaccagat | | CAGCAGCTG | | ATGAGCCCA | |
| | agg | | gc | | GA | | TC | |
| saM111 | gagagctgg | 3176 | catgttcac | 3348 | CGAGAGACC | 3520 | TGATGCTAC | 3692 |
| | agttcatga | | caaccagat | | ATGAGCCCA | | TTCTGCTGG | |
| | agg | | gc | | TC | | CC | |
| saM112 | gagagctgg | 3177 | catgttcac | 3349 | CGAGAGACC | 3521 | TGATGCTAC | 3693 |
| | agttcatga | | caaccagat | | ATGAGCCCA | | TTCTGCTGG | |
| | agg | | gc | | TC | | CC | |
| saM113 | gagagctgg | 3178 | catgttcac | 3350 | CGAGAGACC | 3522 | TGATGCTAC | 3694 |
| | agttcatga | | caaccagat | | ATGAGCCCA | | TTCTGCTGG | |
| | gagagctgg | | gc | | TC | | CC | |
| saM114 | gagagctgg | 3179 | catgttcac | 3351 | GCAACCCAT | 3523 | ACGTGGCTC | 3695 |
| | agttcatga | | caaccagat | | TAGCCCAGA | | AGTAACATG | |
| | agg | | gc | | CT | | GG | |
| saM115 | gagagctgg | 3180 | catgttcac | 3352 | GCAACCCAT | 3524 | ACGTGGCTC | 3696 |
| | agttcatga | | caaccagat | | TAGCCCAGA | | AGTAACATG | |
| | agg | | gc | | CT | | GG | |
| saM116 | gagagctgg | 3181 | catgttcac | 3353 | CAGGAAGCT | 3525 | GCAACCCAT | 3697 |
| | agttcatga | | caaccagat | | GCAGGTCTT | | TAGCCCAGA | |
| | agg | | gc | | CA | | CT | |
| saM117 | gagagctgg | 3182 | catgttcac | 3354 | CAGGAAGCT | 3526 | GGAAGCTAT | 3698 |
| | agttcatga | | caaccagat | | GCAGGTCTT | | ACCCACCAC | |
| | agg | | gc | | CA | | CG | |
| saM118 | gagagctgg | 3183 | catgttcac | 3355 | CAGGAAGCT | 3527 | CAGCCCAGA | 3699 |
| | agttcatga | | caaccagat | | GCAGGTCTT | | CATCCACAT | |
| | agg | | gc | | CA | | GT | |
| saM119 | gagagctgg | 3184 | catgttcac | 3356 | CAGGAAGCT | 3528 | CAGCCCAGA | 3700 |
| | agttcatga | | caaccagat | | GCAGGTCTT | | CATCCACAT | |
| | agg | | gc | | CA | | GT | |
| saM120 | gagagctgg | 3185 | catgttcac | 3357 | CAGGAAGCT | 3529 | CAGCCCAGA | 3701 |
| | agttcatga | | caaccagat | | GCAGGTCTT | | CATCCACAT | |
| | agg | | gc | | CA | | GT | |
| saM121 | gagagctgg | 3186 | catgttcac | 3358 | GACTGAGCC | 3530 | CAGCCCAGA | 3702 |
| | agttcatga | | caaccagat | | TGGGATTTG | | CATCCACAT | |
| | agg | | gc | | CT | | GT | |
| saM122 | gagagctgg | 3187 | catgttcac | 3359 | GACTGAGCC | 3531 | CAGCCCAGA | 3703 |
| | agttcatga | | caaccagat | | TGGGATTTG | | CATCCACAT | |
| | agg | | gc | | CT | | GT | |
| saM123 | gagagctgg | 3188 | catgttcac | 3360 | GACTGAGCC | 3532 | CAGCCCAGA | 3704 |
| | agttcatga | | caaccagat | | TGGGATTTG | | CATCCACAT | |
| | agg | | gc | | CT | | GT | |
| saM124 | gagagctgg | 3189 | catgttcac | 3361 | GATGGGCTC | 3533 | CAGCCCAGA | 3705 |
| | agttcatga | | caaccagat | | ATGGTCTCT | | CATCCACAT | |
| | agg | | gc | | CG | | GT | |
| saM125 | gagagctgg | 3190 | catgttcac | 3362 | GATGGGCTC | 3534 | CAGCCCAGA | 3706 |
| | agttcatga | | caaccagat | | ATGGTCTCT | | CATCCACAT | |
| | agg | | gc | | CG | | GT | |
| saM126 | gagagctgg | 3191 | catgttcac | 3363 | GATGGGCTC | 3535 | CAAGGTGCT | 3707 |
| | agttcatga | | caaccagat | | ATGGTCTCT | | GAGAGCCAA | |
| | agg | | gc | | CG | | GA | |
| saM127 | ggtgctgtt | 3192 | caggagcgc | 3364 | CAAGGTGCT | 3536 | GATGGGCTC | 3708 |
| | cccatgctt | | tttgaaaga | | GAGAGCCAA | | ATGGTCTCT | |
| | tg | | ca | | GA | | CG | |
| saM128 | ggtgctgtt | 3193 | caggagcgc | 3365 | GGTCTCGAG | 3537 | GATGGGCTC | 3709 |
| | cccatgctt | | tttgaaaga | | GTTGTCACT | | ATGGTCTCT | |
| | tg | | ca | | GG | | CG | |
| saM129 | ggtgctgtt | 3194 | caggagcgc | 3366 | GGTCTCGAG | 3538 | GATGGGCTC | 3710 |
| | cccatgctt | | tttgaaaga | | GTTGTCACT | | ATGGTCTCT | |
| | tg | | ca | | GG | | CG | |
| saM130 | ggtgctgtt | 3195 | caggagcgc | 3367 | GGTCTCGAG | 3539 | GATGGGCTC | 3711 |
| | cccatgctt | | tttgaaaga | | GTTGTCACT | | ATGGTCTCT | |
| | tg | | ca | | GG | | CG | |
| saM131 | ggtgctgtt | 3196 | caggagcgc | 3368 | GGTCTCGAG | 3540 | GATGGGCTC | 3712 |
| | cccatgctt | | tttgaaaga | | GTTGTCACT | | ATGGTCTCT | |
| | tg | | ca | | GG | | CG | |
| saM132 | ggtgctgtt | 3197 | caggagcgc | 3369 | GGTCTCGAG | 3541 | GATGGGCTC | 3713 |
| | cccatgctt | | tttgaaaga | | GTTGTCACT | | ATGGTCTCT | |
| | tg | | ca | | GG | | CG | |
| saM133 | ggtgctgtt | 3198 | caggagcgc | 3370 | AGTCTGGGC | 3542 | TCACCAGTT | 3714 |
| | cccatgctt | | tttgaaaga | | TAATGGGTT | | CTGTGGGCA | |
| | tg | | ca | | GC | | TC | |
| saM134 | ggtgctgtt | 3199 | caggagcgc | 3371 | AGTCTGGGC | 3543 | GCCATGAGG | 3715 |
| | cccatgctt | | tttgaaaga | | TAATGGGTT | | TTCAGCTCA | |
| | tg | | ca | | GC | | CT | |
| saM135 | ggtgctgtt | 3200 | caggagcgc | 3372 | AGTCTGGGC | 3544 | GCCATGAGG | 3716 |
| | cccatgctt | | tttgaaaga | | TAATGGGTT | | TTCAGCTCA | |
| | tg | | ca | | GC | | CT | |
| saM136 | ggtgctgtt | 2001 | caggagcgc | 3373 | AGTCTGGGC | 3545 | GCCATGAGG | 3717 |
| | cccatgctt | | tttgaaaga | | TAATGGGTT | | TTCAGCTCA | |
| | tg | | ca | | GC | | CT | |
| saM137 | ggtgctgtt | 2002 | caggagcgc | 3374 | CATGTGGAT | 3546 | AGCAGCTGG | 3718 |
| | cccatgctt | | tttgaaaga | | GTCTGGGCT | | TCCATTTAC | |
| | tg | | ca | | GT | | CC | |
| saM138 | ggtgctgtt | 2003 | caggagcgc | 3375 | CATGTGGAT | 3547 | AGCAGCTGG | 3719 |
| | cccatgctt | | tttgaaaga | | GTCTGGGCT | | TCCATTTAC | |
| | tg | | ca | | GT | | CC | |
| saM139 | ggtgctgtt | 2004 | caggagcgc | 3376 | CATGTGGAT | 3548 | AGCAGCTGG | 3720 |
| | cccatgctt | | tttgaaaga | | GTCTGGGCT | | TCCATTTAC | |
| | tg | | ca | | GT | | CC | |
| saM140 | ggtgctgtt | 2005 | caggagcgc | 3377 | CATGTGGAT | 3549 | AGCAGCTGG | 3721 |
| | cccatgctt | | tttgaaaga | | GTCTGGGCT | | TCCATTTAC | |
| | tg | | ca | | GT | | CC | |
| saM141 | ggtgctgtt | 2006 | caggagcgc | 3378 | CATGTGGAT | 3550 | AGCAGCTGG | 3722 |
| | cccatgctt | | tttgaaaga | | GTCTGGGCT | | TCCATTTAC | |
| | tg | | ca | | GT | | CC | |
| saM142 | ggtgctgtt | 2007 | caggagcgc | 3379 | CATGTGGAT | 3551 | AGCAGCTGG | 3723 |
| | cccatgctt | | tttgaaaga | | GTCTGGGCT | | TCCATTTAC | |
| | tg | | ca | | GT | | CC | |
| saM143 | ggtgctgtt | 2008 | caggagcgc | 3380 | CATGTGGAT | 3552 | AGCAGCTGG | 3724 |
| | cccatgctt | | tttgaaaga | | GTCTGGGCT | | TCCATTTAC | |
| | tg | | ca | | GT | | CC | |
| saM144 | ggtgctgtt | 2009 | caggagcgc | 3381 | AGCAGCTGG | 3553 | CATGTGGAT | 3725 |
| | cccatgctt | | tttgaaaga | | TCCATTTAC | | GTCTGGGCT | |
| | tg | | ca | | CC | | GT | |
| saM145 | ggtgctgtt | 2010 | caggagcgc | 3382 | GAGGTGAGC | 3554 | CATGTGGAT | 3726 |
| | cccatgctt | | tttgaaaga | | AGAGCTTCC | | GTCTGGGCT | |
| | tg | | ca | | TG | | GT | |
| saM146 | ggtgctgtt | 2011 | caggagcgc | 3383 | TTAGTTGGG | 3555 | CATGTGGAT | 3727 |
| | cccatgctt | | tttgaaaga | | CTTGGTGGG | | GTCTGGGCT | |
| | tg | | ca | | AC | | GT | |
| saM147 | ggtgctgtt | 2012 | caggagcgc | 3384 | TTAGTTGGG | 3556 | CATGTGGAT | 3728 |
| | cccatgctt | | tttgaaaga | | CTTGGTGGG | | GTCTGGGCT | |
| | tg | | ca | | AC | | GT | |
| saM148 | ggtgctgtt | 2013 | caggagcgc | 3385 | TTAGTTGGG | 3557 | CATGTGGAT | 3729 |
| | cccatgctt | | tttgaaaga | | CTTGGTGGG | | GTCTGGGCT | |
| | tg | | ca | | AC | | GT | |
| saM149 | ggtgctgtt | 2014 | caggagcgc | 3386 | GATGCCCAC | 3558 | CTCTGTACT | 3730 |
| | cccatgctt | | tttgaaaga | | AGAACTGGT | | CAGGGTGCT | |
| | tg | | ca | | GA | | GC | |
| saM150 | ggtgctgtt | 2015 | caggagcgc | 3387 | GATGCCCAC | 3559 | CTCTGTACT | 3731 |
| | cccatgctt | | tttgaaaga | | AGAACTGGT | | CAGGGTGCT | |
| | tg | | ca | | GA | | GC | |
| saM151 | ggtgctgtt | 2016 | caggagcgc | 3388 | GATGCCCAC | 3560 | CTCTGTACT | 3732 |
| | cccatgctt | | tttgaaaga | | AGAACTGGT | | CAGGGTGCT | |
| | tg | | ca | | GA | | GC | |
| saM152 | ggtgctgtt | 2017 | caggagcgc | 3389 | GATGCCCAC | 3561 | CTCTGTACT | 3733 |
| | cccatgctt | | tttgaaaga | | AGAACTGGT | | CAGGGTGCT | |
| | tg | | ca | | GA | | GC | |
| saM153 | ggtgctgtt | 2018 | caggagcgc | 3390 | AGTGAGCTG | 3562 | CTCTGTACT | 3734 |
| | cccatgctt | | tttgaaaga | | AACCTCATG | | CAGGGTGCT | |
| | tg | | ca | | GC | | GC | |
| saM154 | ggtgctgtt | 2019 | caggagcgc | 3391 | GTTCGAGAC | 3563 | GGGTAAATG | 3735 |
| | cccatgctt | | tttgaaaga | | CAGCCTCAA | | GACCAGCTG | |
| | tg | | ca | | CA | | CT | |
| saM155 | ggtgctgtt | 2020 | caggagcgc | 3392 | GTTCGAGAC | 3564 | GGGTAAATG | 3736 |
| | cccatgctt | | tttgaaaga | | CAGCCTCAA | | GACCAGCTG | |
| | tg | | ca | | CA | | CT | |
| saM156 | ggtgctgtt | 2021 | caggagcgc | 3393 | GTTCGAGAC | 3565 | GGGTAAATG | 3737 |
| | cccatgctt | | tttgaaaga | | CAGCCTCAA | | GACCAGCTG | |
| | tg | | ca | | CA | | CT | |
| saM157 | ggtgctgtt | 2022 | caggagcgc | 3394 | GTTCGAGAC | 3566 | GGGTAAATG | 3738 |
| | cccatgctt | | tttgaaaga | | CAGCCTCAA | | GACCAGCTG | |
| | tg | | ca | | CA | | CT | |
| saM158 | ctggaggga | 2023 | cccagtcag | 3395 | GGAGTCTGA | 3567 | TGTTGAGGC | 3739 |
| | agggttagc | | ccacaaaat | | GGTGGGAGG | | TGGTCTCGA | |
| | tc | | ca | | AT | | AC | |
| saM159 | ctggaggga | 2024 | cccagtcag | 3396 | GGAGTCTGA | 3568 | TGTTGAGGC | 3740 |
| | agggttagc | | ccacaaaat | | GGTGGGAGG | | TGGTCTCGA | |
| | tc | | ca | | AT | | AC | |
| saM160 | ctggaggga | 2025 | cccagtcag | 3397 | GGAGTCTGA | 3569 | TGTTGAGGC | 3741 |
| | agggttagc | | ccacaaaat | | GGTGGGAGG | | TGGTCTCGA | |
| | tc | | ca | | AT | | AC | |
| saM161 | ctggaggga | 2026 | cccagtcag | 3398 | GGAGTCTGA | 3570 | TGTTGAGGC | 3742 |
| | agggttagc | | ccacaaaat | | GGTGGGAGG | | TGGTCTCGA | |
| | tc | | ca | | AT | | AC | |
| saM162 | ctggaggga | 2027 | cccagtcag | 3399 | TGCGCCTGG | 3571 | CAGGTGATT | 3743 |
| | agggttagc | | ccacaaaat | | CTAATTTGT | | TTGCCCAAC | |
| | tc | | ca | | TG | | CG | |
| saM163 | ctggaggga | 2028 | cccagtcag | 3400 | TCCCTGTCT | 3572 | CAGGTGATT | 3744 |
| | agggttagc | | ccacaaaat | | TTCAAAGCG | | TTGCCCAAC | |
| | tc | | ca | | CT | | CG | |
| saM164 | ctggaggga | 2029 | cccagtcag | 3401 | ATTGTTCCG | 3573 | CAGTGCCAG | 3745 |
| | agggttagc | | ccacaaaat | | TGGGTGGAG | | CAAGACTAG | |
| | tc | | ca | | TC | | CT | |
| saM165 | ctggaggga | 2030 | cccagtcag | 3402 | ATTGTTCCG | 3574 | CAGTGCCAG | 3746 |
| | agggttagc | | ccacaaaat | | TGGGTGGAG | | CAAGACTAG | |
| | tc | | ca | | TC | | CT | |
| saM166 | ctggaggga | 2031 | cccagtcag | 3403 | CAGTGCCAG | 3575 | ATTGTTCCG | 3747 |
| | agggttagc | | ccacaaaat | | CAAGACTAG | | TGGGTGGAG | |
| | tc | | ca | | CT | | TC | |
| saM167 | ctggaggga | 2032 | cccagtcag | 3404 | GGTTGCAGT | 3576 | ATTGTTCCG | 3748 |
| | agggttagc | | ccacaaaat | | GAGCTGAGA | | TGGGTGGAG | |
| | tc | | ca | | CT | | TC | |
| saM168 | ctggaggga | 2033 | cccagtcag | 3405 | TGCCTGGTA | 3577 | CCCACTCAC | 3749 |
| | agggttagc | | ccacaaaat | | CACAGCAAA | | CATGGACAA | |
| | tc | | ca | | CA | | CA | |
| saM169 | ctggaggga | 2034 | cccagtcag | 3406 | TGCCTGGTA | 3578 | CCCACTCAC | 3750 |
| | agggttagc | | ccacaaaat | | CACAGCAAA | | CATGGACAA | |
| | tc | | ca | | CA | | CA | |
| saM170 | ctggaggga | 2035 | cccagtcag | 3407 | TGCCTGGTA | 3579 | CCCACTCAC | 3751 |
| | agggttagc | | ccacaaaat | | CACAGCAAA | | CATGGACAA | |
| | tc | | ca | | CA | | CA | |
| saM171 | ctggaggga | 2036 | cccagtcag | 3408 | TGCCTGGTA | 3580 | CCCACTCAC | 3752 |
| | agggttagc | | ccacaaaat | | CACAGCAAA | | CATGGACAA | |
| | tc | | ca | | CA | | CA | |
| saM172 | ctggaggga | 2037 | cccagtcag | 3409 | TGCCTGGTA | 3581 | GGGGAGTGG | 3753 |
| | agggttagc | | ccacaaaat | | CACAGCAAA | | CTAATGTGA | |
| | tc | | ca | | CA | | CC | |

ND = not detectable above assay threshold of detection

TABLE 30
SaCas9 sgRNAs Categorized Based on Cleavage Efficiency

| Total INDEL % | Guides |
|---|---|
| Not detectable above | SaM1, SaM2, SaM3, SaM4, SaM5, SaM6, SaM7, SaM70, SaM80, SaM81, |
| assay threshold of | SaM103, SaM133, SaM139, SaM148, SaM153, SaM154, SaM155, SaM156, |
| detection | SaM157, SaM158, SaM159, SaM160, SaM161, SaM171, SaM172 |
| <15% | SaM9, SaM11 SaM14, SaM18, SaM23, SaM24, SaM26, SaM28, SaM29, |
| | SaM30, SaM32, SaM33, SaM35, SaM36, SaM40, SaM48, SaM49, SaM50, |
| | SaM52, SaM55, SaM56, SaM57, SaM59, SaM60, SaM62, SaM63, SaM64, |
| | SaM65, SaM67, SaM68, SaM72, SaM75, SaM76, SaM77, SaM82, SaM84, |
| | SaM85, SaM90, SaM95, SaM98, SaM99, SaM100, SaM102, SaM106, |
| | SaM107, SaM108, SaM109, SaM110, SaM112, SaM113, SaM114, SaM115, |
| | SaM116, SaM117, SaM118, SaM119, SaM121, SaM125, SaM126, SaM134, |
| | SaM135, SaM137, SaM138, SaM140, SaM141, SaM142, SaM143, SaM144, |
| | SaM147, SaM163, SaM167, SaM168 |
| 15%-25% | SaM8, SaM12, SaM13, SaM15, SaM16, SaM19, SaM21, SaM22, SaM31 |
| | SaM37, SaM38, SaM39, SaM41, SaM42, SaM43, SaM44, SaM45, SaM47, |
| | SaM51, SaM53, SaM61, SaM66, SaM69, SaM73, SaM74, SaM78, SaM79, |
| | SaM83, SaM86, SaM93, SaM94, SaM96, SaM97, SaM104, SaM111, SaM120, |
| | SaM122, SaM123, SaM127, SaM128, SaM130, SaM131, SaM132, SaM146, |
| | SaM149, SaM150, SaM162, SaM164, SaM165, SaM166, SaM169, SaM170 |
| >25% | SaM10, SaM17, SaM20, SaM25, SaM27, SaM34, SaM46, SaM54, SaM58, |
| | SaM71, SaM88, SaM91, SaM92, SaM101, SaM105, SaM124, SaM129, |
| | SaM136, SaM145, SaM151, SaM152 |

A subset of the SaCas9 sgRNAs was selected for inducing a microdeletion in FAAH-OUT. Specifically, 12 SaCas9 sgRNAs with high overall INDEL frequency were selected as left gRNAs (SaM8, SaM10, SaM17, SaM20, SaM25, SaM27, SaM34, SaM38, SaM45, SaM46, SaM54, and SaM58); and 4 SaCas9 sgRNAs with high overall INDEL frequency were selected as right gRNAs (SaM124, SaM151, SaM165, and SaM170). The frequency of INDELs at predicted cut-sites measured for these guides is provided in Table 31.

TABLE 31
Left and Right SaCas9 sgRNAs Targeting FAAH-OUT

| sgRNA Name | Indel % | L/R* |
|---|---|---|
| SaM8 | 15.4 | L |
| SaM10 | 38.875 | L |
| SaM17 | 34.8 | L |
| SaM20 | 26.7 | L |
| SaM25 | 27.3 | L |
| SaM27 | 31 | L |
| SaM34 | 30.2 | L |
| SaM38 | 23.9 | L |
| SaM45 | 23.7 | L |
| SaM46 | 25.9 | L |
| SaM54 | 27.35 | L |
| SaM58 | 30.925 | L |
| SaM124 | 32.45 | R |
| SaM151 | 40.9 | R |
| SaM165 | 23.05 | R |
| SaM170 | 24.3 | R |

*denotes Left (L) or Right (R) gRNA

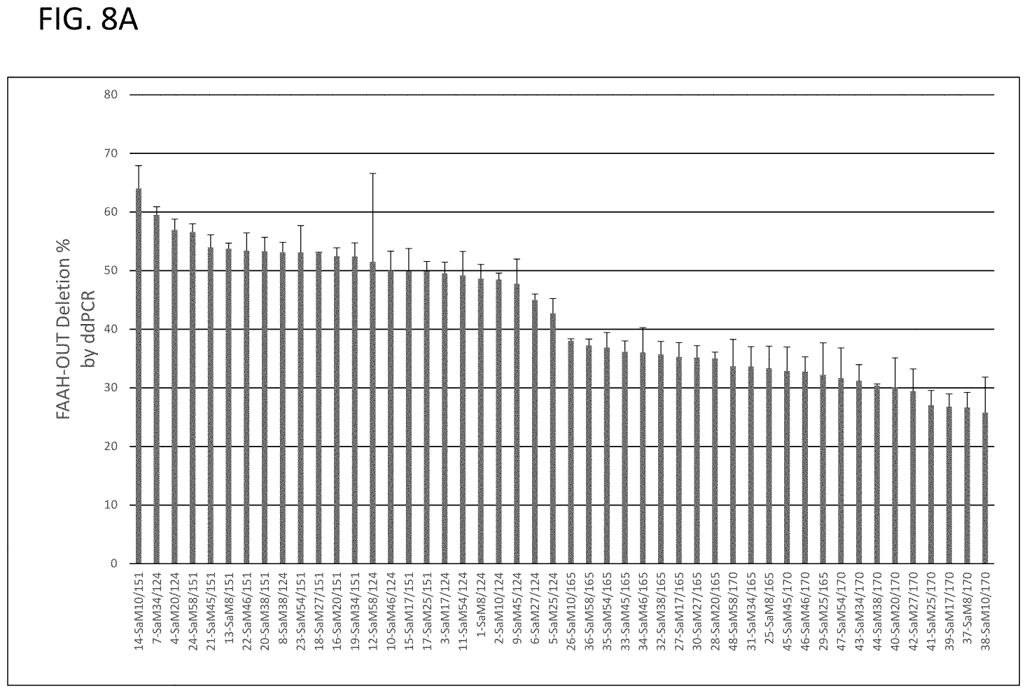

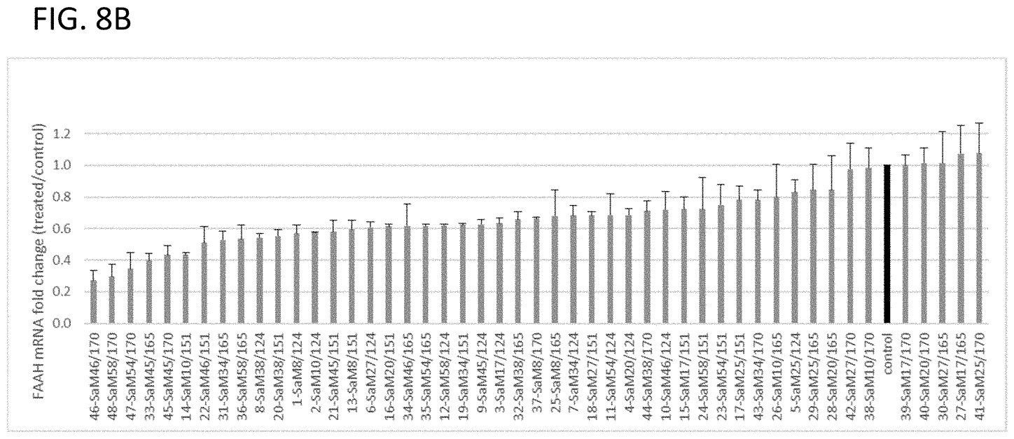

Combinations of SaCas9 sgRNAs identified in Table 32 were evaluated for inducing a microdeletion in FAAH-OUT. Briefly, 0.2×10⁶ MCF7 cells were electroporated with a left and right SaCas9 sgRNA (1.6 μg per each) and 3 μg SaCas9 protein (SEQ ID NO: 1272). The cells were incubated 48-72 hours following electroporation, then harvested. Genomic DNA was extracted for quantification of a deletion in FAAH-OUT by ddPCR as described in Example 6. As shown in FIG. 8A, the majority of sgRNA pairs evaluated resulted in frequency of deletion of FAAH-OUT that exceeded 40%. Quantification of deletion for each sgRNA combination is provided in Table 32.

Edited MCF7 cells were further harvested for RNA extraction and quantification of FAAH mRNA by qPCR as described in Example 2. As shown in FIG. 8B, the FAAH mRNA levels in treated cells, measured as fold change relative to control cells electroporated with SaCas9 only using the 2\hat{ }(−ddCt) method, were reduced by 20% or more for most of the sgRNA combinations tested. Quantification of fold change is provided in Table 32.

TABLE 32
Left and Right SaCas9 sgRNAs Targeting FAAH-OUT

| | Avg deletion | mRNA Fold |
|---|---|---|
| gRNA ID | (%) | Change |
| 1-SaM8/124 | 48.6456 | 0.571958 |
| 2-SaM10/124 | 48.4926 | 0.579198 |
| 3-SaM17/124 | 49.5368 | 0.636002 |
| 4-SaM20/124 | 56.9462 | 0.687604 |
| 5-SaM25/124 | 42.7308 | 0.831251 |
| 6-SaM27/124 | 44.9926 | 0.60729 |
| 7-SaM34/124 | 59.4875 | 0.683977 |
| 8-SaM38/124 | 53.1112 | 0.543927 |
| 9-SaM45/124 | 47.7424 | 0.628248 |
| 10-SaM46/124 | 50.0413 | 0.720223 |
| 11-SaM54/124 | 49.1768 | 0.685975 |
| 12-SaM58/124 | 51.5117 | 0.623613 |
| 13-SaM8/151 | 53.7392 | 0.59501 |
| 14-SaM10/151 | 64.0011 | 0.434573 |
| 15-SaM17/151 | 49.9067 | 0.723646 |
| 16-SaM20/151 | 52.447 | 0.614436 |
| 17-SaM25/151 | 49.8748 | 0.781345 |
| 18-SaM27/151 | 53.0009 | 0.684715 |
| 19-SaM34/151 | 52.4106 | 0.624151 |
| 20-SaM38/151 | 53.2814 | 0.553493 |
| 21-SaM45/151 | 53.9365 | 0.580466 |
| 22-SaM46/151 | 53.4233 | 0.511456 |
| 23-SaM54/151 | 53.104 | 0.750226 |
| 24-SaM58/151 | 56.5762 | 0.723851 |
| 25-SaM8/165 | 33.3402 | 0.681418 |
| 26-SaM10/165 | 38.0323 | 0.800786 |
| 27-SaM17/165 | 35.2796 | 1.074426 |
| 28-SaM20/165 | 35.0252 | 0.846318 |
| 29-SaM25/165 | 32.1994 | 0.845465 |
| 30-SaM27/165 | 35.1626 | 1.016445 |
| 31-SaM34/165 | 33.656 | 0.529662 |
| 32-SaM38/165 | 35.7047 | 0.658315 |
| 33-SaM45/165 | 36.1621 | 0.398656 |
| 34-SaM46/165 | 36.0589 | 0.617158 |
| 35-SaM54/165 | 36.8695 | 0.618408 |
| 36-SaM58/165 | 37.2224 | 0.536667 |
| 37-SaM8/170 | 26.6475 | 0.663469 |
| 38-SaM10/170 | 25.7974 | 0.983808 |
| 39-SaM17/170 | 26.7748 | 1.003524 |
| 40-SaM20/170 | 30.0636 | 1.01508 |
| 41-SaM25/170 | 27.0519 | 1.076097 |
| 42-SaM27/170 | 29.4293 | 0.976224 |
| 43-SaM34/170 | 31.2281 | 0.782584 |
| 44-SaM38/170 | 30.4027 | 0.713765 |
| 45-SaM45/170 | 32.9118 | 0.433929 |
| 46-SaM46/170 | 32.7716 | 0.272587 |
| 47-SaM54/170 | 31.6828 | 0.345486 |
| 48-SaM58/170 | 33.6818 | 0.29687 |

* control = 1.000

### Example 9: Evaluation of In Vitro Gene Editing and Functional Activity of gRNA/SpCas9 and sgRNA/SaCas9 Targeting the FAAH Coding Sequence Using AAV as Delivery System

A subset of SpCas9 and SaCas9 sgRNAs (Table 33) were selected for further evaluation using AAV vectors expressing SpCas9 or SaCas9 and sgRNAs. The vector transduced cells were monitored for indels (TIDE) at the predicted cut site, levels of FAAH mRNA, and FAAH protein. The binding sites for SpCas9 sgRNAs SpCh29, SpCh30, SpCh31, SpCh32 and SpCh34 are located in FAAH exon 2, and for SaCas9 sgRNAs SaCh1, SaCh7, SaCh11, SaCh1 and SaCh13 are located within or outside of FAAH exons 1, 2 and 4.

TABLE 33
Target Sequences for SpCas9 and SaCas9 sgRNAs in
the FAAH Coding Sequence

| | Target Sequence | SEQ | Cut site |
|---|---|---|---|
| Name | PAM in bold underline | ID NO | Location* |
| SpCh29 | GGTGAAGAGCACGGCCTCAGGGG | 29 | 46402176 |
| SpCh30 | GGCCGTGCTCTTCACCTATGTGG | 30 | 46402193 |
| SpCh31 | GCCGTGCTCTTCACCTATGTGGG | 31 | 46402194 |
| SpCh32 | TCCCACATAGGTGAAGAGCACGG | 32 | 46402185 |
| SpCh34 | TGGCCTTACCTTTCCCACATAGG | 34 | 46402197 |
| SaCh1 | TGGGATCCCGGCTGATCCAGTCCGGGT | 149 | 46394264 |
| SaCh7 | GCAGCGCCTCTGAGTCCAGGTCTGGGT | 155 | 46402101 |
| SaCh11 | CATTCAGGCTCAAGCCCAGCGTGGAGT | 159 | 46405388 |
| SaCh12 | GCTGGGCTTGAGCCTGAATGAAGGGGT | 160 | 46405403 |
| SaCh13 | GCCTGAATGAAGGGGTOCCGGCGGAGT | 161 | 46405414 |

*Chromosomal location of guide cut-site in chromosome 1 of human genome Hg38

For SpCas9, all-in-two AAV vectors and for SaCas9, all-in-one vectors were used. To generate AAV-SpCas9 vector, the coding sequence (SEQ ID NO: 3756) under the transcription control of truncated CMV promoter (SEQ ID NO: 3758) was cloned into AAV vector plasmid. SpCas9 sgRNA encoding DNA sequences (Table 33) under the control of U6 promoter (SEQ ID NO: 3756) were cloned into a separate AAV vector plasmid (Table 34). For SaCas9 system, Cas9 expression was placed under the control of CMV promoter (SEQ ID NO: 3759) and sgRNA expression under the control of a U6 promoter (SEQ ID NO: 3756). Spacer and tcrRNA sequences used are shown in Tables 33 and 34. The DNA sequences in the vector constructs were verified by nucleotide sequence determination prior to generation of vectors. AAV vector titers were determined by qPCR.

TABLE 34
Sequences of SpCas9 and SaCas9 tcrRNA sequences used in
AAV plasmid backbones.

| Name | tcrRNA sequences included in AAV plasmid sequences | SEQ ID NO |
|---|---|---|
| Sp | GTTTCAGAGCTATGCTGGAAACAGCATAGCAAGTTGAAATAAGG | 3754 |
| tcrRNA | CTAGTCCGTTATCAACTTGAAAAAGTGGCACCGAGTCGGTGCT | |
| Sa | GTTTAAGTACTCTGTGCTGGAAACAGCACAGAATCTACTTAAA | 3755 |
| tcrRNA | CAAGGCAAAATGCCGTGTTTATCTCGTCAACTTGTTGGCGAGA | |

MCF7 cells were used for transduction experiments as described below. Briefly, 1×10⁵ MCF7 cells were resuspended in 100 ul of Opti-MEM media (ThermoFisher Scientific) and incubated for 20 minutes at 37° C., 5% CO₂ with single (SaCas9-sgRNA) or dual (SpCas9 and U6-sgRNA) AAVs at a multiplicity of infection (MOI) of 50,000 in triplicates. The transduced cells were seeded into a 48-well plate and incubated for 96 hours. Thereafter, the genomic DNA was extracted and purified using a Quick DNA Kit (Zymo #D3011).

The frequency of INDELs induced at predicted cut sites in the genomic DNA was evaluated by TIDE analysis (see, e.g., Brinkman, et al (2014) NUCLEIC ACIDS RESEARCH 42:e168). Specifically, primers flanking the target site of each SpCas9 or SaCas9 sgRNA were used in a PCR reaction with 2 μL (40-70 ng) of genomic DNA to amplify a region 1 of 955 bp, region 2 of 759 bp, and region 4 of 932 bp flanking exon 1, 2 and 4 respectively, surrounding the predicted cut site of each sgRNA. The primers used for amplification corresponding to each SpCas9 and SaCas9 sgRNAs are identified in Table 35 and Table 36, respectively. The PCR product was purified using AMPure XP PCR Purification (Beckman Coulter #A63881) and Sanger sequencing (Genewiz) was performed using the sequencing primers identified in Table 35 and Table 36. The sequence data was analyzed using the Tsunami software to determine the frequency of INDELs at the predicted cut site for each sgRNA/SpCas9 or SaCas9 complex.

TABLE 35
PCR and TIDE Primer Sequences for Analysis of INDEL Frequency at
Cut Site Corresponding to SpCas9 sgRNAs

| | | SEQ | | SEQ | Sequencing | SEQ |
|---|---|---|---|---|---|---|
| sgRNA | PCR primer 1 | ID NO | PCR primer 2 | ID NO | primer | ID NO |
| SpCh29 | CATCAGTCTGGAGCT | 1319 | AGACCAGACTTGTTG | 1353 | AGCATGTGCCTGTAG | 1387 |
| | AGGCA | | CCCAA | | TTC | |
| SpCh30 | CATCAGTCTGGAGCT | 1320 | AGACCAGACTTGTTG | 1354 | AGCATGTGCCTGTAG | 1388 |
| | AGGCA | | CCCAA | | TTC | |
| SpCh31 | CATCAGTCTGGAGCT | 1321 | AGACCAGACTTGTTG | 1355 | AGCATGTGCCTGTAG | 1389 |
| | AGGCA | | CCCAA | | TTC | |
| SpCh32 | CATCAGTCTGGAGCT | 1322 | AGACCAGACTTGTTG | 1356 | AGCATGTGCCTGTAG | 1390 |
| | AGGCA | | CCCAA | | TTC | |
| SpCh34 | CATCAGTCTGGAGCT | 1324 | AGACCAGACTTGTTG | 1358 | AGCATGTGCCTGTAG | 1392 |
| | AGGCA | | CCCAA | | TTC | |

TABLE 36

PCR and TIDE Primer Sequences for Analysis ofINDEL Frequency at Cut Site Corresponding
to SaCas9 sgRNAs

| | PCR | SEQ | PCR | SEQ | Sequencing | SEQ | Sequencing | SEQ | Sequencing | SEQ |
|---|---|---|---|---|---|---|---|---|---|---|
| sgRNA | primer 1 | ID NO | primer 2 | ID NO | primer 1 | ID NO | primer 2 | ID NO | primer 3 | ID NO |
| SaCh1 | TCTAACAG | 1513 | AAGCTCT | 1529 | TCTAACA | 1545 | AAGCTCT | 1561 | CACTACG | 1577 |
| | CTGGCATG | | CCAGATC | | GCTGGCA | | CCAGATC | | CTCCGGC | |
| | TCTG | | CCCTTG | | TGTCTG | | CCCTTG | | AGTCACC | |
| SaCh7 | CATCAGTC | 1519 | AGACCAG | 1535 | CATCAGT | 1551 | AGACCAG | 1567 | AGACCAG | 1567 |
| | TGGAGCTA | | ACTTGTT | | CTGGAGC | | ACTTGTT | | ACTTGTT | |
| | GGCA | | GCCCAA | | TAGGCA | | GCCCAA | | GCCCAA | |
| SaCh11 | GACCAACT | 1523 | TCTGAAC | 1539 | GACCAAC | 1555 | TCTGAAC | 1571 | ACCTACA | 1584 |
| | GTGTGACC | | ACTCACC | | TGTGTGA | | ACTCACC | | AGGTATG | |
| | TCCT | | GCTTTG | | CCTCCT | | GCTTTG | | CTCTGC | |
| SaCh12 | GACCAACT | 1524 | TCTGAAC | 1540 | GACCAAC | 1556 | TCTGAAC | 1572 | ACCTACA | 1585 |
| | GTGTGACC | | ACTCACC | | TGTGTGA | | ACTCACC | | AGGTATG | |
| | TCCT | | GCTTTG | | CCTCCT | | GCTTTG | | CTCTGC | |
| SCh13 | GACCAACT | 1525 | TCTGAAC | 1541 | GACCAAC | 1557 | TCTGAAC | 1573 | ACCTACA | 1586 |
| | GTGTGACC | | ACTCACC | | TGTGTGA | | ACTCACC | | AGGTATG | |
| | TCCT | | GCTTTG | | CCTCCT | | GCTTTG | | CTCTGC | |

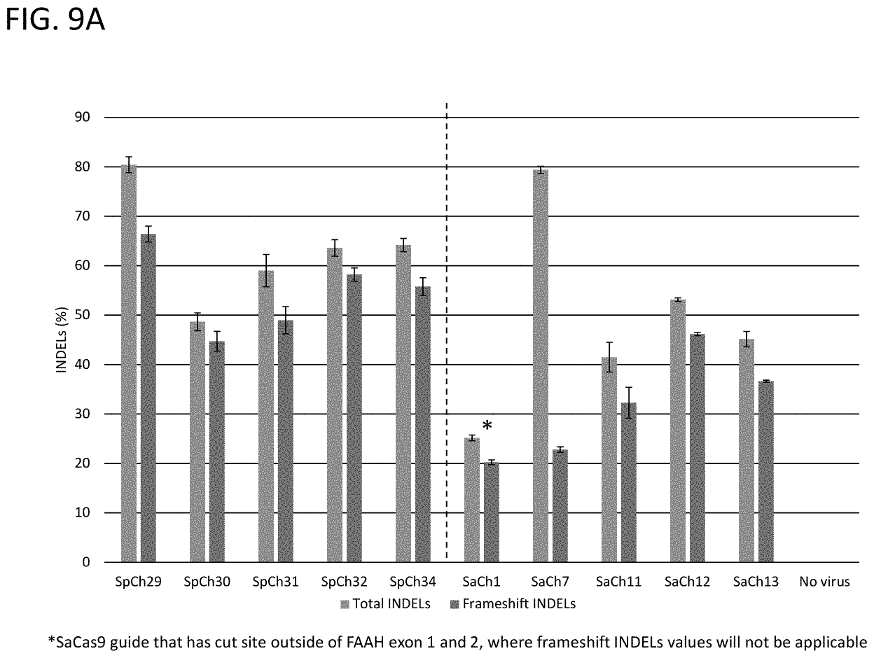

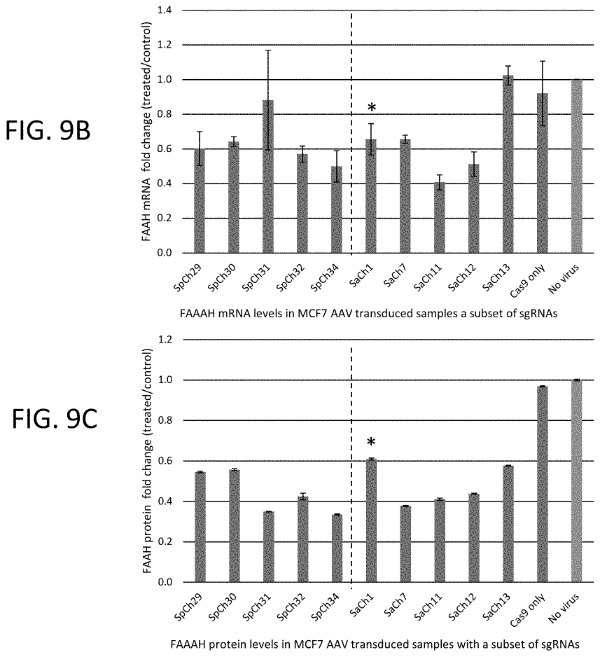

The overall INDEL frequency at the predicted cut sites for each sgRNA is provided in Table 37 and in FIG. 9A. The INDELs resulting in an in-frame mutation (i.e., ±3 nt, ±6 nt, ±9 nt, etc.) were removed to provide the percentage of INDELs expected to produce a frameshift mutation (i.e., ±1 nt, ±2 nt, ±4 nt, etc), is also shown in Table 37. The sgRNA SaCh1 having cut sites outside the exon 1 region of FAAH is shown by asterisk. As a frameshift mutation for these guides is not applicable, the value represented by “frameshift INDELs” refers to the frequency of total INDELs minus the frequency of INDELs that are divisible by 3 (e.g., ±3 nt, ±6 nt, ±9 nt, etc).

To determine FAAH mRNA levels post-editing total RNA was extracted from the cells and subjected to quantitative PCR (qPCR) assay. Specifically, RNA extraction was performed using a Quick-RNA 96 Kit (Zymo Research, #R1052). RNA concentration was measured by DropSense (Trinean) and 250 ng RNA was used for reverse transcription using a QuantiTect Reverse Transcription kit (Qiagen #205311) to prepare cDNA. Subsequently, 40 ng of cDNA was used for qPCR to measure FAAH mRNA levels. For qPCR quantification, TaqMan Fast Advanced Master Mix (ThermoFisher #4444557) was combined with the reagents below. TBP (ThermoFisher #4331182) mRNA levels were used as qPCR internal controls.

(SEQ ID NO: 1273)
Forward primer: TGATATCGGAGGCAGCATCC;
(SEQ ID NO: 1274)
Reverse primer: CTTCAGGCCACTCTTGCTGA ; and
(SEQ ID NO: 1275)
Probe: CTTCCCCTCCTCCTTCTGC.

FAAH mRNA levels were quantified as a fold change between an edited sample and an untreated control sample subjected to electroporation without CRISPR/Cas9 components. Fold change was calculated using the 2\hat{ }(−ddCt) method and is provided for each sgRNA in Table 37 and in FIG. 9B. Most sgRNA achieved at least a 50% reduction in FAAH mRNA levels, with SaCh11, SaCh12, SpCh29, SpCh32 and SpCh34 sgRNAs producing the greatest reduction.

TABLE 37
Quantification of Editing Efficiency and Functional Activity
of SpCas9 sgRNAs Targeting FAAH Coding Sequence

| sgRNA | Indel (%) | | FAAH mRNA | FAAH protein |
|---|---|---|---|---|
| Name | Total | Frameshift* | (fold change) | (FAAH:GAPDH) |
| SpCh29 | 80.4 | 66.4 | 0.602 | 0.545 |
| SpCh30 | 48.7 | 44.7 | 0.642 | 0.557 |
| SpCh31 | 59 | 49.0 | 0.881 | 0.349 |
| SpCh32 | 63.6 | 58.2 | 0.570 | 0.424 |
| SpCh34 | 64.18 | 55.8 | 0.499 | 0.335 |
| SaCh1 | 25.2 | 20.2 | 0.655 | 0.609 |
| SaCh7 | 79.4 | 22.8 | 0.656 | 0.378 |
| SaCh11 | 41.5 | 31.3 | 0.407 | 0.411 |
| SaCh12 | 53.2 | 46.2 | 0.512 | 0.438 |
| SaCh13 | 45.2 | 36.7 | 1.02 | 0.576 |

*Frameshift INDEL % refers to INDELs expected to result in a frameshift mutation in the FAAH coding sequence (i.e., ±1 nt, ±2 nt, ±4 nt). The sgRNAs with values in underline have cut sites outside exon 1 of FAAH, wherein frameshift mutations are not applicable. Thus, Frameshift INDEL % refers to frequency of total INDELs minus frequency of INDELs that are ±3 nt, ±6 nt, ±9 nt, etc. All data entered are a mean of n = 3 replicates.

Edited MCF7 cells were also harvested for total protein extraction to quantify FAAH protein levels by Simple Wes. Protein extraction was performed using RIPA lysis and extraction buffer (ThermoFisher #89900). Subsequently, 0.5 μg of protein was loaded onto Simple Wes and analyzed using a target primary mouse anti-FAAH1 antibody (Abcam #ab54615; 1:25 dilution) and a housekeeping primary rabbit anti-GAPDH mAb 14C10 (CST #2118S; 1:25 dilution) in antibody diluent (ProteinSimple Anti-rabbit and anti-mouse secondary antibody (ProteinSimple #DM-001; ProteinSimple DM-002) were mixed in equal parts for detection. The relative expression level of FAAH protein was compared to GAPDH as internal control. The relative expression level of FAAH protein was then normalized for samples treated with sgRNA/SpCas9 or sgRNA/SaCas9 to a untransduced (no virus) sample. Normalized FAAH protein levels following editing are provided in Table 37 and FIG. 9C. Several of the sgRNAs evaluated, including SpCh31, SpCh32, SpCh34, SaCh7, SaCh11, and SaCh12, resulted in a reduction of FAAH protein expression of 50% or more.

| Name/ | | SEQ |
|---|---|---|
| Identifier | Sequence | ID NO |
| Sp sgRNA | mN*mN*mN*NNNNNNNNNNNNNNNNNGUUUUAGAGCUAGAAAUAGCAAGUUAAAAUAAGG | 1267 |
| backbone | CUAGUCCGUUAUCAACUUGAAAAAGUGGCACCGAGUCGGUmG*mC*mU* | |
| Spcas9 | MHHHHHHHHGSGGSGGSGPKKKRKVGSGGSGGSGKRNYILGLDIGITSV | 1268 |
| Polypeptide | GYGIIDYETRDVIDAGVRLFKEANVENNEGRRSKRGARRLKRRRRHRIQ | |
| | RVKKLLFDYNLLTDHSELSGINPYEARVKGLSQKLSEEEFSAALLHLAK | |
| | RRGVHNVNEVEEDTGNELSTKEQISRNSKALEEKYVAELQLERLKKDGE | |
| | VRGSINRFKTSDYVKEAKQLLKVQKAYHQLDQSFIDTYIDLLETRRTYY | |
| | EGPGEGSPFGWKDIKEWYEMLMGHCTYFPEELRSVKYAYNADLYNALND | |
| | LNNLVITRDENEKLEYYEKFQIIENVFKQKKKPTLKQIAKEILVNEEDI | |
| | KGYRVTSTGKPEFTNLKVYHDIKDITARKEIIENAELLDQIAKILTIYQ | |
| | SSEDIQEELTNLNSELTQEEIEQISNLKGYTGTHNLSLKAINLILDELW | |
| | HTNDNQIAIFNRLKLVPKKVDLSQQKEIPTTLVDDFILSPVVKRSFIQS | |
| | IKVINAIIKKYGLPNDIIIELAREKNSKDAQKMINEMQKRNRQTNERIE | |
| | EIIRTTGKENAKYLIEKIKLHDMQEGKCLYSLEAIPLEDLLNNPFNYEV | |
| | DHIIPRSVSFDNSFNNKVLVKQEENSKKGNRTPFQYLSSSDSKISYETF | |
| | KKHILNLAKGKGRISKTKKEYLLEERDINRFSVQKDFINRNLVDTRYAT | |
| | RGLMNLLRSYFRVNNLDVKVKSINGGFTSFLRRKWKFKKERNKGYKHHA | |
| | EDALIIANADFIFKEWKKLDKAKKVMENQMFEEKQAESMPEIETEQEYK | |
| | EIFITPHQIKHIKDFKDYKYSHRVDKKPNRELINDTLYSTRKDDKGNTL | |
| | IVNNLNGLYDKDNDKLKKLINKSPEKLLMYHHDPQTYQKLKLIMEQYGD | |
| | EKNPLYKYYEETGNYLTKYSKKDNGPVIKKIKYYGNKLNAHLDITDDYP | |
| | NSRNKVVKLSLKPYRFDVYLDNGVYKFVTVKNLDVIKKENYYEVNSKCY | |
| | EEAKKLKKISNQAEFIASFYNNDLIKINGELYRVIGVNNDLLNRIEVNM | |
| | IDITYREYLENMNDKRPPRIIKTIASKTQSIKKYSTDILGNLYEVKSKK | |
| | HPQIIKKGGSAGSGGSGGSGPKKKRKV | |
| Slu sRNA | mN*mN*mN*NNNNNNNNNNNNNNNNNNNGUUUUAGUACUCUGGAAACAG | 1269 |
| backbone | AAUCUACUGAAACAAGACAAUAUGUCGUGUUUAUCCCAUCAAUUUAUUG | |
| | GUGGmG*mA*mU* | |
| Slucas9 | MPKKKRKVGMNQKFILGLDIGITSVGYGLIDYETKNIIDAGVRLFPEAN | 1270 |
| Polypeptide | VENNEGRRSKRGSRRLKRRRIHRLERVKKLLEDYNLLDQSQIPQSTNPY | |
| | AIRVKGLSEALSKDELVIALLHIAKRRGIHKIDVIDSNDDVGNELSTKE | |
| | QLNKNSKLLKDKFVCQIQLERMNEGQVRGEKNRFKTADIIKEIIQLLNV | |
| | QKNFHQLDENFINKYIELVEMRREYFEGPGKGSPYGWEGDPKAWYETLM | |
| | GHCTYFPDELRSVKYAYSADLFNALNDLNNLVIQRDGLSKLEYHEKYHI | |
| | IENVFKQKKKPTLKQIANEINVNPEDIKGYRITKSGKPQFTEFKLYHDL | |
| | KSVLFDQSILENEDVLDQIAEILTIYQDKDSIKSKLTELDILLNEEDKE | |
| | NIAQLTGYTGTHRLSLKCIRLVLEEQWYSSRNQMEIFTHLNIKPKKINL | |
| | TAANKIPKAMIDEFILSPVVKRTFGQAINLINKIIEKYGVPEDIIIELA | |
| | RENNSKDKQKFINEMQKKNENTRKRINEIIGKYGNQNAKRLVEKIRLHD | |
| | EQEGKCLYSLESIPLEDLLNNPNHYEVDHIIPRSVSFDNSYHNKVLVKQ | |
| | SENSKKSNLTPYQYFNSGKSKLSYNQFKQHILNLSKSQDRISKKKKEYL | |
| | LEERDINKFEVQKEFINRNLVDTRYATRELTNYLKAYFSANNMNVKVKT | |
| | INGSFTDYLRKVWKFKKERNHGYKHHAEDALIIANADFLFKENKKLKAV | |
| | NSVLEKPEIETKQLDIQVDSEDNYSEMFIIPKQVQDIKDFRNFKYSHRV | |
| | DKKPNRQLINDTLYSTRKKDNSTYIVQTIKDIYAKDNTTLKKQFDKSPE | |
| | KFLMYQHDPRTFEKLEVIMKQYANEKNPLAKYHEETGEYLTKYSKKNNG | |
| | PIVKSLKYIGNKLGSHLDVTHQFKSSTKKLVKLSIKPYRFDVYLTDKGY | |
| | KFITISYLDVLKKDNYYYIPEQKYDKLKLGKAIDKNAKFIASFYKNDLI | |
| | KLDGEIYKIIGVNSDTRNMIELDLPDIRYKEYCELNNIKGEPRIKKTIG | |
| | KKVNSIEKLTTDVLGNVFTNTQYTKPQLLFKRGNGSGGSGGSGAKRPAA | |
| | TKKAGQAKKKKHHHHHH | |
| Sa sgRNA | mN*mN*mN*NNNNNNNNNNNNNNNNNNGUUUUAGUACUCUGGAAACAGA | 1271 |
| backbone | AUCUACUAAAACAAGGCAAAAUGCCGUGUUUAUCUCGUCAACUUGUUGG | |
| | CGAmG*mA*mU* | |
| SaCas9 | MHHHHHHHHGSGGSGGSGPKKKRKVGSGGSGGSGKRNYILGLDIGITSV | 1272 |
| polypeptide | GYGIIDYETRDVIDAGVRLFKEANVENNEGRRSKRGARRLKRRRRHRIQ | |
| | RVKKLLFDYNLLTDHSELSGINPYEARVKGLSQKLSEEEFSAALLHLAK | |
| | RRGVHNVNEVEEDTGNELSTKEQISRNSKALEEKYVAELQLERLKKDGE | |
| | VRGSINRFKTSDYVKEAKQLLKVQKAYHQLDQSFIDTYIDLLETRRTYY | |
| | EGPGEGSPFGWKDIKEWYEMLMGHCTYFPEELRSVKYAYNADLYNALND | |
| | LNNLVITRDENEKLEYYEKFQIIENVFKQKKKPTLKQIAKEILVNEEDI | |
| | KGYRVTSTGKPEFTNLKVYHDIKDITARKEIIENAELLDQIAKILTIYQ | |
| | SSEDIQEELTNLNSELTQEEIEQISNLKGYTGTHNLSLKAINLILDELW | |
| | HTNDNQIAIFNRLKLVPKKVDLSQQKEIPTTLVDDFILSPVVKRSFIQS | |
| | IKVINAIIKKYGLPNDIIIELAREKNSKDAQKMINEMQKRNRQTNERIE | |
| | EIIRTTGKENAKYLIEKIKLHDMQEGKCLYSLEAIPLEDLLNNPFNYEV | |
| | DHIIPRSVSFDNSFNNKVLVKQEENSKKGNRTPFQYLSSSDSKISYETF | |
| | KKHILNLAKGKGRISKTKKEYLLEERDINRFSVQKDFINRNLVDTRYAT | |
| | RGLMNLLRSYFRVNNLDVKVKSINGGFTSFLRRKWKFKKERNKGYKHHA | |
| | EDALIIANADFIFKEWKKLDKAKKVMENQMFEEKQAESMPEIETEQEYK | |
| | EIFITPHQIKHIKDFKDYKYSHRVDKKPNRELINDTLYSTRKDDKGNTL | |
| | IVNNLNGLYDKDNDKLKKLINKSPEKLLMYHHDPQTYQKLKLIMEQYGD | |
| | EKNPLYKYYEETGNYLTKYSKKDNGPVIKKIKYYGNKLNAHLDITDDYP | |
| | NSRNKVVKLSLKPYRFDVYLDNGVYKFVTVKNLDVIKKENYYEVNSKCY | |
| | EEAKKLKKISNQAEFIASFYNNDLIKINGELYRVIGVNNDLLNRIEVNM | |
| | IDITYREYLENMNDKRPPRIIKTIASKTQSIKKYSTDILGNLYEVKSKK | |
| | HPQIIKKGGSAGSGGSGGSGPKKKRKV | |
| Forward primer | TGATATCGGAGGCAGCATCC | 1273 |
| Reverse primer | CTTCAGGCCACTCTTGCTGA | 1274 |
| Probe | CTTCCCCTCCTCCTTCTGC | 1275 |
| Forward primer | CATAGACTGAGCCTGGGATTTG | 1276 |
| Reverse primer | CAAAGCATGGGAACAGCACC | 1277 |
| Probe | AGGATGTGACAACCCGTCTC | 1278 |
| Forward primer | CCCAGTGACTAGTGTTCAGC | 1279 |
| Reverse primer | CTTTCGCTCGACATCCACTG | 1280 |
| Probe | CTGGATCAGGAGCACAGTAGAC | 1281 |
| Target | N19-21-NGG | 1282 |
| sequence | | |
| Target | N19-22-NNGG | 1283 |
| sequence | | |
| Target | N19-22-NNGRRT | 1284 |
| sequence | | |
| SpCas9 sgRNA | GUUUUAGAGCUAGAAAUAGCAAGUUAAAAUAAGGCUAGUCCGUUAUCAA | |
| backbone | CUUGAAAAAGUGGCACCGAGUCGGUGCU | 1285 |
| SluCas9 sgRNA | GUUUUAGUACUCUGGAAACAGAAUCUACUGAAACAAGACAAUAUGUCGU | |
| backbone | GUUUAUCCCAUCAAUUUAUUGGUGGGAU | 1286 |
| SaCas9 sgRNA | GUUUUAGUACUCUGGAAACAGAAUCUACUAAAACAAGGCAAAAUGCCGU | |
| backbone | GUUUAUCUCGUCAACUUGUUGGCGAGAU | 1287 |
| SV40 NLS 1 | PKKKRKV | 1288 |
| SV40 NLS 2 | PKKKRRV | 1289 |
| nucleoplasmin | KRPAATKKAGQAKKKK | 1290 |
| NLS | | |
| Sp tcrRNA | GTTTCAGAGCTATGCTGGAAACAGCATAGCAAGTTGAAATAAGGCTAGTCCGTTATCAAC | 3754 |
| | TTGAAAAAGTGGCACCGAGTCGGTGCT | |
| Sp tcrRNA | GTTTAAGTACTCTGTGCTGGAAACAGCACAGAATCTACTTAAACAAGGCAAAATGCCGTG | 3755 |
| | TTTATCTCGTCAACTTGTTGGCGAGA | |
| SpCas9 DNA | ATGGGCCCCGCCGCCAAGAGAGTGAAGCTGGACggatccGACAAGAAGTACTCCATTGGG | 3756 |
| sequence | CTGGACATTGGCACTAACTCCGTGGGATGGGCCGTGATCACCGACGAGTACAAAGTGCCC | |
| (SpCAS9 v2) | AGCAAGAAGTTTAAAGTGCTGGGGAATACTGACCGGCACAGCATCAAGAAGAACCTTATA | |
| | GGCGCCCTCCTGTTTGATTCCGGAGAAACCGCTGAAGCCACCCGGCTCAAGAGAACCGCC | |
| | AGACGCCGCTACACCCGGAGGAAGAATCGCATCTGCTATCTGCAAGAGATCTTCTCCAAC | |
| | GAAATGGCCAAGGTGGACGACTCGTTCTTCCATCGGCTGGAGGAGTCCTTTCTGGTGGAA | |
| | GAAGATAAGAAGCATGAGAGACACCCCATCTTCGGCAACATCGTGGATGAAGTGGCCTAC | |
| | CACGAAAAGTACCCTACCATCTACCACCTTCGCAAGAAGCTCGTGGATAGCACTGATAAG | |
| | GCGGACCTCCGCCTGATCTACCTCGCGCTCGCCCATATGATCAAGTTCCGGGGACACTTC | |
| | CTGATCGAGGGGGACCTGAACCCTGACAACAGCGACGTGGATAAGCTGTTCATCCAACTG | |
| | GTGCAAACCTATAACCAGCTGTTCGAGGAGAACCCTATCAACGCCTCCGGAGTGGACGCC | |
| | AAGGCCATCCTGTCGGCTCGCCTGTCCAAGTCGAGAAGGCTGGAAAACCTGATTGCCCAG | |
| | CTCCCGGGAGAAAAGAAGAACGGCCTGTTCGGCAACCTGATCGCTCTCTCCCTGGGCCTG | |
| | ACCCCGAATTTCAAGAGCAACTTCGACCTCGCCGAAGATGCAAAGCTCCAGCTGTCAAAA | |
| | GACACCTACGACGATGACCTGGACAATCTGCTGGCACAGATCGGGGATCAGTACGCTGAC | |
| | CTGTTCCTGGCCGCCAAGAACCTGTCCGACGCGATCCTGCTCTCGGATATTCTGAGGGTC | |
| | AACACCGAGATTACCAAGGCCCCTCTGTCCGCGAGCATGATCAAGCGGTACGATGAACAT | |
| | CACCAGGATCTGACACTCTTGAAGGCCCTTGTCCGCCAACAACTGCCGGAGAAGTACAAG | |
| | GAGATTTTCTTTGATCAGTCCAAGAACGGCTACGCTGGCTACATTGACGGGGGTGCCAGC | |
| | CAGGAAGAATTTTACAAGTTCATTAAGCCTATTCTCGAAAAGATGGACGGAACTGAGGAG | |
| | TTGCTCGTGAAGCTGAACCGGGAGGACCTGTTGAGAAAGCAACGCACCTTCGACAACGGT | |
| | TCGATTCCTCATCAAATTCATCTGGGTGAACTGCACGCCATCCTCCGGCGGCAGGAGGAT | |
| | TTCTATCCATTCCTGAAAGACAACCGAGAGAAGATTGAGAAAATCCTGACCTTCCGGATA | |
| | CCCTACTACGTGGGACCATTGGCTCGGGGGAACAGCAGATTCGCGTGGATGACTAGAAAG | |
| | TCCGAGGAGACTATTACCCCGTGGAACTTCGAGGAGGTGGTCGATAAGGGCGCATCGGCA | |
| | CAGTCCTTCATCGAGCGGATGACCAACTTCGACAAGAACCTTCCCAACGAAAAGGTGCTG | |
| | CCCAAGCACTCGCTGTTGTACGAGTACTTTACCGTGTACAACGAGCTCACTAAAGTGAAA | |
| | TACGTGACCGAGGGAATGAGAAAGCCGGCCTTTCTGTCCGGGGAACAGAAGAAGGCCATC | |
| | GTGGACCTCCTCTTCAAAACCAACAGAAAAGTCACCGTGAAGCAGCTGAAGGAGGACTAC | |
| | TTCAAGAAAATCGAGTGCTTCGACTCGGTCGAGATTTCGGGGGTCGAGGATAGGTTTAAT | |
| | GCCAGCCTGGGTACTTACCACGATCTGCTGAAGATCATTAAGGACAAGGACTTCCTTGAC | |
| | AACGAAGAAAACGAGGACATCCTTGAGGACATTGTCCTGACCCTGACCCTGTTTGAGGAT | |
| | CGGGAGATGATTGAGGAAAGACTTAAGACCTACGCTCATTTGTTCGACGACAAGGTCATG | |
| | AAACAGCTGAAGCGGAGGCGGTACACTGGATGGGGTCGGCTGTCCAGGAAGCTGATCAAC | |
| | GGAATCCGGGACAAGCAATCCGGAAAGACCATCCTGGACTTCCTGAAGTCAGACGGGTTC | |
| | GCCAACCGGAACTTCATGCAGCTCATTCACGACGACAGCCTGACGTTCAAGGAGGACATC | |
| | CAGAAGGCACAAGTGTCGGGACAGGGAGACAGCCTCCACGAACACATTGCGAACCTCGCG | |
| | GGTTCACCGGCTATCAAGAAGGGAATCCTGCAGACTGTGAAGGTGGTGGACGAGTTGGTC | |
| | AAGGTCATGGGCAGGCATAAGCCTGAAAACATCGTGATCGAGATGGCCCGGGAGAACCAG | |
| | ACCACCCAGAAGGGGCAGAAGAACAGCAGAGAGCGCATGAAGCGCATTGAGGAGGGCATC | |
| | AAGGAACTGGGATCACAGATCCTGAAGGAACATCCCGTGGAAAACACGCAGCTGCAGAAC | |
| | GAGAAACTCTACCTGTACTATTTGCAAAACGGCCGCGATATGTACGTGGACCAAGAACTC | |
| | GATATCAACCGCCTGTCCGACTACGACGTGGACCACATCGTGCCGCAGAGCTTCCTGAAG | |
| | GATGATTCTATCGATAACAAGGTCCTCACCCGGTCGGACAAGAATCGGGGGAAGTCAGAT | |
| | AACGTGCCGTCTGAGGAAGTGGTGAAGAAGATGAAGAATTACTGGCGGCAGCTTCTGAAC | |
| | GCGAAACTTATTACCCAGCGGAAATTCGACAACCTGACTAAGGCCGAGCGGGGAGGACTG | |
| | TCAGAACTGGACAAAGCCGGCTTCATTAAGAGACAGCTGGTCGAAACTCGCCAGATCACC | |
| | AAACATGTGGCCCAGATCCTGGACTCCAGGATGAACACCAAGTACGACGAAAACGATAAG | |
| | CTCATTCGGGAAGTGAAAGTGATCACACTGAAGTCCAAGCTGGTGTCCGACTTCCGCAAG | |
| | GACTTCCAGTTCTACAAGGTCCGCGAGATTAACAACTACCACCACGCACACGACGCTTAC | |
| | TTGAACGCCGTCGTGGGCACTGCCTTGATTAAGAAATACCCGAAGCTGGAATCCGAGTTC | |
| | GTGTACGGAGACTACAAGGTGTACGATGTGCGCAAGATGATCGCCAAGTCGGAGCAAGAA | |
| | ATCGGAAAGGCCACCGCTAAGTATTTCTTTTACTCCAACATTATGAACTTCTTCAAGACT | |
| | GAGATCACCCTGGCCAATGGAGAAATCCGCAAGAGGCCGCTGATCGAAACCAATGGAGAG | |
| | ACTGGAGAGATTGTGTGGGATAAGGGACGCGACTTCGCCACCGTGCGCAAGGTGCTGAGC | |
| | ATGCCCCAAGTCAACATTGTGAAAAAGACCGAAGTGCAGACGGGCGGTTTCTCAAAGGAA | |
| | AGCATCCTGCCTAAGCGGAACTCCGATAAGCTGATCGCGCGCAAGAAGGACTGGGACCCG | |
| | AAGAAATATGGCGGCTTCGACTCCCCCACCGTCGCCTACTCGGTGCTCGTCGTGGCTAAA | |
| | GTGGAGAAGGGAAAGTCGAAGAAGCTCAAGTCCGTGAAGGAATTGCTGGGTATTACTATT | |
| | ATGGAACGGTCCAGCTTCGAGAAGAATCCGATCGACTTCCTGGAGGCCAAGGGATACAAG | |
| | GAAGTGAAGAAGGACCTGATCATTAAGCTGCCGAAGTACAGCCTTTTTGAGCTGGAAAAC | |
| | GGACGCAAGCGGATGCTGGCCTCCGCCGGAGAGCTGCAGAAGGGCAACGAACTGGCCCTC | |
| | CCGTCCAAATACGTGAACTTTCTGTACCTGGCCAGCCACTACGAGAAGCTGAAGGGATCA | |
| | CCTGAAGATAACGAGCAGAAGCAGCTGTTCGTGGAACAACATAAGCATTATCTTGACGAG | |
| | ATCATTGAACAGATCTCTGAGTTCTCCAAGAGAGTGATTCTGGCTGACGCTAACCTTGAC | |
| | AAAGTGCTGAGCGCTTACAACAAGCACAGGGACAAGCCCATCCGGGAGCAGGCAGAGAAC | |
| | ATCATTCACCTGTTCACTCTCACCAACTTGGGTGCCCCGGCAGCCTTCAAGTACTTCGAT | |
| | ACCACAATCGACCGCAAGAGGTACACCTCAACCAAGGAGGTCCTTGACGCTACCCTGATC | |
| | CATCAATCCATTACCGGCCTGTACGAAACTAGGATCGACCTGTCGCAGCTGGGTGGCGAC | |
| | AAGCTTCCTGCCGCCAAGAGAGTGAAGCTGGACtaa | |
| SaCas9 DNA | atgCCTGCCGCCAAGAGAGTGAAGCTGGACggatccggaaagcggaactata | 3757 |
| sequence | tcctgggactggacatcggaattacctccgtgggatacggcatcatcgattacgagactagggacgtgat | |
| | tgacgccggcgtgagactctttaaggaggccaacgtggaaaacaacgaaggtcgcagatccaagcgg | |
| | ggtgcaagacgcctgaagcgccggaggagacatcggatacagcgcgtgaagaagctccttttcgacta | |
| | caacctcctcactgaccactcggaattgtccggtatcaacccctacgaagcccgcgtgaaaggcctgag | |
| | ccagaagctgtccgaagaggagtttagcgcagccctgctgcacctggctaagcgaaggggggtgcac | |
| | aacgtgaacgaggtggaggaggacactggcaacgaactgtccaccaaggagcagatttcacggaact | |
| | cgaaggcgctggaagagaaatatgtggccgagctgcagctggagaggctcaagaaggatggcgaag | |
| | tccgggggagcatcaatcgcttcaagacctcggactacgtgaaggaagccaaacagctgttgaaggtg | |
| | cagaaggcctaccaccaactggaccaatcattcattgacacttacatcgatctgcttgaaaccaggcgca | |
| | cctactacgagggtcctggagaaggcagccctttcggatggaaggacatcaaggagtggtatgagatg | |
| | ctgatgggtcattgcacctactttccggaagaactgcgctcagtgaagtacgcgtacaacgctgacctcta | |
| | caacgctctcaacgatctgaacaacctcgtgatcacccgggacgagaacgaaaagctggagtactacg | |
| | aaaagttccagattatcgaaaacgtgttcaagcagaagaagaagcccaccctgaagcagattgcaaag | |
| | gagatccttgtgaacgaggaggatattaagggctaccgggtcacctccaccgggaaaccagagttcact | |
| | aatctcaaggtgtaccatgacattaaggacattactgcccgcaaggagatcattgaaaacgcggaactgc | |
| | tggaccaaatcgcgaagatcctgaccatctatcagagctccgaggatatccaggaggaacttactaacct | |
| | caattccgagctgacgcaggaagaaatcgagcaaattagcaacctgaagggttacactggaacccaca | |
| | acctcagcttgaaagcgattaaccttattttggatgaactttggcacactaatgacaatcagatcgccatttt | |
| | caaccggctgaaactggtgccgaagaaggtggacctgagccaacagaaggaaatcccgaccaccctt | |
| | gtggacgatttcatcctgtcacctgtggtgaagaggagcttcatccagtcgatcaaggtcatcaacgccat | |
| | cataaagaagtacggccttcccaacgacatcatcatcgaactggcccgcgagaagaactccaaagatg | |
| | cccagaagatgatcaacgagatgcagaagcgaaaccggcagacgaacgaacggatcgaggagatca | |
| | tccggaccaccgggaaggaaaacgcgaagtacctgatcgagaaaatcaagctgcatgatatgcagga | |
| | agggaagtgtctctactccctggaggccattccgctggaggatttgctgaacaaccctttcaactacgaa | |
| | gtcgatcatatcattcctcgctccgtgtccttcgataactccttcaacaataaggtcctcgtgaagcaggag | |
| | gagaactcgaagaagggcaacagaaccccgttccagtacctctcgtcgtccgactccaagatcagctac | |
| | gaaactttcaagaagcacattctgaacctggccaagggcaaagggagaattagcaagaccaagaagg | |
| | aatacctcctggaagagagagacatcaaccgcttctcggtgcaaaaggatttcatcaaccgcaacctggt | |
| | cgataccagatacgccaccaggggactgatgaacctcctgcggtcctacttccgggtcaacaatctgga | |
| | cgtgaaggtcaaatccatcaacgggggctttacttctttcctgcgccggaagtggaagttcaagaaggaa | |
| | cggaacaagggatacaagcaccacgctgaagatgccctgattattgccaacgccgacttcatctttaagg | |
| | aatggaaaaagctggacaaggctaagaaggtcatggagaaccagatgttcgaagaaaagcaggccga | |
| | gtccatgcccgaaatcgaaaccgagcaggaatacaaggagatcttcatcacaccgcaccaaatcaagc | |
| | acatcaaggacttcaaggattacaagtacagccaccgggtggacaagaagcctaacagagagcttatc | |
| | aacgacaccctgtactccacgcgcaaggacgacaagggaaacacattgatcgtgaacaacctgaacg | |
| | gactgtatgacaaggacaatgacaaactgaagaagctgatcaacaaatcgccggaaaagctcctgatgt | |
| | accatcacgaccctcaaacctaccagaaactgaagctcatcatggagcagtacggcgacgaaaagaat | |
| | cccctgtacaaatactacgaggagactggaaattacctgactaagtactccaagaaggataacggcccc | |
| | gtgatcaagaagattaagtactacggaaacaaactgaacgcacatctcgacatcaccgatgattatccaa | |
| | actcccgcaacaaagtcgtgaagctctccctcaaaccgtaccgcttcgacgtgtacctggataatggggt | |
| | gtacaagttcgtgaccgtgaagaacctggacgtcattaagaaggaaaactactacgaagtgaactcaaa | |
| | gtgctacgaggaagccaagaagctcaagaagatcagcaaccaggccgagttcatcgcatcgttttacaa | |
| | caatgacctcattaagattaatggagaactgtacagagtgatcggcgtgaacaacgacctcctgaaccg | |
| | gattgaagtgaacatgatcgatattacctaccgggagtatctggagaacatgaacgacaagcgcccacc | |
| | gagaatcatcaaaactattgcctccaagacccaatccattaagaaatactccaccgacatcctgggcaac | |
| | ctgtacgaggtcaagtcgaagaagcacccccagattatcaagaagggaaagcttCCTGCCGCC | |
| | AAGAGAGTGAAGCTGGACtaa | |
| Truncated CMV | GAATTCGTGGTGAGCGTCTGGGCATGTCTGGGCATGTCTGGGC | 3758 |
| promoter | ATGTCTGGGCATGTCGGGCATTCTGGGCGTCTGGGCATGTCTG | |
| | GGCATGTCTGGGCATCTCGAGACTCACGGGGATTTCCAAGTCT | |
| | CCACCCCATTGACGTCAATGGGAGTTTGTTTTGGCACCAAAAT | |
| | CAACGGGACTTTCCAAAATGTCGTAATAACCCCGCCCCGTTGA | |
| | CGCAAATGGGCGGTAGGCGTGTACGGTGGGAGGTCTATATAA | |
| | GCAGAGCTCGTTTAGTGAACCGT | |
| Regular CMV | GGTGTGGAAAGTCCCCAGGCTCCCCAGCAGGCAGAAGTATGC | 3759 |
| promoter | AAAGCATGCATCTCAATTAGTCAGCAACCACGTTACATAACTT | |
| | ACGGTAAATGGCCCGCCTGGCTGACCGCCCAACGACCCCCGCC | |
| | CATTGACGTCAATAATGACGTATGTTCCCATAGTAACGCCAAT | |
| | AGGGACTTTCCATTGACGTCAATGGGTGGAGTATTTACGGTAA | |
| | ACTGCCCACTTGGCAGTACATCAAGTGTATCATATGCCAAGTA | |
| | CGCCCCCTATTGACGTCAATGACGGTAAATGGCCCGCCTGGCA | |
| | TTATGCCCAGTACATGACCTTATGGGACTTTCCTACTTGGCAGT | |
| | ACATCTACGTATTAGTCATCGCTATTACCATGGTGATGCGGTTT | |
| | TGGCAGTACATCAATGGGCGTGGATAGCGGTTTGACTCACGGG | |
| | GATTTCCAAGTCTCCACCCCATTGACGTCAATGGGAGTTTGTTT | |
| | TGGCACCAAAATCAACGGGACTTTCCAAAATGTCGTAACAACT | |
| | CCGCCCCATTGACGCAAATGGGCGGTAGGCGTGTACGGTGGGA | |
| | GGTCTATATAAGCAGAGCTCGTTTAGTGAACCGT | |
| U6 promoter | GAGGGCCTATTTCCCATGATTCCTTCATATTTGCATATACGATA | 3760 |
| | CAAGGCTGTTAGAGAGATAATTGGAATTAATTTGACTGTAAAC | |
| | ACAAAGATATTAGTACAAAATACGTGACGTAGAAAGTAATAA | |
| | TTTCTTGGGTAGTTTGCAGTTTTAAAATTATGTTTTAAAATGGA | |
| | CTATCATATGCTTACCGTAACTTGAAAGTATTTCGATTTCTTGG | |
| | CTTTATATATCTTGTGGAAAGGACGAAACACCG+1 | |

1. A system for introducing a deletion in a genomic DNA molecule comprising a fatty-acid amide hydrolase gene (FAAH) upstream a FAAH pseudogene (FAAH-OUT) in a cell, the system comprising:
(i) a site-directed endonuclease in the form of protein, an mRNA encoding the site-directed endonuclease, or a recombinant expression vector comprising a nucleotide sequence encoding the site-directed endonuclease;
(ii) a first gRNA molecule comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with a site-directed endonuclease that recognizes the PAM, the first gRNA combines with the site-directed endonuclease to induce cleavage proximal the first target sequence with a cleavage efficiency of at least 15%, 20%, 25%, or 30%; and
(iii) a second gRNA molecule comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 15%, 20%, 25%, or 30%,
wherein when the system is introduced to the cell with the site-directed endonuclease, the first gRNA and second gRNA combine with the site-directed endonuclease to induce cleavage proximal the first and second target sequences, to introduce an approximately 2-10 kb deletion in the genomic DNA molecule resulting in a full or a partial removal of a FAAH-OUT promoter (FOP) and a FAAH-OUT conserved (FOC) element, thereby resulting in elimination of FAAH mRNA expression in the cell.

2-100. (canceled)

101. A nucleic acid molecule comprising:
(i) a nucleotide sequence encoding a first gRNA comprising a spacer sequence corresponding to a first target sequence adjacent a first PAM which is downstream of a 3′ terminus of FAAH and upstream a transcriptional start site of FAAH-OUT in the genomic DNA molecule, wherein when the first gRNA is introduced into a cell with the site-directed endonuclease, the first gRNA combines with the site-directed endonuclease to induce cleavage proximal the first target sequence with a cleavage efficiency of at least 30%; and
(ii) a and a nucleotide sequence encoding a second gRNA comprising a spacer sequence corresponding to a second target sequence adjacent a second PAM which is downstream of the FAAH-OUT transcriptional start site and upstream an exon 3 of FAAH-OUT in the genomic DNA molecule, wherein when the second gRNA is introduced into a cell with the site-directed endonuclease, the second gRNA combines with the site-directed endonuclease to induce cleavage proximal the second target sequence with a cleavage efficiency of at least 30%,
wherein when the first and second gRNAs are introduced into a cell with (i) a SluCas9 endonuclease or functional variant thereof or (ii) a SpCas9 endonuclease or functional variant thereof, result in an approximate 2-8 kb deletion in a in a genomic DNA molecule comprising FAAH upstream FAAH-OUT, wherein the deletion results in full removal of a FAAH-OUT promoter (FOP) and a FAAH-OUT conserved (FOC) element in the genomic DNA molecule.

102-119. (canceled)

120. A system for introducing a mutation in a genomic DNA molecule comprising FAAH in a cell, the system comprising:
(i) a site-directed endonuclease in the form of protein, an mRNA encoding the site-directed endonuclease, or a recombinant expression vector comprising a nucleotide sequence encoding the site-directed endonuclease; and
(ii) a gRNA molecule comprising a spacer sequence corresponding to a target sequence within or proximal exon 1, exon 2, exon 3, or exon 4 of the FAAH coding sequence,
wherein when the gRNA is introduced into a cell with the site-directed endonuclease, the gRNA combines with the endonuclease to induce a cleavage proximal the target sequence in the genomic DNA with a cleavage efficiency of at least 15%, 20%, 25%, 30%, 35%, 40%, 45%, or 50%, wherein the cleavage is a double-stranded DNA break (DSB), whereby repair of the DSB results in a mutation, and wherein the mutation provides reduced cellular expression of FAAH mRNA by at least 15%, 20%, 25%, 30%, 35%, 40%, 45%, 50%, 55%, 60%, 65%, 70%, 75%, 80%, 85%, or 90% in the cell.

121. The system of claim 120, wherein the PAM is NNGG, NGG, or NNGRRT.

122. The system of claim 121, wherein the site-directed endonuclease is a SluCas9 endonuclease or a functional derivative thereof, an mRNA encoding the SluCas9 endonuclease or functional derivative thereof, or a recombinant expression vector comprising a nucleotide sequence encoding the SluCas9 endonuclease or functional derivative thereof.

123. The system of claim 121, wherein the site-directed endonuclease is a SpCas9 polypeptide or functional derivative thereof, an mRNA encoding the SpCas9 endonuclease or functional derivative thereof, or a recombinant expression vector comprising a nucleotide sequence encoding the SpCas9 endonuclease or functional derivative thereof.

124. The system of claim 121, wherein the site-directed endonuclease is a SaCas9 polypeptide or functional derivative thereof, an mRNA encoding the SaCas9 endonuclease or functional derivative thereof, or a recombinant expression vector comprising a nucleotide sequence encoding the SaCas9 endonuclease or functional derivative thereof.

125-130. (canceled)

131. The system of claim 123, wherein the target sequence is within exon 1 or exon 2 of FAAH.

132. The system of claim 131, wherein the mutation is an insertion or deletion (INDEL), optionally wherein the mutation is a frameshift mutation, introduction of a stop codon, or a point mutation.

133. The system of claim 131, wherein the spacer sequence comprises:
(a) a nucleotide sequence having up to 1, 2, or 3 nucleotide deletions or substitutions relative to any one of SEQ ID NOs: 42, 43, 60, 63, 64, 65, 66, and 68; or
(b) a nucleotide sequence set forth in SEQ ID NOs: 42, 43, 60, 63, 64, 65, 66, or 68.

134. The system of claim 131, wherein the spacer sequence comprises:
(i) a nucleotide sequence having up to 1 or 2 nucleotide deletions relative to any one of SEQ ID NOs: 63, 64, 65, 66 or 68; or
(ii) a nucleotide sequence set forth in SEQ ID NOs: 63, 64, 65, 66 or 68.

135-168. (canceled)

169. A nucleic acid molecule comprising a nucleotide sequence encoding one or more gRNA molecules targeting a target site in a genomic DNA molecule comprising a fatty-acid amide hydrolase gene (FAAH) in a cell, the gRNA(s) selected from:
(i) a gRNA comprising a spacer sequence corresponding to a target sequence consisting of a nucleotide sequence selected from any one of SEQ ID NOs: 4, 5, 7, 14, and 20;
(ii) a gRNA comprising a spacer sequence corresponding to a target sequence consisting of a nucleotide sequence selected from any one of SEQ ID NOs: 3, 6, 8-13, 16-19, 21-34;
(iii) a gRNA molecule comprising a spacer sequence comprising a nucleotide sequence selected from any one of SEQ ID NOs: 38, 39, 41, 48, and 54; and
(iv) a gRNA molecule comprising a spacer sequence comprising a nucleotide sequence selected from any one of SEQ ID NOs: 37, 40, 42-47, 50-53, 55-68.

170. The nucleic acid molecule of claim 169, wherein the nucleotide sequence encodes one or more gRNA molecule selected from:
(i) a gRNA comprising a spacer sequence corresponding to a target sequence consisting of a nucleotide sequence set forth in SEQ ID NO: 29, 30, 31, 32 or 34; or
(ii) a gRNA molecule comprising a spacer sequence comprising a nucleotide sequence set forth in SEQ ID NO: 63, 64, 65, 66 or 68.

171. (canceled)

172. A nucleic acid molecule comprising a nucleotide sequence encoding one or more gRNA molecules targeting a target site in a genomic DNA molecule comprising a fatty-acid amide hydrolase gene (FAAH) in a cell, the gRNA(s) selected from:
(i) a gRNA comprising a spacer sequence corresponding to a target sequence consisting of a nucleotide sequence selected from any one of SEQ ID NOs: 149, 150, 151, 152, 153, 155, 156, 158, 159, 160, 161, 162, 163 and 164; or
(ii) a gRNA molecule comprising a spacer sequence comprising a nucleotide sequence selected from any one of SEQ ID NOs: 165, 166, 167, 168, 169, 171, 172, 174, 175, 176, 177, 178, 179, and 180.

173. The nucleic acid molecule of claim 172, wherein the nucleotide sequence encodes one or more gRNA molecule selected from:
(i) a gRNA comprising a spacer sequence corresponding to a target sequence consisting of a nucleotide sequence set forth in SEQ ID NO: 149, 155, 159, 160 or 161; or
(ii) a gRNA molecule comprising a spacer sequence comprising a nucleotide sequence set forth in SEQ ID NO: 165, 171, 175, 176 or 177.

174-175. (canceled)

177. A recombinant expression vector comprising the nucleic acid molecule of claim 169.

178. The recombinant expression vector of claim 177 comprising a nucleotide sequence encoding a SpCas9 endonuclease or a functional variant thereof.

179. A recombinant expression vector comprising the nucleic acid molecule of claim 172.

180. The recombinant expression vector of claim 179 comprising a nucleotide sequence encoding a SaCas9 endonuclease or a functional variant thereof.

181. The recombinant expression vector of claim 177, wherein the vector is a viral vector.

182. The recombinant expression vector of claim 181, wherein the vector is an AAV vector.

183. The recombinant expression vector of claim 177, formulated in a lipid nanoparticle.

184. A pharmaceutical composition comprising recombinant expression vector of claim 177, and a pharmaceutically acceptable carrier.

185-191. (canceled)

192. A method for eliminating FAAH expression in a cell, the method comprising: contacting the cell with
the system according to claim 1
wherein when the system contacts the cell, the first gRNA and second gRNA combine with the site-directed endonuclease to induce a deletion in the genomic DNA molecule comprising FAAH upstream FAAH-OUT in the cell, thereby eliminating FAAH expression in the cell.

193-194. (canceled)

195. A method of treating a patient with chronic pain by eliminating FAAH expression in a target cell, the method comprising: administering to the patient an effective amount of
the system according to claim 1
wherein when the system is administered, the first gRNA and second gRNA combine with the site-directed endonuclease to induce a deletion in the genomic DNA molecule comprising FAAH upstream FAAH-OUT in the cell, thereby eliminating FAAH expression in the target cell.

196. (canceled)

197. The method of claim 195, wherein the target cell resides in the brain.

198. The method of claim 195, wherein the target cell resides in the dorsal root ganglion (DRG).

199. The method of claim 198, wherein the target cell is a sensory neuron.

200. The method of claim 195, wherein the route of administration is intra-DRG, intraneural, intrathecal, intra-cisternamagna, and intravenous.

201. The method of claim 195, wherein reduced FAAH expression results in increased levels of one or more N-acyl ethanolamines and/or one or more N-acyl taurines.

202. The method of claim 201, wherein the one or more N-acyl ethanolamine are selected from: N-arachidonoyl ethanolamine (AEA), palmitoylethanolamide (PEA), oleoylethanolamine (OEA), or combination thereof.

203. A system for use with a site-directed endonuclease to introduce a mutation in a genomic DNA molecule comprising FAAH in a cell, the system comprising a recombinant expression vector comprising (i) a nucleotide sequence encoding the site directed endonuclease, and (ii) a nucleotide sequence encoding the gRNA, wherein the gRNA comprises:
(i) a gRNA molecule comprising a spacer sequence comprising a nucleotide sequence set forth in SEQ ID NO: 165, 171, 175, 176 or 177; or; or
(ii) a gRNA comprising a spacer sequence corresponding to a target sequence consisting of a nucleotide sequence set forth in SEQ ID NO: 149, 155, 159, 160 or 161.

204. A system for use with a site-directed endonuclease to introduce a mutation in a genomic DNA molecule comprising FAAH in a cell, the system comprising a recombinant expression vector comprising (i) a nucleotide sequence encoding the site directed endonuclease, and (ii) a nucleotide sequence encoding the gRNA, wherein the gRNA comprises:
(i) a gRNA comprising a spacer sequence corresponding to a target sequence consisting of a nucleotide sequence set forth in SEQ ID NO: 29, 30, 31, 32 or 34; or
(ii) a gRNA molecule comprising a spacer sequence comprising a nucleotide sequence set forth in SEQ ID NO: 63, 64, 65, 66 or 68.

205. The system of claim 204, wherein the system comprises a first recombinant expression vector comprising a nucleotide sequence encoding the site-directed endonuclease, and a second recombinant expression vector comprising a nucleotide sequence encoding the gRNA.

206. The system of claim 204 wherein the vector is a viral vector.

207. The system of claim 206, wherein the vector is an AAV vector.
